(19) United States
(12) Patent Application Publication
(10) Pub. No.: US 2021/0220471 A1
(43) Pub. Date: Jul. 22, 2021

(54) METHODS OF USING PHARMACOLOGIC INHIBITORS OF TYPE 2 CYTOKINE SIGNALING TO TREAT OR PREVENT PANCREATIC CANCER

(71) Applicant: Memorial Sloan Kettering Cancer Center, New York, NY (US)

(72) Inventors: Direna Alonso-Curbelo, New York, NY (US); Scott W. Lowe, New York, NY (US)

(21) Appl. No.: 17/259,800

(22) PCT Filed: Jul. 12, 2019
(86) PCT No.: PCT/US2019/041670

§ 371 Date: Jan. 12, 2021

Related U.S. Application Data
(60) Provisional application No. 62/697,941, filed on Jul. 13, 2018.

(51) Int. Cl. A61K 39/395; C07K 16/28; C07K 16/24; A61K 31/551; A61K 31/4745; A61K 31/517; A61K 31/655; A61K 31/55; A61K 31/4709; A61K 31/5383; A61K 31/473; A61K 31/437; A61K 31/444; A61K 45/06; A61P 35/00

(52) U.S. Cl. CPC A61K 39/3955; A61P 35/00; C07K 16/244; C07K 16/247; C07K 16/245; A61K 31/551; A61K 31/4745; A61K 31/517; A61K 31/655; A61K 31/55; A61K 31/4709; A61K 31/5383; A61K 31/473; A61K 31/437; A61K 31/444; A61K 45/06; C07K 16/2866

(57) ABSTRACT

The present technology provides methods for treating and/or preventing pancreatic cancer using inhibitors of Type 2 cytokine signaling. Also disclosed herein are methods for selecting a pancreatic cancer patient for treatment with an inhibitor of Type 2 cytokine signaling. Kits for use in practicing the methods are also provided.

### CROSS-REFERENCE TO RELATED PATENT APPLICATIONS

This application is a US National Stage Application under 35 U.S.C. § 371 of International Patent Appl. No. PCT/US2019/041670, filed Jul. 12, 2019, which claims the benefit of and priority to U.S. Provisional Appl. No. 62/697,941, filed Jul. 13, 2018, the disclosure of each of which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Jan. 11, 2021, is named 115872-2056_SL.txt and is 92,480 bytes in size.

### TECHNICAL FIELD

The present technology relates to methods for treating or preventing pancreatic cancer using inhibitors of Type 2 cytokine signaling. Kits for use in practicing the methods are also provided.

### BACKGROUND

The following description of the background of the present technology is provided simply as an aid in understanding the present technology and is not admitted to describe or constitute prior art to the present technology.

Pancreatic cancer was the 12^{th} most common type of cancer in the U.S. in 2014, representing about 2.8% of all new cancer cases. However, pancreatic cancer was the 4^{th} most common cause of cancer-related deaths (Schneider G et al., Gastroenterology 128(6):1606-1625 (2005)). In 2014, about 46,420 new cases and 39,590 deaths were attributable to pancreatic cancer in the United States, of which pancreatic ductal adenocarcinoma (PDAC) represents the vast majority. The fact that the annual number of pancreatic cancer-related deaths nearly equals the annual number of new pancreatic cancer cases highlights the lethality of this disease. PDAC, the most common malignancy of the pancreas, is both aggressive and difficult to treat. Complete surgical removal of the tumor remains the only chance for cure, however 80-90% of patients have disease that is surgically incurable at the time of clinical presentation deaths (Schneider G et al., Gastroenterology 128(6):1606-1625 (2005)). Accordingly, there is an urgent need for effective therapies for pancreatic cancer.

### SUMMARY OF THE PRESENT TECHNOLOGY

In one aspect, the present disclosure provides a method for treating or preventing pancreatic cancer in a subject in need thereof comprising administering to the subject an effective amount of an inhibitor of Type 2 cytokine signaling, wherein the subject harbors a KRAS mutation. In some embodiments, the KRAS mutation is selected from the group consisting of G12C, G12D, G12V, G12A, G12S, G12R, G13D, G13C, G13S, G13R, G13A, G13V, Q61H, Q61L, Q61R, Q61K, Q61P, and Q61E. Additionally or alternatively, the inhibitor of Type 2 cytokine signaling inhibits a Type 2 cytokine or Type 2 cytokine receptor signaling protein selected from the group consisting of IL-33, IL-4, IL-13, IL-5, IL-23A, IL-17E, IL-9, IL9R, IL1RL1, IL4RA, IL13RA1, IL13RA2, IL5RA, IL1RL2, IL17RA, IL31RA, IL17RE, IL18R1, IL18RAP, and IL1R1. The inhibitor of Type 2 cytokine signaling may be a small molecule, an inhibitory nucleic acid (e.g., siRNA, antisense nucleic acid, shRNA, sgRNA, ribozymes), a tyrosine kinase inhibitor, a decoy cytokine receptor, or an antibody (e.g., a neutralizing antibody). Additionally or alternatively, in some embodiments, the small molecule, the inhibitory nucleic acid, the tyrosine kinase inhibitor, the decoy cytokine receptor, or the antibody specifically binds to and inhibits/neutralizes the expression or activity of IL-33, IL-4, IL-13, IL-5, IL-23A, IL-17E, IL-9, IL9R, IL1RL1, IL4RA, IL13RA1, IL13RA2, IL5RA, IL1RL2, IL17RA, IL31RA, IL17RE, IL18R1, IL18RAP, or IL1R1. Examples of inhibitors of Type 2 cytokine signaling include, but are not limited to dupilumab, Etokimab (ANB020), Mepolizumab, reslizumab, benralizumab, lebrikizumab, risankizumab, tocilizumab, tralokinumab, anrukinzumab, AMG317, STATE inhibitors, STAT3 inhibitors, Secukinumab, ustekinumab, guselkumab, gevokizumab, or any other agent that inhibits the expression or activity of any of the Type 2 cytokines or Type 2 cytokine receptor signaling proteins disclosed herein. The pancreatic cancer may comprise exocrine tumors. In certain embodiments, the pancreatic cancer is pancreatic ductal adenocarcinoma.

Additionally or alternatively, in some embodiments, the methods further comprise administering to the subject an effective amount of a Brd4 inhibitor. The Brd4 inhibitor may be a small molecule, an inhibitory nucleic acid (e.g., siRNA, antisense nucleic acid, shRNA, sgRNA, ribozymes), or an antibody (e.g., a neutralizing antibody). Examples of Brd4 inhibitors include, but are not limited to, (+)-JQ1, I-BET762, OTX015, I-BET151, CPI203, PFI-1, MS436, CPI-0610, RVX2135, FT-1101, BAY1238097, INCB054329, TEN-010, GSK2820151, ZEN003694, BAY-299, BMS-986158, ABBV-075, GS-5829, and PLX51107. Additionally or alternatively, in some embodiments, the method further comprises sequentially, simultaneously, or separately administering one or more additional therapeutic agents to the subject.

In any and all embodiments of the methods disclosed herein, the subject harbors a mutation in TP53. The subject may have a family history of pancreatic ductal adenocarcinoma or exhibits chronic pancreatitis, Type 2 diabetes or other risk factors for developing pancreatic cancer. Additionally or alternatively, in some embodiments, the subject exhibits elevated expression levels of at least one of IL-33, IL-4, IL-13, IL-5, IL-23A, IL-17E, IL-9, IL9R, IL1RL1, IL4RA, IL13RA1, IL13RA2, IL5RA, IL1RL2, IL17RA, IL31RA, IL17RE, IL18R1, IL18RAP, and IL1R1 compared to that observed in a healthy control subject or a predetermined threshold.

In another aspect, the present disclosure provides a method for selecting pancreatic cancer patients for treatment with an inhibitor of Type 2 cytokine signaling comprising (a) detecting expression levels or chromatin accessibility levels of a Type 2 cytokine or Type 2 cytokine receptor signaling protein in biological samples obtained from pancreatic cancer patients, wherein the Type 2 cytokine or Type 2 cytokine receptor signaling protein is selected from the group consisting of IL-33, IL-4, IL-13, IL-5, IL-23A, IL-17E, IL-9, IL9R, IL1RL1, IL4RA, IL13RA1, IL13RA2, IL5RA, IL1RL2, IL17RA, IL31RA, IL17RE, IL18R1, IL18RAP, and IL1R1; (b) identifying pancreatic cancer patients that exhibit (i) mRNA/protein expression levels of Type 2 cytokine or Type 2 cytokine receptor signaling protein that are elevated compared to that observed in a healthy control subject or a predetermined threshold, and/or (ii) chromatin accessibility levels of Type 2 cytokine or Type 2 cytokine receptor signaling protein that are elevated compared to that observed in a healthy control subject or a predetermined threshold; and (c) administering an inhibitor of Type 2 cytokine signaling to the pancreatic cancer patients of step (b). The inhibitor of Type 2 cytokine signaling may be a small molecule, an inhibitory nucleic acid (e.g., siRNA, antisense nucleic acid, shRNA, sgRNA, ribozymes), a tyrosine kinase inhibitor, a decoy cytokine receptor, or an antibody (e.g., a neutralizing antibody). Additionally or alternatively, in some embodiments, the small molecule, the inhibitory nucleic acid, the tyrosine kinase inhibitor, the decoy cytokine receptor, or the antibody specifically binds to and inhibits/neutralizes the expression or activity of IL-33, IL-4, IL-13, IL-5, IL-23A, IL-17E, IL-9, IL9R, IL1RL1, IL4RA, IL13RA1, IL13RA2, IL5RA, IL1RL2, IL17RA, IL31RA, IL17RE, IL18R1, IL18RAP, or IL1R1. Examples of inhibitors of Type 2 cytokine signaling include, but are not limited to dupilumab, Etokimab (ANB020), Mepolizumab, reslizumab, benralizumab, lebrikizumab, risankizumab, tocilizumab, tralokinumab, anrukinzumab, AMG317, STATE inhibitors, STAT3 inhibitors, Secukinumab, ustekinumab, guselkumab, gevokizumab, or any other agent that inhibits the expression or activity of any of the Type 2 cytokines or Type 2 cytokine receptor signaling proteins disclosed herein.

Additionally or alternatively, in some embodiments, the methods further comprise administering a Brd4 inhibitor to the pancreatic cancer patients of step (b). The Brd4 inhibitor may be a small molecule, an inhibitory nucleic acid (e.g., siRNA, antisense nucleic acid, shRNA, sgRNA, ribozymes), or an antibody (e.g., a neutralizing antibody). Examples of Brd4 inhibitors include, but are not limited to, (+)-JQ1, I-BET762, OTX015, I-BET151, CPI203, PFI-1, MS436, CPI-0610, RVX2135, FT-1101, BAY1238097, INCB054329, TEN-010, GSK2820151, ZEN003694, BAY-299, BMS-986158, ABBV-075, GS-5829, and PLX51107.

In any and all embodiments of the methods disclosed herein, the pancreatic cancer patients harbor a KRAS mutation selected from the group consisting of G12C, G12D, G12V, G12A, G12S, G12R, G13D, G13C, G13S, G13R, G13A, G13V, Q61H, Q61L, Q61R, Q61K, Q61P, and Q61E. Additionally or alternatively, in some embodiments, the pancreatic cancer patients harbor a mutation in TP53. The pancreatic cancer patients may exhibit exocrine tumors. In certain embodiments, the pancreatic cancer patients suffer from or are at risk for pancreatic ductal adenocarcinoma.

In any of the preceding embodiments of the methods disclosed herein, the expression levels or chromatin accessibility levels of the Type 2 cytokine or Type 2 cytokine receptor signaling protein are detected via ChIP, MNase, FAIRE, DNAse, ATAC-seq, RT-PCR, Northern Blotting, RNA-Seq, microarray analysis, High-performance liquid chromatography (HPLC), mass spectrometry, immunohistochemistry (IHC), fluorescence in situ hybridization (FISH), Western Blotting, immunoprecipitation, flow cytometry, Immuno-electron microscopy, immunoelectrophoresis, enzyme-linked immunosorbent assays (ELISA), or multiplex ELISA antibody arrays. In some embodiments, the biological samples are pancreatic cancer specimens, blood, serum, or plasma.

Also disclosed herein are kits comprising at least one inhibitor of Type 2 cytokine signaling and instructions for using the at least one inhibitor of Type 2 cytokine signaling to treat or prevent pancreatic cancer. The inhibitor of Type 2 cytokine signaling may be a small molecule, an inhibitory nucleic acid, a tyrosine kinase inhibitor, a decoy cytokine receptor, or an antibody. In certain embodiments, the inhibitor of Type 2 cytokine signaling inhibits a Type 2 cytokine or Type 2 cytokine receptor signaling protein selected from the group consisting of IL-33, IL-4, IL-13, IL-5, IL-23A, IL-17E, IL-9, IL9R, IL1RL1, IL4RA, IL13RA1, IL13RA2, IL5RA, IL1RL2, IL17RA, IL31RA, IL17RE, IL18R1, IL18RAP, and IL1R1. Additionally or alternatively, in some embodiments of the kits of the present technology, the inhibitor of Type 2 cytokine signaling is selected from the group consisting of dupilumab, Etokimab (ANB020), Mepolizumab, reslizumab, benralizumab, lebrikizumab, risankizumab, tocilizumab, tralokinumab, anrukinzumab, AMG317, STATE inhibitors, STAT3 inhibitors, Secukinumab, ustekinumab, guselkumab, gevokizumab, and any other agent that inhibits the expression or activity of any of the Type 2 cytokines or Type 2 cytokine receptor signaling proteins disclosed herein.

In any of the preceding embodiments, the kits further comprise at least one Brd4 inhibitor, wherein the at least one Brd4 inhibitor is a small molecule, an inhibitory nucleic acid, or an antibody. Examples of Brd4 inhibitors include, but are not limited to, (+)-JQ1, I-BET762, OTX015, I-BET151, CPI203, PFI-1, MS436, CPI-0610, RVX2135, FT-1101, BAY1238097, INCB054329, TEN-010, GSK2820151, ZEN003694, BAY-299, BMS-986158, ABBV-075, GS-5829, and PLX51107. Additionally or alternatively, in some embodiments, the kits further comprise reagents for detecting mRNA or protein expression levels or chromatin accessibility levels of a Type 2 cytokine or Type 2 cytokine receptor signaling in a biological sample.

### BRIEF DESCRIPTION OF THE DRAWINGS

FIG. 1A shows a schematic diagram of the experimental settings used to interrogate oncogenic and regenerative plasticity in the exocrine pancreas. Multi-allelic mice harboring pancreas-specific mutant (G12D) Kras (KC-GEMM) or wild-type (WT) Kras (C-GEMM), subjected to or not subjected to pancreatic injury, served as in vivo models to study mechanisms underlying neoplastic transformation vs normal regeneration and cooperation of mutant KRAS and tissue injury during early neoplasia. The alleles of KC- and C-GEMM mice are summarized in FIG. 1B.

FIG. 1B shows a schematic representation of the genetic configuration used to drive inducible, exocrine pancreas-specific suppression of BRD4 in transgenic mice carrying the indicated alleles. Ptf1a drives Cre recombinase activity in pancreatic exocrine cells, which activates the expression of rtTA3 and mKate2 by removing a transcriptional stop signal. In the presence of doxycycline (dox), rtTA3 drives expression of the GFP-shRNA cassette to enable expression of GFP-linked shRNAs targeting Brd4 (shBrd4) or Renilla luciferase (shRen, control) selectively in either mutant Kras^{G12D/+} or Kras^{+/+}-expressing pancreatic epithelial cells, marked with the fluorescent tag mKate2.

FIG. 1C shows the representative H&E and immunohistochemistry (IHC) or immunofluorescence (IF) analyses of the indicated proteins in pancreata from KC-GEMM (top) or C-GEMM (bottom) mice placed on the dox diet fed at 5 weeks old and analyzed 9 days later. mKate2 staining marks mutant KRAS-expressing (top) or wild-type (bottom) pancreatic exocrine cells where Ptf1a-Cre has been expressed. GFP staining corresponds to shRNA expression and is coupled with Brd4 suppression in that same compartment (but not in surrounding stroma) in mice harboring shRNAs targeting Brd4 (shBrd4) but not Ren (shRen). Mice expressing mutant Kras (KC-GEMM) developed mucinous lesions as detected by Alcian Blue staining (e.g., Hingorani et al., Cancer Cell 4: 437-450 (2003)), whereas Kras wild-type counterparts (C-GEMM) retained a normal acinar compartment. Dashed lines demark boundaries between epithelium and stroma, and arrows point to Brd4-suppressed exocrine pancreas compartment of shBr4 mice.

FIG. 1D shows the Assay for Transposase-Accessible Chromatin using sequencing (ATAC-seq) and RNA-seq tracks of genes previously known to be associated with active enhancers (top) in either acinar (e.g., Cabp2, Il22ra1, (Jiang et al., Mol Cell Biol. 36(23):2945-2955 (2016)) or pancreatic progenitor cells (e.g., Fgfr2, (Cebola et al., Cell Biol 17: 615-626 (2015)) in mKate2⁺ sorted cells isolated from KC-GEMM at the same age and dox-treatment time-point as in FIG. 1C. Brd4 suppression selectively impaired transcription of pancreatic enhancer-associated genes without altering chromatin accessibility at that same loci. Tracks of housekeeping genes (bottom) are shown as specificity controls. See also FIGS. 6A-6E.

FIGS. 2A-2B show the representative immunofluorescence stains showing induction of the acinar-to-ductal metaplasia (ADM) marker SOX9 (FIG. 2B) or the acinar marker CPA1 (FIG. 2A) in GFP-positive (shRNA-expressing) pancreatic epithelial cells from KC-GEMM-shRen or -shBrd4 mice were fed a diet containing doxyxycline (the dox diet) since postnatal day 10 and analyzed at 6 weeks of age. Shown to the left are immunofluorescence stains of pancreata from the 6 weeks old C-GEMM-shRen mice, which acted as a control expressing wild type Kras, which were placed on the dox diet since postnatal day 7.

FIG. 2C shows the representative immunohistochemical stains of mKate2 and Alcian blue positive mucins in pancreata from KC-GEMM-shRen or KC-GEMM-shBrd4 mice placed on the dox diet since postnatal day 10 and analyzed at the indicated time-points after birth. Mutant Kras pancreatic epithelial cells (marked by mKate2-positive) expressing Brd4-shRNA did not contribute to mucinous lesions and were selected against over time.

FIG. 2D shows the quantification of the relative pancreas area with GFP-positive (shRNA-expressing) mucinous pancreatic intraepithelial neoplasia (PanIN) lesions in KC-GEMM-shRen or -shBrd4 mice placed on the dox diet since postnatal day 10 to induce shRNA expression and analyzed at the indicated time-points after birth by immunohistochemical staining of GFP and Alcian blue. Each point represents one animal, and data are presented as the average±SEM. ***p<0.001, **p<0.01.

FIG. 2E shows the representative bright-field and fluorescence images showing gross morphology of pancreata of KC-shRen and -shBrd4 mice placed on the dox diet since postnatal day 10 to induce shRNA expression and analyzed at the indicated time-points after birth. Reduced GFP and mKate2 signals correlated with the loss of mutant Kras pancreatic epithelial cells observed upon Brd4 shRNA expression.

FIG. 2F shows the experimental strategy to address the requirement of Brd4 during accelerated, synchronous pancreatic tumorigenesis driven by mutant Kras and tissue injury. 4-week old KC-GEMM mice were placed on the dox diet to induce acute expression of shRNA targeting Brd4 or Ren in the pancreatic epithelium and, 6 days thereafter, pancreatic injury was induced by caerulein treatment. Pancreata were harvested at day 2 and day 15 and week 5 after caerulein treatment for histological assessment of acinar-to-ductal reprogramming (ADR) leading to mucinous PanIN lesions.

FIG. 2G shows the immunofluorescence (GFP) and immunohistochemical (mKate2, Alcian Blue) stains to visualize Cre-recombined, shRNA-expressing pancreatic epithelial cells (GFP, mKate2) or mucinous states (Alcian Blue) in pancreata from KC-shRen and -shBrd4 mice treated as described in FIG. 2(F) at the indicated time-points. All images are representative of at least 4 independent biological replicates (100% chimerism animals). See also FIGS. 8A-8E.

FIG. 3A shows the experimental strategy to address the requirement of Brd4 during the normal pancreas regeneration after injury. 4 weeks old C-GEMM mice were placed on the dox diet to induce expression of shRNA targeting Brd4 or Ren in the pancreatic epithelium and after 6 days injury was induced by caerulein treatment. Pancreata were harvested 2 and 7 days after caerulein treatment for histological analyses of injury-induced ADM and subsequent regeneration with restoration of acinar differentiation.

FIG. 3B shows the representative bright-field and fluorescence images showing gross morphology of pancreata of C-GEMM-shRen and -shBrd4 mice treated as described in FIG. 3A and analyzed at the indicated time-points in days (d) after treatment with caerulein or vehicle (PBS). Reduced GFP and mKate2 signal denote rapid loss of pancreatic tissue expressing Brd4 shRNA between day 2 and day 7 after injury.

FIG. 3C shows the quantification of pancreatic weight normalized to animal body weight by genotype. Each dot represents one animal. Data are presented as the average±SEM. ****p<0.0001, ***p<0.001, **p<0.01.

FIG. 3D shows the representative H&E staining of pancreata from KC-shRen or KC-shBrd4 mice treated with caerulein or PBS control and harvested at the indicated time-points post-treatment.

FIGS. 3E-3F show the representative immunofluorescence staining of pancreata from KC-shRen or KC-shBrd4 mice treated with Caerulein or PBS control and analyzed at the indicated time-points post treatment for protein expression of the metaplasia marker Krt19 (FIG. 3E) or the acinar marker Cpa1 (FIG. 3F) co-stained with GFP (marking shRNA expressing cells) and DAPI (mark nuclei). All images are representative of at least 4 independent biological replicates (animals). See also FIGS. 9A-9B.

FIG. 4A shows the strategy and experimental conditions for in vivo profiling of chromatin and transcriptional landscapes of lineage-traced pancreatic epithelial cells (mKate2⁺, CD45⁻) undergoing regenerative metaplasia (Reg-ADM, green) vs injury-accelerated Kras-driven neoplasia (Kras*-ADR, orange), isolated by FACS-sorting from 5 weeks old KC- or C-GEMM mice, respectively, treated with caerulein (Caer-) and analyzed 2 days (d2) after treatment. Analogous analyses were performed in lineage-traced cells (mKate2⁺, CD45⁻) isolated from PBS-treated C-GEMM (Normal, grey) or PBS-treated KC-GEMM (Kras*, red) to define effects of injury or mutant Kras alone, and late-stage invasive PDAC cells (PDAC, blue) isolated from KPflC mice (p48Cre;RIK;LSL-KrasG12D;p53fl/⁺) to compare early changes with end-stage cancer.

FIG. 4B shows the Principal Component Analyses (PCA) of RNA-seq data from independent biological replicates (individual mice) of FACS-sorted pancreatic epithelial cells isolated from normal, regenerating, early-neoplasia or cancer tissues described in FIG. 4A. Samples from same stage cluster closely together irrespective of experimental litter or sample collection date. Independent biological replicates (individual mice) representing the same tissue state clustered tightly together, irrespective of experimental litter or sample collection date.

FIG. 4C shows the proportion of differentially expressed genes (DEGs; Fold change>2, padj<0.05) between human PDAC vs normal pancreas that are found transcriptionally altered in mKate2⁺ pancreatic epithelial cells isolated from GEMMs modeling the indicated tissue states vs from normal pancreas, shown for 2 independent human microarray datasets (Moffitt et al., Nat Genet 47, 1168-1178 (2015); Yang et al., Cancer Res 76, 3838-3850 (2016)). hPDAC-DN=genes downregulated in human PDAC vs human normal (black). hPDAC-UP=genes upregulated in human PDAC vs human normal pancreas.

FIG. 4D shows a heatmap representation of unsupervised k-means clustering of genes differentially expressed between lineage-traced pancreatic epithelial cells (mKate2⁺;CD45⁻) from regeneration (Reg-ADM) or from early-neoplastic (Kras*, Kras*-ADR) tissues compared to normal pancreas, and plotted along with PDAC samples to show their transcriptional status in malignant end-stage disease. Representative genes and top-scoring reactome pathways enriched for each of the RNA-Seq clusters (Z1-Z5) are shown in the middle and right panels. Shared down—(Z1, Z2), shared up—(Z4, Z5) and a mutant Kras-specific up—(Z3) regulated gene clusters were identified. DN=cluster downregulated in PDAC vs Normal. UP=cluster upregulated in PDAC vs Normal. Z3-Z5 are upregulated in PDAC vs Normal. Z1 and Z2 clusters are downregulated in PDAC vs Normal, with Z1 being silenced already during Kras*-ADR (Early-DN) whereas Z2 subsequently during tumor progression (Late-DN).

FIG. 4E shows the representative ATAC-seq tracks of the indicated genes defining acinar, metaplasia or neoplasia-specific states in lineage-traced pancreatic epithelial cells isolated from normal (grey), regenerating (Reg-ADM), early-neoplasia (Kras*, Kras*-ADR) and invasive cancer (PDAC) tissues. Relative mRNA levels (DESeq2-normalized counts) are shown to the right. Selected genes are either similarly UP- or down (DN)-regulated in pancreata undergoing regeneration (R) or pro-neoplastic plasticity (N). See also FIGS. 10A-10E and FIGS. 29A-29B.

FIG. 5A shows the Principal Component Analyses (PCA) of ATAC-seq data from independent biological replicates (individual mice) of FACS-sorted pancreatic epithelial cells isolated from normal, regenerating, early-neoplasia or cancer tissues described in FIG. 4A.

FIG. 5B shows the proportion of regions (ATAC peaks) exhibiting a significant gain (black) or loss (grey) in chromatin accessibility (log 2FC>=0.58, FDR<=0.1) in FACS-sorted PDAC vs normal cells, and found similarly altered in cells sorted from regenerating (Reg-ADM) or early neoplastic (Kras*, Kras*-ADR) pancreata. Note the synergistic action of mutant Kras⁺ injury (Kras*-ADR) in promoting early chromatin accessibility gains at regions that are open in PDAC but not normal pancreas.

FIG. 5C shows the overlap of the gained (left) or lost (right) ATAC peaks in the indicated conditions. Numbers in brackets indicate the total number of peaks significantly gained or lost in the indicated tissue states vs normal pancreas. 8793 peaks are uniquely gained by the combination of tissue injury and mutant Kras.

FIG. 5D shows a heatmap representation of k-means clustering illustrating chromatin accessibility levels at peaks gained or lost during regeneration and early neoplasia compared to normal pancreas. Each column represents one independent biological replicate (animal). A1-A4 ATAC peaks are gained (+) compared to normal pancreas in response to injury (A2), mutant Kras (A3), either or them (A1) or the combination of both (A4, during ADR). A5 and A6 ATAC peaks are lost (−) compared to normal pancreas in response to either injury or mutant Kras (A6) or by mutant Kras or the combination of both (A5).

FIG. 5E shows the representative ATAC-seq tracks of loci exhibiting synergistic gain of chromatin accessibility combination of tissue injury and mutant Kras. Each lane is an independent biological replicate.

FIG. 5F shows the transcription factor motifs (identified by HOMER analysis) enriched in regions uniquely gaining chromatin accessibility in mutant Kras pancreatic epithelial cells isolated from tissues undergoing synchronous ADR (Kras*-ADR) but not in Kras^{wt} counterparts undergoing reversible metaplasia during regeneration (Reg-ADM).

FIG. 5G shows the proportion of peaks containing AP1+ motifs (HOMER) or NR5A2-bound sites (Holmstrom et al., 2011) in the indicated ATAC-seq clusters, from (D). Note the inverse correlation between AP1- and NR5A2-associated motifs in chromatin regions displaying accessibility changes during regeneration and early neoplasia. See also FIGS. 11A-1111.

FIG. 6A shows a heatmap representation of differentially expressed genes (DEGs; Fold change>2, padj<0.05) between shRen or shBrd4 pancreatic exocrine cells undergoing regenerative metaplasia (Reg-ADM) or synchronous mutant KRAS-driven transformation (Kras*-ADR) in vivo. shRNA-expressing cells (mKate2⁺;GFP⁺;CD45⁻) were isolated from the GEMMs described in FIG. 1, after acute Brd4 suppression followed by induction of Reg-ADM or Kras*-ADR states as described in FIG. 4A. 3 biological replicates (individual mice) were analyzed per condition. C-shRen samples are shown to the left to show expression levels of DEGs relative to normal pancreas. DEGs were generated by comparing shRen control to Brd4-shRNA (1448).

FIG. 6B shows the overlap of DEGs downregulated upon Brd4 suppression during Reg-ADM or Kras*-ADR settings. Examples of Brd4-dependent genes, shared or unique to each context are shown. DEGs were generated by comparing shRen control to Brd4-shRNA (1448).

FIG. 6C shows the Gene Set Enrichment Analysis (GSEA) comparing the expression of genes upregulated in human PDAC specimens vs human normal pancreas (from 2 independent datasets, (Moffitt et al., Nat Genet 47, 1168-1178 (2015); Yang et al., Cancer Res 76: 3838-3850 (2016)) (top) or in PanIN or PDAC organoids vs normal organoids (from Boj et al., Cell 160: 324-338 (2015)) (bottom) between shBrd4 and shRen cells isolated from KC-GEMM mice (Kras*-ADR condition).

FIG. 6D shows the ATAC-seq and RNA-seq tracks of representative Brd4-regulated gene loci in shRen (black) or shBrd4 (blue) pancreatic epithelial cells isolated from KC-GEMM (Kras*-ADR).

FIG. 6E shows the GSEA comparing the expression of genes selectively induced during early neoplasia (Z3) and associated with mutant Kras-dependent promoter accessibility gains (A3/A4) in shBrd4 vs shRen pancreatic epithelial cells isolated from KC-GEMM mice (Kras*-ADR condition) (right). As comparison, analogous GSEA performed with genes exhibiting no significant changes in expression or chromatin accessibility are shown (left). See also FIGS. 12A-12F.

FIG. 7A shows the representative ATAC-seq tracks of the Il-33 locus in Cre-recombined cells isolated from the normal, regenerating, early-neoplasia or cancer tissues described in FIG. 4A. Note synergistic action of mutant Kras and tissue injury in promoting chromatin accessibility gains (in grey boxes) retained in malignant PDAC cells.

FIG. 7B shows the relative mRNA levels (DESeq2-normalized counts) of Il-33 in FACS-sorted mKate2⁺; CD45⁻ cells isolated from the indicated tissue states. Each dot represents one animal.

FIG. 7C shows the comparison of the effects of Brd4 suppression (Y-axis) in cytokine mRNA levels, with their degree of induction during Kras*-ADR vs normal pancreas (X-axis). Cytokines marked in blue are not similarly induced during normal regeneration. Note the selective impact of Brd4 suppression and mutant Kras-dependent induction in cytokine gene expression.

FIG. 7D shows the qRT-PCR analyses validating significant downregulation of II-33 upon Brd4 suppression in Cre-recombined cells triggered to undergo Kras*-ADR in KC-GEMM mice.

FIG. 7E shows the multiplexed immunoassay detecting the indicated cytokines or chemokines in protein lysates from normal or mutant Kras pancreata, 2 days after induction of caerulein (Caer)-induced tissue injury or treatment with PBS (control).

FIG. 7F shows the immunofluorescence staining of Il-33 (red) and GFP (green) in the indicated models, genotypes and treatment conditions, showing marked rapid induction of Il-33 in pancreatic epithelial cells (marked by GFP) undergoing Kras-ADR driven by combined effects mutant Kras and tissue injury, which is blunted by Brd4 suppression. Nuclei are stained with DAPI (blue).

FIG. 7G shows the qRT-PCR analyses of markers of productive ADR (Agr, Muc6), acinar differentiation (Cpa1) and ductal metaplasia (Sox9) in Cre-recombined pancreatic epithelial cells (mKate2⁺) isolated from Kras-wild type (C) or Kras mutant (KC) mice treated with rIl-33 or Vehicle and analyzed 21 days thereafter. Note selective effects of rIl-33 in Kras-mutated pancreata.

FIG. 7H shows the histological analyses (H&E) and immunohistochemical staining (mKate2, Alcian Blue) in pancreata from Kras-wild type (C-GEMM) or Kras mutant (KC-GEMM) mice treated with rIl-33 or Vehicle and analyzed 21 days thereafter. While normal pancreata retain normal architecture upon rIl-33 treatment, mutant Kras pancreata undergo accelerated ADR with development of mucinous lesions (marked by positive Alcian Blue staining). See also FIGS. 13A-13D.

FIG. 8A shows the quantification of the relative pancreas area with GFP-positive (shRNA-expressing) ADM in KC-GEMM-shRen or -shBrd4 mice placed on the dox diet since postnatal day 10 to induce shRNA expression and analyzed at the indicated time-points after birth by immunohistochemical staining of GFP and Alcian blue. Each dot represents one animal, and data are presented as the average±SEM. **p<0.01. Brd4 suppression does not impair the conversion of acinar-to-ductal metaplasia (ADM) but impairs the further progression and maintenance of ADM lesions.

FIG. 8B shows the representative co-immunofluorescence stains of mKate2 (red, marking Cre-recombined cells) and the acinar marker Amylase (green) in pancreata from 6 week old KC-GEMM-shRen or -shBrd4 mice that were fed on dox diet since postnatal day 10 and then analyzed. Nuclei are counterstained with DAPI (blue). Brd4 suppression accelerates the loss of acinar identity, evidenced by reduced Amylase levels in mKate2-positive compartment in 2 independent KC-GEMMs expressing different Brd4 shRNAs (552 and 1448).

FIG. 8C shows the representative co-immunofluorescence stains of GFP (marking Cre-recombined cells expressing GFP-linked shRNA, green) and the acinar marker Cpa1 (red) in pancreata from 6 weeks old mice KC-GEMM-shBrd4 mice left off dox or placed on the dox diet fed at day 10 after birth. Nuclei are counterstained with DAPI (blue). Dox diet induced shBrd4 expression, which is coupled with GFP induction and an accelerated loss of acinar identity evidenced by reduced Cpa1 levels in GFP-positive cells.

FIG. 8D shows the quantification of pancreatic weight normalized to animal body weight in the indicated genotypes. Each point represents one animal. Data are presented as the average±SEM. ***p<0.0001, ***p<0.001, **p<0.01. Reduced pancreatic weights of C-shBrd4 mice at day 7 post-caerulein treatment were indicative of pancreatic atrophy.

FIG. 8E shows the representative immunohistochemistry stains of mKate2 (top) and Myc (bottom) in pancreata from 6 weeks old mice of the indicated genotypes and placed on the dox diet fed at day 10 after birth. Lower panels show high magnification images of regions marked with dashed line boxes for visualization of Myc nuclear localization.

FIG. 8F shows the representative co-immunofluorescence stains of mKate2 (marking Cre-recombined cells, red) and the acinar marker Cpa1 (green) in pancreata from 6 weeks old mice KC-GEMM mice of the indicated genotypes and placed on the dox diet fed at day 10 after birth. Nuclei are counterstained with DAPI (blue). Right panels show high magnification images of regions marked with dashed line boxes. The reduced levels of Cpa1 observed in KC-shBrd4 mice was not phenocopied in KC-shMyc mice, which retain Cpa1 expression.

FIG. 9A shows the representative co-immunofluorescence stains of the ductal marker Sox9 (red) and GFP (marking shRNA-expressing cells, green) in pancreata from On dox C-GEMM-shRen or -shBrd4 mice treated with PBS or Caerulein (Caer) and analyzed at the indicated time points in days (d) after treatment. In merge images, nuclei are counterstained with DAPI (blue). Brd4 suppression does not impair acinar-to-ductal metaplasia, as evidenced by acquisition of ductal morphology and Sox9 expression at day 2, which is aberrantly maintained at day 7.

FIG. 9B shows the representative co-immunofluorescence stains of the acinar stress marker Clusterin (red) and GFP (marking shRNA-expressing cells, green) pancreata from On dox C-GEMM-shRen or -shBrd4 mice treated with PBS or Caerulein (Caer) and analyzed at the indicated timepoints in days (d) after treatment. Nuclei are counterstained with DAPI (blue). Brd4 suppression does not impair Clusterin induction at Caer-d2 but hampers regeneration as suggested by retained areas of clusterin-positive regions at Caer-day 7.

FIG. 10A shows the representative H&E and mKate2 immunohistochemistry stains in pancreata from the indicated genotypes and treatment groups. Genotype abbreviations are as follows: C=Ptf1a-Cre/RIK; KC=Ptf1a-Cre/RIK/LSL-Kras^{G12D}; KP^{fl}C=Ptf1aCre/RIK/LSL-Kras^{G12D}/p53^{fl/+}. RIK enables tracing of Cre-recombined exocrine pancreas through positive mKate2 expression. mKate2 positive exocrine pancreata acquires duct-like phenotypic changes upon acute tissue injury that are reversible or persistent depending on the presence of mutant Kras, and which are linked to the development of pancreatic ductal adenocarcinoma (PDAC) and have an accelerated progression in the presence of a p53-floxed allele.

FIG. 10B shows the number of differentially expressed genes (DEGs, Fold change>2, padj<0.05), either up (UP)- or down (DN)-regulated in mKate2⁺ cells isolated from the indicated pancreatic tissue states vs normal pancreas state control. R/N-DEGs refer to the transcriptional changes induced in mKate2⁺ pancreatic epithelial cells during injury-induced regeneration or early neoplasia, and are plotted with those induced in end-stage PDAC.

FIG. 10C shows the overlap of the R/N-DEGs down—(left) or up—(right) regulated in mKate2⁺ pancreatic epithelial cells during injury-induced regeneration or early neoplasia compared to normal pancreas. Numbers in brackets indicate the total number of DEGs in the indicated tissue states vs normal pancreas. Examples of DEGs, shared or unique to each context are shown in grey.

FIG. 10D shows the proportion of differentially expressed genes (DEGs) between human PDAC vs normal pancreas (Fold change>2, padj<0.05 (Yang et al., Cancer Res 76: 3838-3850 (2016)) that are included in the R/N-DEGs, separated into the Z1-Z5 clusters from FIG. 4D. Note how the Z1 cluster (early-inactivation cluster) is enriched in genes downregulated in human PDAC vs normal, whereas the Z3 and Z4 clusters (early-activation clusters) are enriched in genes upregulated in human PDAC vs normal.

FIG. 10E shows the proportion of R/N-DEGs (divided into Z1-Z5 clusters) that are associated with chromatin accessibility changes during normal regeneration or early neoplasia (defined as dynamic ATAC peaks between Reg-ADM, Kras* and Kras*-ADR conditions vs normal pancreas). DEGs with ‘dynamic’ chromatin accessibility are those DEGs associated with ATAC peaks that are significantly gained or lost (log 2FC>=0.58 and a FDR<=0.1) in cells undergoing regenerative metaplasia (Reg-ADM) or pro-neoplastic transitions (Kras* or Kras-ADR) vs Normal. DEGs with ‘stable’ chromatin accessibility are those DEGs for which none of the associated ATAC peaks changed in these same stages vs Normal.

FIG. 10F shows a heatmap representation of the mean ATAC signals (top) for Z1-Z5 genes, normalized for each of the indicated tissue states. Bottom panel shows corresponding mean mRNA expression values (DESeq2-normalized counts). Note consistent changes in gene expression and associated chromatin accessibility patterns.

FIG. 11A shows a heatmap representation of the peaks gained or lost during regeneration and early neoplasia vs normal pancreas, separated into the 6 ATAC clusters (A1-A6) and plotted across the indicated conditions, including PDAC. 67% of that peaks selectively gained during Kras*-ADR are retained in invasive disease.

FIG. 11B shows a correlation plot showing genome-wide ATAC-seq size factors used for data normalization with two different normalization methods. PeakNorm uses the in-built DESeq2 normalisation for all filtered reads mapped to the peak atlas, whereas DepthNorm uses the number of filtered mapped reads irrespective of if the reads are within or outside the peak atlas.

FIGS. 11C-11E show the genomic annotations of dynamic peaks in each ATAC cluster (FIG. 11C), gained or lost in the indicated states vs normal pancreas (FIG. 11D), or unique to Reg-ADM or Kras*-ADR vs Normal (FIG. 11E), according to the location of a given peak. In FIG. 11E, the number in the brackets indicates distance of peaks to associate gene TSS in kb.

FIG. 11F shows the HOMER motif analysis showing enriched TF motifs associated with ATAC peaks unique to Reg-ADM (green) or Kras*-ADR (orange) vs Normal. The % of peaks harboring the indicated motifs and the significance of the enrichment is shown to the right.

FIG. 11G shows the relative mRNA levels (DESeq2-normalized counts) of TF linked to ATAC peaks selectively gained (left) or lost (right) in Kras*-ADR or Reg-ADM conditions in FACS-sorted mKate2⁺;CD45⁻ cells isolated from the indicated tissue states. Each dot represents one animal.

FIG. 1111 shows the relative mRNA levels of Foxa1 in FACS-sorted mKate2⁺;CD45⁻ cells isolated from the indicated tissue states. Each dot represents one animal.

FIG. 12A shows the representative immunohistochemical staining (IHC) of Brd4 in pancreata from C-GEMM (left) or KC-GEMM (right) mice harboring shRen or shBrd4 at day 2 post-caerulein, placed on the dox diet fed 6 days before caerulein treatment start. Brd4 is suppressed in metaplastic pancreatic epithelial cells undergoing synchronous regenerative (Reg-ADM) or neoplastic (Kras*-ADR) plasticity.

FIG. 12B shows a heatmap showing the combined score of representative pathways significantly downregulated (blue) or upregulated (red) in shBrd4 vs shRen pancreatic epithelial cells, undergoing synchronous regenerative metaplasia (Reg-ADM, left) or neoplastic transformation (Kras*-ADR, right), respectively.

FIG. 12C shows the GSEA comparing the expression of genes herein identified to be downregulated during early neoplasia (Z1) and associated with mutant Kras-dependent chromatin accessibility losses (A5) between shBrd4 vs shRen, in both Reg-ADM (left) and Kras*-ADR (right) settings.

FIG. 12D shows the GSEA comparing the expression of genes described to be upregulated in murine PDAC vs normal organoids (Boj et al., Cell 160: 324-338 (2015)) between shBrd4 and shRen cells isolated from KC-GEMM mice (Kras*-ADR).

FIG. 12E shows the representative ATAC-seq and RNA-seq tracks of examples of genes herein identified to be induced during Kas*-ADR in a Brd4-independent (left) or Brd4-dependent (right manner).

FIG. 12F shows the GSEA comparing the expression of described AP1 targets in Ras-mutant cancer cells (Vallejo et al., Nat Commun 8: 14294 (2017)) between shBrd4 and shRen cells isolated from KC-GEMM mice (Kras*-ADR).

FIG. 13A shows the identification of high enrichment of genes encoding secreted and plasma membrane-associated factors among the genes induced in a mutant Kras-dependent manner (Z3) associated with peaks selectively gained during Kras*-ADR (A4) and whose expression is blunted by Brd4 knockdown.

FIG. 13B shows the heatmap representation of the relative expression levels of the indicated genes in FACS-sorted shRen or shBrd4 pancreatic epithelial cells isolated from KC-GEMM mice during Kras*-ADR. Each column represents one mouse.

FIG. 13C shows the abundance of the indicated cytokines or chemokines in pancreatic tissue lysates from KC-GEMM mice during Kras*-ADR. Data are presented as the average±SEM of 5 independent biological replicates (individual animals).

FIG. 13D shows a model summarizing key aspects of the regulation of Kras- and injury-driven pancreatic plasticity described herein. In response to mutant Kras or tissue injury, acinar cells undergo de-differentiation and acquire duct-like identity (ADM) that does not require Brd4 function. In contrast, restoration of acinar identity or, alternatively, neoplastic progression in the presence of oncogenic Kras requires Brd4-dependent activation of distinct gene expression programs that are commonly dysregulated in late-stage human PDAC. This differential requirement for Brd4 for metaplasia, regeneration and neoplastic transformation was traced back to key differences in chromatin accessibility dynamics at acinar, metaplasia and neoplasia gene loci in response to mutant Kras and injury. In normal pancreas, Brd4-dependent differentiation programs sustain acinar differentiation programs that counteract effects of injury during metaplasia (ADM), enabling return to tissue homeostasis. During neoplastic transformation (ADR), normal Brd4 function is co-opted to mediate early activation of genes found commonly altered in full-blown human pancreatic cancers. Mutant Kras and injury cooperate to drive rapid chromatin accessibility changes at many of these PDAC-associated genes, detected within 48 hours and before the establishment of precursor lesions, resulting in a chromatin state that is largely retained in invasive disease. Chromatin remodeling functionally contributes to disease pathogenesis by activating known and novel pro-tumorigenic factors, including the alarmin cytokine Il-33, that is sufficient to phenocopy injury's effects in accelerating Kras-driven neoplastic reprogramming in vivo. Thus, the chromatin state produced by the combination of gene mutation and tissue damage represents a bona fide epigenetic mechanism of cancer initiation.

FIG. 14A shows a heatmap of RNA-seq data showing the relative expression of cytokine receptors in lineage-traced (mKate2⁺;CD45⁻) pancreatic epithelial cells isolated for the indicated tissue states. Red box highlights a cluster of receptors that are selectively activated in PDAC from early stages of tumor development (but not in normal regeneration) and that is enriched in Th2 cytokine receptors.

FIG. 14B shows the relative mRNA levels (left) and chromatin accessibility profiles (right) of the Th2 cytokine receptors Il4ra and Il 13ra1 in pancreatic epithelial cells isolated for the indicated tissue states. The grey box marks peaks gained de novo during early pancreatic neoplasia and retained in invasive PDAC.

FIG. 14C shows the pro-proliferative effects of recombinant IL-4 and IL-13 cytokines in pre-malignant mutant Kras^{G12D} assessed by Cell Titer Glo after a 6-day growth. These effects are further enhanced by pre-treatment with the cytokines during 2 weeks.

FIG. 15 shows the approach for pancreas-specific, inducible and traceable Brd4 silencing. Transgenic mice carrying the indicated alleles permit doxycycline (dox)-inducible expression of GFP-linked shRNAs targeting Brd4 or Renilla luciferase (Ren, control), selectively in KRAS mutant pancreatic acinar cells marked with the fluorescent tag mKate2 (KC-GEMM). Analagous mice lacking the LSL-KRAS^{G12D} allele (C-GEMM) enable Brd4 suppression in normal exocrine pancreas.

FIG. 16A shows a histological analysis of the pancreatic ductal cells in KC-GEMM-shRen or KC-GEMM-shBrd4 mice fed on doxycycline (dox) diet since postnatal day 7 and euthanized at 6 weeks after birth.

FIG. 16B shows a histological analysis of the pancreatic acinar cells in KC-GEMM-shRen or KC-GEMM-shBrd4 mice fed on doxycycline (dox) diet since postnatal day 7 and euthanized at 6 weeks after birth.

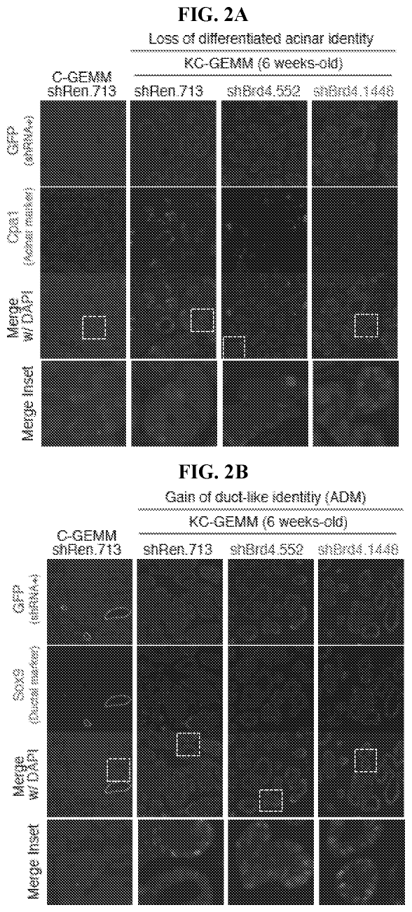

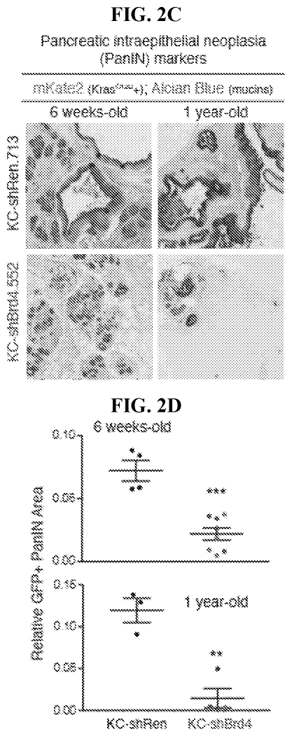

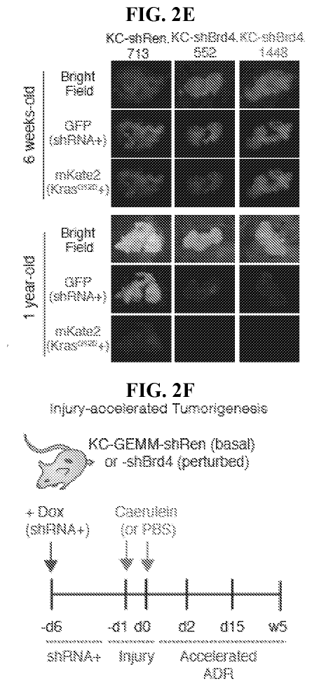

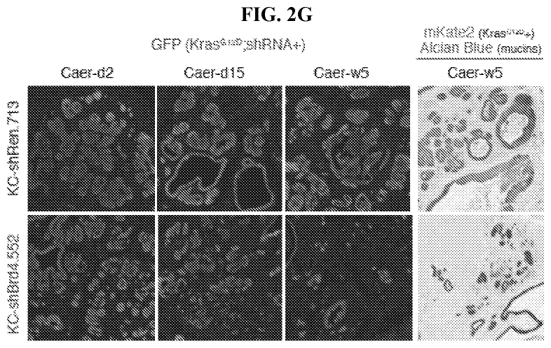

FIG. 17 shows the quantification of the histological analyses in FIG. 2C.

FIG. 18 shows the Myc expression (as assessed by Myc IF) in Brd4-suppressed pancreatic epithelial cells undergoing KRAS-driven acinar-to-ductal metaplasia, (shown at day 2 post-Caer treatment).

FIG. 19 demonstrates a lack of regeneration with concomitant exocrine tissue loss of shBrd4-expressing normal pancreas after Caer-induced tissue damage, as assessed by pancreas-to-body weight ratios at the indicated days post-Caer treatment. Mice were placed on dox diet at 4 weeks of age, and treated i.p. with Caer or vehicle control (PBS) one week thereafter.

FIG. 20 shows a strategy for unbiased dissection of chromatin and transcriptional landscapes programs of mutant KRAS and wild-type pancreatic epithelial cells (mKate2⁺, Cd45⁻) undergoing injury-induced regeneration or KRAS-dependent tumorigenesis in 5 weeks old C-GEMM or KC-GEMM mice, respectively. When indicated, epithelial plasticity was induced in a synchronous manner by caerulein treatment (acute protocol) and subsequent molecular analyses were performed at day 2 post-treatment to enrich for early events.

FIG. 21A shows a principal component analysis (PCA) of genome-wide RNA-Seq data (left), and heat map of supervised hierarchical clustering of Brd4-regulated genes in FACS-sorted mutant KRAS pancreatic epithelial cells (right).

FIG. 21B shows GSEA plots for gene signatures associated with pancreatic tumorigenesis in FACS-sorted mutant KRAS pancreatic epithelial cells expressing shRNAs against Renilla (shRen) or Brd4 (shBrd4). Gene signatures were extracted from Boj et al. Cell 2014 and include differentially upregulated (top) or downregulated (bottom) genes in PanIN-derived or PDAC-derived mouse organoids relative to normal pancreatic organoids.

FIG. 21C shows supervised clustering of differentially expressed genes (DEGs) upon Brd4 suppression in wild-type or mutant KRAS pancreatic epithelial cells treated with caerulein, revealing “tumor-specific” Brd4 targets. In this case, DEGs are extracted from FIG. 21A analyses, comparing shRen control to both of the Brd4-shRNAs (shBrd4-1=Brd4-shRNA.1448; and shBrd4-2-Brd4-shRNA.552).

FIG. 21D shows the overlap of differentially expressed Brd4 targets involved in pancreatic regeneration and tumorigenesis. DEGs are extracted from FIG. 21A analyses, comparing shRen control to both Brd4-shRNAs (shBrd4-1=Brd4-shRNA.1448; and shBrd4-2-Brd4-shRNA.552).

FIG. 22A shows supervised clustering of differentially accessible chromatin regions from ATAC-seq profiles of pancreatic epithelial cells undergoing injury-induced regeneration or KRAS-dependent tumorigenesis vs normal pancreas. Heatmaps were generated using Depth-norm normalization.

FIG. 22B shows the corresponding distribution of affected genomic elements inferred from ATAC-seq profiles (depth normalized) of pancreatic epithelial cells undergoing injury-induced regeneration or KRAS-dependent tumorigenesis vs normal pancreas.

FIG. 22C shows ATAC-Seq plots (top) or RNA-Seq plots (bottom) showing differential chromatin dynamics (depth-normalized profiles) and Brd4-dependent expression of representative “metaplasia” or “neoplasia”-associated genes.

FIG. 23A shows a principal component analysis (PCA) of ATAC-Seq data (depth-normalized profiles) from wild-type or mutant KRAS pancreata, 2 days after treatment with caerulein or PBS (control).

FIG. 23B shows representative ATAC-Seq plots from wild-type or mutant KRAS pancreata, 2 days after treatment with caerulein or PBS (control).

FIG. 24A shows a qPCR analysis of the expression of the indicated genes in pancreatic epithelial cells sorted from KC- and C-GEMM animals treated with -shBrd4/-shRen at day 2 post-Caerulein exposure.

FIG. 24B shows Dclk1 (left), Il-33 (middle) and Agr2 (right) protein expression assessed by IF in pancreatic tissues from KC- and C-GEMM animals treated with -shBrd4/-shRen at day 2 post-Caerulein exposure.

FIG. 25A shows supervised clustering of differentially accessible chromatin regions inferred from ATAC-seq profiles of sorted pancreatic epithelial cells from normal, injury induced Acinar-to-ductal metaplasia (ADM), KRAS-driven Acinar-to-ductal reprogramming (ADR), and established PDAC GEMM samples. *C-d2=Day 2-post Caerulein.

FIG. 25B shows the distribution of genomic elements affected by differential chromatin accessibility in ADR (vs acinar) in (FIG. 25(A)). *C-d2=Day 2-post Caerulein.

FIG. 25C shows representative ATAC-Seq plots showing the synergistic increase in chromatin accessibility at the IL-33 locus in KRAS-driven ADR and PDAC, but not in injury-associated ADM, oncogenic KRAS, or normal pancreas. *C-d2=Day 2-post Caerulein.

FIG. 25D shows a motif analysis in the top 500 chromatin sites gained in KRAS-induced ADR (not overlapping with ADM) using HOMER to predict the top-ranking TFs (right column) involved in chromatin remodeling.

FIG. 25E shows the quantification of IL-33 mRNA expression levels (a Brd4 target) during pancreatic tumorigenesis vs. injury-induced regeneration.

FIG. 26A is a heatmap showing differentially expressed cytokines in KC-shRen and KC-shBrd4 (n=2 indep. shRNAs) at day 2 post-caerulein treatment. IL-33 and IL-23 are the top differentially expressed cytokines that are impacted by Brd4 silencing.

FIG. 26B shows representative ATAC-Seq plots of FACS-sorted pancreatic epithelial cells showing increased chromatin accessibility at the IL-33 (top) or IL1rl1 (bottom) loci in KRAS-driven ADR and PDAC, but not in injury-induced ADM or normal pancreas.

FIG. 27A shows the treatment of KC-GEMM mice with recombinant IL-33 (1 μg/mouse, 5 consecutive days) promoted tumorigenesis and cancer stem cell expansion, as assessed by H&E staining (left), alcian blue staining of Kate-marked KRAS mutant cells (middle) and Dclk1 immunofluorescence (right, marker for “PDAC stem cells”). Histological analyses were performed at day 21 post-treatment.

FIG. 27B shows the initial characterization of new mouse model enabling pancreas-specific suppression of IL-33 via shRNA: validation of IL-33 silencing in GFP-labeled epithelial cells (left) and Masson's trichome staining showing reduced fibrosis upon IL-33 silencing (On Dox, 6 weeks old mice) (right).

FIG. 28A shows that the treatment with recombinant IL-33 accelerates mutant KRAS-driven tumorigenesis, having no effect in wild-type pancreas, as assessed by H&E staining and alcian blue staining in mKate2⁺ pancreatic epithelial cells.

FIG. 28B shows that the treatment with recombinant IL-33 accelerates mutant KRAS-driven tumorigenesis, having no effect in wild-type pancreas, as assessed by gene expression analyses of the indicated PanIN markers (Agr2, Muc6 and Dclk1) in mKate2⁺ pancreatic epithelial cells.

FIG. 29A shows the overlap between human differentially expressed genes (DEGs) in human PDAC vs normal samples (published by Yang et al., Cancer Res 76: 3838-3850 (2016)) and the DEGs disclosed herein between normal, regenerating, early neoplastic and malignant murine pancreatic epithelial cells. Differential expression analysis was performed to define differentially expressed genes (DEGs) between PDAC, using>2-fold change and adjusted P-value<0.05 cut-off.

FIG. 29B shows the overlap between human differentially expressed genes (DEGs) in human PDAC vs normal samples (published by Moffitt et al., Nat Genet 47, 1168-1178 (2015)) and the DEGs disclosed herein between normal, regenerating, early neoplastic and malignant murine pancreatic epithelial cells.

FIG. 30 shows details of the ATAC models used to identify open chromatin regions (peaks) gained or lost in the settings of regeneration, early neoplasia, and full-blown PDAC in each state compared to corresponding controls.

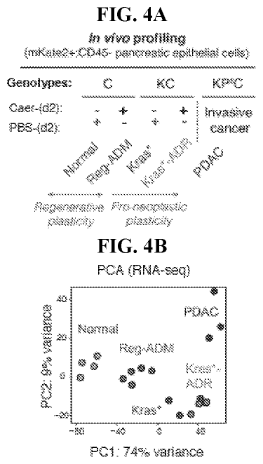

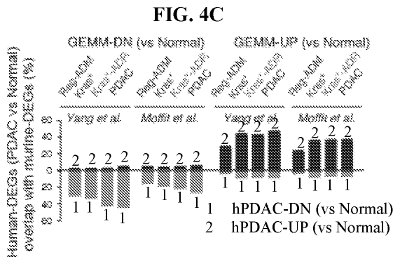

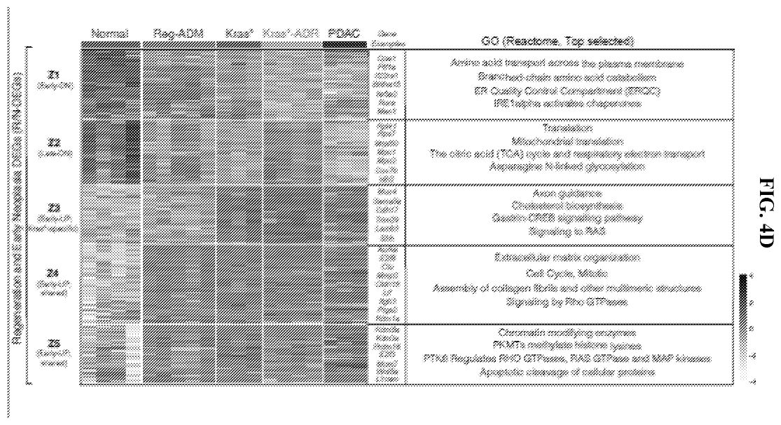

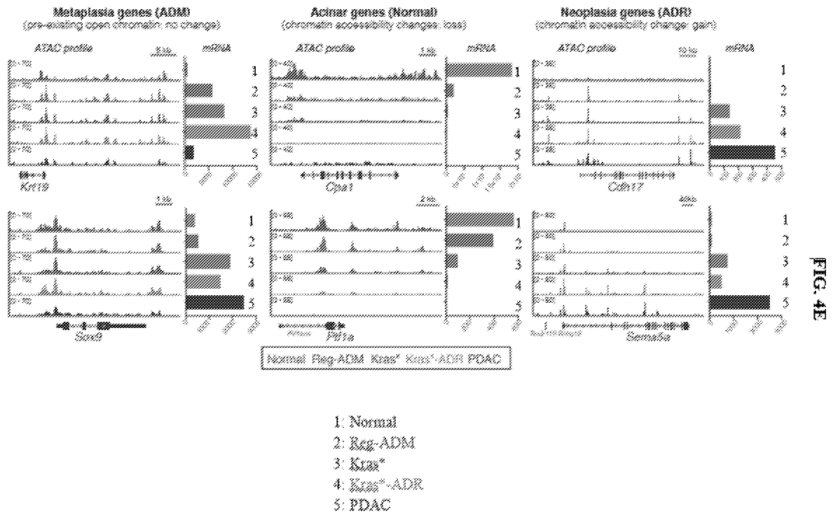

FIG. 31 shows a summary of RNA-seq and ATAC-seq features for selected cytokine and cytokine receptor genes, indicating chromatin-mediated activation of Type 2 cytokine signaling at early stages of pancreatic cancer development. Altered expression is maintained in late stage disease (PDAC). Related to FIGS. 4D, 11A, and 14A.

FIGS. 32A-32B show chromatin accessibility changes for the indicated loci associated to Type 2 cytokines or cytokine receptors, detected in pancreatic epithelial cells undergoing mutant Kras-driven neoplastic transformation (Kras*-ADR) (FIG. 32A) and in advanced cancer cells (PDAC) (FIG. 32B) versus normal healthy pancreatic epithelial cells. Note marked chromatin accessibility changes are detected both during tumor initiation and in advanced malignant disease.

### DETAILED DESCRIPTION

It is to be appreciated that certain aspects, modes, embodiments, variations and features of the present technology are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

The present disclosure identifies the shared and specific transcriptional programs that underlie normal tissue regeneration and early neoplasia. Thus, while there are notable similarities between the cell fate transitions accompanying neoplastic transformation and regeneration, the underlying transcriptional programs and chromatin states are distinct. Specifically, IL-33, an ‘alarmin’ cytokine that plays a central role in triggering inflammation and tissue remodeling in response to injury, was identified to be a specific mediator of pancreatic tumorigenesis downstream of the described epigenetic alterations associated with KRAS mutation, showing features of enhancer-dependent activation in early neoplasia but not in normal tissue regeneration. Along with the activation of IL33, tumor-specific epigenetic alterations also induce aberrant expression of Th2 cytokine receptors (e.g., IL4RA, IL13RA1, IL13RA2, IL17RE, IL18R1, IL18RAP, IL31RA) in cells undergoing mutant KRAS-driven neoplastic transformation and in advanced pancreatic cancer cells. The present disclosure demonstrates that the inhibitors of Type 2 cytokine signaling disclosed herein are useful in methods for detecting, treating, and/or preventing pancreatic cancer in a subject in need thereof.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including orally, intranasally, parenterally (intravenously, intramuscularly, intraperitoneally, or subcutaneously), intratumorally, or topically. Administration includes self-administration and the administration by another.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease or condition, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of pancreatic cancer. As used herein, a “therapeutically effective amount” of a composition refers to composition levels in which the physiological effects of a disease or condition are ameliorated or eliminated. A therapeutically effective amount can be given in one or more administrations.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the terms “individual”, “patient”, or “subject” are used interchangeably and refer to an individual organism, a vertebrate, a mammal, or a human. In certain embodiments, the individual, patient or subject is a human.

As used herein, “prevention”, “prevent”, or “preventing” of a disorder or condition refers to one or more compounds that, in a statistical sample, reduces the occurrence of the disorder or condition in the treated sample relative to an untreated control sample, or delays the onset of one or more symptoms of the disorder or condition relative to the untreated control sample. As used herein, preventing PDAC, includes preventing or delaying the initiation of symptoms of PDAC. As used herein, prevention of PDAC also includes preventing a recurrence of one or more signs or symptoms of PDAC.

As used herein, a “sample” or “biological sample” refers to a body fluid or a tissue sample isolated from a subject. In some cases, a biological sample may consist of or comprise whole blood, platelets, red blood cells, white blood cells, plasma, sera, urine, feces, epidermal sample, vaginal sample, skin sample, cheek swab, sperm, amniotic fluid, cultured cells, bone marrow sample, tumor biopsies, aspirate and/or chorionic villi, cultured cells, endothelial cells, synovial fluid, lymphatic fluid, ascites fluid, interstitial or extracellular fluid and the like. The term “sample” may also encompass the fluid in spaces between cells, including gingival crevicular fluid, bone marrow, cerebrospinal fluid (CSF), saliva, mucus, sputum, semen, sweat, urine, or any other bodily fluids. Samples can be obtained from a subject by any means including, but not limited to, venipuncture, excretion, ejaculation, massage, biopsy, needle aspirate, lavage, scraping, surgical incision, or intervention or other means known in the art. A blood sample can be whole blood or any fraction thereof, including blood cells (red blood cells, white blood cells or leukocytes, and platelets), serum and plasma.

As used herein, the term “separate” therapeutic use refers to an administration of at least two active ingredients at the same time or at substantially the same time by different routes.

As used herein, the term “sequential” therapeutic use refers to administration of at least two active ingredients at different times. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

“Treating”, “treat”, or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment or prevention of medical diseases and conditions as described are intended to mean “substantial,” which includes total but also less than total treatment or prevention, and wherein some biologically or medically relevant result is achieved. The treatment may be a continuous prolonged treatment for a chronic disease or a single, or few time administrations for the treatment of an acute condition.

As used herein “type 2 cytokines” refer to cytokines that promote a strong T helper type 2 (Th2) immune response, characterized by the production of interleukin-4 (IL-4), IL-5 and IL-13, and classically drive recruitment and activation of mast cells, basophils and eosinophils, and goblet cell hyperplasia in airway and intestinal epithelia. Type 2 cytokines are generally produced by Th2 T-helper cells, CD8⁺ T cells, and non-T cell leukocytes such as monocytes, ILC2, B cells, eosinophils, mast cells, and basophils. See Lucey et al., Clinical Microbiology Reviews 9(4): 532-562 (1996). Examples of type 2 cytokines include but are not limited to IL-4, IL-5, IL-6, IL-9, IL-10, IL-13, IL17E, IL-31, IL-33 etc.

### Inhibitors of Type 2 Cytokine Signaling and Brd4 Inhibitors

The present disclosure provides inhibitors of Type 2 cytokine signaling. In some embodiments, the inhibitor of Type 2 cytokine signaling inhibits the activity or expression of a Type 2 cytokine or Type 2 cytokine receptor signaling protein selected from the group consisting of IL-33, IL-4, IL-13, IL-5, IL-23A, IL-17E, IL-9, IL9R, IL1RL1, IL4RA, IL13RA1, IL13RA2, IL5RA, IL1RL2, IL17RA, IL31RA, IL17RE, IL18R1, IL18RAP, and IL1R1.

Exemplary mRNA sequences of Type 2 cytokines or Type 2 cytokine receptor signaling proteins are provided below, represented by SEQ ID NOs: 23-38 and 46-51:

>NM_033439.4 Homo sapiens interleukin 33 (IL-33), transcript variant 1,
mRNA
(SEQ ID NO: 23)
ACAGAGCTGCAGCTCTTCAGGGAAGAAATCAAAACAAGATCACAAGAATACTGAAAAATGAAGCCTAAAA
TGAAGTATTCAACCAACAAAATTTCCACAGCAAAGTGGAAGAACACAGCAAGCAAAGCCTTGTGTTTCAA
GCTGGGAAAATCCCAACAGAAGGCCAAAGAAGTTTGCCCCATGTACTTTATGAAGCTCCGCTCTGGCCTT
ATGATAAAAAAGGAGGCCTGTTACTTTAGGAGAGAAACCACCAAAAGGCCTTCACTGAAAACAGGTAGAA
AGCACAAAAGACATCTGGTACTCGCTGCCTGTCAACAGCAGTCTACTGTGGAGTGCTTTGCCTTTGGTAT
ATCAGGGGTCCAGAAATATACTAGAGCACTTCATGATTCAAGTATCACAGGAATTTCACCTATTACAGAG
TATCTTGCTTCTCTAAGCACATACAATGATCAATCCATTACTTTTGCTTTGGAGGATGAAAGTTATGAGA
TATATGTTGAAGACTTGAAAAAAGATGAAAAGAAAGATAAGGTGTTACTGAGTTACTATGAGTCTCAACA
CCCCTCAAATGAATCAGGTGACGGTGTTGATGGTAAGATGTTAATGGTAACCCTGAGTCCTACAAAAGAC
TTCTGGTTGCATGCCAACAACAAGGAACACTCTGTGGAGCTCCATAAGTGTGAAAAACCACTGCCAGACC
AGGCCTTCTTTGTCCTTCATAATATGCACTCCAACTGTGTTTCATTTGAATGCAAGACTGATCCTGGAGT
GTTTATAGGTGTAAAGGATAATCATCTTGCTCTGATTAAAGTAGACTCTTCTGAGAATTTGTGTACTGAA
AATATCTTGTTTAAGCTCTCTGAAACTTAGTTGATGGAAACCTGTGAGTCTTGGGTTGAGTACCCAAATG
CTACCACTGGAGAAGGAATGAGAGATAAAGAAAGAGACAGGTGACATCTAAGGGAAATGAAGAGTGCTTA
GCATGTGTGGAATGTTTTCCATATTATGTATAAAAATATTTTTTCTAATCCTCCAGTTATTCTTTTATTT
CCCTCTGTATAACTGCATCTTCAATACAAGTATCAGTATATTAAATAGGGTATTGGTAAAGAAACGGTCA
ACATTCTAAAGAGATACAGTCTGACCTTTACTTTTCTCTAGTTTCAGTCCAGAAAGAACTTCATATTTAG
AGCTAAGGCCACTGAGGAAAGAGCCATAGCTTAAGTCTCTATGTAGACAGGGATCCATTTTAAAGAGCTA
CTTAGAGAAATAATTTTCCACAGTTCCAAACGATAGGCTCAAACACTAGAGCTGCTAGTAAAAAGAAGAC
CAGATGCTTCACAGAATTATCATTTTTTCAACTGGAATAAAACACCAGGTTTGTTTGTAGATGTCTTAGG
CAACACTCAGAGCAGATCTCCCTTACTGTCAGGGGATATGGAACTTCAAAGGCCCACATGGCAAGCCAGG
TAACATAAATGTGTGAAAAAGTAAAGATAACTAAAAAATTTAGAAAAATAAATCCAGTATTTGTAAAGTG
AATAACTTCATTTCTAATTGTTTAATTTTTAAAATTCTGATTTTTATATATTGAGTTTAAGCAAGGCATT
CTTACACGAGGAAGTGAAGTAAATTTTAGTTCAGACATAAAATTTCACTTATTAGGAATATGTAACATGC
TAAAACTTTTTTTTTTTTAAAGAGTACTGAGTCACAACATGTTTTAGAGCATCCAAGTACCATATAATCC
AACTATCATGGTAAGGCCAGAAATCTTCTAACCTACCAGAGCCTAGATGAGACACCGAATTAACATTAAA
ATTTCAGTAACTGACTGTCCCTCATGTCCATGGCCTACCATCCCTTCTGACCCTGGCTTCCAGGGACCTA
TGTCTTTTAATACTCACTGTCACATTGGGCAAAGTTGCTTCTAATCCTTATTTCCCATGTGCACAAGTCT
TTTTGTATTCCAGCTTCCTGATAACACTGCTTACTGTGGAATATTCATTTGACATCTGTCTCTTTTCATT
TCTTTTAACTACCATGCCCTTGATATATCTTTTGCACCTGCTGAACTTCATTTCTGTATCACCTGACCTC
TGGATGCCAAAACGTTTATTCTGCTTTGTCTGTTGTAGAATTTTAGATAAAGCTATTAATGGCAATATTT
TTTTGCTAAACGTTTTTGTTTTTTACTGTCACTAGGGCAATAAAATTTATACTCAACCATATAATAACAT
TTTTTAACTACTAAAGGAGTAGTTTTTATTTTAAAGTCTTAGCAATTTCTATTACAACTTTTCTTAGACT
TAACACTTATGATAAATGACTAACATAGTAACAGAATCTTTATGAAATATGACCTTTTCTGAAAATACAT
ACTTTTACATTTCTACTTTATTGAGACCTATTAGATGTAAGTGCTAGTAGAATATAAGATAAAAGAGGCT
GAGAATTACCATACAAGGGTATTACAACTGTAAAACAATTTATCTTTGTTTCATTGTTCTGTCAATAATT
GTTACCAAAGAGATAAAAATAAAAGCAGAATGTATATCATCCCATCTGAAAAACACTAATTATTGACATG
TGCATCTGTACAATAAACTTAAAATGATTATTAAATAATCAAATATATCTACTACATTGTTTATATTATT
GAATAAAGTATATTTTCCAAATGTA
>NM_000589.4 Homo sapiens interleukin 4 (IL4), transcript variant 1, mRNA
(SEQ ID NO: 24)
ATCGTTAGCTTCTCCTGATAAACTAATTGCCTCACATTGTCACTGCAAATCGACACCTATTAATGGGTCT
CACCTCCCAACTGCTTCCCCCTCTGTTCTTCCTGCTAGCATGTGCCGGCAACTTTGTCCACGGACACAAG
TGCGATATCACCTTACAGGAGATCATCAAAACTTTGAACAGCCTCACAGAGCAGAAGACTCTGTGCACCG
AGTTGACCGTAACAGACATCTTTGCTGCCTCCAAGAACACAACTGAGAAGGAAACCTTCTGCAGGGCTGC
GACTGTGCTCCGGCAGTTCTACAGCCACCATGAGAAGGACACTCGCTGCCTGGGTGCGACTGCACAGCAG
TTCCACAGGCACAAGCAGCTGATCCGATTCCTGAAACGGCTCGACAGGAACCTCTGGGGCCTGGCGGGCT
TGAATTCCTGTCCTGTGAAGGAAGCCAACCAGAGTACGTTGGAAAACTTCTTGGAAAGGCTAAAGACGAT
CATGAGAGAGAAATATTCAAAGTGTTCGAGCTGAATATTTTAATTTATGAGTTTTTGATAGCTTTATTTT
TTAAGTATTTATATATTTATAACTCATCATAAAATAAAGTATATATAGAATCTAA
>NM_002188.3 Homo sapiens interleukin 13 (IL13), transcript variant 1,
mRNA
(SEQ ID NO: 25)
AAGCCACCCAGCCTATGCATCCGCTCCTCAATCCTCTCCTGTTGGCACTGGGCCTCATGGCGCTTTTGTT
GACCACGGTCATTGCTCTCACTTGCCTTGGCGGCTTTGCCTCCCCAGGCCCTGTGCCTCCCTCTACAGCC
CTCAGGGAGCTCATTGAGGAGCTGGTCAACATCACCCAGAACCAGAAGGCTCCGCTCTGCAATGGCAGCA
TGGTATGGAGCATCAACCTGACAGCTGGCATGTACTGTGCAGCCCTGGAATCCCTGATCAACGTGTCAGG
CTGCAGTGCCATCGAGAAGACCCAGAGGATGCTGAGCGGATTCTGCCCGCACAAGGTCTCAGCTGGGCAG
TTTTCCAGCTTGCATGTCCGAGACACCAAAATCGAGGTGGCCCAGTTTGTAAAGGACCTGCTCTTACATT
TAAAGAAACTTTTTCGCGAGGGACAGTTCAACTGAAACTTCGAAAGCATCATTATTTGCAGAGACAGGAC
CTGACTATTGAAGTTGCAGATTCATTTTTCTTTCTGATGTCAAAAATGTCTTGGGTAGGCGGGAAGGAGG
GTTAGGGAGGGGTAAAATTCCTTAGCTTAGACCTCAGCCTGTGCTGCCCGTCTTCAGCCTAGCCGACCTC
AGCCTTCCCCTTGCCCAGGGCTCAGCCTGGTGGGCCTCCTCTGTCCAGGGCCCTGAGCTCGGTGGACCCA
GGGATGACATGTCCCTACACCCCTCCCCTGCCCTAGAGCACACTGTAGCATTACAGTGGGTGCCCCCCTT
GCCAGACATGTGGTGGGACAGGGACCCACTTCACACACAGGCAACTGAGGCAGACAGCAGCTCAGGCACA
CTTCTTCTTGGTCTTATTTATTATTGTGTGTTATTTAAATGAGTGTGTTTGTCACCGTTGGGGATTGGGG
AAGACTGTGGCTGCTAGCACTTGGAGCCAAGGGTTCAGAGACTCAGGGCCCCAGCACTAAAGCAGTGGAC
ACCAGGAGTCCCTGGTAATAAGTACTGTGTACAGAATTCTGCTACCTCACTGGGGTCCTGGGGCCTCGGA
GCCTCATCCGAGGCAGGGTCAGGAGAGGGGCAGAACAGCCGCTCCTGTCTGCCAGCCAGCAGCCAGCTCT
CAGCCAACGAGTAATTTATTGTTTTTCCTTGTATTTAAATATTAAATATGTTAGCAAAGAGTTAATATAT
AGAAGGGTACCTTGAACACTGGGGGAGGGGACATTGAACAAGTTGTTTCATTGACTATCAAACTGAAGCC
AGAAATAAAGTTGGTGACAGATA
>NM_000879.3 Homo sapiens interleukin 5 (IL5), mRNA
(SEQ ID NO: 26)
ATGCACTTTCTTTGCCAAAGGCAAACGCAGAACGTTTCAGAGCCATGAGGATGCTTCTGCATTTGAGTTT
GCTAGCTCTTGGAGCTGCCTACGTGTATGCCATCCCCACAGAAATTCCCACAAGTGCATTGGTGAAAGAG
ACCTTGGCACTGCTTTCTACTCATCGAACTCTGCTGATAGCCAATGAGACTCTGAGGATTCCTGTTCCTG
TACATAAAAATCACCAACTGTGCACTGAAGAAATCTTTCAGGGAATAGGCACACTGGAGAGTCAAACTGT
GCAAGGGGGTACTGTGGAAAGACTATTCAAAAACTTGTCCTTAATAAAGAAATACATTGACGGCCAAAAA
AAAAAGTGTGGAGAAGAAAGACGGAGAGTAAACCAATTCCTAGACTACCTGCAAGAGTTTCTTGGTGTAA
TGAACACCGAGTGGATAATAGAAAGTTGAGACTAAACTGGTTTGTTGCAGCCAAAGATTTTGGAGGAGAA
GGACATTTTACTGCAGTGAGAATGAGGGCCAAGAAAGAGTCAGGCCTTAATTTTCAGTATAATTTAACTT
CAGAGGGAAAGTAAATATTTCAGGCATACTGACACTTTGCCAGAAAGCATAAAATTCTTAAAATATATTT
CAGATATCAGAATCATTGAAGTATTTTCCTCCAGGCAAAATTGATATACTTTTTTCTTATTTAACTTAAC
ATTCTGTAAAATGTCTGTTAACTTAATAGTATTTATGAAATGGTTAAGAATTTGGTAAATTAGTATTTAT
TTAATGTTATGTTGTGTTCTAATAAAACAAAAATAGACAACTGTT
>NM_000600.5 Homo sapiens interleukin 6 (IL6), transcript variant 1, mRNA
(SEQ ID NO: 27)
ATTCTGCCCTCGAGCCCACCGGGAACGAAAGAGAAGCTCTATCTCCCCTCCAGGAGCCCAGCTATGAACT
CCTTCTCCACAAGCGCCTTCGGTCCAGTTGCCTTCTCCCTGGGGCTGCTCCTGGTGTTGCCTGCTGCCTT
CCCTGCCCCAGTACCCCCAGGAGAAGATTCCAAAGATGTAGCCGCCCCACACAGACAGCCACTCACCTCT
TCAGAACGAATTGACAAACAAATTCGGTACATCCTCGACGGCATCTCAGCCCTGAGAAAGGAGACATGTA
ACAAGAGTAACATGTGTGAAAGCAGCAAAGAGGCACTGGCAGAAAACAACCTGAACCTTCCAAAGATGGC
TGAAAAAGATGGATGCTTCCAATCTGGATTCAATGAGGAGACTTGCCTGGTGAAAATCATCACTGGTCTT
TTGGAGTTTGAGGTATACCTAGAGTACCTCCAGAACAGATTTGAGAGTAGTGAGGAACAAGCCAGAGCTG
TGCAGATGAGTACAAAAGTCCTGATCCAGTTCCTGCAGAAAAAGGCAAAGAATCTAGATGCAATAACCAC
CCCTGACCCAACCACAAATGCCAGCCTGCTGACGAAGCTGCAGGCACAGAACCAGTGGCTGCAGGACATG
ACAACTCATCTCATTCTGCGCAGCTTTAAGGAGTTCCTGCAGTCCAGCCTGAGGGCTCTTCGGCAAATGT
AGCATGGGCACCTCAGATTGTTGTTGTTAATGGGCATTCCTTCTTCTGGTCAGAAACCTGTCCACTGGGC
ACAGAACTTATGTTGTTCTCTATGGAGAACTAAAAGTATGAGCGTTAGGACACTATTTTAATTATTTTTA
ATTTATTAATATTTAAATATGTGAAGCTGAGTTAATTTATGTAAGTCATATTTATATTTTTAAGAAGTAC
CACTTGAAACATTTTATGTATTAGTTTTGAAATAATAATGGAAAGTGGCTATGCAGTTTGAATATCCTTT
GTTTCAGAGCCAGATCATTTCTTGGAAAGTGTAGGCTTACCTCAAATAAATGGCTAACTTATACATATTT
TTAAAGAAATATTTATATTGTATTTATATAATGTATAAATGGTTTTTATACCAATAAATGGCATTTTAAA
AAATTCA
>NM_000590.2 Homo sapiens interleukin 9 (IL9), mRNA
(SEQ ID NO: 28)
AAGCGAGCTCCAGTCCGCTGTCAAGATGCTTCTGGCCATGGTCCTTACCTCTGCCCTGCTCCTGTGCTCC
GTGGCAGGCCAGGGGTGTCCAACCTTGGCGGGGATCCTGGACATCAACTTCCTCATCAACAAGATGCAGG
AAGATCCAGCTTCCAAGTGCCACTGCAGTGCTAATGTGACCAGTTGTCTCTGTTTGGGCATTCCCTCTGA
CAACTGCACCAGACCATGCTTCAGTGAGAGACTGTCTCAGATGACCAATACCACCATGCAAACAAGATAC
CCACTGATTTTCAGTCGGGTGAAAAAATCAGTTGAAGTACTAAAGAACAACAAGTGTCCATATTTTTCCT
GTGAACAGCCATGCAACCAAACCACGGCAGGCAACGCGCTGACATTTCTGAAGAGTCTTCTGGAAATTTT
CCAGAAAGAAAAGATGAGAGGGATGAGAGGCAAGATATGAAGATGAAATATTATTTATCCTATTTATTAA
ATTTAAAAAGCTTTCTCTTTAAGTTGCTACAATTTAAAAATCAAGTAAGCTACTCTAAATCAGTATCAGT
TGTGATTATTTGTTTAACATTGTATGTCTTTATTTTGAAATAAAT
>NM_000572.3 Homo sapiens interleukin 10 (IL10), mRNA
(SEQ ID NO: 29)
ACACATCAGGGGCTTGCTCTTGCAAAACCAAACCACAAGACAGACTTGCAAAAGAAGGCATGCACAGCTC
AGCACTGCTCTGTTGCCTGGTCCTCCTGACTGGGGTGAGGGCCAGCCCAGGCCAGGGCACCCAGTCTGAG
AACAGCTGCACCCACTTCCCAGGCAACCTGCCTAACATGCTTCGAGATCTCCGAGATGCCTTCAGCAGAG
TGAAGACTTTCTTTCAAATGAAGGATCAGCTGGACAACTTGTTGTTAAAGGAGTCCTTGCTGGAGGACTT
TAAGGGTTACCTGGGTTGCCAAGCCTTGTCTGAGATGATCCAGTTTTACCTGGAGGAGGTGATGCCCCAA
GCTGAGAACCAAGACCCAGACATCAAGGCGCATGTGAACTCCCTGGGGGAGAACCTGAAGACCCTCAGGC
TGAGGCTACGGCGCTGTCATCGATTTCTTCCCTGTGAAAACAAGAGCAAGGCCGTGGAGCAGGTGAAGAA
TGCCTTTAATAAGCTCCAAGAGAAAGGCATCTACAAAGCCATGAGTGAGTTTGACATCTTCATCAACTAC
ATAGAAGCCTACATGACAATGAAGATACGAAACTGAGACATCAGGGTGGCGACTCTATAGACTCTAGGAC
ATAAATTAGAGGTCTCCAAAATCGGATCTGGGGCTCTGGGATAGCTGACCCAGCCCCTTGAGAAACCTTA
TTGTACCTCTCTTATAGAATATTTATTACCTCTGATACCTCAACCCCCATTTCTATTTATTTACTGAGCT
TCTCTGTGAACGATTTAGAAAGAAGCCCAATATTATAATTTTTTTCAATATTTATTATTTTCACCTGTTT
TTAAGCTGTTTCCATAGGGTGACACACTATGGTATTTGAGTGTTTTAAGATAAATTATAAGTTACATAAG
GGAGGAAAAAAAATGTTCTTTGGGGAGCCAACAGAAGCTTCCATTCCAAGCCTGACCACGCTTTCTAGCT
GTTGAGCTGTTTTCCCTGACCTCCCTCTAATTTATCTTGTCTCTGGGCTTGGGGCTTCCTAACTGCTACA
AATACTCTTAGGAAGAGAAACCAGGGAGCCCCTTTGATGATTAATTCACCTTCCAGTGTCTCGGAGGGAT
TCCCCTAACCTCATTCCCCAACCACTTCATTCTTGAAAGCTGTGGCCAGCTTGTTATTTATAACAACCTA
AATTTGGTTCTAGGCCGGGCGCGGTGGCTCACGCCTGTAATCCCAGCACTTTGGGAGGCTGAGGCGGGTG
GATCACTTGAGGTCAGGAGTTCCTAACCAGCCTGGTCAACATGGTGAAACCCCGTCTCTACTAAAAATAC
AAAAATTAGCCGGGCATGGTGGCGCGCACCTGTAATCCCAGCTACTTGGGAGGCTGAGGCAAGAGAATTG
CTTGAACCCAGGAGATGGAAGTTGCAGTGAGCTGATATCATGCCCCTGTACTCCAGCCTGGGTGACAGAG
CAAGACTCTGTCTCAAAAAATAAAAATAAAAATAAATTTGGTTCTAATAGAACTCAGTTTTAACTAGAAT
TTATTCAATTCCTCTGGGAATGTTACATTGTTTGTCTGTCTTCATAGCAGATTTTAATTTTGAATAAATA
AATGTATCTTATTCACATCA
>NM_000879.3 Homo sapiens interleukin 5 (IL5), mRNA
(SEQ ID NO: 30)
ATGCACTTTCTTTGCCAAAGGCAAACGCAGAACGTTTCAGAGCCATGAGGATGCTTCTGCATTTGAGTTT
GCTAGCTCTTGGAGCTGCCTACGTGTATGCCATCCCCACAGAAATTCCCACAAGTGCATTGGTGAAAGAG
ACCTTGGCACTGCTTTCTACTCATCGAACTCTGCTGATAGCCAATGAGACTCTGAGGATTCCTGTTCCTG
TACATAAAAATCACCAACTGTGCACTGAAGAAATCTTTCAGGGAATAGGCACACTGGAGAGTCAAACTGT
GCAAGGGGGTACTGTGGAAAGACTATTCAAAAACTTGTCCTTAATAAAGAAATACATTGACGGCCAAAAA
AAAAAGTGTGGAGAAGAAAGACGGAGAGTAAACCAATTCCTAGACTACCTGCAAGAGTTTCTTGGTGTAA
TGAACACCGAGTGGATAATAGAAAGTTGAGACTAAACTGGTTTGTTGCAGCCAAAGATTTTGGAGGAGAA
GGACATTTTACTGCAGTGAGAATGAGGGCCAAGAAAGAGTCAGGCCTTAATTTTCAGTATAATTTAACTT
CAGAGGGAAAGTAAATATTTCAGGCATACTGACACTTTGCCAGAAAGCATAAAATTCTTAAAATATATTT
CAGATATCAGAATCATTGAAGTATTTTCCTCCAGGCAAAATTGATATACTTTTTTCTTATTTAACTTAAC
ATTCTGTAAAATGTCTGTTAACTTAATAGTATTTATGAAATGGTTAAGAATTTGGTAAATTAGTATTTAT
TTAATGTTATGTTGTGTTCTAATAAAACAAAAATAGACAACTGTT
>NM_016584.3 Homo sapiens interleukin 23 subunit alpha (IL23A), mRNA
(SEQ ID NO: 31)
AACAGGAAGCAGCTTACAAACTCGGTGAACAACTGAGGGAACCAAACCAGAGACGCGCTGAACAGAGAGA
ATCAGGCTCAAAGCAAGTGGAAGTGGGCAGAGATTCCACCAGGACTGGTGCAAGGCGCAGAGCCAGCCAG
ATTTGAGAAGAAGGCAAAAAGATGCTGGGGAGCAGAGCTGTAATGCTGCTGTTGCTGCTGCCCTGGACAG
CTCAGGGCAGAGCTGTGCCTGGGGGCAGCAGCCCTGCCTGGACTCAGTGCCAGCAGCTTTCACAGAAGCT
CTGCACACTGGCCTGGAGTGCACATCCACTAGTGGGACACATGGATCTAAGAGAAGAGGGAGATGAAGAG
ACTACAAATGATGTTCCCCATATCCAGTGTGGAGATGGCTGTGACCCCCAAGGACTCAGGGACAACAGTC
AGTTCTGCTTGCAAAGGATCCACCAGGGTCTGATTTTTTATGAGAAGCTGCTAGGATCGGATATTTTCAC
AGGGGAGCCTTCTCTGCTCCCTGATAGCCCTGTGGGCCAGCTTCATGCCTCCCTACTGGGCCTCAGCCAA
CTCCTGCAGCCTGAGGGTCACCACTGGGAGACTCAGCAGATTCCAAGCCTCAGTCCCAGCCAGCCATGGC
AGCGTCTCCTTCTCCGCTTCAAAATCCTTCGCAGCCTCCAGGCCTTTGTGGCTGTAGCCGCCCGGGTCTT
TGCCCATGGAGCAGCAACCCTGAGTCCCTAAAGGCAGCAGCTCAAGGATGGCACTCAGATCTCCATGGCC
CAGCAAGGCCAAGATAAATCTACCACCCCAGGCACCTGTGAGCCAACAGGTTAATTAGTCCATTAATTTT
AGTGGGACCTGCATATGTTGAAAATTACCAATACTGACTGACATGTGATGCTGACCTATGATAAGGTTGA
GTATTTATTAGATGGGAAGGGAAATTTGGGGATTATTTATCCTCCTGGGGACAGTTTGGGGAGGATTATT
TATTGTATTTATATTGAATTATGTACTTTTTTCAATAAAGTCTTATTTTTGTGGCTA
>NM_016232.5 Homo sapiens interleukin 1 receptor like 1 (IL1RL1),
transcript variant 1, mRNA
(SEQ ID NO: 32)
GAGTTGTGAAACTGTGGGCAGAAAGTTGAGGAAGAAAGAACTCAAGTACAACCCAATGAGGTTGAGATAT
AGGCTACTCTTCCCAACTCAGTCTTGAAGAGTATCACCAACTGCCTCATGTGTGGTGACCTTCACTGTCG
TATGCCAGTGACTCATCTGGAGTAATCTCAACAACGAGTTACCAATACTTGCTCTTGATTGATAAACAGA
ATGGGGTTTTGGATCTTAGCAATTCTCACAATTCTCATGTATTCCACAGCAGCAAAGTTTAGTAAACAAT
CATGGGGCCTGGAAAATGAGGCTTTAATTGTAAGATGTCCTAGACAAGGAAAACCTAGTTACACCGTGGA
TTGGTATTACTCACAAACAAACAAAAGTATTCCCACTCAGGAAAGAAATCGTGTGTTTGCCTCAGGCCAA
CTTCTGAAGTTTCTACCAGCTGCAGTTGCTGATTCTGGTATTTATACCTGTATTGTCAGAAGTCCCACAT
TCAATAGGACTGGATATGCGAATGTCACCATATATAAAAAACAATCAGATTGCAATGTTCCAGATTATTT
GATGTATTCAACAGTATCTGGATCAGAAAAAAATTCCAAAATTTATTGTCCTACCATTGACCTCTACAAC
TGGACAGCACCTCTTGAGTGGTTTAAGAATTGTCAGGCTCTTCAAGGATCAAGGTACAGGGCGCACAAGT
CATTTTTGGTCATTGATAATGTGATGACTGAGGACGCAGGTGATTACACCTGTAAATTTATACACAATGA
AAATGGAGCCAATTATAGTGTGACGGCGACCAGGTCCTTCACGGTCAAGGATGAGCAAGGCTTTTCTCTG
TTTCCAGTAATCGGAGCCCCTGCACAAAATGAAATAAAGGAAGTGGAAATTGGAAAAAACGCAAACCTAA
CTTGCTCTGCTTGTTTTGGAAAAGGCACTCAGTTCTTGGCTGCCGTCCTGTGGCAGCTTAATGGAACAAA
AATTACAGACTTTGGTGAACCAAGAATTCAACAAGAGGAAGGGCAAAATCAAAGTTTCAGCAATGGGCTG
GCTTGTCTAGACATGGTTTTAAGAATAGCTGACGTGAAGGAAGAGGATTTATTGCTGCAGTACGACTGTC
TGGCCCTGAATTTGCATGGCTTGAGAAGGCACACCGTAAGACTAAGTAGGAAAAATCCAATTGATCATCA
TAGCATCTACTGCATAATTGCAGTATGTAGTGTATTTTTAATGCTAATCAATGTCCTGGTTATCATCCTA
AAAATGTTCTGGATTGAGGCCACTCTGCTCTGGAGAGACATAGCTAAACCTTACAAGACTAGGAATGATG
GAAAGCTCTATGATGCTTATGTTGTCTACCCACGGAACTACAAATCCAGTACAGATGGGGCCAGTCGTGT
AGAGCACTTTGTTCACCAGATTCTGCCTGATGTTCTTGAAAATAAATGTGGCTATACCTTATGCATTTAT
GGGAGAGATATGCTACCTGGAGAAGATGTAGTCACTGCAGTGGAAACCAACATACGAAAGAGCAGGCGGC
ACATTTTCATCCTGACCCCTCAGATCACTCACAATAAGGAGTTTGCCTACGAGCAGGAGGTTGCCCTGCA
CTGTGCCCTCATCCAGAACGACGCCAAGGTGATACTTATTGAGATGGAGGCTCTGAGCGAGCTGGACATG
CTGCAGGCTGAGGCGCTTCAGGACTCCCTCCAGCATCTTATGAAAGTACAGGGGACCATCAAGTGGAGGG
AGGACCACATTGCCAATAAAAGGTCCCTGAATTCTAAATTCTGGAAGCACGTGAGGTACCAAATGCCTGT
GCCAAGCAAAATTCCCAGAAAGGCCTCTAGTTTGACTCCCTTGGCTGCCCAGAAGCAATAGTGCCTGCTG
TGATGTGCAAAGGCATCTGAGTTTGAAGCTTTCCTGACTTCTCCTAGCTGGCTTATGCCCCTGCACTGAA
GTGTGAGGAGCAGGAATATTAAAGGGATTCAGGCCTC
>NM_000418.4 Homo sapiens interleukin 4 receptor (IL4R), transcript
variant 1, mRNA
(SEQ ID NO: 33)
ACTTCCCGCTTGGGCGCCCGGACGGCGAATGGAGCAGGGGCGCGCAGATAATTAAAGATTTACACACAGC
TGGAAGAAATCATAGAGAAGCCGGGCGTGGTGGCTCATGCCTATAATCCCAGCACTTTTGGAGGCTGAGG
CGGGCAGATCACTTGAGATCAGGAGTTCGAGACCAGCCTGGTGCCTTGGCATCTCCCAATGGGGTGGCTT
TGCTCTGGGCTCCTGTTCCCTGTGAGCTGCCTGGTCCTGCTGCAGGTGGCAAGCTCTGGGAACATGAAGG
TCTTGCAGGAGCCCACCTGCGTCTCCGACTACATGAGCATCTCTACTTGCGAGTGGAAGATGAATGGTCC
CACCAATTGCAGCACCGAGCTCCGCCTGTTGTACCAGCTGGTTTTTCTGCTCTCCGAAGCCCACACGTGT
ATCCCTGAGAACAACGGAGGCGCGGGGTGCGTGTGCCACCTGCTCATGGATGACGTGGTCAGTGCGGATA
ACTATACACTGGACCTGTGGGCTGGGCAGCAGCTGCTGTGGAAGGGCTCCTTCAAGCCCAGCGAGCATGT
GAAACCCAGGGCCCCAGGAAACCTGACAGTTCACACCAATGTCTCCGACACTCTGCTGCTGACCTGGAGC
AACCCGTATCCCCCTGACAATTACCTGTATAATCATCTCACCTATGCAGTCAACATTTGGAGTGAAAACG
ACCCGGCAGATTTCAGAATCTATAACGTGACCTACCTAGAACCCTCCCTCCGCATCGCAGCCAGCACCCT
GAAGTCTGGGATTTCCTACAGGGCACGGGTGAGGGCCTGGGCTCAGTGCTATAACACCACCTGGAGTGAG
TGGAGCCCCAGCACCAAGTGGCACAACTCCTACAGGGAGCCCTTCGAGCAGCACCTCCTGCTGGGCGTCA
GCGTTTCCTGCATTGTCATCCTGGCCGTCTGCCTGTTGTGCTATGTCAGCATCACCAAGATTAAGAAAGA
ATGGTGGGATCAGATTCCCAACCCAGCCCGCAGCCGCCTCGTGGCTATAATAATCCAGGATGCTCAGGGG
TCACAGTGGGAGAAGCGGTCCCGAGGCCAGGAACCAGCCAAGTGCCCACACTGGAAGAATTGTCTTACCA
AGCTCTTGCCCTGTTTTCTGGAGCACAACATGAAAAGGGATGAAGATCCTCACAAGGCTGCCAAAGAGAT
GCCTTTCCAGGGCTCTGGAAAATCAGCATGGTGCCCAGTGGAGATCAGCAAGACAGTCCTCTGGCCAGAG
AGCATCAGCGTGGTGCGATGTGTGGAGTTGTTTGAGGCCCCGGTGGAGTGTGAGGAGGAGGAGGAGGTAG
AGGAAGAAAAAGGGAGCTTCTGTGCATCGCCTGAGAGCAGCAGGGATGACTTCCAGGAGGGAAGGGAGGG
CATTGTGGCCCGGCTAACAGAGAGCCTGTTCCTGGACCTGCTCGGAGAGGAGAATGGGGGCTTTTGCCAG
CAGGACATGGGGGAGTCATGCCTTCTTCCACCTTCGGGAAGTACGAGTGCTCACATGCCCTGGGATGAGT
TCCCAAGTGCAGGGCCCAAGGAGGCACCTCCCTGGGGCAAGGAGCAGCCTCTCCACCTGGAGCCAAGTCC
TCCTGCCAGCCCGACCCAGAGTCCAGACAACCTGACTTGCACAGAGACGCCCCTCGTCATCGCAGGCAAC
CCTGCTTACCGCAGCTTCAGCAACTCCCTGAGCCAGTCACCGTGTCCCAGAGAGCTGGGTCCAGACCCAC
TGCTGGCCAGACACCTGGAGGAAGTAGAACCCGAGATGCCCTGTGTCCCCCAGCTCTCTGAGCCAACCAC
TGTGCCCCAACCTGAGCCAGAAACCTGGGAGCAGATCCTCCGCCGAAATGTCCTCCAGCATGGGGCAGCT
GCAGCCCCCGTCTCGGCCCCCACCAGTGGCTATCAGGAGTTTGTACATGCGGTGGAGCAGGGTGGCACCC
AGGCCAGTGCGGTGGTGGGCTTGGGTCCCCCAGGAGAGGCTGGTTACAAGGCCTTCTCAAGCCTGCTTGC
CAGCAGTGCTGTGTCCCCAGAGAAATGTGGGTTTGGGGCTAGCAGTGGGGAAGAGGGGTATAAGCCTTTC
CAAGACCTCATTCCTGGCTGCCCTGGGGACCCTGCCCCAGTCCCTGTCCCCTTGTTCACCTTTGGACTGG
ACAGGGAGCCACCTCGCAGTCCGCAGAGCTCACATCTCCCAAGCAGCTCCCCAGAGCACCTGGGTCTGGA
GCCGGGGGAAAAGGTAGAGGACATGCCAAAGCCCCCACTTCCCCAGGAGCAGGCCACAGACCCCCTTGTG
GACAGCCTGGGCAGTGGCATTGTCTACTCAGCCCTTACCTGCCACCTGTGCGGCCACCTGAAACAGTGTC
ATGGCCAGGAGGATGGTGGCCAGACCCCTGTCATGGCCAGTCCTTGCTGTGGCTGCTGCTGTGGAGACAG
GTCCTCGCCCCCTACAACCCCCCTGAGGGCCCCAGACCCCTCTCCAGGTGGGGTTCCACTGGAGGCCAGT
CTGTGTCCGGCCTCCCTGGCACCCTCGGGCATCTCAGAGAAGAGTAAATCCTCATCATCCTTCCATCCTG
CCCCTGGCAATGCTCAGAGCTCAAGCCAGACCCCCAAAATCGTGAACTTTGTCTCCGTGGGACCCACATA
CATGAGGGTCTCTTAGGTGCATGTCCTCTTGTTGCTGAGTCTGCAGATGAGGACTAGGGCTTATCCATGC
CTGGGAAATGCCACCTCCTGGAAGGCAGCCAGGCTGGCAGATTTCCAAAAGACTTGAAGAACCATGGTAT
GAAGGTGATTGGCCCCACTGACGTTGGCCTAACACTGGGCTGCAGAGACTGGACCCCGCCCAGCATTGGG
CTGGGCTCGCCACATCCCATGAGAGTAGAGGGCACTGGGTCGCCGTGCCCCACGGCAGGCCCCTGCAGGA
AAACTGAGGCCCTTGGGCACCTCGACTTGTGAACGAGTTGTTGGCTGCTCCCTCCACAGCTTCTGCAGCA
GACTGTCCCTGTTGTAACTGCCCAAGGCATGTTTTGCCCACCAGATCATGGCCCACGTGGAGGCCCACCT
GCCTCTGTCTCACTGAACTAGAAGCCGAGCCTAGAAACTAACACAGCCATCAAGGGAATGACTTGGGCGG
CCTTGGGAAATCGATGAGAAATTGAACTTCAGGGAGGGTGGTCATTGCCTAGAGGTGCTCATTCATTTAA
CAGAGCTTCCTTAGGTTGATGCTGGAGGCAGAATCCCGGCTGTCAAGGGGTGTTCAGTTAAGGGGAGCAA
CAGAGGACATGAAAAATTGCTATGACTAAAGCAGGGACAATTTGCTGCCAAACACCCATGCCCAGCTGTA
TGGCTGGGGGCTCCTCGTATGCATGGAACCCCCAGAATAAATATGCTCAGCCACCCTGTGGGCCGGGCAA
TCCAGACAGCAGGCATAAGGCACCAGTTACCCTGCATGTTGGCCCAGACCTCAGGTGCTAGGGAAGGCGG
GAACCTTGGGTTGAGTAATGCTCGTCTGTGTGTTTTAGTTTCATCACCTGTTATCTGTGTTTGCTGAGGA
GAGTGGAACAGAAGGGGTGGAGTTTTGTATAAATAAAGTTTCTTTGTCTCTTTA
>NM_001560.3 Homo sapiens interleukin 13 receptor subunit alpha 1
(IL13RA1), mRNA
(SEQ ID NO: 34)
CCAGCCCGGCCGGGCTCCGAGGCGAGAGGCTGCATGGAGTGGCCGGCGCGGCTCTGCGGGCTGTGGGCGC
TGCTGCTCTGCGCCGGCGGCGGGGGCGGGGGCGGGGGCGCCGCGCCTACGGAAACTCAGCCACCTGTGAC
AAATTTGAGTGTCTCTGTTGAAAACCTCTGCACAGTAATATGGACATGGAATCCACCCGAGGGAGCCAGC
TCAAATTGTAGTCTATGGTATTTTAGTCATTTTGGCGACAAACAAGATAAGAAAATAGCTCCGGAAACTC
GTCGTTCAATAGAAGTACCCCTGAATGAGAGGATTTGTCTGCAAGTGGGGTCCCAGTGTAGCACCAATGA
GAGTGAGAAGCCTAGCATTTTGGTTGAAAAATGCATCTCACCCCCAGAAGGTGATCCTGAGTCTGCTGTG
ACTGAGCTTCAATGCATTTGGCACAACCTGAGCTACATGAAGTGTTCTTGGCTCCCTGGAAGGAATACCA
GTCCCGACACTAACTATACTCTCTACTATTGGCACAGAAGCCTGGAAAAAATTCATCAATGTGAAAACAT
CTTTAGAGAAGGCCAATACTTTGGTTGTTCCTTTGATCTGACCAAAGTGAAGGATTCCAGTTTTGAACAA
CACAGTGTCCAAATAATGGTCAAGGATAATGCAGGAAAAATTAAACCATCCTTCAATATAGTGCCTTTAA
CTTCCCGTGTGAAACCTGATCCTCCACATATTAAAAACCTCTCCTTCCACAATGATGACCTATATGTGCA
ATGGGAGAATCCACAGAATTTTATTAGCAGATGCCTATTTTATGAAGTAGAAGTCAATAACAGCCAAACT
GAGACACATAATGTTTTCTACGTCCAAGAGGCTAAATGTGAGAATCCAGAATTTGAGAGAAATGTGGAGA
ATACATCTTGTTTCATGGTCCCTGGTGTTCTTCCTGATACTTTGAACACAGTCAGAATAAGAGTCAAAAC
AAATAAGTTATGCTATGAGGATGACAAACTCTGGAGTAATTGGAGCCAAGAAATGAGTATAGGTAAGAAG
CGCAATTCCACACTCTACATAACCATGTTACTCATTGTTCCAGTCATCGTCGCAGGTGCAATCATAGTAC
TCCTGCTTTACCTAAAAAGGCTCAAGATTATTATATTCCCTCCAATTCCTGATCCTGGCAAGATTTTTAA
AGAAATGTTTGGAGACCAGAATGATGATACTCTGCACTGGAAGAAGTACGACATCTATGAGAAGCAAACC
AAGGAGGAAACCGACTCTGTAGTGCTGATAGAAAACCTGAAGAAAGCCTCTCAGTGATGGAGATAATTTA
TTTTTACCTTCACTGTGACCTTGAGAAGATTCTTCCCATTCTCCATTTGTTATCTGGGAACTTATTAAAT
GGAAACTGAAACTACTGCACCATTTAAAAACAGGCAGCTCATAAGAGCCACAGGTCTTTATGTTGAGTCG
CGCACCGAAAAACTAAAAATAATGGGCGCTTTGGAGAAGAGTGTGGAGTCATTCTCATTGAATTATAAAA
GCCAGCAGGCTTCAAACTAGGGGACAAAGCAAAAAGTGATGATAGTGGTGGAGTTAATCTTATCAAGAGT
TGTGACAACTTCCTGAGGGATCTATACTTGCTTTGTGTTCTTTGTGTCAACATGAACAAATTTTATTTGT
AGGGGAACTCATTTGGGGTGCAAATGCTAATGTCAAACTTGAGTCACAAAGAACATGTAGAAAACAAAAT
GGATAAAATCTGATATGTATTGTTTGGGATCCTATTGAACCATGTTTGTGGCTATTAAAACTCTTTTAAC
AGTCTGGGCTGGGTCCGGTGGCTCACGCCTGTAATCCCAGCAATTTGGGAGTCCGAGGCGGGCGGATCAC
TCGAGGTCAGGAGTTCCAGACCAGCCTGACCAAAATGGTGAAACCTCCTCTCTACTAAAACTACAAAAAT
TAACTGGGTGTGGTGGCGCGTGCCTGTAATCCCAGCTACTCGGGAAGCTGAGGCAGGTGAATTGTTTGAA
CCTGGGAGGTGGAGGTTGCAGTGAGCAGAGATCACACCACTGCACTCTAGCCTGGGTGACAGAGCAAGAC
TCTGTCTAAAAAACAAAACAAAACAAAACAAAACAAAAAAACCTCTTAATATTCTGGAGTCATCATTCCC
TTCGACAGCATTTTCCTCTGCTTTGAAAGCCCCAGAAATCAGTGTTGGCCATGATGACAACTACAGAAAA
ACCAGAGGCAGCTTCTTTGCCAAGACCTTTCAAAGCCATTTTAGGCTGTTAGGGGCAGTGGAGGTAGAAT
GACTCCTTGGGTATTAGAGTTTCAACCATGAAGTCTCTAACAATGTATTTTCTTCACCTCTGCTACTCAA
GTAGCATTTACTGTGTCTTTGGTTTGTGCTAGGCCCCCGGGTGTGAAGCACAGACCCCTTCCAGGGGTTT
ACAGTCTATTTGAGACTCCTCAGTTCTTGCCACTTTTTTTTTTAATCTCCACCAGTCATTTTTCAGACCT
TTTAACTCCTCAATTCCAACACTGATTTCCCCTTTTGCATTCTCCCTCCTTCCCTTCCTTGTAGCCTTTT
GACTTTCATTGGAAATTAGGATGTAAATCTGCTCAGGAGACCTGGAGGAGCAGAGGATAATTAGCATCTC
AGGTTAAGTGTGAGTAATCTGAGAAACAATGACTAATTCTTGCATATTTTGTAACTTCCATGTGAGGGTT
TTCAGCATTGATATTTGTGCATTTTCTAAACAGAGATGAGGTGGTATCTTCACGTAGAACATTGGTATTC
GCTTGAGAAAAAAAGAATAGTTGAACCTATTTCTCTTTCTTTACAAGATGGGTCCAGGATTCCTCTTTTC
TCTGCCATAAATGATTAATTAAATAGCTTTTGTGTCTTACATTGGTAGCCAGCCAGCCAAGGCTCTGTTT
ATGCTTTTGGGGGGCATATATTGGGTTCCATTCTCACCTATCCACACAACATATCCGTATATATCCCCTC
TACTCTTACTTCCCCCAAATTTAAAGAAGTATGGGAAATGAGAGGCATTTCCCCCACCCCATTTCTCTCC
TCACACACAGACTCATATTACTGGTAGGAACTTGAGAACTTTATTTCCAAGTTGTTCAAACATTTACCAA
TCATATTAATACAATGATGCTATTTGCAATTCCTGCTCCTAGGGGAGGGGAGATAAGAAACCCTCACTCT
CTACAGGTTTGGGTACAAGTGGCAACCTGCTTCCATGGCCGTGTAGAAGCATGGTGCCCTGGCTTCTCTG
AGGAAGCTGGGGTTCATGACAATGGCAGATGTAAAGTTATTCTTGAAGTCAGATTGAGGCTGGGAGACAG
CCGTAGTAGATGTTCTACTTTGTTCTGCTGTTCTCTAGAAAGAATATTTGGTTTTCCTGTATAGGAATGA
GATTAATTCCTTTCCAGGTATTTTATAATTCTGGGAAGCAAAACCCATGCCTCCCCCTAGCCATTTTTAC
TGTTATCCTATTTAGATGGCCATGAAGAGGATGCTGTGAAATTCCCAACAAACATTGATGCTGACAGTCA
TGCAGTCTGGGAGTGGGGAAGTGATCTTTTGTTCCCATCCTCTTCTTTTAGCAGTAAAATAGCTGAGGGA
AAAGGGAGGGAAAAGGAAGTTATGGGAATACCTGTGGTGGTTGTGATCCCTAGGTCTTGGGAGCTCTTGG
AGGTGTCTGTATCAGTGGATTTCCCATCCCCTGTGGGAAATTAGTAGGCTCATTTACTGTTTTAGGTCTA
GCCTATGTGGATTTTTTCCTAACATACCTAAGCAAACCCAGTGTCAGGATGGTAATTCTTATTCTTTCGT
TCAGTTAAGTTTTTCCCTTCATCTGGGCACTGAAGGGATATGTGAAACAATGTTAACATTTTTGGTAGTC
TTCAACCAGGGATTGTTTCTGTTTAACTTCTTATAGGAAAGCTTGAGTAAAATAAATATTGTCTTTTTGT
ATGTCA
>NM_000564.4 Homo sapiens interleukin 5 receptor subunit alpha (IL5RA),
transcript variant 1, mRNA
(SEQ ID NO: 35)
AAACAAAACAGAAATGCAACGCTTTAGAGTACCCACGGAAAACTTGTTTACCTTGTCACCATGAGTAAAA
GTTAATTCCCACTCCTGAAGAGAGCAAACCAACTCTGAAAGAGAGTGAAAATGCAGACAAGACAGTTATC
AGATAATGGCTATCTGGACGAGAGATTCTTTCGTTTGACAGCAGTTTGGTTGTTGGGAGTTCCAGTTCAG
CTCCTGCACAGTTGCTCTGTACAAATCCTCCTCCATATTTGCTTAGAGAAAACGTGTTGCCATCCCATCA
TGAAGGAAGCTGCCTGAGAGTTTTTAACCATTACAGCCGTGATGATGAAAGAGTGAAGAACCGCCTCTAA
GTTAAAAAGTGCACCCAGAGATAAGGTTCGTTCTCAATGCCCTGCCGCTGCTTCTCATCGCATGGCCACC
GCATTTCTCAGGCCAGGCACATTGAGCATTGGTCCTGTGCCTGACGCTATGCTAGATGCTGGGGTTGCAG
CCACGAGCATAGACACGACAGACACGGTCCTCGCCATCTTCTGTTGAGTACTGGTCGGAACAAGAGGATC
GTCTGTAGACAGGCTACAGATTGTTTTAGATTGAAGTTTCCTGTCATGTTCACTCATCTTTAAATCCTCA
TAGTGAAAAAGGATATGATCATCGTGGCGCATGTATTACTCATCCTTTTGGGGGCCACTGAGATACTGCA
AGCTGACTTACTTCCTGATGAAAAGATTTCACTTCTCCCACCTGTCAATTTCACCATTAAAGTTACTGGT
TTGGCTCAAGTTCTTTTACAATGGAAACCAAATCCTGATCAAGAGCAAAGGAATGTTAATCTAGAATATC
AAGTGAAAATAAACGCTCCAAAAGAAGATGACTATGAAACCAGAATCACTGAAAGCAAATGTGTAACCAT
CCTCCACAAAGGCTTTTCAGCAAGTGTGCGGACCATCCTGCAGAACGACCACTCACTACTGGCCAGCAGC
TGGGCTTCTGCTGAACTTCATGCCCCACCAGGGTCTCCTGGAACCTCAATTGTGAATTTAACTTGCACCA
CAAACACTACAGAAGACAATTATTCACGTTTAAGGTCATACCAAGTTTCCCTTCACTGCACCTGGCTTGT
TGGCACAGATGCCCCTGAGGACACGCAGTATTTTCTCTACTATAGGTATGGCTCTTGGACTGAAGAATGC
CAAGAATACAGCAAAGACACACTGGGGAGAAATATCGCATGCTGGTTTCCCAGGACTTTTATCCTCAGCA
AAGGGCGTGACTGGCTTGCGGTGCTTGTTAACGGCTCCAGCAAGCACTCTGCTATCAGGCCCTTTGATCA
GCTGTTTGCCCTTCACGCCATTGATCAAATAAATCCTCCACTGAATGTCACAGCAGAGATTGAAGGAACT
CGTCTCTCTATCCAATGGGAGAAACCAGTGTCTGCTTTTCCAATCCATTGCTTTGATTATGAAGTAAAAA
TACACAATACAAGGAATGGATATTTGCAGATAGAAAAATTGATGACCAATGCATTCATCTCAATAATTGA
TGATCTTTCTAAGTACGATGTTCAAGTGAGAGCAGCAGTGAGCTCCATGTGCAGAGAGGCAGGGCTCTGG
AGTGAGTGGAGCCAACCTATTTATGTGGGAAATGATGAACACAAGCCCTTGAGAGAGTGGTTTGTCATTG
TGATTATGGCAACCATCTGCTTCATCTTGTTAATTCTCTCGCTTATCTGTAAAATATGTCATTTATGGAT
CAAGTTGTTTCCACCAATTCCAGCACCAAAAAGTAATATCAAAGATCTCTTTGTAACCACTAACTATGAG
AAAGCTGGGTCCAGTGAGACGGAAATTGAAGTCATCTGTTATATAGAGAAGCCTGGAGTTGAGACCCTGG
AGGATTCTGTGTTTTGACTGTCACTTTGGCATCCTCTGATGAACTCACACATGCCTCAGTGCCTCAGTGA
AAAGAACAGGGATGCTGGCTCTTGGCTAAGAGGTGTTCAGAATTTAGGCAACACTCAATTTACCTGCGAA
GCAATACACCCAGACACACCAGTCTTGTATCTCTTAAAAGTATGGATGCTTCATCCAAATCGCCTCACCT
ACAGCAGGGAAGTTGACTCATCCAAGCATTTTGCCATGTTTTTTCTCCCCATGCCGTACAGGGTAGCACC
TCCTCACCTGCCAATCTTTGCAATTTGCTTGACTCACCTCAGACTTTTCATTCACAACAGACAGCTTTTA
AGGCTAACGTCCAGCTGTATTTACTTCTGGCTGTGCCCGTTTGGCTGTTTAAGCTGCCAATTGTAGCACT
CAGCTACCATCTGAGGAAGAAAGCATTTTGCATCAGCCTGGAGTGAACCATGAACTTGGATTCAAGACTG
TCTTTTCTATAGCAAGTGAGAGCCACAAATTCCTCACCCCCCTACATTCTAGAATGATCTTTTTCTAGGT
AGATTGTGTATGTGTGTGTATGAGAGAGAGAGAGAGAGAGAGAGAGAGAGAAATTATCTCAAGCTCCAGA
GGCCTGATCCAGGATACATCATTTGAAACCAACTAATTTAAAAGCATAATAGAGCTAATATATCACCCCA
TATCAAAAGAGACGACAGATTTTGTTCAAATATTATATTCTTGAAGGAAGCCTTAATATTCTGGAAATGT
GCTGAGGAAGTCATTGAGCATAATTCAATCATGAAAGGGACCACTGAATCAAGAATGAACTTTCTGAAGA
GATGTTTGCTGCCAAAGACTTCAGAACAACTTGTTGGTCAAGTAATGCCAGATCAATCCTAGTCTCTCAA
GTTTGGAAAAGTTTCTACAAGGCCATTCCATCAAATATCCCAATATACACAGAAATTCTTCTTCCTAACA
TCTTCATATATGAGTCTGCATCCAGATTGGAGGCCTTTGTGGTGGGTGGTGGACTGCAGGTGGAACCAAG
CACAGGTGTGCCCTGGGCAGAGGTGGGCAAGAGGAAGGAACCCAAACAGTGCTGATTCCACATGGCCCTT
GGTCAAGAAGCTGAAGGCCTAGAAAGGAGGTAACAGTGCCCAGAATCCCCACCTGGCACACTCTTTAAAT
GCTTTCTGGAGGTGTTTTAGTCAGTTTTGATCCATCGGTGGGGACACACTGACTACCATATATTTCCAGG
TTTGTTTGTTTTCTTAACTAACCTGTAATTATCCACTGTAACTAGCAGAGGGTTGAACCCTGGGTAATAG
GCACAGAAACATCTTGAATCTTGACAAGCCTGCACTGTCTTATAGTAATCTGTATTACTGTCCCATTTTA
TAGTTGCAGTTACTAAGGTTCAGAGAAGTTGATAGGAAATGGCATAGTGAGGAGTTGAATACCAGGCTCA
GGTAAAAGTGACCCCAGAAATGTGCAGAGGAGTGTCAGGGAAAGCTGAGGCCTGCTTAGGGGTGTGGCTG
AGAAGGCAACACCCATGGAGGTGCTGCAATAGTTGGCAAAATGTTTAAAATGTTTGGTCACTCAAATGAT
TCATAATCTCAAACCTGTTTTTGTTTTTTTTTTTTTTTTTTTTTGTGGGGTGGGTGGTGGAGACAGTCTT
GTTCTGTCTCCCAGGCTAGAGTGTAGTGATGTGATCTTAGCTCACTACAACCTCCCTGTCCTGGGCTCAA
GCCATCCTCCTCCCTCACCCTCCCAAGTAGCTGGGACTACAGGTGCATAACACCATTCCCAGCTCTTTTT
TGTATTTTTTTTTGTAAAGCTGGGGTTTTGCCATGCTGTCTAGGTTCATCTCGAACTCCTGGGCTCAAGC
GATACACCGGCCTCAGCCTTCCAAAGTACTGGGATTACAGGCATGAACCACCATTCCTGGCCAGGAAACT
GTATTTGTAATGAACACCTTAGGTCAGAATCAGAGGCGCCTGGAGGGCTCCTTAAGACACAGAGGAGTGG
GCTCCATTCTTAGAGTCTCTAACTGAATAGGTCTGGGTGAGGTCTGAAAAGCTGCGTTTCTAACAAGTTC
TCAGGTGATGCTCATGCTGCTGGTCTCCAGGACACACTCGGAGAACTGCTGCTCCAGGAGAGTCTTATGA
ATACCAAAATTTGAAAATCAGCCGCTGAAGGAAGATGTGACAGAAAAGTCCTGTCCCCAGAAAACTGTCC
TTTTGGGGCGGCTTGGAGCACCCTGTCTGATCTCTTTCAAACCACAACCCAGACAGTTGGCATTGTGGAA
ACTGCCTCTGCTTAGGAGGCAGGAACAGAAGCAGGGAGCTCCCCCTCCACAAATGTGGCCAGCTCTTCAG
TGCTTGGTGTGGTGCCCACTTCAGACTCCTCCCTCCACTCAGCAGCCATAGATCACCTGGGCTCTGTGGA
GCTCCTTGCCACACTTCCCTGAGCTGGTACCGTTATCTGGCCCTTGGTATATGTGTTTCAGGAGCAACAC
AAAGTAGCCCATTGGCCTCCTTGGTGGATTGGGAACTCCTTCCTCCCTTCTTGCCCTGTGCAACTCATAG
TCATTCTTTGGGAACCCGTAACCATCATCACTTCCTCCATGAAGCCTTCAAAAGCAGAGTTGGTTGATCC
CGCCTCTGGATTCTCATTCTGGGCCTCATACCCGTGTTAGAGCTACTGCATTGTAGAATATACCATTAAC
AGCTGGGAGGCCTCCCCTCTAGACTGCGGCTTCCAGGAGGATGGAGCTTGTGTGAAATACGTCTTTTTAT
CTGCAGTGTCTAGGACAGTATCTGACTTAAGAAAATATAAATGTTTCTTAAACCCTCTGTCAGCCCTGAT
ACGGCACCTTGTATATAGGTTAGACTGAACAGTATTTGGCAGAGATCCTCAAGGCCTACAATAATGACAG
GGGGAGGAAGCTGGGTATCTAATGAGAATGCGGTTAGTTAGAAGCAACCAGGAGACACATGTGCCTAACA
ACTGGGCTACTGCAACCTGGATGTTTTTGCTGCCAAAGTGTCTAAATGGTTATGTATAAATTGCTGTGTA
AGAGGCATTTCAGAAGTGGCTCACTGTGAGTGGTCTATACTGTTCTCACCAGCCTTGTCATAGCAAGTCA
AAGAGTTTGAAATTCACTTAACAGTAAACACCATCAGCAGCAGGTTGATGTAAGGTAAAGTCATGATATT
GGCTCCCATCTCTTGCCATCTCTCACTAGGACGAGTGTAGAGAGTCACTTTTCTCCTAGAGCCTCCACAG
CCACGGGATCAGAACAAACATGCTTTTCCAAGATATCCGTTCCAATAGGGATTTTTAGCCCAATTTCTTT
CCTTGGCATTTGGAATTCACTGCGTGCAGCATGGGGGAGTGCTGGGGAGACTGGAGGGTGAAGGTCACAT
TTGCCAACAGCCCGACCACCTGAGCCTCATTGAAATGCCTCAGACCTCAAGCTATTCATGCAGGCTGGGG
AAGAATCCACGTGGGCAAAGAACATCCTGATTAAAAATCCAAACGTTTTCATGGTGAGACTTCTATGTGT
CAGGACCATCTGCTGGTGGAATTAAGAAGCCTGAGGTGCTAATTTGTTAAAGAAAAAGTAGATATCTGGA
AGTGAGAACAGAGAACTGAAAACAATCTCTGATGGCACAAGTAAAGGTGAAAAACCTAATTAGAATAGGA
CTTGGTGCTAGCCTAAGGACTGTAGCTATTCCTGCTTCTACATCCTTCAAATTGAGTACCAGGGTGCAAA
TTTTTTCAAAACCATTCCTTCCTTTGGAATTTTGTATTGTACTATAAGACAATCATGCTTATCTCCTGTT
TTGTAGGTAATAGATGTGAATATAAATATGTTTCAGAATGAGAAATATTAAACATAAATGACAGTGAAAA
AAAAAAAAAAA
>NM_003854.4 Homo sapiens interleukin 1 receptor like 2 (IL1RL2),
transcript variant 1, mRNA
(SEQ ID NO: 36)
GCTCGTGTCCCTGTCATATTTTCCACTCTCCACGAGGTCCTGCGCGCTTCAATCCTGCAGGCAGCCCGGT
TTGGGGATGTGGTCCTTGCTGCTCTGCGGGTTGTCCATCGCCCTTCCACTGTCTGTCACAGCAGATGGAT
GCAAGGACATTTTTATGAAAAATGAGATACTTTCAGCAAGCCAGCCTTTTGCTTTTAATTGTACATTCCC
TCCCATAACATCTGGGGAAGTCAGTGTAACATGGTATAAAAATTCTAGCAAAATCCCAGTGTCCAAAATC
ATACAGTCTAGAATTCACCAGGACGAGACTTGGATTTTGTTTCTCCCCATGGAATGGGGGGACTCAGGAG
TCTACCAATGTGTTATAAAGGGTAGAGACAGCTGTCATAGAATACATGTAAACCTAACTGTTTTTGAAAA
ACATTGGTGTGACACTTCCATAGGTGGTTTACCAAATTTATCAGATGAGTACAAGCAAATATTACATCTT
GGAAAAGATGATAGTCTCACATGTCATCTGCACTTCCCGAAGAGTTGTGTTTTGGGTCCAATAAAGTGGT
ATAAGGACTGTAACGAGATTAAAGGGGAGCGGTTCACTGTTTTGGAAACCAGGCTTTTGGTGAGCAATGT
CTCGGCAGAGGACAGAGGGAACTACGCGTGTCAAGCCATACTGACACACTCAGGGAAGCAGTACGAGGTT
TTAAATGGCATCACTGTGAGCATTACAGAAAGAGCTGGATATGGAGGAAGTGTCCCTAAAATCATTTATC
CAAAAAATCATTCAATTGAAGTACAGCTTGGTACCACTCTGATTGTGGACTGCAATGTAACAGACACCAA
GGATAATACAAATCTACGATGCTGGAGAGTCAATAACACTTTGGTGGATGATTACTATGATGAATCCAAA
CGAATCAGAGAAGGGGTGGAAACCCATGTCTCTTTTCGGGAACATAATTTGTACACAGTAAACATCACCT
TCTTGGAAGTGAAAATGGAAGATTATGGCCTTCCTTTCATGTGCCACGCTGGAGTGTCCACAGCATACAT
TATATTACAGCTCCCAGCTCCGGATTTTCGAGCTTACTTGATAGGAGGGCTTATCGCCTTGGTGGCTGTG
GCTGTGTCTGTTGTGTACATATACAACATTTTTAAGATCGACATTGTTCTTTGGTATCGAAGTGCCTTCC
ATTCTACAGAGACCATAGTAGATGGGAAGCTGTATGACGCCTATGTCTTATACCCCAAGCCCCACAAGGA
AAGCCAGAGGCATGCCGTGGATGCCCTGGTGTTGAATATCCTGCCCGAGGTGTTGGAGAGACAATGTGGA
TATAAGTTGTTTATATTCGGCAGAGATGAATTCCCTGGACAAGCCGTGGCCAATGTCATCGATGAAAACG
TTAAGCTGTGCAGGAGGCTGATTGTCATTGTGGTCCCCGAATCGCTGGGCTTTGGCCTGTTGAAGAACCT
GTCAGAAGAACAAATCGCGGTCTACAGTGCCCTGATCCAGGACGGGATGAAGGTTATTCTCATTGAGCTG
GAGAAAATCGAGGACTACACAGTCATGCCAGAGTCAATTCAGTACATCAAACAGAAGCATGGTGCCATCC
GGTGGCATGGGGACTTCACGGAGCAGTCACAGTGTATGAAGACCAAGTTTTGGAAGACAGTGAGATACCA
CATGCCGCCCAGAAGGTGTCGGCCGTTTCCTCCGGTCCAGCTGCTGCAGCACACACCTTGCTACCGCACC
GCAGGCCCAGAACTAGGCTCAAGAAGAAAGAAGTGTACTCTCACGACTGGCTAAGACTTGCTGGACTGAC
ACCTATGGCTGGAAGATGACTTGTTTTGCTCCATGTCTCCTCATTCCTACACCTATTTTCTGCTGCAGGA
TGAGGCTAGGGTTAGCATTCTAGACACCCAGTTGAGCTCAGGCGTAGAGAAGAGGAGGATGGGATAAGAA
CTGGGGCCATCCCCATGTCATGGTGGGTGAGAGCTGGGGCCATCCCCGTGGTCATGGAGGGTGAGAGCTG
GGGGTTATCCCCATGGTCATGGAGGGTGAGGGCTGGTCGGGGGAGGCATCCCCAAGTCATGGTGGGTGAG
AGCTCGGAGCATCCCCATGTCATGGTGGGTGAGATCTGGGGGTATCCCTGTGTCATGGTGGGTGAGGGCG
GGTGGTCATCCACATGGTCATAGTGGGTGAGAGCTGGGGGTATCCCTACATCATGGTGGGTGAGAGCTGG
GAGCATCCCCATGTCATGGTGGGCGAGATCTGGGGGGTATCCCCACGTCATGGTGGATGAGAGCTGGGGG
AATCACCATGTCATGGTGGGTGAGATCTTGGGGGATCACCTGTCATGGTGGGTGAGAGCTGGGGGGATCA
CCTGTCATGGTGGGTGAGAGTTGGGATTCATCCCCATGTCATGGTGGGCTGAGCCCACATGGAAGCCTGT
GCTTGGACAGCGTATGCCCTTTTCTCTGTTTTTCCACAATGAACAATTAAACTGTAAATGTTAAAAATAT
CAGTAATTTGTGAAATAAATTTTATTCTCATTTGAGCAACATAAA
>NM_014339.6 Homo sapiens interleukin 17 receptor A (IL17RA), transcript
variant 1, mRNA
(SEQ ID NO: 37)
CTGGGCCCGGGCTGGAAGCCGGAAGCGAGCAAAGTGGAGCCGACTCGAACTCCACCGCGGAAAAGAAAGC
CTCAGAACGTTCGTTCGCTGCGTCCCCAGCCGGGGCCGAGCCCTCCGCGACGCCAGCCGGGCCATGGGGG
CCGCACGCAGCCCGCCGTCCGCTGTCCCGGGGCCCCTGCTGGGGCTGCTCCTGCTGCTCCTGGGCGTGCT
GGCCCCGGGTGGCGCCTCCCTGCGACTCCTGGACCACCGGGCGCTGGTCTGCTCCCAGCCGGGGCTAAAC
TGCACGGTCAAGAATAGTACCTGCCTGGATGACAGCTGGATTCACCCTCGAAACCTGACCCCCTCCTCCC
CAAAGGACCTGCAGATCCAGCTGCACTTTGCCCACACCCAACAAGGAGACCTGTTCCCCGTGGCTCACAT
CGAATGGACACTGCAGACAGACGCCAGCATCCTGTACCTCGAGGGTGCAGAGTTATCTGTCCTGCAGCTG
AACACCAATGAACGTTTGTGCGTCAGGTTTGAGTTTCTGTCCAAACTGAGGCATCACCACAGGCGGTGGC
GTTTTACCTTCAGCCACTTTGTGGTTGACCCTGACCAGGAATATGAGGTGACCGTTCACCACCTGCCCAA
GCCCATCCCTGATGGGGACCCAAACCACCAGTCCAAGAATTTCCTTGTGCCTGACTGTGAGCACGCCAGG
ATGAAGGTAACCACGCCATGCATGAGCTCAGGCAGCCTGTGGGACCCCAACATCACCGTGGAGACCCTGG
AGGCCCACCAGCTGCGTGTGAGCTTCACCCTGTGGAACGAATCTACCCATTACCAGATCCTGCTGACCAG
TTTTCCGCACATGGAGAACCACAGTTGCTTTGAGCACATGCACCACATACCTGCGCCCAGACCAGAAGAG
TTCCACCAGCGATCCAACGTCACACTCACTCTACGCAACCTTAAAGGGTGCTGTCGCCACCAAGTGCAGA
TCCAGCCCTTCTTCAGCAGCTGCCTCAATGACTGCCTCAGACACTCCGCGACTGTTTCCTGCCCAGAAAT
GCCAGACACTCCAGAACCAATTCCGGACTACATGCCCCTGTGGGTGTACTGGTTCATCACGGGCATCTCC
ATCCTGCTGGTGGGCTCCGTCATCCTGCTCATCGTCTGCATGACCTGGAGGCTAGCTGGGCCTGGAAGTG
AAAAATACAGTGATGACACCAAATACACCGATGGCCTGCCTGCGGCTGACCTGATCCCCCCACCGCTGAA
GCCCAGGAAGGTCTGGATCATCTACTCAGCCGACCACCCCCTCTACGTGGACGTGGTCCTGAAATTCGCC
CAGTTCCTGCTCACCGCCTGCGGCACGGAAGTGGCCCTGGACCTGCTGGAAGAGCAGGCCATCTCGGAGG
CAGGAGTCATGACCTGGGTGGGCCGTCAGAAGCAGGAGATGGTGGAGAGCAACTCTAAGATCATCGTCCT
GTGCTCCCGCGGCACGCGCGCCAAGTGGCAGGCGCTCCTGGGCCGGGGGGCGCCTGTGCGGCTGCGCTGC
GACCACGGAAAGCCCGTGGGGGACCTGTTCACTGCAGCCATGAACATGATCCTCCCGGACTTCAAGAGGC
CAGCCTGCTTCGGCACCTACGTAGTCTGCTACTTCAGCGAGGTCAGCTGTGACGGCGACGTCCCCGACCT
GTTCGGCGCGGCGCCGCGGTACCCGCTCATGGACAGGTTCGAGGAGGTGTACTTCCGCATCCAGGACCTG
GAGATGTTCCAGCCGGGCCGCATGCACCGCGTAGGGGAGCTGTCGGGGGACAACTACCTGCGGAGCCCGG
GCGGCAGGCAGCTCCGCGCCGCCCTGGACAGGTTCCGGGACTGGCAGGTCCGCTGTCCCGACTGGTTCGA
ATGTGAGAACCTCTACTCAGCAGATGACCAGGATGCCCCGTCCCTGGACGAAGAGGTGTTTGAGGAGCCA
CTGCTGCCTCCGGGAACCGGCATCGTGAAGCGGGCGCCCCTGGTGCGCGAGCCTGGCTCCCAGGCCTGCC
TGGCCATAGACCCGCTGGTCGGGGAGGAAGGAGGAGCAGCAGTGGCAAAGCTGGAACCTCACCTGCAGCC
CCGGGGTCAGCCAGCGCCGCAGCCCCTCCACACCCTGGTGCTCGCCGCAGAGGAGGGGGCCCTGGTGGCC
GCGGTGGAGCCTGGGCCCCTGGCTGACGGTGCCGCAGTCCGGCTGGCACTGGCGGGGGAGGGCGAGGCCT
GCCCGCTGCTGGGCAGCCCGGGCGCTGGGCGAAATAGCGTCCTCTTCCTCCCCGTGGACCCCGAGGACTC
GCCCCTTGGCAGCAGCACCCCCATGGCGTCTCCTGACCTCCTTCCAGAGGACGTGAGGGAGCACCTCGAA
GGCTTGATGCTCTCGCTCTTCGAGCAGAGTCTGAGCTGCCAGGCCCAGGGGGGCTGCAGTAGACCCGCCA
TGGTCCTCACAGACCCACACACGCCCTACGAGGAGGAGCAGCGGCAGTCAGTGCAGTCTGACCAGGGCTA
CATCTCCAGGAGCTCCCCGCAGCCCCCCGAGGGACTCACGGAAATGGAGGAAGAGGAGGAAGAGGAGCAG
GACCCAGGGAAGCCGGCCCTGCCACTCTCTCCCGAGGACCTGGAGAGCCTGAGGAGCCTCCAGCGGCAGC
TGCTTTTCCGCCAGCTGCAGAAGAACTCGGGCTGGGACACGATGGGGTCAGAGTCAGAGGGGCCCAGTGC
ATGAGGGCGGCTCCCCAGGGACCGCCCAGATCCCAGCTTTGAGAGAGGAGTGTGTGTGCACGTATTCATC
TGTGTGTACATGTCTGCATGTGTATATGTTCGTGTGTGAAATGTAGGCTTTAAAATGTAAATGTCTGGAT
TTTAATCCCAGGCATCCCTCCTAACTTTTCTTTGTGCAGCGGTCTGGTTATCGTCTATCCCCAGGGGAAT
CCACACAGCCCGCTCCCAGGAGCTAATGGTAGAGCGTCCTTGAGGCTCCATTATTCGTTCATTCAGCATT
TATTGTGCACCTACTATGTGGCGGGCATTTGGGATACCAAGATAAATTGCATGCGGCATGGCCCCAGCCA
TGAAGGAACTTAACCGCTAGTGCCGAGGACACGTTAAACGAACAGGATGGGCCGGGCACGGTGGCTCACG
CCTGTAATCCCAGCACACTGGGAGGCCGAGGCAGGTGGATCACTCTGAGGTCAGGAGTTTGAGCCAGCCT
GGCCAACATGGTGAAACCCCATCTCCACTAAAAATAGAAAAATTAGCCGGGCATGGTGACACATGCCTGT
AGTCCTAGCTACTTGGGAGGCTGAGGCAGGAGAATTGCTTGAATCTGGGAGGCAGAGGTTGCAGTGAGCC
GAGATTGTGCCATTGCACTGCAGCCTGGATGACAGAGCGAGACTCTATCTCAAAAAAAAAAAAAAAAAAA
GATGGTCACGCGGGATGTAAACGCTGAATGGGCCAGGTGCAGTGGCTCATGCTTGTAATCCCAGCACTTT
GGGAAGGCGAGGCAGGTGGATTGCTTGAGCTCAGGAGTTCAAGACCAGCCTGGGCGACATAGTGAGACCT
CATCTCTACCTAAATTTTTTTTTAGTCAGTCATGGTGGCACATGCCTGTAGTCCCAGCTACTCGGGAGGC
TGATGCCAGATGATCACTTGAGCCCAGGAGGTAGAGGCTGCAGTGAGCTATAATGGTACCATTGCAATCC
AGCCTGGGCAGCAGAGTGAGACCCTGTCTCAAAAAAAATAAAAAAGTAGAAAGATGGAGTGGAAGCCTGC
CCAGGGTTGTGAGCATGCACGGGAAAGGCACCCAGGTCAGGGGGGATCCCCGAGGAGATGCCTGAGCTGA
AGGATTGTGGTTGGGGAAAGCGTAGTCCCAGCAAGGAAGCAGTTTGTGGGTAAGTGCTGGGAGGTGAGTG
GAGTGAGCTTGTCAGGGAGCTGCTGGTGGAGCCTGGAGGGGAAGGAGGGAGGCAGTGAGAGAGATCGGGG
TGGGGGGTGGGGGGATGTCGCCAGAGCTCAGGGGTGGGGACAGCCTTGTGCGCATCAGTCCTGAGGCCTG
GGGCACCTTTCGTCTGATGAGCCTCTGCATGGAGAGAGGCTGAGGGCTAAACACAGCTGGATGTCACCTG
AGTTCATTTATAGGAAGAGAGAAATGTCGAGGTGAAACGTAAAAGCATCTGGCAGGAAGGTGAGTCTGAA
GCCCTGCACCCGCGTTCCGACTATCAGTGGGGAGCTGTTAGCACGTAGGATTCTTCAGAGCAGCTGGGCT
GGAGCTCCCCTGAGCTCAGGAAGCCCCAGGGTGCAAGGGCAAGGAAATGAGGGGTGGTGGGTCAGTGAAG
ATCTGGGCAGACCTTGTGTGGGGAAGGGGTGCTGCTGTGACTTCAGGGTCTGAGGTCCAAAGACAGCATT
TGAAAAGAGGCTCTGAAGCCAGTGTTTGAAGAATTTGTTCCTGAAGTACCTCCTGGGGGTAGGCTAGAGG
CTTCTGGCTTCAGGGTCCTGAAGAACACATTGAGGTGCCGTCTGACACTGGAATAGGGTGCCCTTCATTC
CTATGCCTGAGTCCTTAACTATATTTCCAACCTCCAGTGAGGAGGAGAAGATTCGGAAATGTGACAGGAG
AGCAAACAGGACAGTTTGCATGTGTGTGTGCGCACACATACATGTGCGTGAAAGATTATCAATAAAAGTG
CATAAATTTGTTGATCTGGTAAGAGTTTCTAGCAGGAAGGTCGAGCCACTTACTGTAGGTCAAGAAGTTG
CTAGTTGCGGAGTTTTTTCTTGCAGTTAGACTTTACCTAGTGGTAGCAGGGCCACCAAAGCTCTGTGTCC
CAGATGGTGTATGGCCCATAATCCACCCAACAGCAGCAAAGGACCAGGCAAAGGAGAACAGGAGCAGAAG
CCTCCCAGCCACTAGCCTTTTGGGCTCAGTCTCTCCAATAATCCTGGAGAGGGGCTTCGTTGGGTCTGGA
CACCTACCATGCATTCTGTGACCTTTCCCTAGCTTCCAATAAATAACTGTTTGACGCCCAGAGTACAGGA
TACCACAATGCACTCTTCCTGCGTAGAGCACATGTTCCCATCTGCTCCCATTCCTCAGGAACCTTGAATT
CTAGCTCTGCTGGCCTTTGAGCCCATGCCAGTAAATGTCCTGATGGGCATTGCCTACTATCTCCAGGGCA
GCTGCCTTTGTCCTCCTAACAGCTTTATTGGAGTACAGTTCACTTACCATACAATCCACAATTGACCCTG
CACAATTTGATGCCGGTTTAGTATAGTCACAGTTCAGCAGCCATCAGCACAGTCAGTCTTAGAGTTTACT
ACCCCCAAAAGAAATCCAGCCCCCCTTAGTCACCACCCCAACCTCCCCATCCCTAGGCACCCCTAGGCTA
CTTTGATCTCTGTAGACTTGCCTCTTCTGGACATGACATAGAGAAAGGAGTCATAAATTCTCCAAGGTGT
CTGTTTCTTCTTTAATGTCATTCCCTGTTTCTCCTCACATTCCCTCCCCATTTCCTGGGCCCAGTCTCAC
ACTGGTCCTTGCTTACCCTAAATGCTATTAATTCCATCACTCTGAGTATGGTGTTTGCTGTCCGCTGAAT
GCCAAGAGCTTCAAGAGTGTGTGTAAATAAAGCCACACCTTTATTTTTGTATTATTCTGAACCATGGCTA
ATAAATTGTTTCACCAAGAAATGTCTCTCTAAGAACAGGTGCCCTCCACGCTGTGCCCCTCCCACCTCTT
CAGCTCGTCTCCTGAGTGTGCAGAGGTGGTTCCGGTTGGGAAAGAAGCAGCGGAGCATCTAACCATGCCT
GTGTCCAGGCCGATTATGCACGCAGCCACCAACAAGCTCCCAACTCCCGCGTAGAGTTTCATGACTTTTT
CCTGCCTACTATCTTGATCCTAGTTTTTTTTTTGTTTTTTTTTTTTTTAAGGAATAATTACTTTGATTCA
AAACCAGTTTCTCTTTTCTGCATAGGAAGGTCCTTGAAGGTGTTTAGGGTCTAAAAAGGGTGGTGTTCGG
TCTCTGAAACATCCATTCAGCAGTTTGAGCTGGGATCTCTGAATGCAAGGGTATGATGGATATACTTCTT
TCTTGCTTTTGTTGTGTTTTGGTTTTTTGTTTGTTTTTAAGTCAGGGTCTCTCTGTCACCAGGCTGTATT
ACAGTGGTGCAATCATGGCTCACTGCAGCCTCGACCTCCCAGGCTCAAGCCATCTTTCCACCTCAGCCTG
CCAGTGGCTAGAACTACAGGCGTGCACCACTGTGCCCGGCTAATTTGTGTGTATATATTTTGTAGAAATG
GGGTTTCACCATGTTGTCCAGGCTGGTCACGAACTCCTGGGCTCAAGCCATCTGCCCGCCTCATCCTCCC
AAAGTGCTGGGATTATAGGCCTGAGCCCACCGTGCCTGGCCTTTCCTGTTTATCTTTGAAAATTAAATAG
GGCATAAGAGAGAAGAAGATGTACTTACAATGCAGTGGGTGGTTTTAACTCTATAGCCTTTGGGCTCTGT
GGTTGGTGCTCCCCTTCCTAAATAAATGAGGTGTATGCAGGGCCCTCTTCTGCCTTAGCGCCCTGCCAGC
TGGGACTCCAGCAAGGCCCGGGGCACCTGAGGACAGAGTGAGATGGAGGGCCGCTGCTCCAGCAGCCGGG
CCTGCATCCCACAAGTCAACTGTGTCGGACAGAGGATCCTTACAAAGAAGAGGCAGCAGGGTTGGGGGCT
GGCCAGCTGCTCGTCCGCCCTAGGTAGCTTGCTCATCTGTAAAGTGGGTGGGGCAGGAGTTCCCACCTCA
TGGGGTCCTGGCAAGCCTGCAGTATCCCCGAGTGGCACCAGCCTGCTTCTGGGGCAGAGCAGTTTGTGCC
CCCTGAGGTACCACTGATCCTCTTTCCCTGCTATTAGGTATTGCTCTCTTCCTCCGGTGTTTGCCTTTTC
AGATTATAGAAGTAATATGTGTTCCCATATTTGGCGTCTCTCAGGAGCTCAGGAAGTACTTGGCTGAGTG
AACATGTCCATTGTGGAAAAATGGCAACAATATGGATTCCATGGGTATATTTTATAGAAGAATATGAAGA
AAAGCAGCTACCCCTAAACCCATTGCACAAGCTGTTCATGTTAATTCTGTACCCGACGCTTTCCCCACGG
GGCCTCCCCTCACTCTGAAATGGCATCCAGGTCCATCTTGCCCTCCACCTCTGCATGGCTCTCCATGCCC
CATCGCCTCTCCCAGATCCTAGCACTGGGTCCACACTCTCGCCCTGTCCATTTAGGTTGATGAAAGCAGG
CAGTCACCCGGGTGGGCCAGTCTTGCCTGTGGGAGGAACATGCAGTCTCCTGTCTCATGGTTTGAAGTGT
GCCAGGAAGCCTGGCCCAGCCCACCTCCCCCTGGAGTCCTTCCCAGGAGGAATAACCCCTTAGGTCATTG
ACTATAAGATGAGTTCGCTCACTGGATCCTTCCTCTCTGATGAGACAGGAAGAAGGTACACAGTGACCAG
GTAGGAGGAGGAGAGGGAGTAGAAAGGAGGGATGCGGGTGGCTGGTCCCTGCATTTGCCTGCTTCCCTGC
ACGGGTGTCCCACTGGCCGCCTCTGCTCACCAGTGTCATGGGATTCTCTCAGAAGATGAAAACAGCCCCT
GCTTTTTTGCTAGAATGGCTGAGCTTTCATGGAAAGGAAGCTGGACCCAAGCAACAGCCGACTACCGAAG
GTTGCCTGGAGCAGTGCAGATGTGGGAGGAAGAAGGGCCTTGGTGCACACTGGCTTTTCTTCCTGACTGC
AATGTGGCATTGTGCCAGCTACCTCCTCTTTCTCGGCCTCAGGAAAATGGAGAGAAAGCAGCCCTGAAGG
TGGCTGTGACGAGGGAAGGGGCAGAGGGCCTGACAGTCAACCACGCGCTATATTTTCCTGTTCTTCCTTA
GGGCAAGAACTGCATGGCCAGACTCAGGCAAGGCCTAGGTGTGGGCTGGGCATTGCCTACACGTGAAGAG
ATCACTCCGCGTCCCTACTGCACCTGTCACAAAGTGCCTTCTGATATGCCTGGCAAACCAAAATCGGTGA
GCGCCAGCTTGCTTCCCTAGAAGACATTTCTAAATATTCATAACATGCTTGCTCAAATCAATCACCTTAT
TTTACATCCGCTCCAGGGAGAAATGAAGACATGGTCCTACGTTGTTCTGTAATTATTTTCTATGTAAATT
TTGTTCCTTGTTACAATTATATATGTCTTAGGGGAAAGGACCATTTCACATGTGTCACCTCATGTGATTC
TCACCACAGCCCTGTGATTGCTCCTGTTTTATAAATAATGACATAGTTCCAGTTGATGGCCAAAGCCACA
GCTAACGAGAGGCAGAGAGAGCTCAGGCTCCCAGGAGCTTCCACTCTCAGACCTTGCCTCCCGGGCTGCC
CTGAGTGAAACGCCTGCTTAGCATTTGGCACAGCCAGAAGCAGCAAGCTAGGGTCACAACACAGAGAGGG
GCTGTGTAATACTGGCTGCCTCTGTGCTAAGAAAAAAAAAAAATCACTGTGTGTTTGTTTATTTTGGTGC
AGGCCCAGTGTTCTTGCTTAGACTTAATACTACCCTTCATGTTAAAATAAAACCAAACAAAAACCCAT
>NM_000877.4 Homo sapiens interleukin 1 receptor type 1 (IL1R1),
transcript variant 1, mRNA
(SEQ ID NO: 38)
AGTTCCCGGCCGCGAGGGCGGGCGCAGCTTGTGGCCGGCGGCCGGAGCCGACTCGGAGCGCGCGGCGCCG
GCCGGGAGGAGCCGGAGAGCGGCCGGGCCGGGCGGTGGGGGCGCCGGCCTGCCCCGCGCGCCCCAGGGAG
CGGCAGGAATGTGACAATCGCGCGCCCGCGCACCGAAGCACTCCTCGCTCGGCTCCTAGGGCTCTCGCCC
CTCTGAGCTGAGCCGGGTTCCGCCCGGGGCTGGGATCCCATCACCCTCCACGGCCGTCCGTCCAGGTAGA
CGCACCCTCTGAAGATGGTGACTCCCTCCTGAGAAGCTGGACCCCTTGGTAAAAGACAAGGCCTTCTCCA
AGAAGAATATGAAAGTGTTACTCAGACTTATTTGTTTCATAGCTCTACTGATTTCTTCTCTGGAGGCTGA
TAAATGCAAGGAACGTGAAGAAAAAATAATTTTAGTGTCATCTGCAAATGAAATTGATGTTCGTCCCTGT
CCTCTTAACCCAAATGAACACAAAGGCACTATAACTTGGTATAAAGATGACAGCAAGACACCTGTATCTA
CAGAACAAGCCTCCAGGATTCATCAACACAAAGAGAAACTTTGGTTTGTTCCTGCTAAGGTGGAGGATTC
AGGACATTACTATTGCGTGGTAAGAAATTCATCTTACTGCCTCAGAATTAAAATAAGTGCAAAATTTGTG
GAGAATGAGCCTAACTTATGTTATAATGCACAAGCCATATTTAAGCAGAAACTACCCGTTGCAGGAGACG
GAGGACTTGTGTGCCCTTATATGGAGTTTTTTAAAAATGAAAATAATGAGTTACCTAAATTACAGTGGTA
TAAGGATTGCAAACCTCTACTTCTTGACAATATACACTTTAGTGGAGTCAAAGATAGGCTCATCGTGATG
AATGTGGCTGAAAAGCATAGAGGGAACTATACTTGTCATGCATCCTACACATACTTGGGCAAGCAATATC
CTATTACCCGGGTAATAGAATTTATTACTCTAGAGGAAAACAAACCCACAAGGCCTGTGATTGTGAGCCC
AGCTAATGAGACAATGGAAGTAGACTTGGGATCCCAGATACAATTGATCTGTAATGTCACCGGCCAGTTG
AGTGACATTGCTTACTGGAAGTGGAATGGGTCAGTAATTGATGAAGATGACCCAGTGCTAGGGGAAGACT
ATTACAGTGTGGAAAATCCTGCAAACAAAAGAAGGAGTACCCTCATCACAGTGCTTAATATATCGGAAAT
TGAAAGTAGATTTTATAAACATCCATTTACCTGTTTTGCCAAGAATACACATGGTATAGATGCAGCATAT
ATCCAGTTAATATATCCAGTCACTAATTTCCAGAAGCACATGATTGGTATATGTGTCACGTTGACAGTCA
TAATTGTGTGTTCTGTTTTCATCTATAAAATCTTCAAGATTGACATTGTGCTTTGGTACAGGGATTCCTG
CTATGATTTTCTCCCAATAAAAGCTTCAGATGGAAAGACCTATGACGCATATATACTGTATCCAAAGACT
GTTGGGGAAGGGTCTACCTCTGACTGTGATATTTTTGTGTTTAAAGTCTTGCCTGAGGTCTTGGAAAAAC
AGTGTGGATATAAGCTGTTCATTTATGGAAGGGATGACTACGTTGGGGAAGACATTGTTGAGGTCATTAA
TGAAAACGTAAAGAAAAGCAGAAGACTGATTATCATTTTAGTCAGAGAAACATCAGGCTTCAGCTGGCTG
GGTGGTTCATCTGAAGAGCAAATAGCCATGTATAATGCTCTTGTTCAGGATGGAATTAAAGTTGTCCTGC
TTGAGCTGGAGAAAATCCAAGACTATGAGAAAATGCCAGAATCGATTAAATTCATTAAGCAGAAACATGG
GGCTATCCGCTGGTCAGGGGACTTTACACAGGGACCACAGTCTGCAAAGACAAGGTTCTGGAAGAATGTC
AGGTACCACATGCCAGTCCAGCGACGGTCACCTTCATCTAAACACCAGTTACTGTCACCAGCCACTAAGG
AGAAACTGCAAAGAGAGGCTCACGTGCCTCTCGGGTAGCATGGAGAAGTTGCCAAGAGTTCTTTAGGTGC
CTCCTGTCTTATGGCGTTGCAGGCCAGGTTATGCCTCATGCTGACTTGCAGAGTTCATGGAATGTAACTA
TATCATCCTTTATCCCTGAGGTCACCTGGAATCAGATTATTAAGGGAATAAGCCATGACGTCAATAGCAG
CCCAGGGCACTTCAGAGTAGAGGGCTTGGGAAGATCTTTTAAAAAGGCAGTAGGCCCGGTGTGGTGGCTC
ACGCCTATAATCCCAGCACTTTGGGAGGCTGAAGTGGGTGGATCACCAGAGGTCAGGAGTTCGAGACCAG
CCCAGCCAACATGGCAAAACCCCATCTCTACTAAAAATACAAAAATGAGCTAGGCATGGTGGCACACGCC
TGTAATCCCAGCTACACCTGAGGCTGAGGCAGGAGAATTGCTTGAACCGGGGAGACGGAGGTTGCAGTGA
GCCGAGTTTGGGCCACTGCACTCTAGCCTGGCAACAGAGCAAGACTCCGTCTCAAAAAAAGGGCAATAAA
TGCCCTCTCTGAATGTTTGAACTGCCAAGAAAAGGCATGGAGACAGCGAACTAGAAGAAAGGGCAAGAAG
GAAATAGCCACCGTCTACAGATGGCTTAGTTAAGTCATCCACAGCCCAAGGGCGGGGCTATGCCTTGTCT
GGGGACCCTGTAGAGTCACTGACCCTGGAGCGGCTCTCCTGAGAGGTGCTGCAGGCAAAGTGAGACTGAC
ACCTCACTGAGGAAGGGAGACATATTCTTGGAGAACTTTCCATCTGCTTGTATTTTCCATACACATCCCC
AGCCAGAAGTTAGTGTCCGAAGACCGAATTTTATTTTACAGAGCTTGAAAACTCACTTCAATGAACAAAG
GGATTCTCCAGGATTCCAAAGTTTTGAAGTCATCTTAGCTTTCCACAGGAGGGAGAGAACTTAAAAAAGC
AACAGTAGCAGGGAATTGATCCACTTCTTAATGCTTTCCTCCCTGGCATGACCATCCTGTCCTTTGTTAT
TATCCTGCATTTTACGTCTTTGGAGGAACAGCTCCCTAGTGGCTTCCTCCGTCTGCAATGTCCCTTGCAC
AGCCCACACATGAACCATCCTTCCCATGATGCCGCTCTTCTGTCATCCCGCTCCTGCTGAAACACCTCCC
AGGGGCTCCACCTGTTCAGGAGCTGAAGCCCATGCTTTCCCACCAGCATGTCACTCCCAGACCACCTCCC
TGCCCTGTCCTCCAGCTTCCCCTCGCTGTCCTGCTGTGTGAATTCCCAGGTTGGCCTGGTGGCCATGTCG
CCTGCCCCCAGCACTCCTCTGTCTCTGCTCTTGCCTGCACCCTTCCTCCTCCTTTGCCTAGGAGGCCTTC
TCGCATTTTCTCTAGCTGATCAGAATTTTACCAAAATTCAGAACATCCTCCAATTCCACAGTCTCTGGGA
GACTTTCCCTAAGAGGCGACTTCCTCTCCAGCCTTCTCTCTCTGGTCAGGCCCACTGCAGAGATGGTGGT
GAGCACATCTGGGAGGCTGGTCTCCCTCCAGCTGGAATTGCTGCTCTCTGAGGGAGAGGCTGTGGTGGCT
GTCTCTGTCCCTCACTGCCTTCCAGGAGCAATTTGCACATGTAACATAGATTTATGTAATGCTTTATGTT
TAAAAACATTCCCCAATTATCTTATTTAATTTTTGCAATTATTCTAATTTTATATATAGAGAAAGTGACC
TATTTTTTAAAAAAATCACACTCTAAGTTCTATTGAACCTAGGACTTGAGCCTCCATTTCTGGCTTCTAG
TCTGGTGTTCTGAGTACTTGATTTCAGGTCAATAACGGTCCCCCCTCACTCCACACTGGCACGTTTGTGA
GAAGAAATGACATTTTGCTAGGAAGTGACCGAGTCTAGGAATGCTTTTATTCAAGACACCAAATTCCAAA
CTTCTAAATGTTGGAATTTTCAAAAATTGTGTTTAGATTTTATGAAAAACTCTTCTACTTTCATCTATTC
TTTCCCTAGAGGCAAACATTTCTTAAAATGTTTCATTTTCATTAAAAATGAAAGCCAAATTTATATGCCA
CCGATTGCAGGACACAAGCACAGTTTTAAGAGTTGTATGAACATGGAGAGGACTTTTGGTTTTTATATTT
CTCGTATTTAATATGGGTGAACACCAACTTTTATTTGGAATAATAATTTTCCTCCTAAACAAAAACACAT
TGAGTTTAAGTCTCTGACTCTTGCCTTTCCACCTGCTTTCTCCTGGGCCCGCTTTGCCTGCTTGAAGGAA
CAGTGCTGTTCTGGAGCTGCTGTTCCAACAGACAGGGCCTAGCTTTCATTTGACACACAGACTACAGCCA
GAAGCCCATGGAGCAGGGATGTCACGTCTTGAAAAGCCTATTAGATGTTTTACAAATTTAATTTTGCAGA
TTATTTTAGTCTGTCATCCAGAAAATGTGTCAGCATGCATAGTGCTAAGAAAGCAAGCCAATTTGGAAAC
TTAGGTTAGTGACAAAATTGGCCAGAGAGTGGGGGTGATGATGACCAAGAATTACAAGTAGAATGGCAGC
TGGAATTTAAGGAGGGACAAGAATCAATGGATAAGCGTGGGTGGAGGAAGATCCAAACAGAAAAGTGCAA
AGTTATTCCCCATCTTCCAAGGGTTGAATTCTGGAGGAAGAAGACACATTCCTAGTTCCCCGTGAACTTC
CTTTGACTTATTGTCCCCACTAAAACAAAACAAAAAACTTTTAATGCCTTCCACATTAATTAGATTTTCT
TGCAGTTTTTTTATGGCATTTTTTTAAAGATGCCCTAAGTGTTGAAGAAGAGTTTGCAAATGCAACAAAA
TATTTAATTACCGGTTGTTAAAACTGGTTTAGCACAATTTATATTTTCCCTCTCTTGCCTTTCTTATTTG
CAATAAAAGGTATTGAGCCATTTTTTAAATGACATTTTTGATAAATTATGTTTGTACTAGTTGATGAAGG
AGTTTTTTTTAACCTGTTTATATAATTTTGCAGCAGAAGCCAAATTTTTTGTATATTAAAGCACCAAATT
CATGTACAGCATGCATCACGGATCAATAGACTGTACTTATTTTCCAATAAAATTTTCAAACTTTGTACTG
TTA
>NM_022789.3 Homo sapiens interleukin 25 (IL25), transcript variant 1,
mRNA
(SEQ ID NO: 46)
GGCTTGCTGAAAATAAAATCAGGACTCCTAACCTGCTCCAGTCAGCCTGCTTCCACGAGGCCTGTCAGTC
AGTGCCCCACTTGTGACTGAGTGTGCAGTGCCCAGCATGTACCAGGTCAGTGCAGAGGGCTGCCTGAGGG
CTGTGCTGAGAGGGAGAGGAGCAGAGATGCTGCTGAGGGTGGAGGGAGGCCAAGCTGCCAGGTTTGGGGC
TGGGGGCCAAGTGGAGTGAGAAACTGGGATCCCAGGGGGAGGGTGCAGATGAGGGAGCGACCCAGATTAG
GTGAGGACAGTTCTCTCATTAGCCTTTTCCTACAGGTGGTTGCATTCTTGGCAATGGTCATGGGAACCCA
CACCTACAGCCACTGGCCCAGCTGCTGCCCCAGCAAAGGGCAGGACACCTCTGAGGAGCTGCTGAGGTGG
AGCACTGTGCCTGTGCCTCCCCTAGAGCCTGCTAGGCCCAACCGCCACCCAGAGTCCTGTAGGGCCAGTG
AAGATGGACCCCTCAACAGCAGGGCCATCTCCCCCTGGAGATATGAGTTGGACAGAGACTTGAACCGGCT
CCCCCAGGACCTGTACCACGCCCGTTGCCTGTGCCCGCACTGCGTCAGCCTACAGACAGGCTCCCACATG
GACCCCCGGGGCAACTCGGAGCTGCTCTACCACAACCAGACTGTCTTCTACCGGCGGCCATGCCATGGCG
AGAAGGGCACCCACAAGGGCTACTGCCTGGAGCGCAGGCTGTACCGTGTTTCCTTAGCTTGTGTGTGTGT
GCGGCCCCGTGTGATGGGCTAGCCGGACCTGCTGGAGGCTGGTCCCTTTTTGGGAAACCTGGAGCCAGGT
GTACAACCACTTGCCATGAAGGGCCAGGATGCCCAGATGCTTGGCCCCTGTGAAGTGCTGTCTGGAGCAG
CAGGATCCCGGGACAGGATGGGGGGCTTTGGGGAAAGCCTGCACTTCTGCACATTTTGAAAAGAGCAGCT
GCTGCTTAGGGCCGCCGGAAGCTGGTGTCCTGTCATTTTCTCTCAGGAAAGGTTTTCAAAGTTCTGCCCA
TTTCTGGAGGCCACCACTCCTGTCTCTTCCTCTTTTCCCATCCCCTGCTACCCTGGCCCAGCACAGGCAC
TTTCTAGATATTTCCCCCTTGCTGGAGAAGAAAGAGCCCCTGGTTTTATTTGTTTGTTTACTCATCACTC
AGTGAGCATCTACTTTGGGTGCATTCTAGTGTAGTTACTAGTCTTTTGACATGGATGATTCTGAGGAGGA
AGCTGTTATTGAATGTATAGAGATTTATCCAAATAAATATCTTTATTTAAAAATGAAAAAAAAAAAAAAA
AAAAA
>NM_139017.6 Homo sapiens interleukin 31 receptor A (IL31RA), transcript
variant 1, mRNA
(SEQ ID NO: 47)
ACAGCCTCCTTCTGCTTAGGAACACCAGACAGCACTCCAGCACTCTGCTTGGGGGGCATTCGAAACAGCA
AAATCACTCATAAAAGGCAAAAAATTGCAAAAAAAAATAGTAATAACCAGCATGGCACTAAATAGACCAT
GAAAAGACATGTGTGTGCAGTATGAAAATTGAGACAGGAAGGCAGAGTGTCAGCTTGTTCCACCTCAGCT
GGGAATGTGCATCAGGCAACTCAAGTTTTTCACCACGGCATGTGTCTGTGAATGTCCGCAAAACATTCTC
TCTCCCCAGCCTTCATGTGTTAACCTGGGGATGATGTGGACCTGGGCACTGTGGATGCTCCCCTCACTCT
GCAAATTCAGCCTGGCAGCTCTGCCAGCTAAGCCTGAGAACATTTCCTGTGTCTACTACTATAGGAAAAA
TTTAACCTGCACTTGGAGTCCAGGAAAGGAAACCAGTTATACCCAGTACACAGTTAAGAGAACTTACGCT
TTTGGAGAAAAACATGATAATTGTACAACCAATAGTTCTACAAGTGAAAATCGTGCTTCGTGCTCTTTTT
TCCTTCCAAGAATAACGATCCCAGATAATTATACCATTGAGGTGGAAGCTGAAAATGGAGATGGTGTAAT
TAAATCTCATATGACATACTGGAGATTAGAGAACATAGCGAAAACTGAACCACCTAAGATTTTCCGTGTG
AAACCAGTTTTGGGCATCAAACGAATGATTCAAATTGAATGGATAAAGCCTGAGTTGGCGCCTGTTTCAT
CTGATTTAAAATACACACTTCGATTCAGGACAGTCAACAGTACCAGCTGGATGGAAGTCAACTTCGCTAA
GAACCGTAAGGATAAAAACCAAACGTACAACCTCACGGGGCTGCAGCCTTTTACAGAATATGTCATAGCT
CTGCGATGTGCGGTCAAGGAGTCAAAGTTCTGGAGTGACTGGAGCCAAGAAAAAATGGGAATGACTGAGG
AAGAAGCTCCATGTGGCCTGGAACTGTGGAGAGTCCTGAAACCAGCTGAGGCGGATGGAAGAAGGCCAGT
GCGGTTGTTATGGAAGAAGGCAAGAGGAGCCCCAGTCCTAGAGAAAACACTTGGCTACAACATATGGTAC
TATCCAGAAAGCAACACTAACCTCACAGAAACAATGAACACTACTAACCAGCAGCTTGAACTGCATCTGG
GAGGCGAGAGCTTTTGGGTGTCTATGATTTCTTATAATTCTCTTGGGAAGTCTCCAGTGGCCACCCTGAG
GATTCCAGCTATTCAAGAAAAATCATTTCAGTGCATTGAGGTCATGCAGGCCTGCGTTGCTGAGGACCAG
CTAGTGGTGAAGTGGCAAAGCTCTGCTCTAGACGTGAACACTTGGATGATTGAATGGTTTCCGGATGTGG
ACTCAGAGCCCACCACCCTTTCCTGGGAATCTGTGTCTCAGGCCACGAACTGGACGATCCAGCAAGATAA
ATTAAAACCTTTCTGGTGCTATAACATCTCTGTGTATCCAATGTTGCATGACAAAGTTGGCGAGCCATAT
TCCATCCAGGCTTATGCCAAAGAAGGCGTTCCATCAGAAGGTCCTGAGACCAAGGTGGAGAACATTGGCG
TGAAGACGGTCACGATCACATGGAAAGAGATTCCCAAGAGTGAGAGAAAGGGTATCATCTGCAACTACAC
CATCTTTTACCAAGCTGAAGGTGGAAAAGGATTCTCCAAGACAGTCAATTCCAGCATCTTGCAGTACGGC
CTGGAGTCCCTGAAACGAAAGACCTCTTACATTGTTCAGGTCATGGCCAGCACCAGTGCTGGGGGAACCA
ACGGGACCAGCATAAATTTCAAGACATTGTCATTCAGTGTCTTTGAGATTATCCTCATAACTTCTCTGAT
TGGTGGAGGCCTTCTTATTCTCATTATCCTGACAGTGGCATATGGTCTCAAAAAACCCAACAAATTGACT
CATCTGTGTTGGCCCACCGTTCCCAACCCTGCTGAAAGTAGTATAGCCACATGGCATGGAGATGATTTCA
AGGATAAGCTAAACCTGAAGGAGTCTGATGACTCTGTGAACACAGAAGACAGGATCTTAAAACCATGTTC
CACCCCCAGTGACAAGTTGGTGATTGACAAGTTGGTGGTGAACTTTGGGAATGTTCTGCAAGAAATTTTC
ACAGATGAAGCCAGAACGGGTCAGGAAAACAATTTAGGAGGGGAAAAGAATGGGTATGTGACCTGCCCCT
TCAGGCCTGATTGTCCCCTGGGGAAAAGTTTTGAGGAGCTCCCAGTTTCACCTGAGATTCCGCCCAGAAA
ATCCCAATACCTACGTTCGAGGATGCCAGAGGGGACCCGCCCAGAAGCCAAAGAGCAGCTTCTCTTTTCT
GGTCAAAGTTTAGTACCAGATCATCTGTGTGAGGAAGGAGCCCCAAATCCATATTTGAAAAATTCAGTGA
CAGCCAGGGAATTTCTTGTGTCTGAAAAACTTCCAGAGCACACCAAGGGAGAAGTCTAAATGCGACCATA
GCATGAGACCCTCGGGGCCTCAGTGTGGATGGCCCTTGCCAGAGAAGATGTCAAGACTCGGCACGCAGCG
CTTGCTTGGCCCTGCCACATCCTGCCTAGGTTAAAGTTTCCCCTGCCCCTTGAGCTGCCAGTTGAACTTG
GTCGGCAAAGATGCGACCTTGTACTGGGAAGAAGGGATGGTGATAAGCCCGAGTTTTGTAAAGGAA
>NM_153480.2 Homo sapiens interleukin 17 receptor E (IL17RE), transcript
variant 1, mRNA
(SEQ ID NO: 48)
GTGTTCGCTGCTGCACAGCAAGGCCCTGCCACCCACCTTCAGGCCATGCAGCCATGTTCCGGGAGCCCTA
ATTGCACAGAAGCCCATGGGGAGCTCCAGACTGGCAGCCCTGCTCCTGCCTCTCCTCCTCATAGTCATCG
ACCTCTCTGACTCTGCTGGGATTGGCTTTCGCCACCTGCCCCACTGGAACACCCGCTGTCCTCTGGCCTC
CCACACGGATGACAGTTTCACTGGAAGTTCTGCCTATATCCCTTGCCGCACCTGGTGGGCCCTCTTCTCC
ACAAAGCCTTGGTGTGTGCGAGTCTGGCACTGTTCCCGCTGTTTGTGCCAGCATCTGCTGTCAGGTGGCT
CAGGTCTTCAACGGGGCCTCTTCCACCTCCTGGTGCAGAAATCCAAAAAGTCTTCCACATTCAAGTTCTA
TAGGAGACACAAGATGCCAGCACCTGCTCAGAGGAAGCTGCTGCCTCGTCGTCACCTGTCTGAGAAGAGC
CATCACATTTCCATCCCCTCCCCAGACATCTCCCACAAGGGACTTCGCTCTAAAAGGACCCAACCTTCGG
ATCCAGAGACATGGGAAAGTCTTCCCAGATTGGACTCACAAAGGCATGGAGGACCCGAGTTCTCCTTTGA
TTTGCTGCCTGAGGCCCGGGCTATTCGGGTGACCATATCTTCAGGCCCTGAGGTCAGCGTGCGTCTTTGT
CACCAGTGGGCACTGGAGTGTGAAGAGCTGAGCAGTCCCTATGATGTCCAGAAAATTGTGTCTGGGGGCC
ACACTGTAGAGCTGCCTTATGAATTCCTTCTGCCCTGTCTGTGCATAGAGGCATCCTACCTGCAAGAGGA
CACTGTGAGGCGCAAAAAATGTCCCTTCCAGAGCTGGCCAGAAGCCTATGGCTCGGACTTCTGGAAGTCA
GTGCACTTCACTGACTACAGCCAGCACACTCAGATGGTCATGGCCCTGACACTCCGCTGCCCACTGAAGC
TGGAAGCTGCCCTCTGCCAGAGGCACGACTGGCATACCCTTTGCAAAGACCTCCCGAATGCCACAGCTCG
AGAGTCAGATGGGTGGTATGTTTTGGAGAAGGTGGACCTGCACCCCCAGCTCTGCTTCAAGTTCTCTTTT
GGAAACAGCAGCCATGTTGAATGCCCCCACCAGACTGGGTCTCTCACATCCTGGAATGTAAGCATGGATA
CCCAAGCCCAGCAGCTGATTCTTCACTTCTCCTCAAGAATGCATGCCACCTTCAGTGCTGCCTGGAGCCT
CCCAGGCTTGGGGCAGGACACTTTGGTGCCCCCCGTGTACACTGTCAGCCAGGCCCGGGGCTCAAGCCCA
GTGTCACTAGACCTCATCATTCCCTTCCTGAGGCCAGGGTGCTGTGTCCTGGTGTGGCGGTCAGATGTCC
AGTTTGCCTGGAAGCACCTCTTGTGTCCGGATGTCTCTTACAGACACCTGGGGCTCTTGATCCTGGCACT
GCTGGCCCTCCTCACCCTACTGGGTGTTGTTCTGGCCCTCACCTGCCGGCGCCCACAGTCAGGCCCGGGC
CCAGCGCGGCCAGTGCTCCTCCTGCACGCGGCGGACTCGGAGGCGCAGCGGCGCCTGGTGGGAGCGCTGG
CTGAACTGCTACGGGCAGCGCTGGGCGGCGGGCGCGACGTGATCGTGGACCTGTGGGAGGGGAGGCACGT
GGCGCGCGTGGGCCCGCTGCCGTGGCTCTGGGCGGCGCGGACGCGCGTAGCGCGGGAGCAGGGCACTGTG
CTGCTGCTGTGGAGCGGCGCCGACCTTCGCCCGGTCAGCGGCCCCGACCCCCGCGCCGCGCCCCTGCTCG
CCCTGCTCCACGCTGCCCCGCGCCCGCTGCTGCTGCTCGCTTACTTCAGTCGCCTCTGCGCCAAGGGCGA
CATCCCCCCGCCGCTGCGCGCCCTGCCGCGCTACCGCCTGCTGCGCGACCTGCCGCGTCTGCTGCGGGCG
CTGGACGCGCGGCCTTTCGCAGAGGCCACCAGCTGGGGCCGCCTTGGGGCGCGGCAGCGCAGGCAGAGCC
GCCTAGAGCTGTGCAGCCGGCTCGAACGAGAGGCCGCCCGACTTGCAGACCTAGGTTGAGCAGAGCTCCA
CCGCAGTCCCGGGTGTCTGCGGCCGCAACGCAACGGACACTGGCTGGAACCCCGGAATGAGCCTTCGACC
CTGAAATCCTTGGGGTGCCTCGAGGACGACTGGCCGAAAAGCCGCATTCCCTGCCTCACAGGCCGGAAGT
CCCAGCCCAGTCCCCGCGCGCGTCCCTCTTCCTCCTCATACTTTCCCTTGACTGAGAGCTCCTCTAACCC
CTGTTCTGATGGGGGAGGGCGGTCTTCCCACTTCCTCTCCAGAACTCCAGAAAGAGCAGTGTGCTTATGC
TTCAGTCCAGGCTGGAGAGGTTGGGGCCGGGGTAGGGAGGCAGGAGCCATGTCAGTTCTGAAGGAGGGTG
AGGCGGTGGGGGATTGCAGGGGGCGGCTGAGAGAAAACCTCCTTGGGGGCCAGGGATTCCCTTTCCCACT
CTGAGGCTCTGGCCAGAGGGAGAGAGGACTCTGGACCTAGGAAAAGAGGCTTTTGGCTCCAGGTGGTCAG
GACAGTGGGGGTTGGGGGTGGGGTGGGTGGGTGCTGGCGGTGGGGACCAAGATCCGGAAAGATGAATAAA
GACAAACATGACAAACTAA
>NM_003855.3 Homo sapiens interleukin 18 receptor 1 (IL18R1), transcript
variant 1, mRNA
(SEQ ID NO: 49)
TCAGGAGGCGGAGATCGCTGCTTCTCACCTACTTTCTGAACTTGGCCTCCGCAGTCGCGACCTGGCGTGA
AGGAGGAGCTGCCGCCCCCGCCCCAGCCTCGGGGACGCCTCTCTGAAGAGAAGCCATTTGAAGCAGAATC
CAAACCATGAATTGTAGAGAATTACCCTTGACCCTTTGGGTGCTTATATCTGTAAGCACTGCAGAATCTT
GTACTTCACGTCCCCACATTACTGTGGTTGAAGGGGAACCTTTCTATCTGAAACATTGCTCGTGTTCACT
TGCACATGAGATTGAAACAACCACCAAAAGCTGGTACAAAAGCAGTGGATCACAGGAACATGTGGAGCTG
AACCCAAGGAGTTCCTCGAGAATTGCTTTGCATGATTGTGTTTTGGAGTTTTGGCCAGTTGAGTTGAATG
ACACAGGATCTTACTTTTTCCAAATGAAAAATTATACTCAGAAATGGAAATTAAATGTCATCAGAAGAAA
TAAACACAGCTGTTTCACTGAAAGACAAGTAACTAGTAAAATTGTGGAAGTTAAAAAATTTTTTCAGATA
ACCTGTGAAAACAGTTACTATCAAACACTGGTCAACAGCACATCATTGTATAAGAACTGTAAAAAGCTAC
TACTGGAGAACAATAAAAACCCAACGATAAAGAAGAACGCCGAGTTTGAAGATCAGGGGTATTACTCCTG
CGTGCATTTCCTTCATCATAATGGAAAACTATTTAATATCACCAAAACCTTCAATATAACAATAGTGGAA
GATCGCAGTAATATAGTTCCGGTTCTTCTTGGACCAAAGCTTAACCATGTTGCAGTGGAATTAGGAAAAA
ACGTAAGGCTCAACTGCTCTGCTTTGCTGAATGAAGAGGATGTAATTTATTGGATGTTCGGGGAAGAAAA
TGGATCGGATCCTAATATACATGAAGAGAAAGAAATGAGAATTATGACTCCAGAAGGCAAATGGCATGCT
TCAAAAGTATTGAGAATTGAAAATATTGGTGAAAGCAATCTAAATGTTTTATATAATTGCACTGTGGCCA
GCACGGGAGGCACAGACACCAAAAGCTTCATCTTGGTGAGAAAAGCAGACATGGCTGATATCCCAGGCCA
CGTCTTCACAAGAGGAATGATCATAGCTGTTTTGATCTTGGTGGCAGTAGTGTGCCTAGTGACTGTGTGT
GTCATTTATAGAGTTGACTTGGTTCTATTTTATAGACATTTAACGAGAAGAGATGAAACATTAACAGATG
GAAAAACATATGATGCTTTTGTGTCTTACCTAAAAGAATGCCGACCTGAAAATGGAGAGGAGCACACCTT
TGCTGTGGAGATTTTGCCCAGGGTGTTGGAGAAACATTTTGGGTATAAGTTATGCATATTTGAAAGGGAT
GTAGTGCCTGGAGGAGCTGTTGTTGATGAAATCCACTCACTGATAGAGAAAAGCCGAAGACTAATCATTG
TCCTAAGTAAAAGTTATATGTCTAATGAGGTCAGGTATGAACTTGAAAGTGGACTCCATGAAGCATTGGT
GGAAAGAAAAATTAAAATAATCTTAATTGAATTTACACCTGTTACTGACTTCACATTCTTGCCCCAATCA
CTAAAGCTTTTGAAATCTCACAGAGTTCTGAAGTGGAAGGCCGATAAATCTCTTTCTTATAACTCAAGGT
TCTGGAAGAACCTTCTTTACTTAATGCCTGCAAAAACAGTCAAGCCAGGTAGAGACGAACCGGAAGTCTT
GCCTGTTCTTTCCGAGTCTTAATCTTCAGAAACAGTGAACGCCAAAAAGAACTCAAGATATTCTGGGGAC
TGAGCATATGAACCTGTTCATAACAAAGGCTGTGACTCGAAATAATTAACTTTGTCAAAATCCTGCTCAC
AATTTGAAGATGAAACTTGTCATTAGGTTGGCGGGAATGAGACTAAAGATTGCGCTGTGGGCTGTGGTCA
CGTGCTCCCAGAAGACCTGGAATTCAAAAGAAATGGAGCTATTCTTTTTCTCCCTCTTTCATAACTGGAT
GCAGCTGCTCATACTCAATCCCATATTCAGCAAGTGTGAAGCTGGACGTGATGCAAAATAACCGATGCCC
TACAAAAAGGGCGCATCTTTAAGAGTTTTAATGCCAGTGCTTAATTCGAATGAGGGGATTTTAAGTGTCT
GAAGAGGCATTTTCTAGGGACCAGTGGGTGACTGAGTAACTGAAATGCTGCTTTCACTCCCTAACACCAT
GGATCTGGTTGTGCATAGGATGTGGGAGGAGGGGCTGGCAGGGCCGCCTTCAGAGGCTGCAGGGCCTCAG
CCTCAGGATGCATTTAATGTATCCTGGCCACAGTTGCAGCCAACGGTTCTTGAAAGCTCGGTAAGGCCCT
GCAACGCAGAGCCTGCTTATGTGGATCTATTTATGGGAACTTCTTAAAAGGACCCCAGAATAGCTCTTTA
TCTTTCACAAGAGACACAAATTCTAATTGAGTTAATTATCTGGGCCTTTCACTTTGGATGCTCTGAAACA
TTTGTTGATTTTGTGTGAATGTTTATATCAAAATGTTTGCCAGGTTGTATTAGCCATTGAATAGCAAAAA
ACTGATAGTTACTTGCTTGTTTTTTAAAAATTACATATTAAAAATGCCCTTGGCATAAGGCAGCATGGTG
TGGCAGTTAAGAGATGGGCTGTGCAGCCCATCCTGAGCTCCAGTCCTGAGTTTGCTACTTACTTCTGTGG
CCTCTGGAACCTTATCCAACCTCTTGGTGCTTCAGTTTCCTCATCTGTGAAATTAGAATTTATAATAATT
GCACCTACCTCCCAGGGGTAACTAAATGAATAAATATAATAAAGTACTTACAGTGGTTCCTGACACAGAC
TCAGCACTCCGTCAGTGTTGCCATGACTATTTTTATTATCATTATTAATGATTACTTAGATCAATTATTT
AGCAGTGGACTAATGGAAGCTACAGAGCAGGGAAGGGAAGCAGATCTAGGGAGGAAGGCAGTTTTGATTT
GAGGAGGTTTGCACATGTAGAGAAGCATACTGGAGAAGCATATCCAGAGGGCGAAAGATATCTCTCCATT
GTGCATCTGCCTCTTTTGACGTTGGAAGACACATGTCTTACTCCCCAAAGGGAGCCCAGCACTGGGAGCC
TTCTTGATGATCTCAAAAATAATAGCTATTCAAGAAAATCACCAAGTGACTGTGAAACCGTCAGTTCGGA
AGGCTGGTTAGAACATGTGGGAGCAACATGAATGTTCTACAAAAGTTTAAAGCAGAGATTGTTTCAAATG
GGTGTAGTAGATATTACTGAAAACCAAAAAAGAGTGAGATTGTCAGTGTAAGAATGTGATTTAATGTTTG
TAGTGCTTACAATTTTGTGTACCAACTGGATGACTAAAAAGAGTAAAATAATTTAATTAATAGCTCATAT
TTTATGTGTGAAAACATGTTAGTGAACATATATAATCAAAATAGATTTCATTGCTATTGCATAGTCTCTA
ATACATAGAATGATTTTGCTTTTCTCTTTTATTATACTTGCTTTAAAATACTTGAAATATATTTTGCATT
AAATGCATTTCAAGTTAAATGTCTTAAATGTATACATTAGATGTGTGTTTTAAAATGCATAAAACACGTT
GAAATACATTAATGAACCATT
>NM_003853.3 Homo sapiens interleukin 18 receptor accessory protein
(IL18RAP), mRNA
(SEQ ID NO: 50)
GTATCTCTCTGGATAGGAAGAAATATAGTAGAACCCTTTGAAAATGGATATTTTCACATATTTTCGTTCA
GATACAAAAGCTGGCAGTTACTGAAATAAGGACTTGAAGTTCCTTCCTCTTTTTTTTATGTCTTAAGAGC
AGGAAATAAAGAGACAGCTGAAGGTGTAGCCTTGACCAACTGAAAGGGAAATCTTCATCCTCTGAAAAAA
CATATGTGATTCTCAAAAAACGCATCTGGAAAATTGATAAAGAAGCGATTCTGTAGATTCTCCCAGCGCT
GTTGGGCTCTCAATTCCTTCTGTGAAGGACAACATATGGTGATGGGGAAATCAGAAGCTTTGAGACCCTC
TACACCTGGATATGAATCCCCCTTCTAATACTTACCAGAAATGAAGGGGATACTCAGGGCAGAGTTCTGA
ATCTCAAAACACTCTACTCTGGCAAAGGAATGAAGTTATTGGAGTGATGACAGGAACACGGGAGAACAAT
GCTCTGTTTGGGCTGGATATTTCTTTGGCTTGTTGCAGGAGAGCGAATTAAAGGATTTAATATTTCAGGT
TGTTCCACAAAAAAACTCCTTTGGACATATTCTACAAGGAGTGAAGAGGAATTTGTCTTATTTTGTGATT
TACCAGAGCCACAGAAATCACATTTCTGCCACAGAAATCGACTCTCACCAAAACAAGTCCCTGAGCACCT
GCCCTTCATGGGTAGTAACGACCTATCTGATGTCCAATGGTACCAACAACCTTCGAATGGAGATCCATTA
GAGGACATTAGGAAAAGCTATCCTCACATCATTCAGGACAAATGTACCCTTCACTTTTTGACCCCAGGGG
TGAATAATTCTGGGTCATATATTTGTAGACCCAAGATGATTAAGAGCCCCTATGATGTAGCCTGTTGTGT
CAAGATGATTTTAGAAGTTAAGCCCCAGACAAATGCATCCTGTGAGTATTCCGCATCACATAAGCAAGAC
CTACTTCTTGGGAGCACTGGCTCTATTTCTTGCCCCAGTCTCAGCTGCCAAAGTGATGCACAAAGTCCAG
CGGTAACCTGGTACAAGAATGGAAAACTCCTCTCTGTGGAAAGGAGCAACCGAATCGTAGTGGATGAAGT
TTATGACTATCACCAGGGCACATATGTATGTGATTACACTCAGTCGGATACTGTGAGTTCGTGGACAGTC
AGAGCTGTTGTTCAAGTGAGAACCATTGTGGGAGACACTAAACTCAAACCAGATATTCTGGATCCTGTCG
AGGACACACTGGAAGTAGAACTTGGAAAGCCTTTAACTATTAGCTGCAAAGCACGATTTGGCTTTGAAAG
GGTCTTTAACCCTGTCATAAAATGGTACATCAAAGATTCTGACCTAGAGTGGGAAGTCTCAGTACCTGAG
GCGAAAAGTATTAAATCCACTTTAAAGGATGAAATCATTGAGCGTAATATCATCTTGGAAAAAGTCACTC
AGCGTGATCTTCGCAGGAAGTTTGTTTGCTTTGTCCAGAACTCCATTGGAAACACAACCCAGTCCGTCCA
ACTGAAAGAAAAGAGAGGAGTGGTGCTCCTGTACATCCTGCTTGGCACCATCGGGACCCTGGTGGCCGTG
CTGGCGGCGAGTGCCCTCCTCTACAGGCACTGGATTGAAATAGTGCTGCTGTACCGGACCTACCAGAGCA
AGGATCAGACGCTTGGGGATAAAAAGGATTTTGATGCTTTCGTATCCTATGCAAAATGGAGCTCTTTTCC
AAGTGAGGCCACTTCATCTCTGAGTGAAGAACACTTGGCCCTGAGCCTATTTCCTGATGTTTTAGAAAAC
AAATATGGATATAGCCTGTGTTTGCTTGAAAGAGATGTGGCTCCAGGAGGAGTGTATGCAGAAGACATTG
TGAGCATTATTAAGAGAAGCAGAAGAGGAATATTTATCTTGAGCCCCAACTATGTCAATGGACCCAGTAT
CTTTGAACTACAAGCAGCAGTGAATCTTGCCTTGGATGATCAAACACTGAAACTCATTTTAATTAAGTTC
TGTTACTTCCAAGAGCCAGAGTCTCTACCTCATCTCGTGAAAAAAGCTCTCAGGGTTTTGCCCACAGTTA
CTTGGAGAGGCTTAAAATCAGTTCCTCCCAATTCTAGGTTCTGGGCCAAAATGCGCTACCACATGCCTGT
GAAAAACTCTCAGGGATTCACGTGGAACCAGCTCAGAATTACCTCTAGGATTTTTCAGTGGAAAGGACTC
AGTAGAACAGAAACCACTGGGAGGAGCTCCCAGCCTAAGGAATGGTGAAATGAGCCCTGGAGCCCCCTCC
AGTCCAGTCCCTGGGATAGAGATGTTGCTGGACAGAACTCACAGCTCTGTGTGTGTGTGTTCAGGCTGAT
AGGAAATTCAAAGAGTCTCCTGCCAGCACCAAGCAAGCTTGATGGACAATGGAGTGGGATTGAGACTGTG
GTTTAGAGCCTTTGATTTCCTGGACTGGACTGACGGCGAGTGAATTCTCTAGACCTTGGGTACTTTCAGT
ACACAACACCCCTAAGATTTCCCAGTGGTCCGAGCAGAATCAGAAAATACAGCTACTTCTGCCTTATGGC
TAGGGAACTGTCATGTCTACCATGTATTGTACATATGACTTTATGTATACTTGCAATCAAATAAATATTA
TTTTATTAGAAA

Also disclosed herein are Brd4 inhibitors. An exemplary mRNA sequence of Brd4 is provided below, represented by SEQ ID NO: 39:

>NM_058243.2 Homo sapiens
bromodomain containing 4 (BRD4),
transcript variant long, mRNA
(SEQ ID NO: 39)
ATTCTTTGGAATACTACTGCTAGAAGTCTGACTTA
AGACCCAGCTTATGGGCCACATGGCACCCAGCTGC
TTCTGCAGAGAAGGCAGGCCACTGATGGGTACAGC
AAAGTGTGGTGCTGCTGGCCAAGCCAAAGACCCGT
GTAGGATGACTGGGCCTCTGCCCCTTGTGGGTGTT
GCCACTGTGCTTGAGTGCCTGGTGAAGAATGTGAT
GGGATCACTAGCATGTCTGCGGAGAGCGGCCCTGG
GACGAGATTGAGAAATCTGCCAGTAATGGGGGATG
GACTAGAAACTTCCCAAATGTCTACAACACAGGCC
CAGGCCCAACCCCAGCCAGCCAACGCAGCCAGCAC
CAACCCCCCGCCCCCAGAGACCTCCAACCCTAACA
AGCCCAAGAGGCAGACCAACCAACTGCAATACCTG
CTCAGAGTGGTGCTCAAGACACTATGGAAACACCA
GTTTGCATGGCCTTTCCAGCAGCCTGTGGATGCCG
TCAAGCTGAACCTCCCTGATTACTATAAGATCATT
AAAACGCCTATGGATATGGGAACAATAAAGAAGCG
CTTGGAAAACAACTATTACTGGAATGCTCAGGAAT
GTATCCAGGACTTCAACACTATGTTTACAAATTGT
TACATCTACAACAAGCCTGGAGATGACATAGTCTT
AATGGCAGAAGCTCTGGAAAAGCTCTTCTTGCAAA
AAATAAATGAGCTACCCACAGAAGAAACCGAGATC
ATGATAGTCCAGGCAAAAGGAAGAGGACGTGGGAG
GAAAGAAACAGGGACAGCAAAACCTGGCGTTTCCA
CGGTACCAAACACAACTCAAGCATCGACTCCTCCG
CAGACCCAGACCCCTCAGCCGAATCCTCCTCCTGT
GCAGGCCACGCCTCACCCCTTCCCTGCCGTCACCC
CGGACCTCATCGTCCAGACCCCTGTCATGACAGTG
GTGCCTCCCCAGCCACTGCAGACGCCCCCGCCAGT
GCCCCCCCAGCCACAACCCCCACCCGCTCCAGCTC
CCCAGCCCGTACAGAGCCACCCACCCATCATCGCG
GCCACCCCACAGCCTGTGAAGACAAAGAAGGGAGT
GAAGAGGAAAGCAGACACCACCACCCCCACCACCA
TTGACCCCATTCACGAGCCACCCTCGCTGCCCCCG
GAGCCCAAGACCACCAAGCTGGGCCAGCGGCGGGA
GAGCAGCCGGCCTGTGAAACCTCCAAAGAAGGACG
TGCCCGACTCTCAGCAGCACCCAGCACCAGAGAAG
AGCAGCAAGGTCTCGGAGCAGCTCAAGTGCTGCAG
CGGCATCCTCAAGGAGATGTTTGCCAAGAAGCACG
CCGCCTACGCCTGGCCCTTCTACAAGCCTGTGGAC
GTGGAGGCACTGGGCCTACACGACTACTGTGACAT
CATCAAGCACCCCATGGACATGAGCACAATCAAGT
CTAAACTGGAGGCCCGTGAGTACCGTGATGCTCAG
GAGTTTGGTGCTGACGTCCGATTGATGTTCTCCAA
CTGCTATAAGTACAACCCTCCTGACCATGAGGTGG
TGGCCATGGCCCGCAAGCTCCAGGATGTGTTCGAA
ATGCGCTTTGCCAAGATGCCGGACGAGCCTGAGGA
GCCAGTGGTGGCCGTGTCCTCCCCGGCAGTGCCCC
CTCCCACCAAGGTTGTGGCCCCGCCCTCATCCAGC
GACAGCAGCAGCGATAGCTCCTCGGACAGTGACAG
TTCGACTGATGACTCTGAGGAGGAGCGAGCCCAGC
GGCTGGCTGAGCTCCAGGAGCAGCTCAAAGCCGTG
CACGAGCAGCTTGCAGCCCTCTCTCAGCCCCAGCA
GAACAAACCAAAGAAAAAGGAGAAAGACAAGAAGG
AAAAGAAAAAAGAAAAGCACAAAAGGAAAGAGGAA
GTGGAAGAGAATAAAAAAAGCAAAGCCAAGGAACC
TCCTCCTAAAAAGACGAAGAAAAATAATAGCAGCA
ACAGCAATGTGAGCAAGAAGGAGCCAGCGCCCATG
AAGAGCAAGCCCCCTCCCACGTATGAGTCGGAGGA
AGAGGACAAGTGCAAGCCTATGTCCTATGAGGAGA
AGCGGCAGCTCAGCTTGGACATCAACAAGCTCCCC
GGCGAGAAGCTGGGCCGCGTGGTGCACATCATCCA
GTCACGGGAGCCCTCCCTGAAGAATTCCAACCCCG
ACGAGATTGAAATCGACTTTGAGACCCTGAAGCCG
TCCACACTGCGTGAGCTGGAGCGCTATGTCACCTC
CTGTTTGCGGAAGAAAAGGAAACCTCAAGCTGAGA
AAGTTGATGTGATTGCCGGCTCCTCCAAGATGAAG
GGCTTCTCGTCCTCAGAGTCGGAGAGCTCCAGTGA
GTCCAGCTCCTCTGACAGCGAAGACTCCGAAACAG
AGATGGCTCCGAAGTCAAAAAAGAAGGGGCACCCC
GGGAGGGAGCAGAAGAAGCACCATCATCACCACCA
TCAGCAGATGCAGCAGGCCCCGGCTCCTGTGCCCC
AGCAGCCGCCCCCGCCTCCCCAGCAGCCCCCACCG
CCTCCACCTCCGCAGCAGCAACAGCAGCCGCCACC
CCCGCCTCCCCCACCCTCCATGCCGCAGCAGGCAG
CCCCGGCGATGAAGTCCTCGCCCCCACCCTTCATT
GCCACCCAGGTGCCCGTCCTGGAGCCCCAGCTCCC
AGGCAGCGTCTTTGACCCCATCGGCCACTTCACCC
AGCCCATCCTGCACCTGCCGCAGCCTGAGCTGCCC
CCTCACCTGCCCCAGCCGCCTGAGCACAGCACTCC
ACCCCATCTCAACCAGCACGCAGTGGTCTCTCCTC
CAGCTTTGCACAACGCACTACCCCAGCAGCCATCA
CGGCCCAGCAACCGAGCCGCTGCCCTGCCTCCCAA
GCCCGCCCGGCCCCCAGCCGTGTCACCAGCCTTGA
CCCAAACACCCCTGCTCCCACAGCCCCCCATGGCC
CAACCCCCCCAAGTGCTGCTGGAGGATGAAGAGCC
ACCTGCCCCACCCCTCACCTCCATGCAGATGCAGC
TGTACCTGCAGCAGCTGCAGAAGGTGCAGCCCCCT
ACGCCGCTACTCCCTTCCGTGAAGGTGCAGTCCCA
GCCCCCACCCCCCCTGCCGCCCCCACCCCACCCCT
CTGTGCAGCAGCAGCTGCAGCAGCAGCCGCCACCA
CCCCCACCACCCCAGCCCCAGCCTCCACCCCAGCA
GCAGCATCAGCCCCCTCCACGGCCCGTGCACTTGC
AGCCCATGCAGTTTTCCACCCACATCCAACAGCCC
CCGCCACCCCAGGGCCAGCAGCCCCCCCATCCGCC
CCCAGGCCAGCAGCCACCCCCGCCGCAGCCTGCCA
AGCCTCAGCAAGTCATCCAGCACCACCATTCACCC
CGGCACCACAAGTCGGACCCCTACTCAACCGGTCA
CCTCCGCGAAGCCCCCTCCCCGCTTATGATACATT
CCCCCCAGATGTCACAGTTCCAGAGCCTGACCCAC
CAGTCTCCACCCCAGCAAAACGTCCAGCCTAAGAA
ACAGGAGCTGCGTGCTGCCTCCGTGGTCCAGCCCC
AGCCCCTCGTGGTGGTGAAGGAGGAGAAGATCCAC
TCACCCATCATCCGCAGCGAGCCCTTCAGCCCCTC
GCTGCGGCCGGAGCCCCCCAAGCACCCGGAGAGCA
TCAAGGCCCCCGTCCACCTGCCCCAGCGGCCGGAA
ATGAAGCCTGTGGATGTCGGGAGGCCTGTGATCCG
GCCCCCAGAGCAGAACGCACCGCCACCAGGGGCCC
CTGACAAGGACAAACAGAAACAGGAGCCGAAGACT
CCAGTTGCGCCCAAAAAGGACCTGAAAATCAAGAA
CATGGGCTCCTGGGCCAGCCTAGTGCAGAAGCATC
CGACCACCCCCTCCTCCACAGCCAAGTCATCCAGC
GACAGCTTCGAGCAGTTCCGCCGCGCCGCTCGGGA
GAAAGAGGAGCGTGAGAAGGCCCTGAAGGCTCAGG
CCGAGCACGCTGAGAAGGAGAAGGAGCGGCTGCGG
CAGGAGCGCATGAGGAGCCGAGAGGACGAGGATGC
GCTGGAGCAGGCCCGGCGGGCCCATGAGGAGGCAC
GTCGGCGCCAGGAGCAGCAGCAGCAGCAGCGCCAG
GAGCAACAGCAGCAGCAGCAACAGCAAGCAGCTGC
GGTGGCTGCCGCCGCCACCCCACAGGCCCAGAGCT
CCCAGCCCCAGTCCATGCTGGACCAGCAGAGGGAG
TTGGCCCGGAAGCGGGAGCAGGAGCGAAGACGCCG
GGAAGCCATGGCAGCTACCATTGACATGAATTTCC
AGAGTGATCTATTGTCAATATTTGAAGAAAATCTT
TTCTGAGCGCACCTAGGTGGCTTCTGACTTTGATT
TTCTGGCAAAACATTGACTTTCCATAGTGTTAGGG
GCGGTGGTGGAGGTGGGATCAGCGGCCAGGGGATG
CCTCAGGGCCTGGCCCTCCTGCATGCTATGCCCGG
GGCAGGCCTGACGGGCAGCTGAGGATTGCAGAGCC
TGTCTGCCTTACGGCCAGTCGGACAGACGTCCCGC
CACCCACCACCCCTCACAGGACGTCCGCTCAGCAC
ACGCCTTGTTACGAGCAAGTGCCGGCTGGACCCAA
GCCCTGCATCCCCACATGCGGGGCAGAGGCCCTTC
TCTCCGCCAAATGTCTACACAGTATACACAGGACA
TCGTTGCTGCCGCCGTGACTGGTTTTCTGTCCCCA
AGAACGTGACGTTCGTGATGTCCTGCCCGCCGGGA
GTCTTTCCCCACACCCCAGCCATCGCCGCCCGCTC
CCAGGAGGCCAGGGCAGGCCTGCGTGGGCTGGAGG
CGGGCGAGGCCGGCCCACCCCCTCGCTGGCACTGA
CTTTGCCTTGAACAGACCCCCCGACCCTCCCCCAC
AAGCCTTTAATTGAGAGCCGCTCTCTGTAAGTGTT
TGCTTGTGCAAAAGGGAATAGTGCCGTGGAGGTGT
GTGTGTCCATGGCATCCGGAGCGAGGCGACTGTCC
TGCGTGGGTAGCCCTCGGCCGGGGAGTGAGGCCAC
CAACCAAAGTCAGTTCCTTCCCACCTGTGTTTCTG
TTTCGTTTTTTTTTTTCTTTTTTTTCTATATATAT
TTTTTGTTGAATTCTATTTTATTTTTAATTCTCTC
TTCTCCTCCAGACACAATGGCACTGCTTATCTCCG
AAATGGTGTGATCGTCTCCTCATTGAGCAGCGGCT
GCCACCGCGCTGTGGGTA
>NM_002186.3 Homo sapiens
interleukin 9 receptor (IL9R),
transcript variant 1, mRNA
(SEQ ID NO: 51)
GCTGTGCACCCAGAGATAGTTGGGTGACAAATCAC
CTCCAGGTTGGGGATGCCTCAGACTTGTGATGGGA
CTGGGCAGATGCATCTGGGAAGGCTGGACCTTGGA
GAGTGAGGCCCTGAGGCGAGACATGGGCACCTGGC
TCCTGGCCTGCATCTGCATCTGCACCTGTGTCTGC
TTGGGAGTCTCTGTCACAGGGGAAGGACAAGGGCC
AAGGTCTAGAACCTTCACCTGCCTCACCAACAACA
TTCTCAGGATCGATTGCCACTGGTCTGCCCCAGAG
CTGGGACAGGGCTCCAGCCCCTGGCTCCTCTTCAC
CAGCAACCAGGCTCCTGGCGGCACACATAAGTGCA
TCTTGCGGGGCAGTGAGTGCACCGTCGTGCTGCCA
CCTGAGGCAGTGCTCGTGCCATCTGACAATTTCAC
CATCACTTTCCACCACTGCATGTCTGGGAGGGAGC
AGGTCAGCCTGGTGGACCCGGAGTACCTGCCCCGG
AGACACGTTAAGCTGGACCCGCCCTCTGACTTGCA
GAGCAACATCAGTTCTGGCCACTGCATCCTGACCT
GGAGCATCAGTCCTGCCTTGGAGCCAATGACCACA
CTTCTCAGCTATGAGCTGGCCTTCAAGAAGCAGGA
AGAGGCCTGGGAGCAGGCCCAGCACAGGGATCACA
TTGTCGGGGTGACCTGGCTTATACTTGAAGCCTTT
GAGCTGGACCCTGGCTTTATCCATGAGGCCAGGCT
GCGTGTCCAGATGGCCACACTGGAGGATGATGTGG
TAGAGGAGGAGCGTTATACAGGCCAGTGGAGTGAG
TGGAGCCAGCCTGTGTGCTTCCAGGCTCCCCAGAG
ACAAGGCCCTCTGATCCCACCCTGGGGGTGGCCAG
GCAACACCCTTGTTGCTGTGTCCATCTTTCTCCTG
CTGACTGGCCCGACCTACCTCCTGTTCAAGCTGTC
GCCCAGGGTGAAGAGAATCTTCTACCAGAACGTGC
CCTCTCCAGCGATGTTCTTCCAGCCCCTCTACAGT
GTACACAATGGGAACTTCCAGACTTGGATGGGGGC
CCACGGGGCCGGTGTGCTGTTGAGCCAGGACTGTG
CTGGCACCCCACAGGGAGCCTTGGAGCCCTGCGTC
CAGGAGGCCACTGCACTGCTCACTTGTGGCCCAGC
GCGTCCTTGGAAATCTGTGGCCCTGGAGGAGGAAC
AGGAGGGCCCTGGGACCAGGCTCCCGGGGAACCTG
AGCTCAGAGGATGTGCTGCCAGCAGGGTGTACGGA
GTGGAGGGTACAGACGCTTGCCTATCTGCCACAGG
AGGACTGGGCCCCCACGTCCCTGACTAGGCCGGCT
CCCCCAGACTCAGAGGGCAGCAGGAGCAGCAGCAG
CAGCAGCAGCAGCAACAACAACAACTACTGTGCCT
TGGGCTGCTATGGGGGATGGCACCTCTCAGCCCTC
CCAGGAAACACACAGAGCTCTGGGCCCATCCCAGC
CCTGGCCTGTGGCCTTTCTTGTGACCATCAGGGCC
TGGAGACCCAGCAAGGAGTTGCCTGGGTGCTGGCT
GGTCACTGCCAGAGGCCTGGGCTGCATGAGGACCT
CCAGGGCATGTTGCTCCCTTCTGTCCTCAGCAAGG
CTCGGTCCTGGACATTCTAGGTCCCTGACTCGCCA
GATGCATCATGTCCATTTTGGGAAAATGGACTGAA
GTTTCTGGAGCCCTTGTCTGAGACTGAACCTCCTG
AGAAGGGGCCCCTAGCAGCGGTCAGAGGTCCTGTC
TGGATGGAGGCTGGAGGCTCCCCCCTCAACCCCTC
TGCTCAGTGCCTGTGGGGAGCAGCCTCTACCCTCA
GCATCCTGGCCACAAGTTCTTCCTTCCATTGTCCC
TTTTCTTTATCCCTGACCTCTCTGAGAAGTGGGGT
GTGGTCTCTCAGCTGTTCTGCCCTCATACCCTTAA
AGGGCCAGCCTGGGCCCAGTGGACACAGGTAAGGC
ACCATGACCACCTGGTGTGACCTCTCTGTGCCTTA
CTGAGGCACCTTTCTAGAGATTAAAAGGGGCTTGA
TGGCTGTT

In some embodiments, the inhibitor of Type 2 cytokine signaling is a small molecule, an inhibitory nucleic acid (e.g., siRNA, antisense nucleic acid, shRNA, sgRNA, ribozymes), a tyrosine kinase inhibitor, a decoy cytokine receptor, or an antibody (e.g., a neutralizing antibody). Additionally or alternatively, in some embodiments, the Brd4 inhibitor is a small molecule, an inhibitory nucleic acid (e.g., siRNA, antisense nucleic acid, shRNA, sgRNA, ribozymes), or an antibody (e.g., a neutralizing antibody).

In one aspect, the present disclosure provides Type 2 cytokine-specific, Type 2 cytokine receptor signaling protein-specific, or Brd4-specific inhibitory nucleic acids comprising a nucleic acid molecule which is complementary to a portion of a Type 2 cytokine nucleic acid sequence or a Brd4 nucleic acid sequence selected from the group consisting of SEQ ID NOs: 23-39 and 46-51.

The present disclosure also provides an antisense nucleic acid comprising a nucleic acid sequence that is complementary to and specifically hybridizes with a portion of any one of SEQ ID NOs: 23-39 or SEQ ID NOs: 46-51 (mRNA of Type 2 cytokine, Type 2 cytokine receptor signaling protein or Brd4), thereby reducing or inhibiting expression of Type 2 cytokine, Type 2 cytokine receptor signaling protein, or Brd4. The antisense nucleic acid may be antisense RNA, or antisense DNA. Antisense nucleic acids based on the known Type 2 cytokine, Type 2 cytokine receptor signaling protein, or Brd4 gene sequence can be readily designed and engineered using methods known in the art. In some embodiments, the antisense nucleic acid comprises the nucleic acid sequence of any one of SEQ ID NOs: 3, 4, 5, 6, or a complement thereof.

Antisense nucleic acids are molecules which are complementary to a sense nucleic acid strand, e.g., complementary to the coding strand of a double-stranded DNA molecule (or cDNA) or complementary to an mRNA sequence. Accordingly, an antisense nucleic acid can form hydrogen bonds with a sense nucleic acid. The antisense nucleic acid can be complementary to an entire Type 2 cytokine, Type 2 cytokine receptor signaling protein or Brd4 coding strand, or to a portion thereof, e.g., all or part of the protein coding region (or open reading frame). In some embodiments, the antisense nucleic acid is an oligonucleotide which is complementary to only a portion of the mRNA coding region of Type 2 cytokine, Type 2 cytokine receptor signaling protein, or Brd4. In certain embodiments, an antisense nucleic acid molecule can be complementary to a noncoding region of the Type 2 cytokine, Type 2 cytokine receptor signaling protein or Brd4 coding strand. In some embodiments, the noncoding region refers to the 5′ and 3′ untranslated regions that flank the coding region and are not translated into amino acids. For example, the antisense oligonucleotide can be complementary to the region surrounding the translation start site of Type 2 cytokine, Type 2 cytokine receptor signaling protein or Brd4. An antisense oligonucleotide can be, for example, about 5, 10, 15, 20, 25, 30, 35, 40, 45 or 50 nucleotides in length.

An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or modified nucleotides designed to increase the biological stability of the molecules or to increase the physical stability of the duplex formed between the antisense and sense nucleic acids, e.g., phosphorothioate derivatives and acridine substituted nucleotides. Examples of modified nucleotides which can be used to generate the antisense nucleic acid include 5-fluorouracil, 5-bromouracil, 5-chlorouracil, 5-hodouracil, hypoxanthine, xanthine, 4-acetylcytosine, 5-(carboxyhydroxylmethyl)uracil, 5-carboxymethylaminomethyl-2-thouridine, 5-carboxymethylaminometh-yluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-metnylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopenten-yladenine, uracil-5-oxyacetic acid (v), wybutosine, pseudouracil, queosine, 2-thiocytosine, 5-methyl-2-thiouracil, 2-thlouracil, 4-thiouracil, 5-methyluracil, uracil-5-oxyacetic acid methylester, uracil-5-cxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

The antisense nucleic acid molecules may be administered to a subject or generated in situ such that they hybridize with or bind to cellular mRNA and/or genomic DNA encoding the protein of interest to thereby inhibit expression of the protein, e.g., by inhibiting transcription and/or translation. The hybridization can occur via Watson-Crick base pairing to form a stable duplex, or in the case of an antisense nucleic acid molecule which binds to DNA duplexes, through specific interactions in the major groove of the double helix.

In some embodiments, the antisense nucleic acid molecules are modified such that they specifically bind to receptors or antigens expressed on a selected cell surface, e.g., by linking the antisense nucleic acid molecules to peptides or antibodies which bind to cell surface receptors or antigens. In some embodiments, the antisense nucleic acid molecule is an alpha-anomeric nucleic acid molecule. An alpha-anomeric nucleic acid molecule forms specific double-stranded hybrids with complementary RNA in which, contrary to the usual β-units, the strands run parallel to each other (Gaultier et al., Nucleic Acids. Res. 15:6625-6641(1987)). The antisense nucleic acid molecule can also comprise a 2′-O -methylribonucleotide (Inoue et al., Nucleic Acids Res. 15:6131-6148 (1987)) or a chimeric RNA-DNA analogue (Inoue et al., FEBS Lett. 215:327-330 (1987)).

The present disclosure also provides a short hairpin RNA (shRNA) or small interfering RNA (siRNA) comprising a nucleic acid sequence that is complementary to and specifically hybridizes with a portion of any one of SEQ ID NOs: 23-39 or SEQ ID NOs: 46-51 (mRNA of Type 2 cytokine, Type 2 cytokine receptor signaling protein or Brd), thereby reducing or inhibiting expression of a Type 2 cytokine or Brd4. In some embodiments, the shRNA or siRNA is about 18, 19, 20, 21, 22, 23, 24, 25, 26, 27, 28 or 29 base pairs in length. Double-stranded RNA (dsRNA) can induce sequence-specific post-transcriptional gene silencing (e.g., RNA interference (RNAi)) in many organisms such as C. elegans, Drosophila, plants, mammals, oocytes and early embryos. RNAi is a process that interferes with or significantly reduces the number of protein copies made by an mRNA. For example, a double-stranded siRNA or shRNA molecule is engineered to complement and hydridize to a mRNA of a target gene. Following intracellular delivery, the siRNA or shRNA molecule associates with an RNA-induced silencing complex (RISC), which then binds and degrades a complementary target mRNA (such as mRNA of a Type 2 cytokine, Type 2 cytokine receptor signaling protein or Brd4). In some embodiments, the shRNA or siRNA comprises the nucleic acid sequence of any one of SEQ ID NOs: 3-6.

The present disclosure also provides a ribozyme comprising a nucleic acid sequence that is complementary to and specifically hybridizes with a portion of any one of SEQ ID NOs: 23-39 or SEQ ID NOs: 46-51 (mRNA of Type 2 cytokine, Type 2 cytokine receptor signaling protein or Brd4), thereby reducing or inhibiting expression of Type 2 cytokine, Type 2 cytokine receptor signaling protein or Brd4. Ribozymes are catalytic RNA molecules with ribonuclease activity which are capable of cleaving a complementary single-stranded nucleic acid, such as an mRNA. Thus, ribozymes (e.g., hammerhead ribozymes (described in Haselhoff and Gerlach, Nature 334:585-591 (1988))) can be used to catalytically cleave Type 2 cytokine, Type 2 cytokine receptor signaling protein or Brd4 transcripts, thereby inhibiting translation of a Type 2 cytokine, Type 2 cytokine receptor signaling protein, or Brd4.

A ribozyme having specificity for a Type 2 cytokine-, Type 2 cytokine receptor signaling protein- or Brd4-encoding nucleic acid can be designed based upon nucleic acid sequence of Brd4, a Type 2 cytokine, or Type 2 cytokine receptor signaling protein disclosed herein. For example, a derivative of a Tetrahymena L-19 IVS RNA can be constructed in which the nucleotide sequence of the active site is complementary to the nucleotide sequence to be cleaved in a Type 2 cytokine-, Type 2 cytokine receptor signaling protein- or Brd4-encoding mRNA. See, e.g., U.S. Pat. Nos. 4,987,071 and 5,116,742. Alternatively, mRNA of a Type 2 cytokine, Type 2 cytokine receptor signaling protein or Brd4 can be used to select a catalytic RNA having a specific ribonuclease activity from a pool of RNA molecules. See, e.g., Bartel and Szostak (1993) Science 261:1411-1418, incorporated herein by reference.

The present disclosure also provides a synthetic guide RNA (sgRNA) comprising a nucleic acid sequence that is complementary to and specifically hybridizes with a portion of any one of SEQ ID NOs: 23-39 or SEQ ID NOs: 46-51 (mRNA of Type 2 cytokine, Type 2 cytokine receptor signaling protein or Brd4). Guide RNAs for use in CRISPR-Cas systems are typically generated as a single guide RNA comprising a crRNA segment and a tracrRNA segment. The crRNA segment and a tracrRNA segment can also be generated as separate RNA molecules. The crRNA segment comprises the targeting sequence that binds to a portion of any one of SEQ ID NOs: 23-39 or SEQ ID NOs: 46-51, and a stem portion that hybridizes to a tracrRNA. The tracrRNA segment comprises a nucleotide sequence that is partially or completely complementary to the stem sequence of the crRNA and a nucleotide sequence that binds to the CRISPR enzyme. In some embodiments, the crRNA segment and the tracrRNA segment are provided as a single guide RNA. In some embodiments, the crRNA segment and the tracrRNA segment are provided as separate RNAs. The combination of the CRISPR enzyme with the crRNA and tracrRNA make up a functional CRISPR-Cas system. Exemplary CRISPR-Cas systems for targeting nucleic acids, are described, for example, in WO2015/089465.

In some embodiments, a synthetic guide RNA is a single RNA represented as comprising the following elements:
5′-X1-X2-Y-Z-3′

where X1 and X2 represent the crRNA segment, where X1 is the targeting sequence that binds to a portion of any one of SEQ ID NOs: 23-39 or SEQ ID NOs: 46-51, X2 is a stem sequence the hybridizes to a tracrRNA, Z represents a tracrRNA segment comprising a nucleotide sequence that is partially or completely complementary to X2, and Y represents a linker sequence. In some embodiments, the linker sequence comprises two or more nucleotides and links the crRNA and tracrRNA segments. In some embodiments, the linker sequence comprises 2, 3, 4, 5, 6, 7, 8, 9, 10 or more nucleotides. In some embodiments, the linker is the loop of the hairpin structure formed when the stem sequence hybridized with the tracrRNA.

In some embodiments, a synthetic guide RNA is provided as two separate RNAs where one RNA represents a crRNA segment: 5′-X1-X2-3′ where X1 is the targeting sequence that binds to a portion of any one of SEQ ID NOs: 23-39 or SEQ ID NOs: 46-51, X2 is a stem sequence the hybridizes to a tracrRNA, and one RNA represents a tracrRNA segment, Z, that is a separate RNA from the crRNA segment and comprises a nucleotide sequence that is partially or completely complementary to X2 of the crRNA.

Exemplary crRNA stem sequences and tracrRNA sequences are provided, for example, in WO/2015/089465, which is incorporated by reference herein. In general, a stem sequence includes any sequence that has sufficient complementarity with a complementary sequence in the tracrRNA to promote formation of a CRISPR complex at a target sequence, wherein the CRISPR complex comprises the stem sequence hybridized to the tracrRNA. In general, degree of complementarity is with reference to the optimal alignment of the stem and complementary sequence in the tracrRNA, along the length of the shorter of the two sequences. Optimal alignment may be determined by any suitable alignment algorithm, and may further account for secondary structures, such as self-complementarity within either the stem sequence or the complementary sequence in the tracrRNA. In some embodiments, the degree of complementarity between the stem sequence and the complementary sequence in the tracrRNA along the length of the shorter of the two when optimally aligned is about or more than about 25%, 30%, 40%, 50%, 60%, 70%, 80%, 90%, 95%, 97.5%, 99%, or higher. In some embodiments, the stem sequence is about or more than about 5, 6, 7, 8, 9, 10, 11, 12, 13, 14, 15, 16, 17, 18, 19, 20, 25, 30, 40, 50, or more nucleotides in length. In some embodiments, the stem sequence and complementary sequence in the tracrRNA are contained within a single RNA, such that hybridization between the two produces a transcript having a secondary structure, such as a hairpin. In some embodiments, the tracrRNA has additional complementary sequences that form hairpins. In some embodiments, the tracrRNA has at least two or more hairpins. In some embodiments, the tracrRNA has two, three, four or five hairpins. In some embodiments, the tracrRNA has at most five hairpins.

In a hairpin structure, the portion of the sequence 5′ of the final “N” and upstream of the loop corresponds to the crRNA stem sequence, and the portion of the sequence 3′ of the loop corresponds to the tracrRNA sequence. Further non-limiting examples of single polynucleotides comprising a guide sequence, a stem sequence, and a tracr sequence are as follows (listed 5′ to 3′), where “N” represents a base of a guide sequence (e.g. a modified oligonucleotide provided herein), the first block of lower case letters represent stem sequence, and the second block of lower case letters represent the tracrRNA sequence, and the final poly-T sequence represents the transcription terminator:

(a)
(SEQ ID NO: 40)
NNNNNNNNNNNNNNNNNNNNgtttttgtactctcaagattt
aGAAAtaaatcttgcagaagctacaaagataaggcttcatg
ccgaaatcaacaccctgtcattttatggcagggtgttttcg
ttatttaaTTTTTT;
(b)
(SEQ ID NO: 41)
NNNNNNNNNNNNNNNNNNNNgtttttgtactctcaGAAAtg
cagaagctacaaagataaggcttcatgccgaaatcaacacc
ctgtcattttatggcagggtgttttcgttatttaaTTTTTT;
(c)
(SEQ ID NO: 42)
NNNNNNNNNNNNNNNNNNNNgtttttgtactctcaGAAAtg
cagaagctacaaagataaggcttcatgccgaaatcaacacc
ctgtcattttatggcagggtgtTTTTTT;
(d)
(SEQ ID NO: 43)
NNNNNNNNNNNNNNNNNNNNgttttagagctaGAAAtagca
agttaaaataaggctagtccgttatcaacttgaaaaagtgg
caccgagtcggtgcTTTTTT;
(e)
(SEQ ID NO: 44)
NNNNNNNNNNNNNNNNNNNNgttttagagctaGAAATAGca
agttaaaataaggctagtccgttatcaacttgaaaaagtgT
TTTTTT;
and
(f)
(SEQ ID NO: 45)
NNNNNNNNNNNNNNNNNNNNgttttagagctagAAATAGca
agttaaaataaggctagtccgttatcaTTTTTTTT.

Selection of suitable oligonucleotides for use in as a targeting sequence in a CRISPR Cas system depends on several factors including the particular CRISPR enzyme to be used and the presence of corresponding proto-spacer adjacent motifs (PAMs) downstream of the target sequence in the target nucleic acid. The PAM sequences direct the cleavage of the target nucleic acid by the CRISPR enzyme. In some embodiments, a suitable PAM is 5′-NRG or 5′-NNGRR (where N is any Nucleotide) for SpCas9 or SaCas9 enzymes (or derived enzymes), respectively. Generally the PAM sequences should be present between about 1 to about 10 nucleotides of the target sequence to generate efficient cleavage of the target nucleic acid. Thus, when the guide RNA forms a complex with the CRISPR enzyme, the complex locates the target and PAM sequence, unwinds the DNA duplex, and the guide RNA anneals to the complementary sequence on the opposite strand. This enables the Cas9 nuclease to create a double-strand break.

A variety of CRISPR enzymes are available for use in conjunction with the disclosed guide RNAs of the present disclosure. In some embodiments, the CRISPR enzyme is a Type II CRISPR enzyme. In some embodiments, the CRISPR enzyme catalyzes DNA cleavage. In some embodiments, the CRISPR enzyme catalyzes RNA cleavage. In some embodiments, the CRISPR enzyme is any Cas9 protein, for instance any naturally-occurring bacterial Cas9 as well as any chimeras, mutants, homologs or orthologs. Non-limiting examples of Cas proteins include Cas1, Cas1B, Cas2, Cas3, Cas4, Cas5, Cash, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified variants thereof. In some embodiments, the CRISPR enzyme cleaves both strands of the target nucleic acid at the Protospacer Adjacent Motif (PAM) site. In some embodiments, the CRISPR enzyme is a nickase, which cleaves only one strand of the target nucleic acid.

Other exemplary inhibitors of Type 2 cytokine signaling include, but are not limited to, dupilumab, Etokimab (ANB020), Mepolizumab, reslizumab, benralizumab, lebrikizumab, risankizumab, tocilizumab, tralokinumab, anrukinzumab, AMG317, STAT6 inhibitors, STAT3 inhibitors, Secukinumab, ustekinumab, guselkumab, and gevokizumab. Examples of STAT6 inhibitors are described in Nat Rev Drug Discov. 12(8): 611-629 (2013), and include, but are not limited to AS1517499, Niflumic acid, AS1810722, YM-341619, TMC-264, Leflunomide, berbamine, (R)-76, (R)-84, STAT6BP, and STAT6-IP. Examples of STAT3 inhibitors are described in Nat Rev Drug Discov. 12(8): 611-629 (2013), and include, but are not limited to ISS-610, PM-73G, CJ-1383, ISS-840, peptide inhibitors (e.g., PY*LKTK (SEQ ID NO: 52), Ac-pTyr-Leu-Pro-Gln-Thr-Val-NH2 (SEQ ID NO: 53)), 531-M2001, STA-21, Stattic, LLL12, FLLL32, S3I-201, BP-1-102, S3I-201.1066, SC-1, SC-49, Indirubin, Berbamine, Honokiol, Cryptotanshinone, Evodiamine, paclitaxel, Vinorelbine, Oleanolic acid/CDDO-Me, Cucurbitacin E, Emodin, Resveratrol, Capsaicin, Avicin D, Piceatannol, Sanguarine, Celastrol, Withaferin A, Cucurbitacin I, Cucurbitacin B, 3,3′-diindolyl-methane, Caffeic acid, and the like. Other Type 2 cytokine inhibitors include, but are not limited to, anti-mouse IL-4 (clone 11B11) (Bio X Cell, West Lebanon N.H.), Mouse IL-13 Neutralizing antibody (clone 8H8) (Invivogen, San Diego Calif.), Mouse IL-33 MAb (Clone 396118) (R&D Systems, Minneapolis, Minn.), anti-mouse/human IL-5 (Clone TRFK5) (Bio X Cell, West Lebanon N.H.) or Mouse ST2/IL-33R antibody (Clone 245707) (R&D Systems, Minneapolis, Minn.).

Exemplary Brd4 inhibitors include but are not limited to, (+)-JQ1, I-BET762, OTX015, I-BET151, CPI203, PFI-1, MS436, CPI-0610, RVX2135, FT-1101, BAY1238097, INCB054329, TEN-010, GSK2820151, ZEN003694, BAY-299, BMS-986158, ABBV-075, GS-5829, and PLX51107.

### Methods of Treatment of the Present Technology

In one aspect, the present disclosure provides a method for treating or preventing pancreatic cancer in a subject in need thereof comprising administering to the subject an effective amount of an inhibitor of Type 2 cytokine signaling, wherein the subject harbors a KRAS mutation. The three major isoforms of RAS (KRAS, NRAS, and HRAS) together are mutated in about 20% of human cancers, primarily in the active site at residues G12, G13, and Q61 near the g-phosphate of the guanosine triphosphate (GTP) substrate (See Marcus & Mattos, Clin Cancer Res 21(8): 1810-1818 (2015)). In certain embodiments, the KRAS mutation selected from the group consisting of G12C, G12D, G12V, G12A, G12S, G12R, G13D, G13C, G13S, G13R, G13A, G13V, Q61H, Q61L, Q61R, Q61K, Q61P, and Q61E.

Additionally or alternatively, the inhibitor of Type 2 cytokine signaling inhibits a Type 2 cytokine or Type 2 cytokine receptor signaling protein selected from the group consisting of IL-33, IL-4, IL-13, IL-5, IL-23A, IL-17E, IL-9, IL9R, IL1RL1, IL4RA, IL13RA1, IL13RA2, IL5RA, IL1RL2, IL17RA, IL31RA, IL17RE, IL18R1, IL18RAP, and IL1R1. The inhibitor of Type 2 cytokine signaling may be a small molecule, an inhibitory nucleic acid (e.g., siRNA, antisense nucleic acid, shRNA, sgRNA, ribozymes), a tyrosine kinase inhibitor, a decoy cytokine receptor, or an antibody (e.g., a neutralizing antibody). Additionally or alternatively, in some embodiments, the small molecule, the inhibitory nucleic acid, the tyrosine kinase inhibitor, the decoy cytokine receptor, or the antibody specifically binds to and inhibits/neutralizes the expression or activity of IL-33, IL-4, IL-13, IL-5, IL-23A, IL-17E, IL-9, IL9R, IL1RL1, IL4RA, IL13RA1, IL13RA2, IL5RA, IL1RL2, IL17RA, IL31RA, IL17RE, IL18R1, IL18RAP, or IL1R1. Examples of inhibitors of Type 2 cytokine signaling include, but are not limited to dupilumab, Etokimab (ANB020), Mepolizumab, reslizumab, benralizumab, lebrikizumab, risankizumab, tocilizumab, tralokinumab, anrukinzumab, AMG317, STATE inhibitors, STAT3 inhibitors, Secukinumab, ustekinumab, guselkumab, gevokizumab, or any other agent that inhibits the expression or activity of any of the Type 2 cytokines or Type 2 cytokine receptor signaling proteins disclosed herein. The pancreatic cancer may comprise exocrine tumors. In certain embodiments, the pancreatic cancer is pancreatic ductal adenocarcinoma.

Additionally or alternatively, in some embodiments, the methods further comprise administering to the subject an effective amount of a Brd4 inhibitor. The Brd4 inhibitor may be a small molecule, an inhibitory nucleic acid (e.g., siRNA, antisense nucleic acid, shRNA, sgRNA, ribozymes), or an antibody (e.g., a neutralizing antibody). Examples of Brd4 inhibitors include, but are not limited to, (+)-JQ1, I-BET762, OTX015, I-BET151, CPI203, PFI-1, MS436, CPI-0610, RVX2135, FT-1101, BAY1238097, INCB054329, TEN-010, GSK2820151, ZEN003694, BAY-299, BMS-986158, ABBV-075, GS-5829, and PLX51107. Additionally or alternatively, in some embodiments, the method further comprises sequentially, simultaneously, or separately administering one or more additional therapeutic agents to the subject.

In any and all embodiments of the methods disclosed herein, the subject harbors a mutation in TP53. The subject may have a family history of pancreatic ductal adenocarcinoma or exhibits chronic pancreatitis, Type 2 diabetes or other risk factors for developing pancreatic cancer. Additionally or alternatively, in some embodiments, the subject exhibits elevated expression levels of at least one of IL-33, IL-4, IL-13, IL-5, IL-23A, IL-17E, IL-9, IL9R, IL1RL1, IL4RA, IL13RA1, IL13RA2, IL5RA, IL1RL2, IL17RA, IL31RA, IL17RE, IL18R1, IL18RAP, and IL1R1 compared to that observed in a healthy control subject or a predetermined threshold.

In another aspect, the present disclosure provides a method for selecting pancreatic cancer patients for treatment with an inhibitor of Type 2 cytokine signaling comprising (a) detecting expression levels or chromatin accessibility levels of a Type 2 cytokine or Type 2 cytokine receptor signaling protein in biological samples obtained from pancreatic cancer patients, wherein the Type 2 cytokine or the Type 2 cytokine receptor signaling protein is selected from the group consisting of IL-33, IL-4, IL-13, IL-5, IL-23A, IL-17E, IL-9, IL9R, IL1RL1, IL4RA, IL13RA1, IL13RA2, IL5RA, IL1RL2, IL17RA, IL31RA, IL17RE, IL18R1, IL18RAP, and IL1R1; (b) identifying pancreatic cancer patients that exhibit (i) mRNA/protein expression levels of Type 2 cytokine or Type 2 cytokine receptor signaling protein that are elevated compared to that observed in a healthy control subject or a predetermined threshold, and/or (ii) chromatin accessibility levels of Type 2 cytokine or Type 2 cytokine receptor signaling protein that are elevated compared to that observed in a healthy control subject or a predetermined threshold; and (c) administering an inhibitor of Type 2 cytokine signaling to the pancreatic cancer patients of step (b). The inhibitor of Type 2 cytokine signaling may be a small molecule, an inhibitory nucleic acid (e.g., siRNA, antisense nucleic acid, shRNA, sgRNA, ribozymes), a tyrosine kinase inhibitor, a decoy cytokine receptor, or an antibody (e.g., a neutralizing antibody). Additionally or alternatively, in some embodiments, the small molecule, the inhibitory nucleic acid, the tyrosine kinase inhibitor, the decoy cytokine receptor, or the antibody specifically binds to and inhibits/neutralizes the expression or activity of IL-33, IL-4, IL-13, IL-5, IL-23A, IL-17E, IL-9, IL9R, IL1RL1, IL4RA, IL13RA1, IL13RA2, IL5RA, IL1RL2, IL17RA, IL31RA, IL17RE, IL18R1, IL18RAP, or IL1R1. Examples of inhibitors of Type 2 cytokine signaling include, but are not limited to dupilumab, Etokimab (ANB020), Mepolizumab, reslizumab, benralizumab, lebrikizumab, risankizumab, tocilizumab, tralokinumab, anrukinzumab, AMG317, STATE inhibitors, STAT3 inhibitors, Secukinumab, ustekinumab, guselkumab, gevokizumab, or any other agent that inhibits the expression or activity of any of the Type 2 cytokines or Type 2 cytokine receptor signaling proteins disclosed herein.

Additionally or alternatively, in some embodiments, the methods further comprise administering a Brd4 inhibitor to the pancreatic cancer patients of step (b). The Brd4 inhibitor may be a small molecule, an inhibitory nucleic acid (e.g., siRNA, antisense nucleic acid, shRNA, sgRNA, ribozymes), or an antibody (e.g., a neutralizing antibody). Examples of Brd4 inhibitors include, but are not limited to, (+)-JQ1, I-BET762, OTX015, I-BET151, CPI203, PFI-1, MS436, CPI-0610, RVX2135, FT-1101, BAY1238097, INCB054329, TEN-010, GSK2820151, ZEN003694, BAY-299, BMS-986158, ABBV-075, GS-5829, and PLX51107.

In any and all embodiments of the methods disclosed herein, the pancreatic cancer patients harbor a KRAS mutation selected from the group consisting of G12C, G12D, G12V, G12A, G12S, G12R, G13D, G13C, G13S, G13R, G13A, G13V, Q61H, Q61L, Q61R, Q61K, Q61P, and Q61E. Additionally or alternatively, in some embodiments, the pancreatic cancer patients harbor a mutation in TP53. The pancreatic cancer patients may exhibit exocrine tumors. In certain embodiments, the pancreatic cancer patients suffer from or are at risk for pancreatic ductal adenocarcinoma.

In any of the preceding embodiments of the methods disclosed herein, the expression levels or chromatin accessibility levels of the Type 2 cytokine or the Type 2 cytokine receptor signaling proteins are detected via ChIP, MNase, FAIRE, DNAse, ATAC-seq, RT-PCR, Northern Blotting, RNA-Seq, microarray analysis, High-performance liquid chromatography (HPLC), mass spectrometry, immunohistochemistry (IHC), fluorescence in situ hybridization (FISH), Western Blotting, immunoprecipitation, flow cytometry, Immuno-electron microscopy, immunoelectrophoresis, enzyme-linked immunosorbent assays (ELISA), or multiplex ELISA antibody arrays. In some embodiments, the biological samples are pancreatic cancer specimens, blood, serum, or plasma.

### Modes of Administration and Effective Dosages

Any method known to those in the art for contacting a cell, organ or tissue with an inhibitor of Type 2 cytokine signaling and/or a Brd4 inhibitor may be employed. Suitable methods include in vitro, ex vivo, or in vivo methods. In vivo methods typically include the administration of an inhibitor of Type 2 cytokine signaling and/or a Brd4 inhibitor, such as those described above, to a mammal, suitably a human. When used in vivo for therapy, the inhibitors of Type 2 cytokine signaling and/or the Brd4 inhibitors are administered to the subject in effective amounts (i.e., amounts that have desired therapeutic effect). The dose and dosage regimen will depend upon the degree of the infection in the subject, the characteristics of the particular inhibitor of Type 2 cytokine signaling and/or Brd4 inhibitor used, e.g., its therapeutic index, the subject, and the subject's history.

The effective amount may be determined during pre-clinical trials and clinical trials by methods familiar to physicians and clinicians. An effective amount of an inhibitor of Type 2 cytokine signaling and/or a Brd4 inhibitor useful in the methods may be administered to a mammal in need thereof by any of a number of well-known methods for administering pharmaceutical compounds. The inhibitor of Type 2 cytokine signaling and/or the Brd4 inhibitor may be administered systemically or locally.

By way of an example, inhibitors of Type 2 cytokine signaling and/or Brd4 inhibitors of the present technology may be formulated in a simple delivery vehicle. In certain embodiments, inhibitors of Type 2 cytokine signaling and/or Brd4 inhibitors of the present technology may be lyophilized or incorporated in a gel, cream, biomaterial, sustained release delivery vehicle.

Inhibitors of Type 2 cytokine signaling and/or Brd4 inhibitors of the present technology are generally combined with a pharmaceutically acceptable carrier. The term “pharmaceutically acceptable carrier” refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids and amino acid copolymers. Such carriers are well known to those of ordinary skill in the art. Pharmaceutically acceptable carriers in therapeutic compositions can include liquids such as water, saline, glycerol and ethanol. Auxiliary substances, such as wetting or emulsifying agents, pH buffering substances, and the like, can also be present in such vehicles. Pharmaceutically acceptable salts can also be present in the pharmaceutical composition, e.g. mineral acid salts such as hydrochlorides, hydrobromides, phosphates, sulfates, and the like; and the salts of organic acids such as acetates, propionates, malonates, benzoates, and the like.

The inhibitor of Type 2 cytokine signaling and/or the Brd4 inhibitor may be formulated as a pharmaceutically acceptable salt. The term “pharmaceutically acceptable salt” means a salt prepared from a base or an acid which is acceptable for administration to a patient, such as a mammal (e.g., salts having acceptable mammalian safety for a given dosage regime). However, it is understood that the salts are not required to be pharmaceutically acceptable salts, such as salts of intermediate compounds that are not intended for administration to a patient. Pharmaceutically acceptable salts can be derived from pharmaceutically acceptable inorganic or organic bases and from pharmaceutically acceptable inorganic or organic acids. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine, trimethylamine, tripropylamine, tromethamine and the like. Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucuronic, mandelic, mucic, nicotinic, orotic, pamoic, pantothenic, sulfonic acids (e.g., benzenesulfonic, camphosulfonic, edisylic, ethanesulfonic, isethionic, methanesulfonic, naphthalenesulfonic, naphthalene-1,5-disulfonic, naphthalene-2,6-disulfonic and p-toluenesulfonic acids), xinafoic acid, and the like.

The inhibitors of Type 2 cytokine signaling and/or the Brd4 inhibitors of the present technology described herein can be incorporated into pharmaceutical compositions for administration, singly or in combination, to a subject for the treatment or prevention of a disorder described herein. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., 7 days of treatment).

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The inhibitor of Type 2 cytokine signaling compositions and/or Brd4 inhibitor compositions can include a carrier, which can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, isotonic agents are included, for example, sugars, polyalcohols such as mannitol, sorbitol, or sodium chloride in the composition. Prolonged absorption of the injectable compositions can be brought about by including in the composition an agent which delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the inhibitors of Type 2 cytokine signaling and/or the Brd4 inhibitors of the present technology can be delivered in the form of an aerosol spray from a pressurized container or dispenser which contains a suitable propellant, e.g., a gas such as carbon dioxide, or a nebulizer. Such methods include those described in U.S. Pat. No. 6,468,798.

Systemic administration of an inhibitor of Type 2 cytokine signaling and/or a Brd4 inhibitor of the present technology as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

An inhibitor of Type 2 cytokine signaling and/or a Brd4 inhibitor of the present technology can be formulated in a carrier system. The carrier can be a colloidal system. The colloidal system can be a liposome, a phospholipid bilayer vehicle. In one embodiment, the inhibitor of Type 2 cytokine signaling and/or the Brd4 inhibitor is encapsulated in a liposome while maintaining integrity. As one skilled in the art would appreciate, there are a variety of methods to prepare liposomes. (See Lichtenberg et al., Methods Biochem. Anal., 33:337-462 (1988); Anselem et al., Liposome Technology, CRC Press (1993)). Liposomal formulations can delay clearance and increase cellular uptake (See Reddy, Ann. Pharmacother 34(7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the inhibitor of Type 2 cytokine signaling and/or the Brd4 inhibitor of the present technology can be embedded in the polymer matrix, while maintaining protein integrity. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy et al.), U.S. Pat. Nos. 5,674,534 and 5,716,644 (both to Zale et al.), PCT publication WO 96/40073 (Zale et al.), and PCT publication WO 00/38651 (Shah et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the inhibitors of Type 2 cytokine signaling and/or the Brd4 inhibitors of the present technology are prepared with carriers that will protect the inhibitors of Type 2 cytokine signaling and/or the Brd4 inhibitors of the present technology against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The inhibitors of Type 2 cytokine signaling and/or the Brd4 inhibitors of the present technology can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi et al., Cancer Lett., 100:63-69 (1996), describes the use of fusogenic liposomes to deliver a protein to cells both in vivo and in vitro.

Dosage, toxicity and therapeutic efficacy of the inhibitor of Type 2 cytokine signaling and/or the Brd4 inhibitor of the present technology can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. In some embodiments, the inhibitor of Type 2 cytokine signaling and/or the Brd4 inhibitor of the present technology exhibit high therapeutic indices. While an inhibitor of Type 2 cytokine signaling and/or a Brd4 inhibitor of the present technology that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies within a range of circulating concentrations that include the ED50 with little or no toxicity. The dosage may vary within this range depending upon the dosage form employed and the route of administration utilized. For any an inhibitor of Type 2 cytokine signaling and/or a Brd4 inhibitor of the present technology used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Typically, an effective amount of the inhibitor of Type 2 cytokine signaling and/or the Brd4 inhibitors of the present technology, sufficient for achieving a therapeutic or prophylactic effect, range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example dosages can be 1 mg/kg body weight or 10 mg/kg body weight every day, every two days or every three days or within the range of 1-10 mg/kg every week, every two weeks or every three weeks. In one embodiment, a single dosage of an inhibitor of Type 2 cytokine signaling and/or a Brd4 inhibitor ranges from 0.001-10,000 micrograms per kg body weight. In one embodiment, inhibitor of Type 2 cytokine signaling and/or Brd4 inhibitor concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, and until the subject shows partial or complete amelioration of symptoms of disease. Thereafter, the patient can be administered a prophylactic regime.

In some embodiments, a therapeutically effective amount of an inhibitor of Type 2 cytokine signaling and/or a Brd4 inhibitor of the present technology may be defined as a concentration of the inhibitor of Type 2 cytokine signaling and/or the Brd4 inhibitor at the target tissue of 10¹² to 10′ molar, e.g., approximately 10′ molar. This concentration may be delivered by systemic doses of 0.001 to 100 mg/kg or equivalent dose by body surface area. The schedule of doses would be optimized to maintain the therapeutic concentration at the target tissue. In some embodiments, the doses are administered by single daily or weekly administration, but may also include continuous administration (e.g., parenteral infusion or transdermal application). In some embodiments, the dosage of the inhibitor of Type 2 cytokine signaling and/or the Brd4 inhibitor of the present technology is provided at a “low,” “mid,” or “high” dose level. In one embodiment, the low dose is provided from about 0.0001 to about 0.5 mg/kg/h, suitably from about 0.001 to about 0.1 mg/kg/h. In one embodiment, the mid-dose is provided from about 0.01 to about 1.0 mg/kg/h, suitably from about 0.01 to about 0.5 mg/kg/h. In one embodiment, the high dose is provided from about 0.5 to about 10 mg/kg/h, suitably from about 0.5 to about 2 mg/kg/h.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance present methods can be any mammal, including, for example, farm animals, such as sheep, pigs, cows, and horses; pet animals, such as dogs and cats; laboratory animals, such as rats, mice and rabbits. In some embodiments, the mammal is a human.

### Combination Therapy

In any and all embodiments of the methods disclosed herein, one or more inhibitors of Type 2 cytokine signaling of the present technology and/or one or more Brd4 inhibitors of the present technology may be separately, sequentially or simultaneously administered with at least one additional therapeutic agent. The at least one therapeutic agent may be selected from the group consisting of immunotherapeutic agents, alkylating agents, topoisomerase inhibitors, endoplasmic reticulum stress inducing agents, antimetabolites, mitotic inhibitors, nitrogen mustards, nitrosoureas, alkylsulfonates, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, endocrine/hormonal agents, bisphosphonate therapy agents, phenphormin and targeted biological therapy agents (e.g., therapeutic peptides described in U.S. Pat. No. 6,306,832, WO 2012007137, WO 2005000889, WO 2010096603 etc.). In some embodiments, the at least one additional therapeutic agent is a chemotherapeutic agent.

Specific chemotherapeutic agents include, but are not limited to, cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), cladribine, midostaurin, bevacizumab, oxaliplatin, melphalan, etoposide, mechlorethamine, bleomycin, microtubule poisons, annonaceous acetogenins, chlorambucil, ifosfamide, streptozocin, carmustine, lomustine, busulfan, dacarbazine, temozolomide, altretamine, 6-mercaptopurine (6-MP), cytarabine, floxuridine, fludarabine, hydroxyurea, pemetrexed, epirubicin, idarubicin, SN-38, ARC, NPC, campothecin, 9-nitrocamptothecin, 9-aminocamptothecin, rubifen, gimatecan, diflomotecan, BN80927, DX-8951f, MAG-CPT, amsacnne, etoposide phosphate, teniposide, azacitidine (Vidaza), decitabine, accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, streptozotocin, nimustine, ranimustine, bendamustine, uramustine, estramustine, mannosulfan, camptothecin, exatecan, lurtotecan, lamellarin D9-aminocamptothecin, amsacrine, ellipticines, aurintricarboxylic acid, HU-331, or combinations thereof.

Examples of antimetabolites include 5-fluorouracil (5-FU), 6-mercaptopurine (6-MP), capecitabine, cytarabine, floxuridine, fludarabine, gemcitabine, hydroxyurea, methotrexate, pemetrexed, and mixtures thereof.

Examples of taxanes include accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, and mixtures thereof.

Examples of DNA alkylating agents include cyclophosphamide, chlorambucil, melphalan, bendamustine, uramustine, estramustine, carmustine, lomustine, nimustine, ranimustine, streptozotocin; busulfan, mannosulfan, and mixtures thereof.

Examples of topoisomerase I inhibitor include SN-38, ARC, NPC, camptothecin, topotecan, 9-nitrocamptothecin, exatecan, lurtotecan, lamellarin D9-aminocamptothecin, rubifen, gimatecan, diflomotecan, BN80927, DX-8951f, MAG-CPT, and mixtures thereof. Examples of topoisomerase II inhibitors include amsacrine, etoposide, etoposide phosphate, teniposide, daunorubicin, mitoxantrone, amsacrine, ellipticines, aurintricarboxylic acid, doxorubicin, and HU-331 and combinations thereof.

Examples of immunotherapeutic agents include immune checkpoint inhibitors (e.g., antibodies targeting CTLA-4, PD-1, PD-L1), ipilimumab, 90Y-Clivatuzumab tetraxetan, pembrolizumab, nivolumab, trastuzumab, cixutumumab, ganitumab, demcizumab, cetuximab, nimotuzumab, dalotuzumab, sipuleucel-T, CRS-207, and GVAX.

In any case, the multiple therapeutic agents may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents.

### Kits

The present technology provides kits containing components suitable for treating or preventing pancreatic cancer in a patient in need thereof. In one aspect, the kits comprise at least one inhibitor of Type 2 cytokine signaling disclosed herein and/or at least one a Brd4 inhibitor disclosed herein, in combination with instructions for using the same to treat or prevent pancreatic cancer. Optionally, the above described components of the kits of the present technology are packed in suitable containers and labeled for the prevention and/or treatment of pancreatic cancer.

The above-mentioned components may be stored in unit or multi-dose containers, for example, sealed ampoules, vials, bottles, syringes, and test tubes, as an aqueous, preferably sterile, solution or as a lyophilized, preferably sterile, formulation for reconstitution. The kit may further comprise a second container which holds a diluent suitable for diluting the pharmaceutical composition towards a higher volume. Suitable diluents include, but are not limited to, the pharmaceutically acceptable excipient of the pharmaceutical composition and a saline solution. Furthermore, the kit may comprise instructions for diluting the pharmaceutical composition and/or instructions for administering the pharmaceutical composition, whether diluted or not. The containers may be formed from a variety of materials such as glass or plastic and may have a sterile access port (for example, the container may be an intravenous solution bag or a vial having a stopper which may be pierced by a hypodermic injection needle). The kit may further comprise more containers comprising a pharmaceutically acceptable buffer, such as phosphate-buffered saline, Ringer's solution and dextrose solution. It may further include other materials desirable from a commercial and user standpoint, including other buffers, diluents, filters, needles, syringes, culture medium for one or more of the suitable hosts. The kits may optionally include instructions customarily included in commercial packages of therapeutic or diagnostic products, that contain information about, for example, the indications, usage, dosage, manufacture, administration, contraindications and/or warnings concerning the use of such therapeutic or diagnostic products.

The kit can also comprise, e.g., a buffering agent, a preservative or a stabilizing agent. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present technology may contain a written product on or in the kit container. The written product describes how to use the reagents contained in the kit. In certain embodiments, the use of the reagents can be according to the methods of the present technology.

In some embodiments, the kit contains additional reagents suitable for detecting mRNA, chromatin accessibility, or protein expression levels of a Type 2 cytokine or Type 2 cytokine receptor signaling (e.g., IL-33, IL-4, IL-13, IL-5, IL-23A, IL-17E, IL-9, IL9R, IL1RL1, IL4RA, IL13RA1, IL13RA2, IL5RA, IL1RL2, IL17RA, IL31RA, IL17RE, IL18R1, IL18RAP, and IL1R1) including an antibody that specifically binds to a Type 2 cytokine or Type 2 cytokine receptor signaling protein, primers and/or probes that specifically hybridize to a nucleic acid sequence that encodes a Type 2 cytokine or Type 2 cytokine receptor signaling protein, or any combination thereof, in biological samples obtained from a patient diagnosed with, or suspected of having pancreatic cancer. Additionally or alternatively, the kits of the present technology may also include instructions for performing assays that measure chromatin accessibility at gene loci (e.g., at intergenic or intronic regions) encoding a Type 2 cytokine or Type 2 cytokine receptor signaling protein (e.g., ChIP, MNase, FAIRE, DNAse, ATAC-based approaches). For example, the kits may contain a positive control sample that contains a reference level of a particular Type 2 cytokine or Type 2 cytokine receptor signaling protein (e.g., IL-33, IL-4, IL-13, IL-5, IL-23A, IL-17E, IL-9, IL9R, IL1RL1, IL4RA, IL13RA1, IL13RA2, IL5RA, IL1RL2, IL17RA, IL31RA, IL17RE, IL18R1, IL18RAP, and IL1R1) and/or a negative control sample that lacks a particular Type 2 cytokine or Type 2 cytokine receptor signaling protein (e.g., IL-33, IL-4, IL-13, IL-5, IL-23A, IL-17E, IL-9, IL9R, IL1RL1, IL4RA, IL13RA1, IL13RA2, IL5RA, IL1RL2, IL17RA, IL31RA, IL17RE, IL18R1, IL18RAP, and IL1R1). The test sample used in the above-described method will vary based on the assay format, nature of the detection method and the tissues, cells or extracts used as the sample to be assayed.

The kit components, (e.g., reagents) can be packaged in a suitable container. The kit can also contain, e.g., a buffering agent, a preservative or a protein-stabilizing agent. The kit can further comprise, or alternatively consist essentially of, or yet further consist of components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit.

As amenable, these suggested kit components may be packaged in a manner customary for use by those of skill in the art. For example, these suggested kit components may be provided in solution or as a liquid dispersion or the like.

### EXAMPLES

The present technology is further illustrated by the following Examples, which should not be construed as limiting in any way.

### Example 1: Experimental Procedures

Generation and authentication of KC-shBrd4 ESC clones. KC-shBrd4 ESCs (Ptf1a-Cre;LSL-KrasG12D;RIK;CHC (Saborowski et al., Genes Dev 28: 85-97 (2014)) were targeted with 2 independent GFP-linked Brd4-shRNAs (shBrd4.552 and shBrd4.1448) (Tasdemir et al., Cancer Discov 6: 612-629 (2016); Zuber et al., Nature 478: 524-528 (2011)) cloned into mir30-based targeting constructs (Dow et al. Nat Protoc 7: 374-393 (2012)), as previously described (Dow et al. Nat Protoc 7: 374-393 (2012); Saborowski et al., Genes Dev 28: 85-97 (2014)). Targeted ESCs were selected and functionally tested for single intregation of the GFP-linked shRNA element into the CHC locus as previously described (Livshits et al., Elife 7: e35216 (2018)). The KC-shRen ESC control clone used in this study has been previously described (Livshits et al., Elife 7: e35216 (2018); Saborowski et al., Genes Dev 28: 85-97 (2014)). Before injection, ESCs were cultured briefly for expansion in KOSR+2i medium (Gertsenstein et al., PLoS One 5: e11260 (2010)). The identity and genotype of the ESC, resulting chimeric mice and their progeny was authenticated by genomic PCR using a common Col1a1 primer CACCCTGAAAACTTTGCCCC (SEQ ID NO: 1) paired with a transgene specific primer: shRen.713: GTATAGATAAGCATTATAATTCCTA (SEQ ID NO: 2); shBrd4.552: TATTGTTCCCATATCCAT (SEQ ID NO: 3); shBrd4.1448: CTAGTTTAGACTTGATTGTG (SEQ ID NO: 4), yielding an ˜250-bp product. ESC were confirmed to be negative for mycoplasma and other microorganisms before injection.

Animal models. All animal experiments in this study were performed in accordance with a protocol approved by the Memorial Sloan-Kettering Institutional Animal Care and Use Committee. Mice were maintained under specific pathogen-free conditions, and food and water were provided ad libitum. All mice strains have been previously described. p48-Cre, LSL-KrasG12D, CHC, CAGs-LSL-RIK, and TRE-GFP-shRen strains were interbred and maintained on mixed background. Chimeric cohorts of KC-shRNA mice derived from the ESCs above described were generated by the Center for Pancreatic Cancer Research (CPCR) at MSKCC or the Rodent Genetic Engineering Core at NYU as previously described (Saborowski et al., Genes Dev 28: 85-97 (2014)). ESC-derived KC-shMyc mice have been previously described (Saborowski et al., Genes Dev 28: 85-97 (2014)). Only KC-shRNA mice with a coat color chimerism of >95% were included for experiments. For induction of shRNA expression, mice were switched to a doxycycline diet (625 mg/kg, Harlan Teklad) that was changed twice weekly.

To compare the effects of tissue injury in the transcriptional and chromatin accessibility landscapes of mutant Kras-expressing or Kras wild-type pancreatic epithelial cells, KC;RIK (p48-Cre;RIK;LSLKrasG12D) or C;RIK (p48-Cre;RIK) male mice were treated with 8 hourly intraperitoneal injections of 80 μg/kg caerulein (Bachem) or PBS for 2 consecutive days, using littermates when possible. To characterize invasive disease, pancreatic ductal adenocarcinoma (PDAC) cells were isolated from cancer lesions arising in autochthonous transgenic models (KPflC;RIK (p48Cre;RIK;LSL-KrasG12D;p53fl/⁺) that were macro-dissected away from pre-malignant tissue. As an orthogonal approach, C57Bl/6 female mice (Harlan) were subjected to for orthotopic transplantations with syngeneic ductal organoids harboruing mutant Kras and inactivated Trp53 gene (see below). Prior to transplantation, organoid cultures were dissociated with TrypLE (Gibco) after mechanical dissociation by pipetting and 1-2×10⁵ cells in serum-free advanced DMEM/F12 (Life Technologies) supplemented with 2 mM glutamine and penn-strep were mixed 1:1 with growth factor reduced matrigel (Corning) and injected into the exposed pancreas of 8-10 weeks old C57B16/N mice using a Hamilton syringe fitted with a 26 gauge needle. For treatment with recombinant Il-33, 5 weeks-old C or KC mice were injected intraperitoneally once daily doses with 1 ug of murine recombinant Il-33 (#580504, R&D Systems) or vehicle (PBS) for 5 consecutive days.

Pancreatic epithelial cell isolation. For RNA-seq and ATAC-seq analyes in lineage-traced epithelial cells isolated directly from KC, KPflC, or KC-shRNA mice, pancreata were finely chopped with scissors and incubated with digestion buffer containing 1 mg/ml Collagenase V (C9263, Sigma-Aldrich), 2 U/mL Dispase (17105041, Life Technologies) dissolved in HBSS with Mg²⁺ and Ca²⁺ (14025076, Thermo Fisher Scientific) supplemented with 0.1 mg/ml DNase I (Sigma, DN25-100MG) and 0.1 mg/ml Soybean Trypsin Inhibitor (STI) (T9003, Sigma), in gentleMACS C Tubes (Miltenyi Biotec) for 42 min at 37° C. using the gentleMACS Octo Dissociator. Normal (non-fibrotic) pancreas samples were dissociated as above, except that the digestion buffer contained 1 mg/mL Collagease D (11088858001, Sigma-Aldrich). After enzymatic dissociation, samples were washed with PBS and further digested with a 0.05% solution of Trypsin-EDTA (Ser. No. 15/400,054, Thermo Fisher Scientific) diluted in PBS for 5 min at 37° C. Trypsin digestion was neutralized with FACS buffer (10 mM EGTA and 2% FBS in PBS) containing STI. Samples were then washed in FACS buffer containing DNase I and STI, filtered through a 100 μm strainer. Cell suspensions were blocked for 5 min at room temperature with rat anti-mouse CD16/CD32 with Fcblock (Clone 2.4G2, BD Biosciences) in FACS buffer containing DNase I and STI, and APC-conjugated CD45 antibody was then added (Clone 30-F11,BD Biosciences) and incubated for 10 min at 4° C. Cells were then washed once with in FACS buffer containing DNase I and STI, filtered through a 40 μm strainer, and resuspended in FACS buffer containing DNase I and STI and 300 nM DAPI as live-cell marker. Sorts were performed on a BD FACSAria III cell sorter (Becton Dickinson) for mKate2 (co-expressing GFP for on dox-shRNA mice), excluding CD45⁺ cells. Cells were sorted directly into Trizol LS (Thermo Fisher Scientific) for RNA-seq or collected in 2% FBS in PBS for ATAC-seq.

Immunofluorescence, immunohistochemistry and histological analyses. Tissues were fixed overnight in 10% neutral buffered formalin (Richard-Allan Scientific), embedded in paraffin and cut into 5 μm sections. Slides were heated for 30 min at 55° C., deparaffinized, rehydrated with an alcohol series and subjected to antigen retrieval with citrate buffer (Vector Laboratories Unmasking Solution, H-3300) for 25 min in a pressure cooker set on high. Sections were treated with 3% H₂O₂ for 10 min followed by a wash in dionized water (for immunohistochemistry only), washed in PBS, then blocked in PBS/0.1% Triton X-100 containing 1% BSA. Primary antibodies were incubated overnight at 4° C. in blocking buffer. The following primary antibodies were used: mKate2 (Evrogen, AB233), GFP (ab13970, Abcam and 2956S, Cell Signaling Technology), Brd4 (HPA015055, Sigma-Aldrich), Myc (ab32072, Abcam), Cpa1 (R&D, AF2765), Clusterin (SCBT sc-6419), SOX9 (Millipore AB553), Amylase (sc-31869, Santa Cruz), Krt19 (Troma III, Developmental Studies Hybridoma Bank) and Il-33 (AF3626, R&D). For mKate2, GFP and cMyc immunohistochemistry, Vector ImmPress HRP kits and ImmPact DAB (Vector Laboratories) were used for secondary detection. Tissues were then counterstained with Haematoxylin or when indicated Alcian blue (pH 2.5) and 0.1% Nuclear Fast Red Solution, dehydrated and mounted with Permount (Fisher). The immunohistochemistry detection of Brd4 was performed at the Molecular Cytology Core Facility of Memorial Sloan Kettering Cancer Center using Discovery XT processor (Ventana Medical Systems-Roche). The tissue sections were blocked for 30 min in 10% normal goat serum, 2% BSA in PBS. A rabbit polyclonal anti-Brd4 antibody (HPA015055, Sigma-Aldrich) was used in 1 ug/ml (1:100) concentrations. The incubation with the primary antibody was done for 6 hours, followed by 60 minutes incubation with biotinylated goat anti-rabbit IgG (PK610, Vector labs) in 5.75 ug/mL concentration. Blocker D, Streptavidin-HRP and DAB detection kit (760-124, Ventana Medical Systems-Roche) were used according to the manufacturer instructions. Slides were counterstained with Hematoxylin (760-2021, Ventana), Bluing Reagent (760-2037, Ventana) and coverslipped with Permount (Fisher Scientific).

For immunofluorescence, the following secondary antibodies were used: donkey anti-chicken CF488 (SAB4600031, Sigma-Aldrich), goat anti-chicken AF488 (A11039, Invitrogen), donkey anti-rabbit AF594 (A21207, Invitrogen), goat anti-rabbit AF594 (A11037, Thermo Fisher Scientific), donkey anti-goat AF488 (A11055, Invitrogen) and donkey anti-goat AF594 (A32758, Thermo Fisher Scientific). Slides were counterstained with DAPI and mounted in ProLong Gold (Life Technologies). Hematoxylin and eosin (H&E) was performed using standard protocols.

Images were acquired on a Zeiss AxioImager microscope using a 10×(Zeiss NA 0.3) or 20×(Zeiss NA 0.17) objective, an ORCA/ER CCD camera (Hamamatsu Photonics, Hamamatsu, Japan), and Axiovision software. For histological analysis of lesions in KC-shRen or KC-shBrd4 mice, lesions were classified and graded by a veterinary pathologist blinded to genotype into ADM (ductal metaplasia) or mucinous (Alcian Blue+) PanIN lesions using established criteria. The GFP⁺ area was quantified using “SpotR software”. All lesions in at least 3 representative 20× fields per section were measured and counted. The results were averaged and normalized to total tissue area analyzed. Statistical analyses were performed using unpaired t-test in Prism 7. Graphs displayed averages±SEM of independent biological replicates (mice).

Quantitative Real-Time polymerase chain reaction (qRT-PCR) analysis Total RNA was isolated from mKate2⁺,CD45-DAPI-sorted primary pancreatic epithelial cells using the Trizol LS (Thermo Fisher Scientific), and cDNA was obtained from 500 ng of RNA using the Transcriptor First Strand cDNA Synthesis Kit (Roche) after treatment with DNAse I (Invitrogen) following manufacturer's instructions. The following primer sets for mouse sequences were used: Il-33_F GCTGCGTCTGTTGACACATT (SEQ ID NO: 5), Il-33_R GACTTGCAGGACAGGGAGAC (SEQ ID NO: 6), Agr2_F ACAACTGACAAGCACCTTTCTC (SEQ ID NO: 7), Agr2_R GTTTGAGTATCGTCCAGTGATGT (SEQ ID NO: 8), Muc6_F AGCCCACATTCCCTATCAGC (SEQ ID NO: 9), Muc6_R CACAGTGGAAGATTGCGAGAG (SEQ ID NO: 10), Cpa1_F CAGTCTTCGGCAATGAGAACT (SEQ ID NO: 11), Cpa1_R GGGAAGGGCACTCGAACATC (SEQ ID NO: 12), Sox9_F CGTGCAGCACAAGAAAGACCA (SEQ ID NO: 13), Sox9_R GCAGCGCCTTGAAGATAGCAT (SEQ ID NO: 14), Hprt_F TCAGTCAACGGGGGACATAAA (SEQ ID NO: 15), Hprt_R GGGGCTGTACTGCTTAACCAG (SEQ ID NO: 16), Rplp0_F GCTCCAAGCAGATGCAGCA (SEQ ID NO: 17) and Rplp0_R CCGGATGTGAGGCAGCAG (SEQ ID NO: 18) qRT-PCR was carried out in triplicate (5 cDNA ng/reaction) using SYBR Green PCR Master Mix (Applied Biosystems) on the ViiA 7 Real-Time PCR System (Life technologies). Hprt or Rplp0 (aka 36b4) served as endogenous normalization controls.

RNA-Seq Analysis.

RNA extraction, RNA-seq library preparation and sequencing: Total RNA was isolated from primary mKate2⁺,CD45-DAPI-pancreatic epithelial cells isolated from normal, regenerating (Reg-ADM), early neoplastic (Kras*, Kras*-ADR) and cancer (PDAC) tissues into TRIzolLS and assessed using a Agilent 2100 Bioanalyzer. Sequencing and library preparation was performed at the Integrated Genomics Operation (IGO) at MSKCC. RNA-seq libraries were prepared from total RNA. After RiboGreen quantification and quality control by Agilent BioAnalyzer, 100-500 ng of total RNA underwent polyA selection and TruSeq library preparation according to instructions provided by Illumina (TruSeq Stranded mRNA LT Kit, RS-122-2102), with 8 cycles of PCR. Samples were barcoded and run on a HiSeq 4000 or HiSeq 2500 in a 50 bp/50 bp paired end run, using the HiSeq 3000/4000 SBS Kit or TruSeq SBS Kit v4 (Illumina). An average of 41 million paired-end reads was generated per sample. At the most the ribosomal reads represented 0.01% of the total reads generated and the percent of mRNA bases averaged 53%.

RNA-seq read mapping and differential expression analysis: Resulting RNA-Seq data was analyzed by removing adaptor sequences using Trimmomatic. RNA-Seq reads were then aligned to GRCm38.91 (mm10) with STAR and transcript count was quantified using featureCounts to generate raw count matrix. Differential gene expression analysis was performed using DESeq2 package between experimental conditions, using 3-5 independent biological replicates (individual mouse) per condition, implemented in R. Principal component analysis (PCA) was performed using the DESeq2 package in R. Differentially expressed genes (DEGs) were determined by >2-fold change in gene expression with adjusted P-value<0.05.

RNA-Seq heatmap clustering and pathway enrichment analysis of gene clusters: The DEGs of regeneration and early neoplasia (determined by comparing Reg-ADM, Kras*, and Kras*-ADR conditions to Normal pancreas; FC>2, padj<0.05) were clustered using kmeans clustering of 5 and plotted using pheatmap package in R, with Euclidean measure to obtain distance matrix and ward.D2 agglomeration method for clustering. Pathway enrichment analysis was performed in the resulting gene clusters with the Reactome database using enrichR. Significance of the test was assessed using combined score, described as c=log(p)*z, where c is the combined score, p is Fisher exact test p-value, and z is z-score for deviation from expected rank.

Gene set enrichment analysis (GSEA): GSEA was performed using the GSEAPreranked tool for conducting gene set enrichment analysis of data derived from RNA-seq experiments (version 2.07) against signatures in the MSigDB database and published expression signatures in organoid models and human samples. The metric scores were calculated using the sign of the fold change multiplied by the inverse of the p-value.

Overlap with human gene expression datasets: 2 independent publicly datasets of microarray data from human PDAC and normal pancreas samples (GSE71729, Moffitt et al., Nat Genet 47, 1168-1178 (2015); and GSE62452, Yang et al., Cancer Res 76, 3838-3850 (2016)) were used. Differential expression analysis was then applied using the limma package to define differentially expressed genes (DEGs) between PDAC vs normal samples, using>2-fold change and adjusted P-value<0.05 cut-off. The overlap between human DEGS with DEGs identified in GEMMS is summarized in FIGS. 29A and 29B.

ATAC-Seq Analysis

Cell preparation, transpositon reaction, ATAC-seq library construction and sequencing: 65,000 mKate2⁺ cells isolated by FACS, washed once with 50 uL of cold PBS, and resuspended in 50 ul cold lysis buffer (Buenrostro et al., 2015). Cells were then centrifuged immediately for 10 min at 500 g, 4° C. and nuclei pellet was subjected to transposition with Nextera Tn5 transposase (FC-121-1030, Illumina) for 30 min at 37° C., according to manufacturer's instructions. DNA was eluted using MinElute PCR Purification Kit in 11.5 ul elution buffer (Qiagen). ATAC-seq libraries were prepared using the NEBNext High-Fidelity 2× PCR Master Mix (NEB M0541) as previously described (Livshits et al., 2018). Purified libraries were assessed using a Bioanalyzer High-Sensitivity DNA Analysis kit (Agilent). Approximately 200 million paired-end 50 bp reads were sequenced per replicate on a HiSeq 2500 (High Output) at the New York Genome Center.

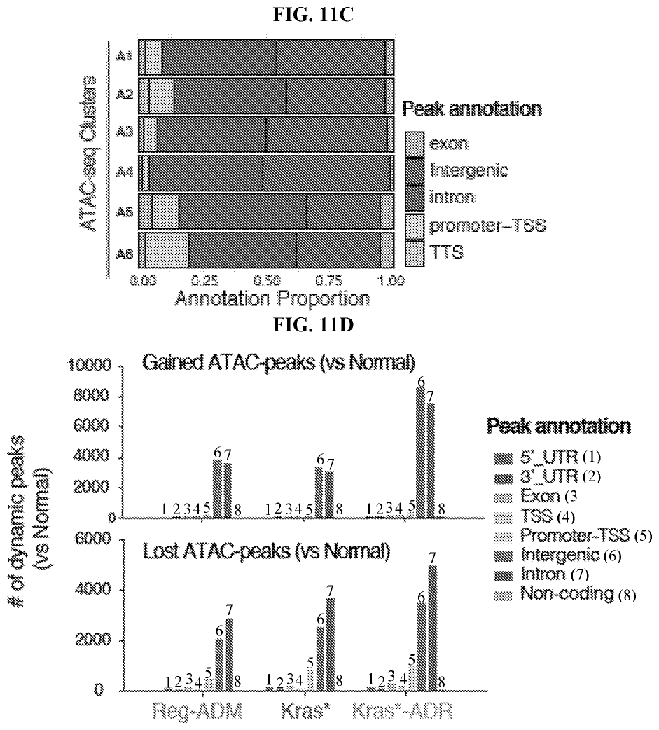

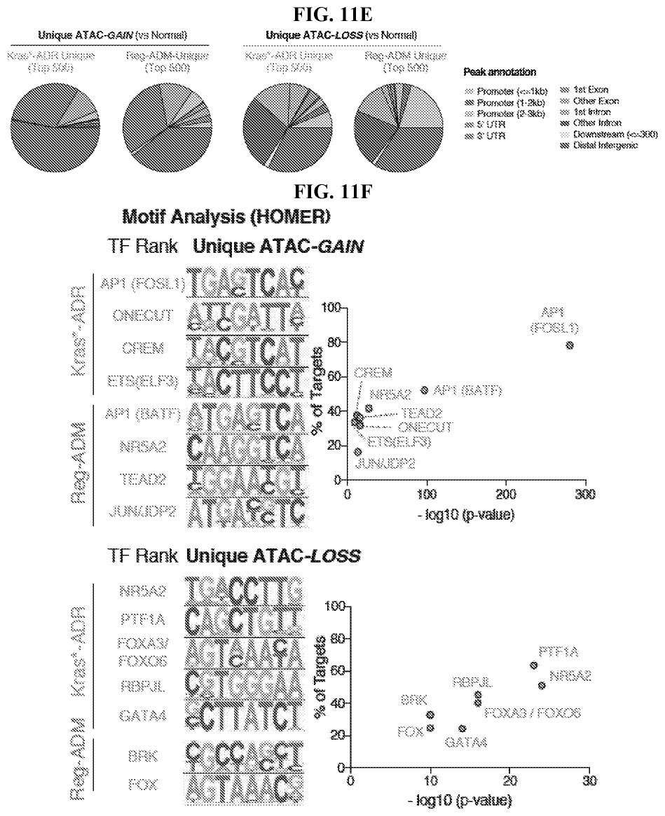

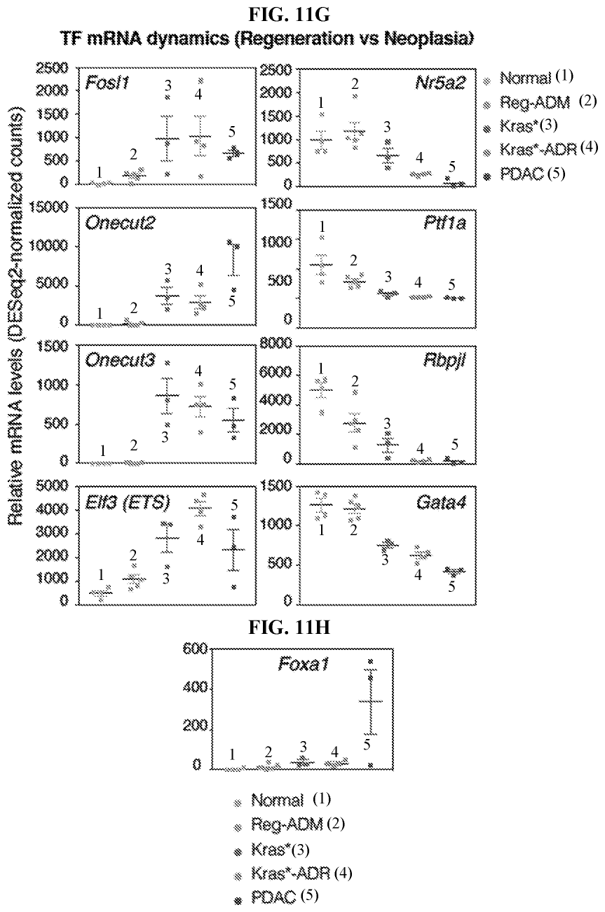

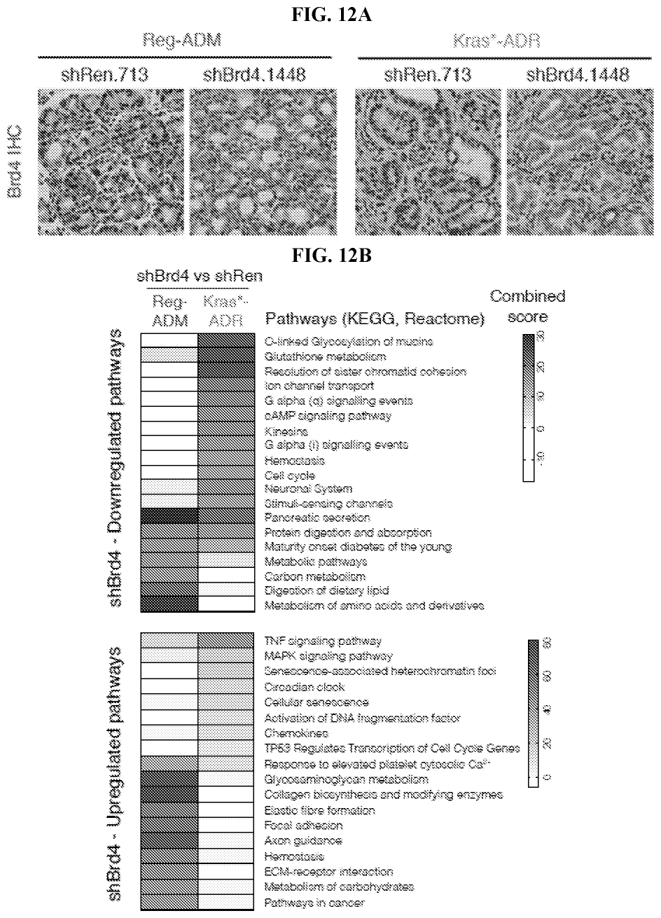

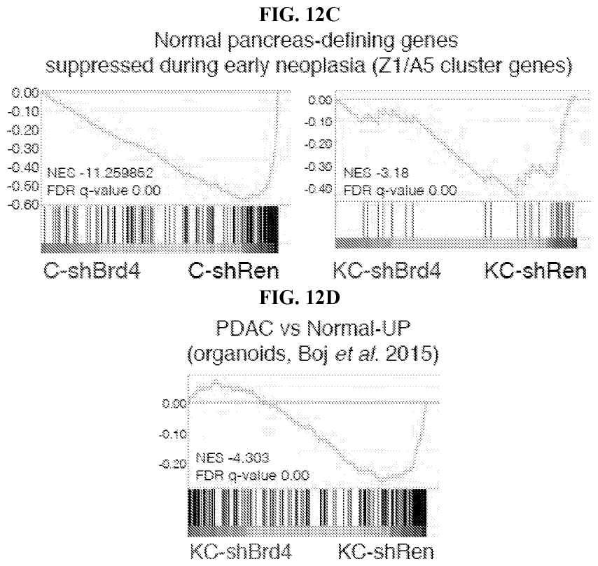

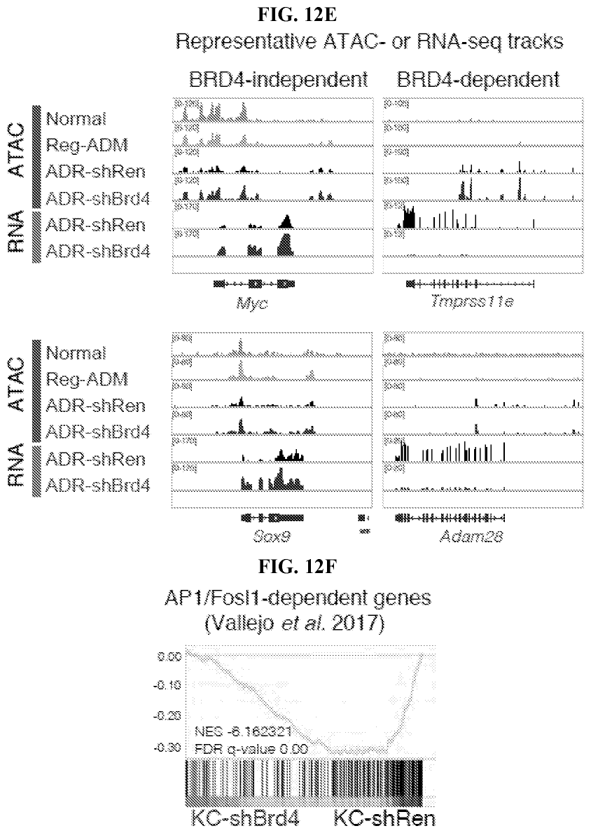

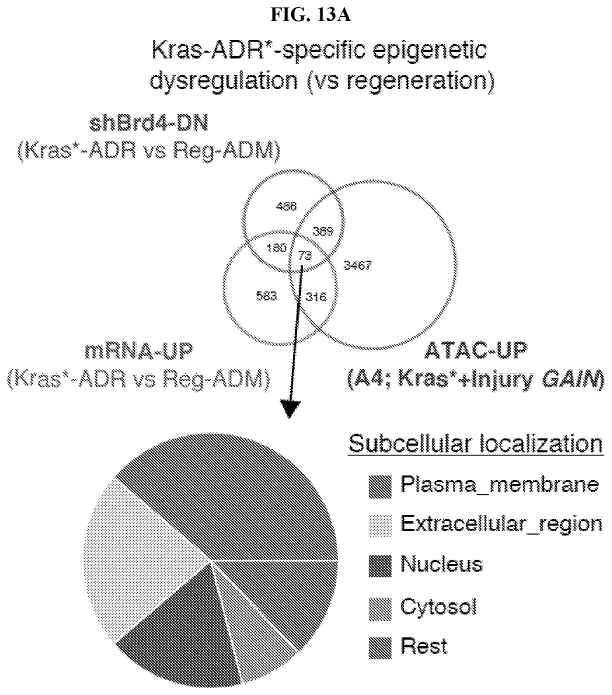

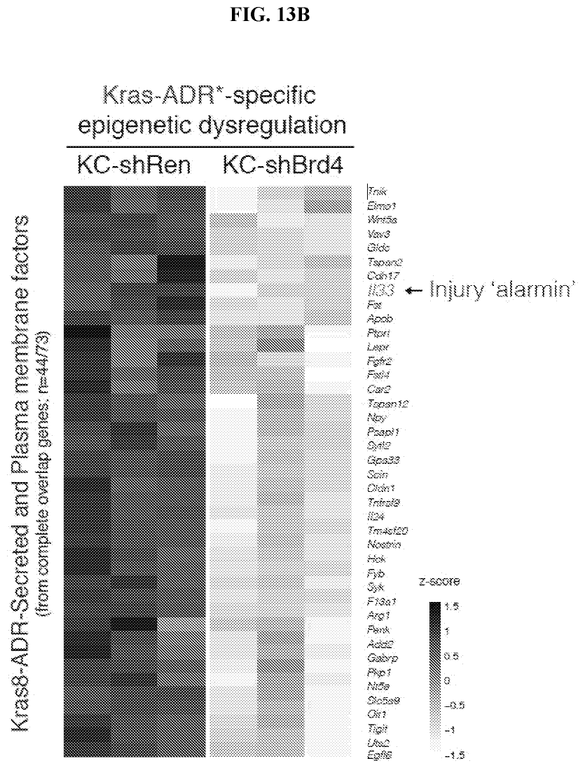

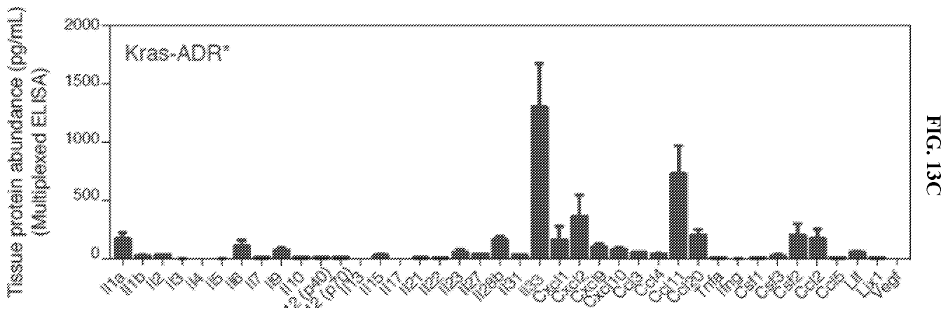

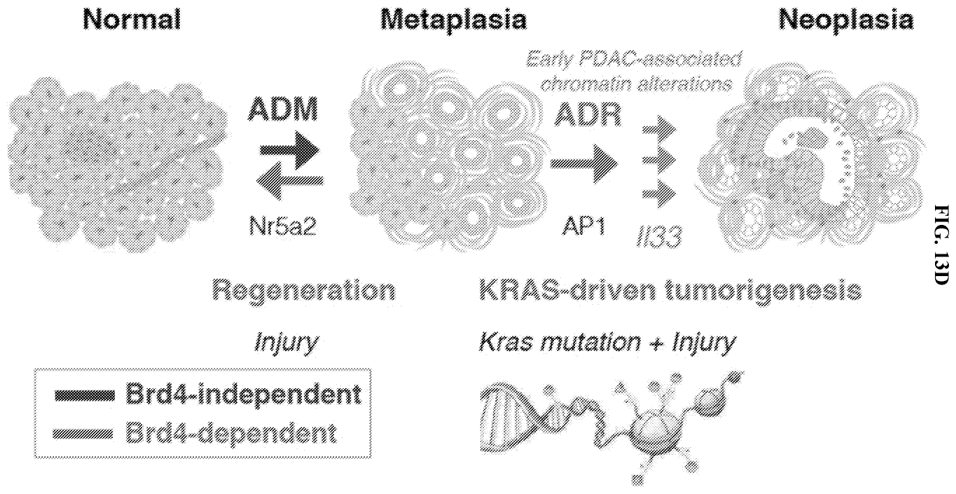

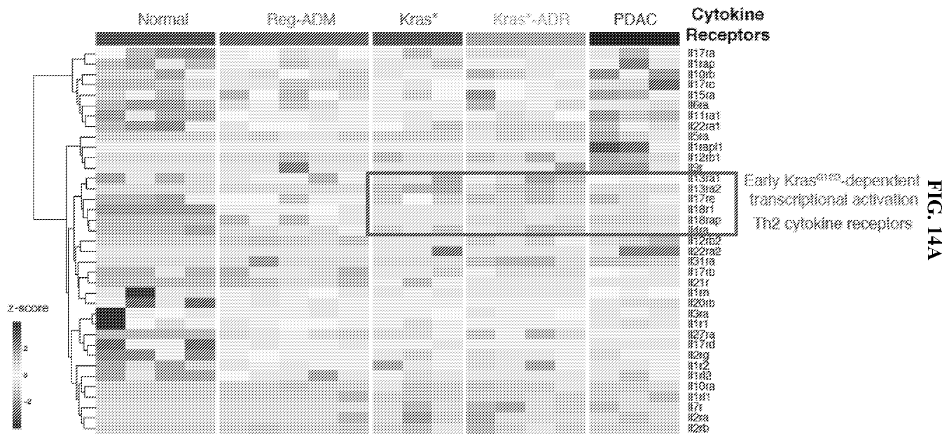

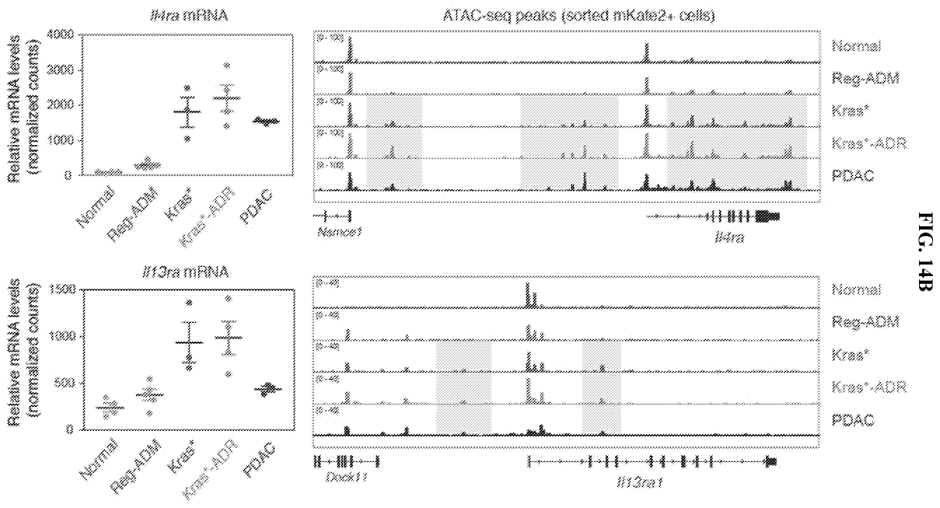

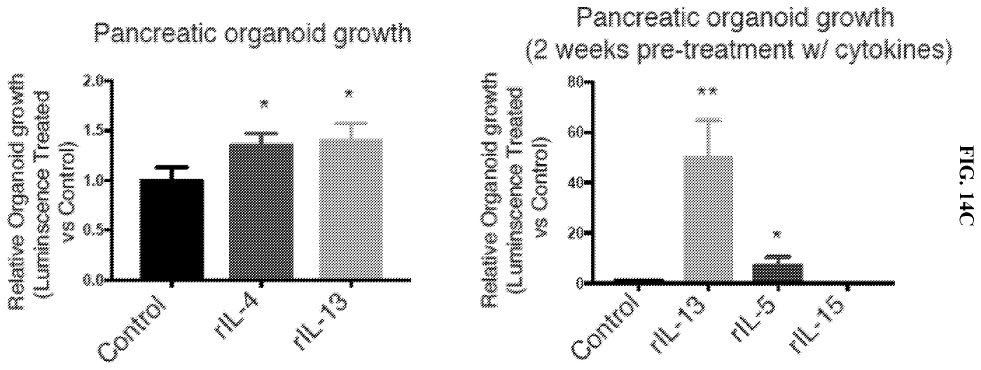

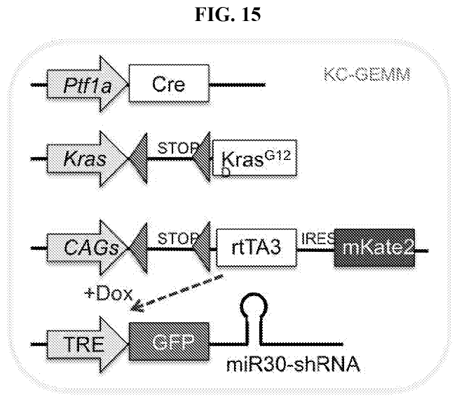

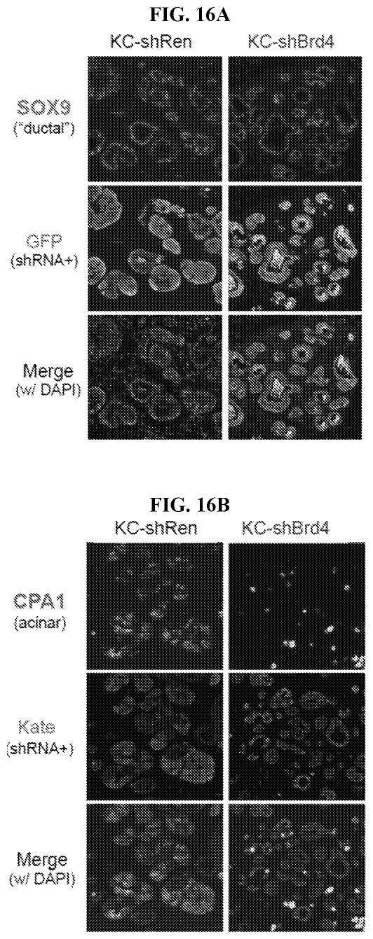

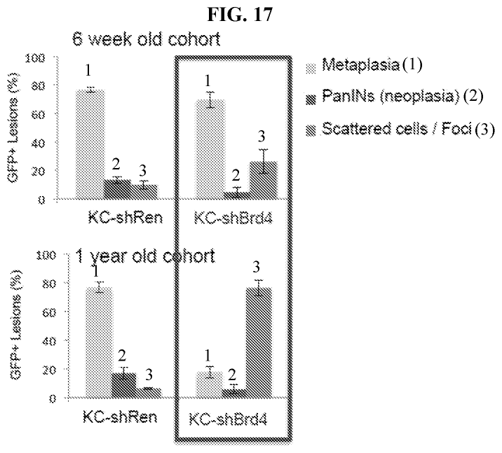

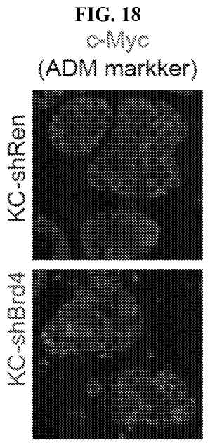

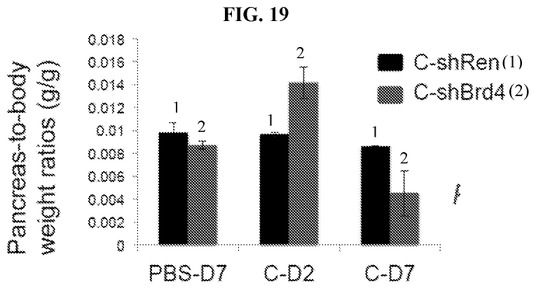

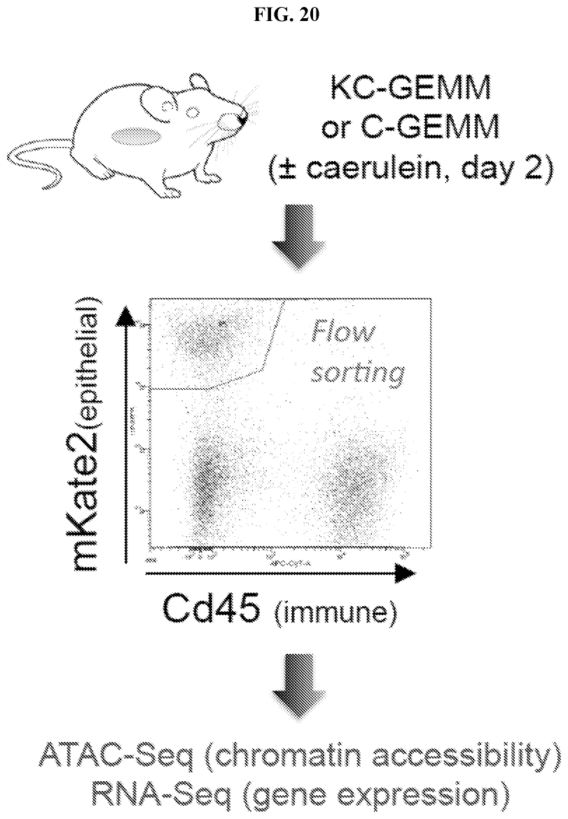

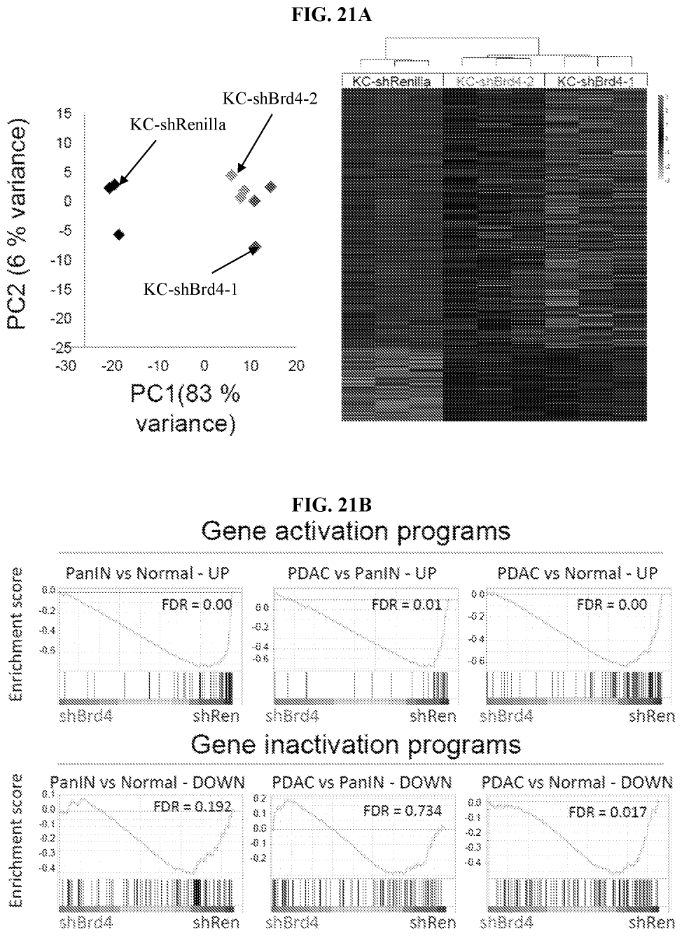

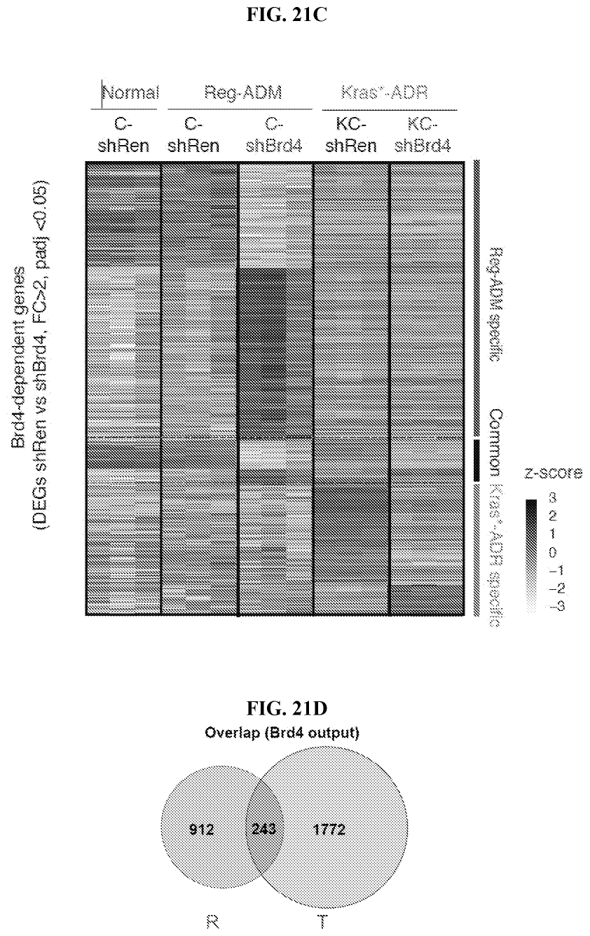

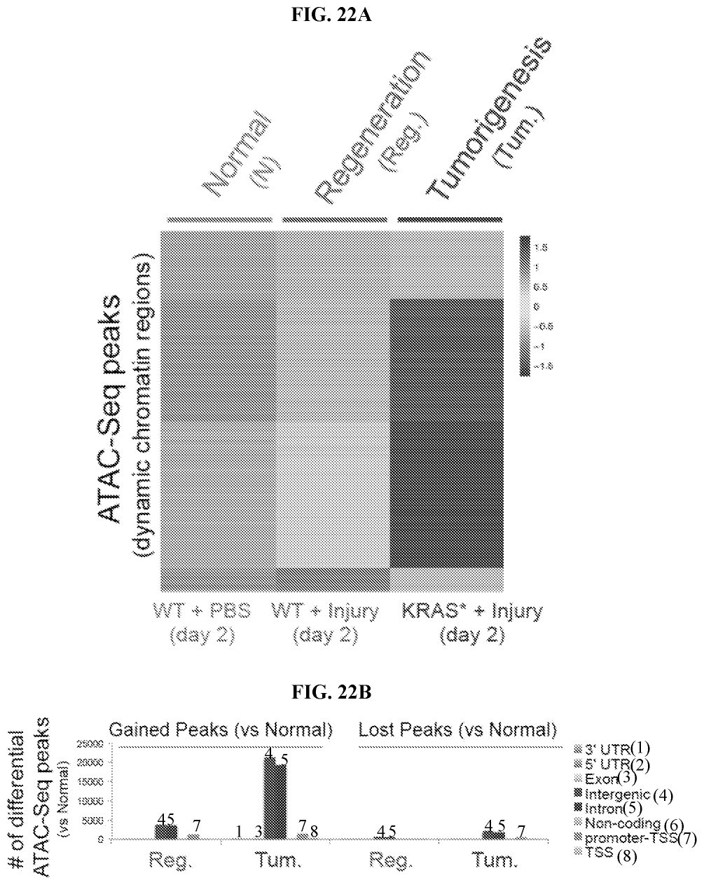

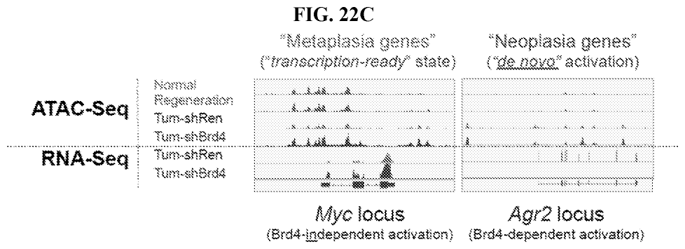

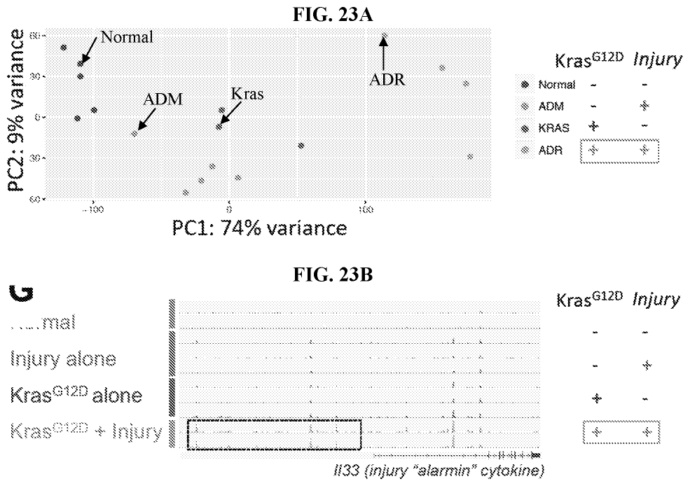

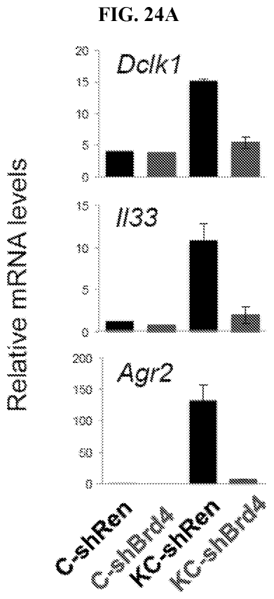

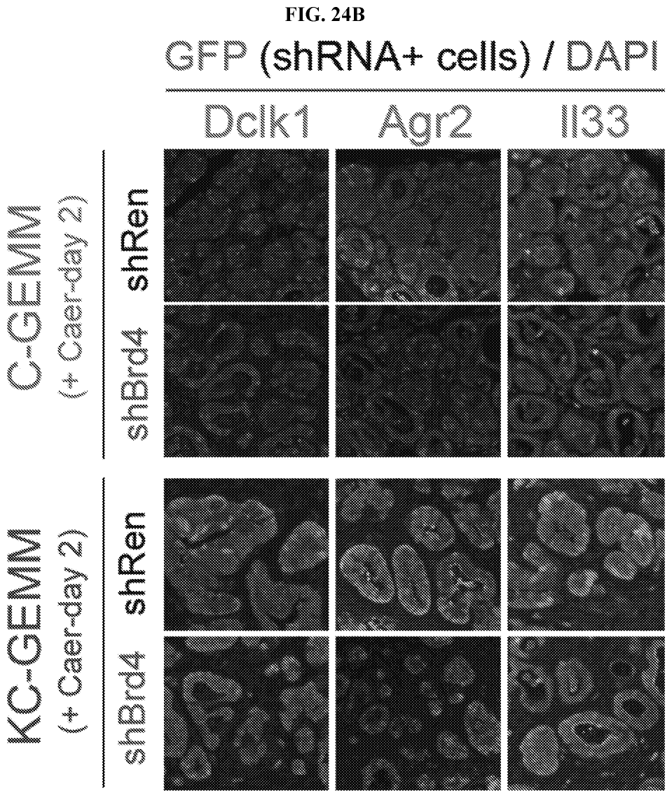

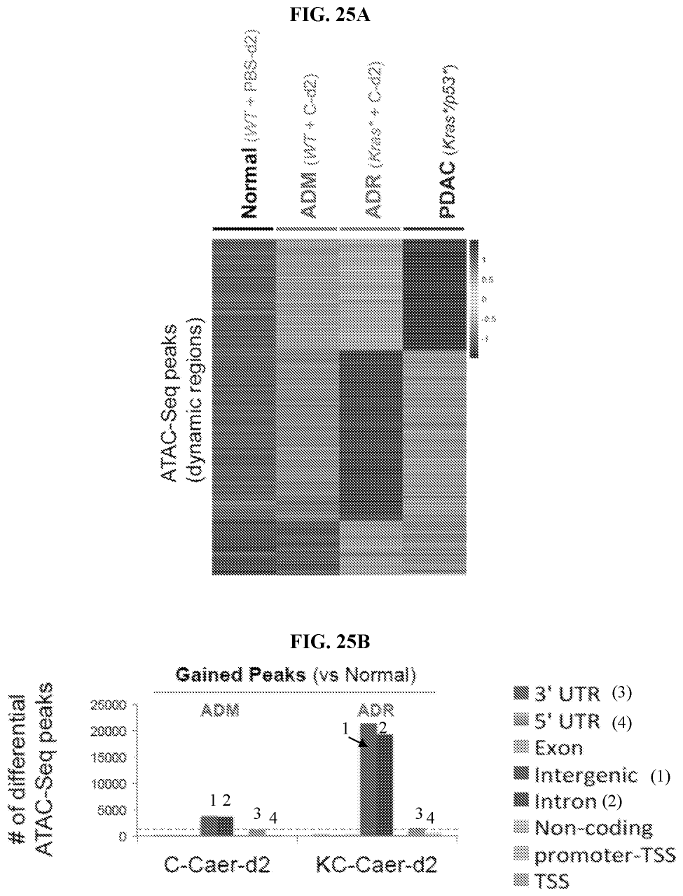

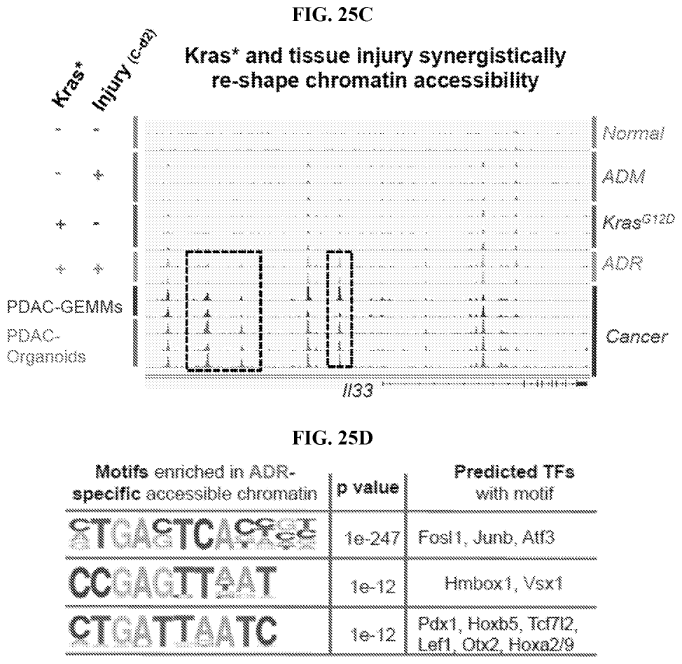

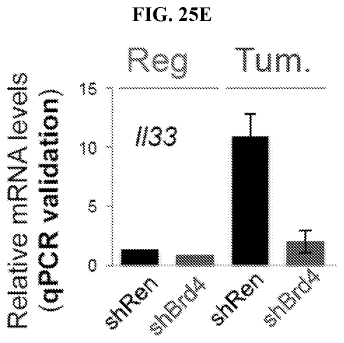

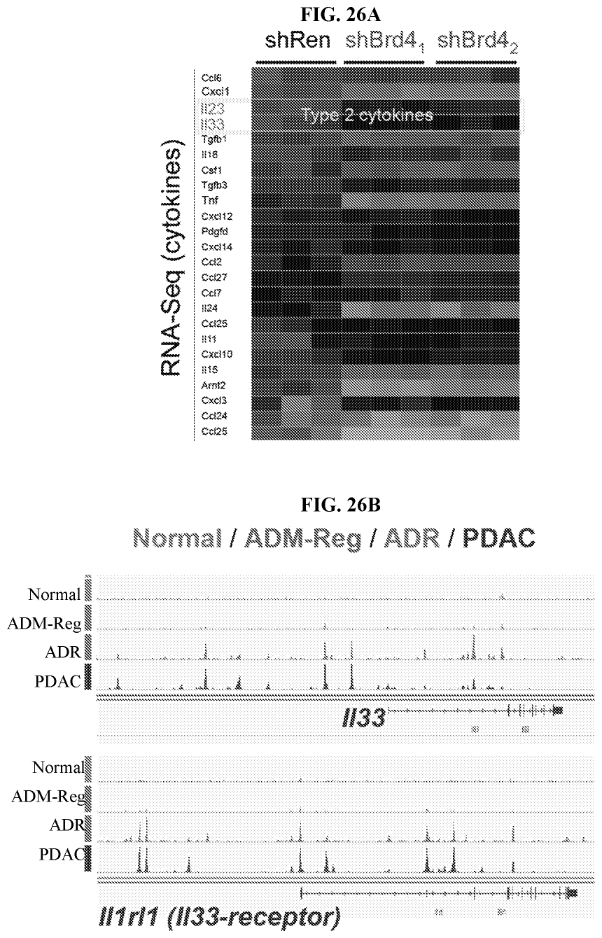

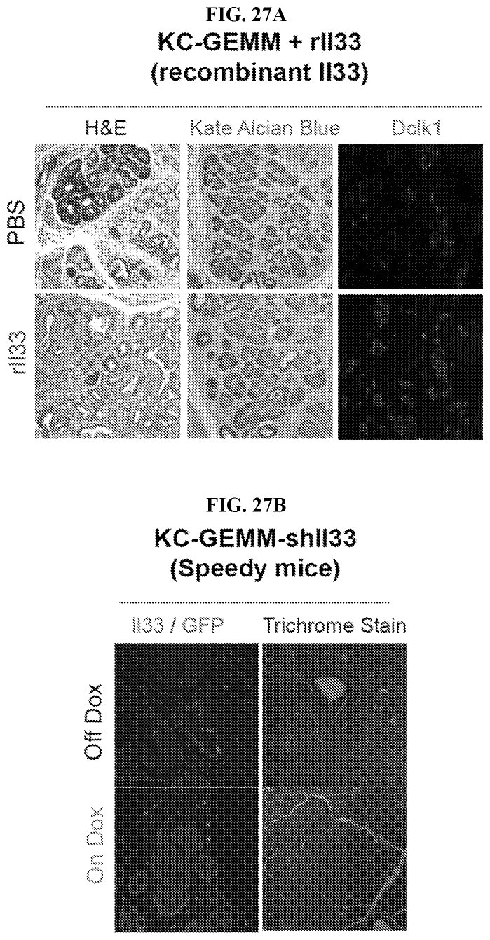

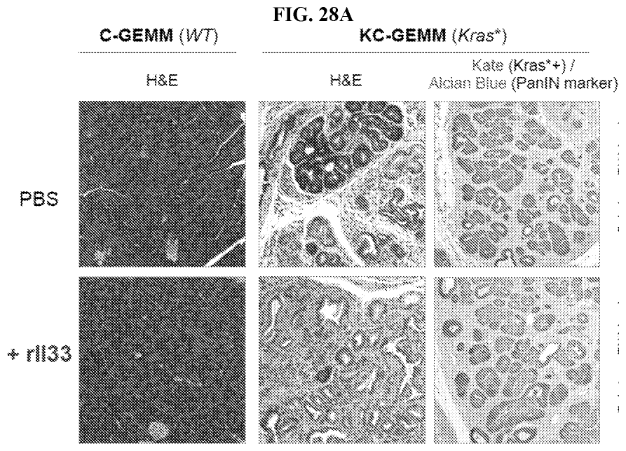

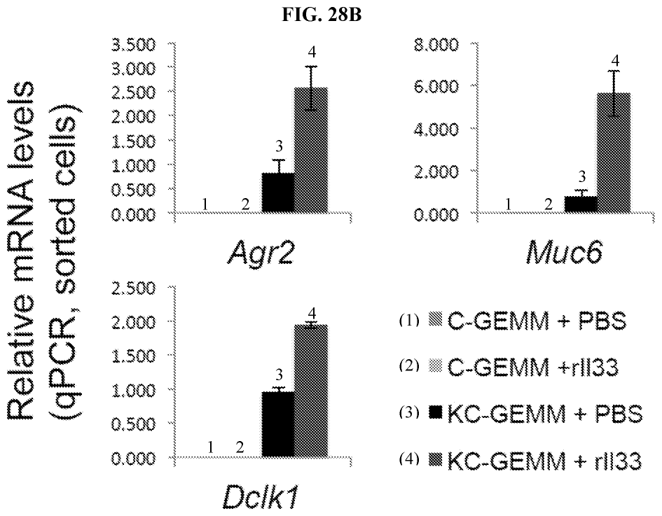

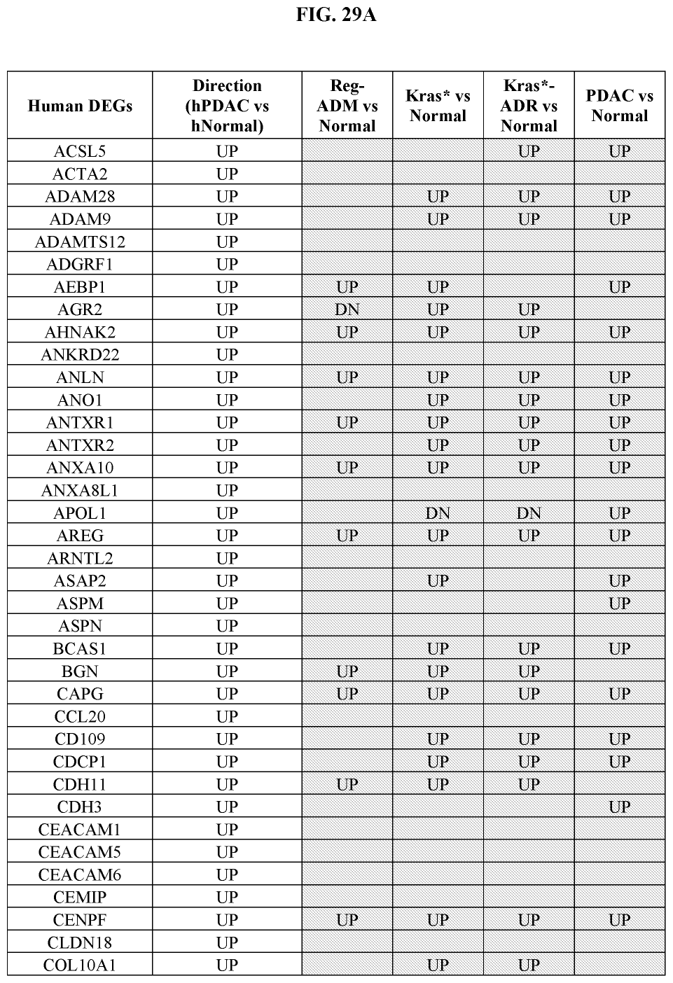

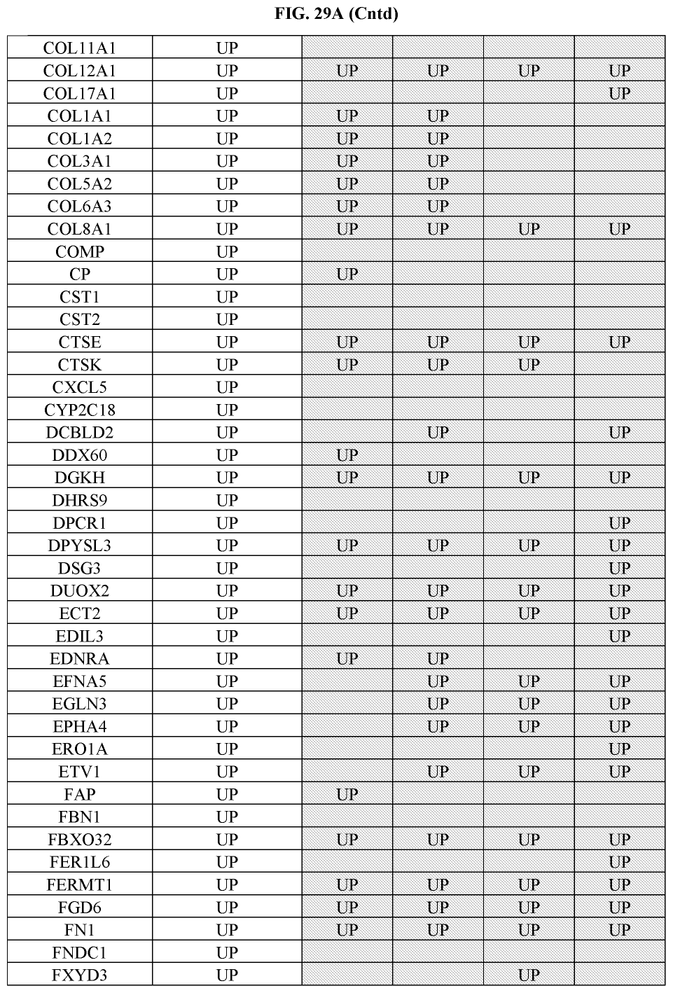

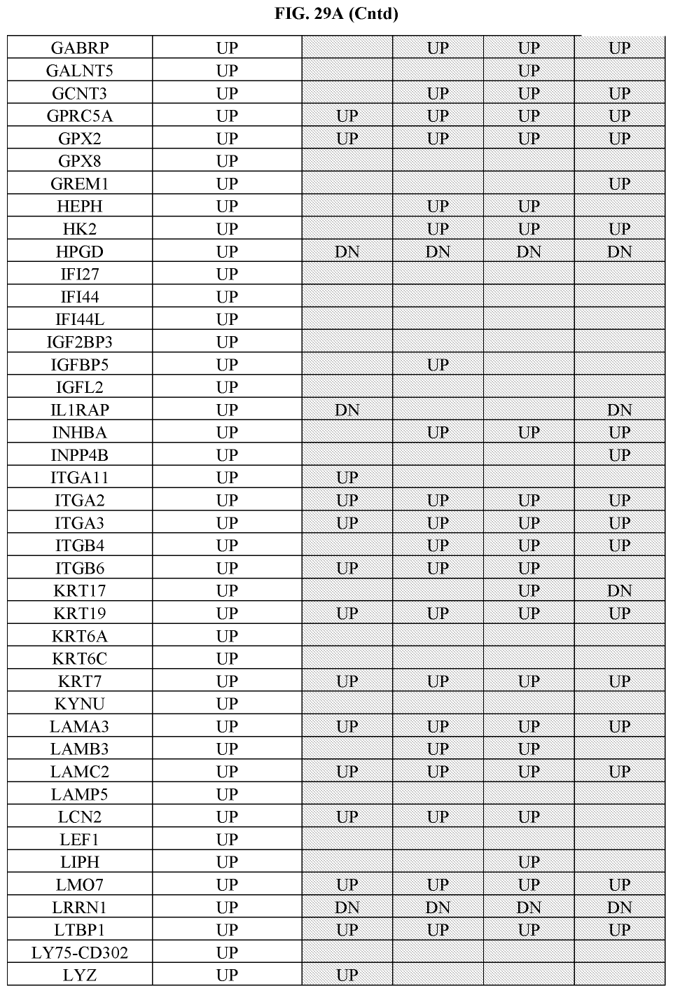

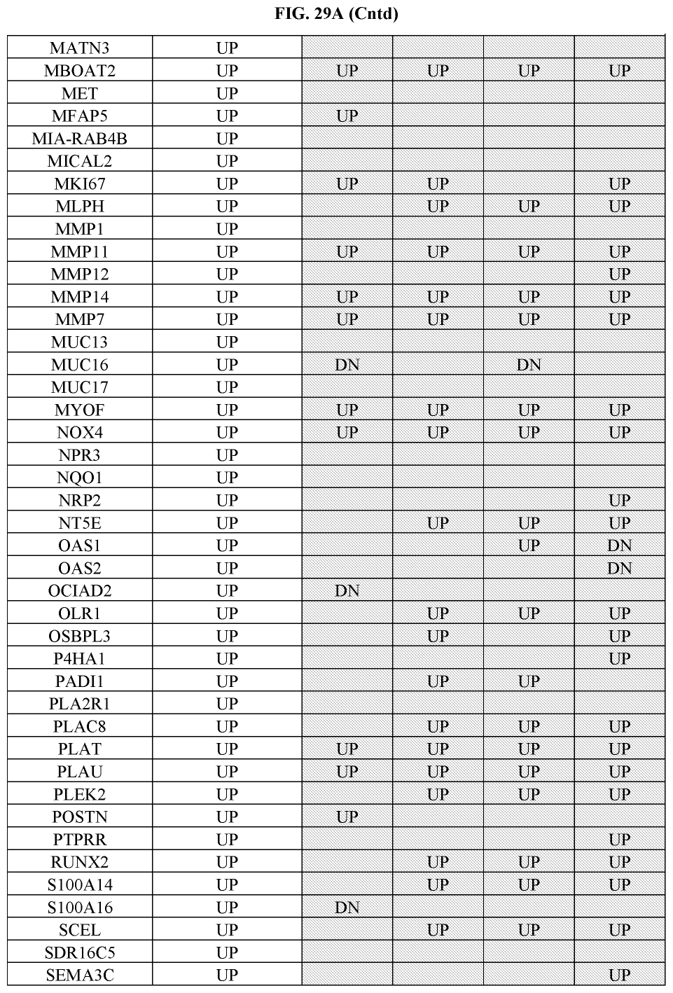

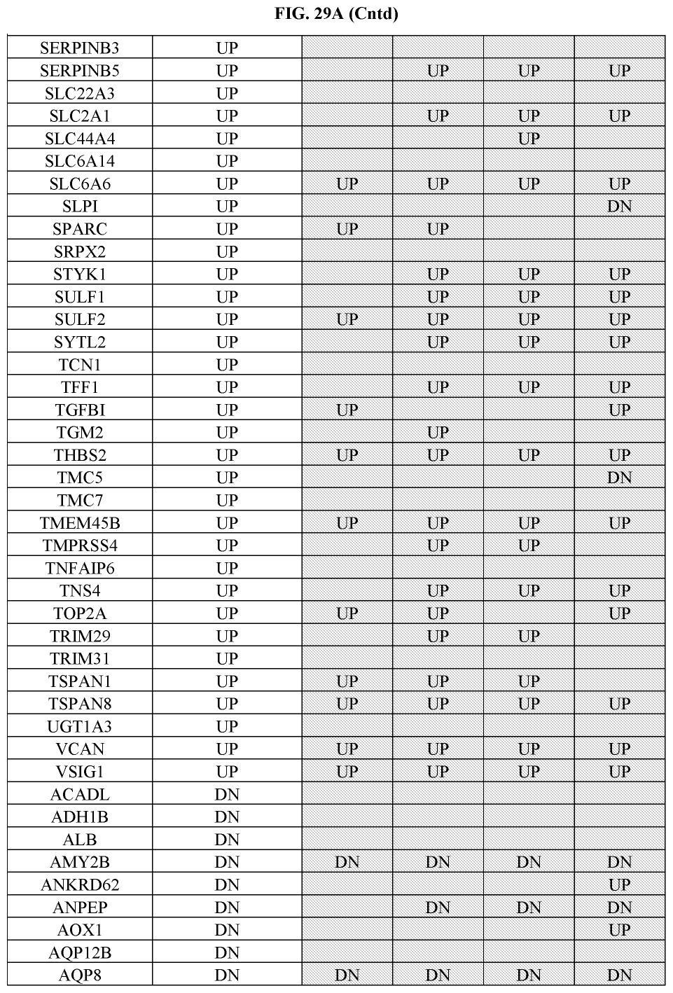

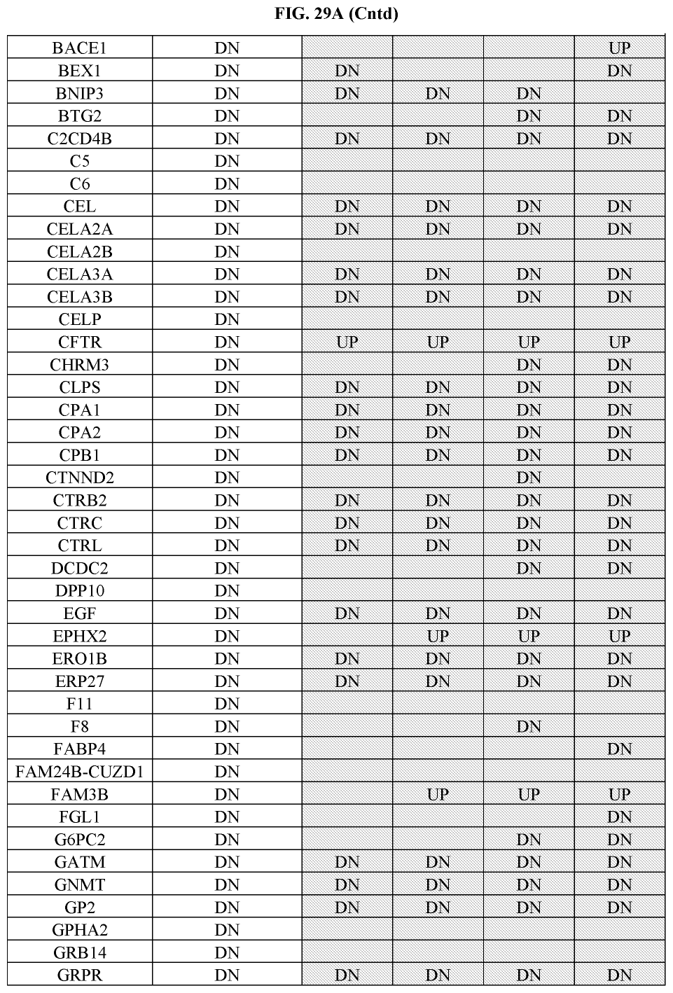

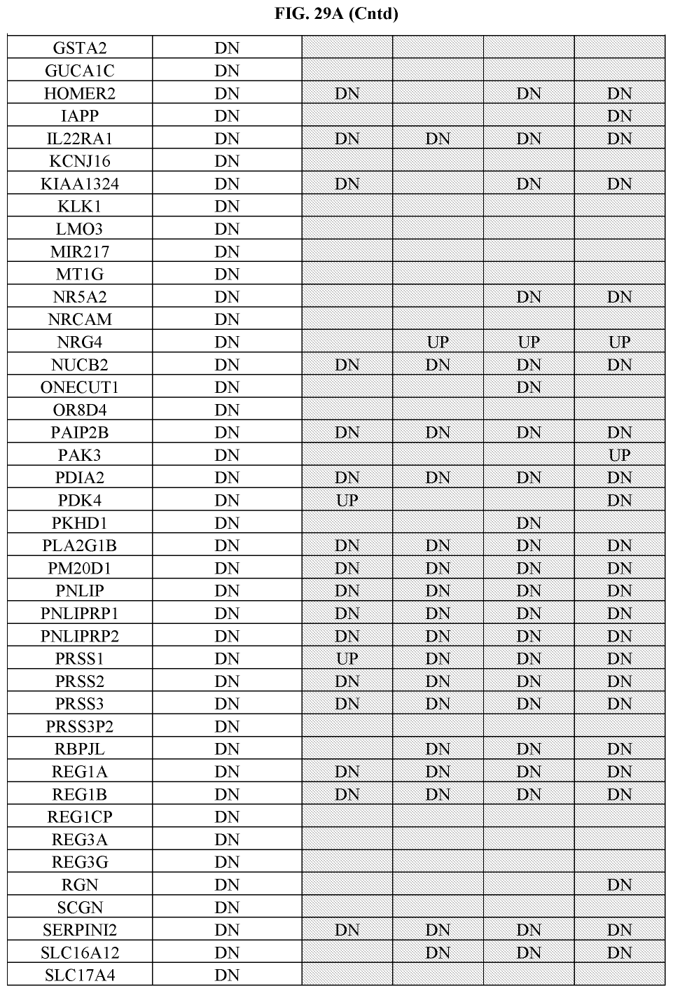

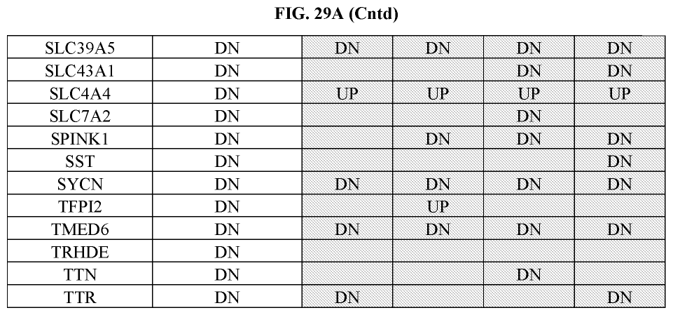

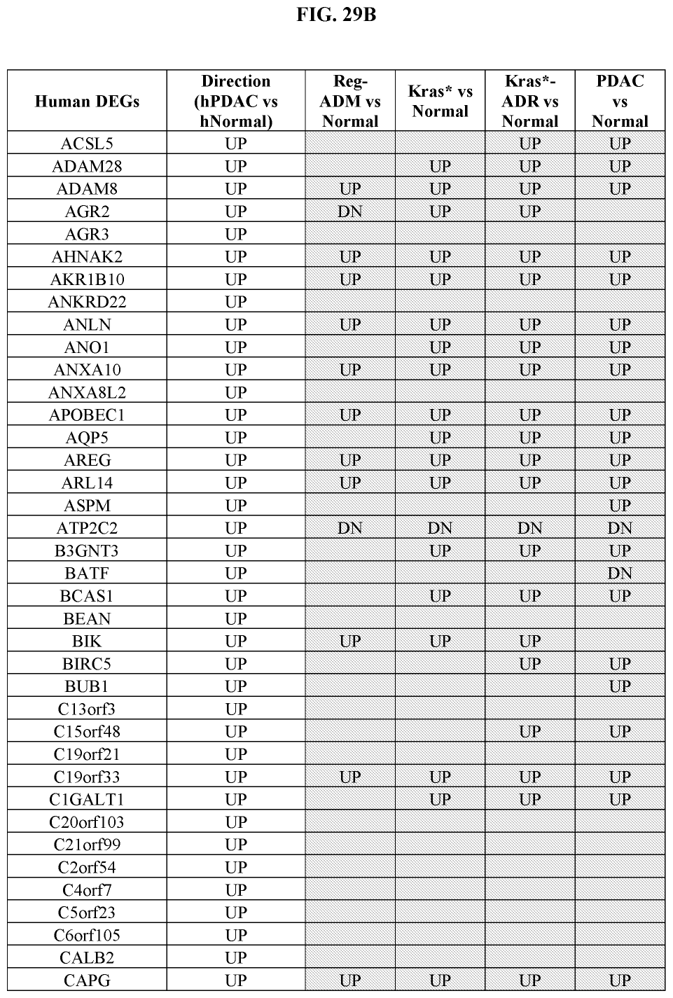

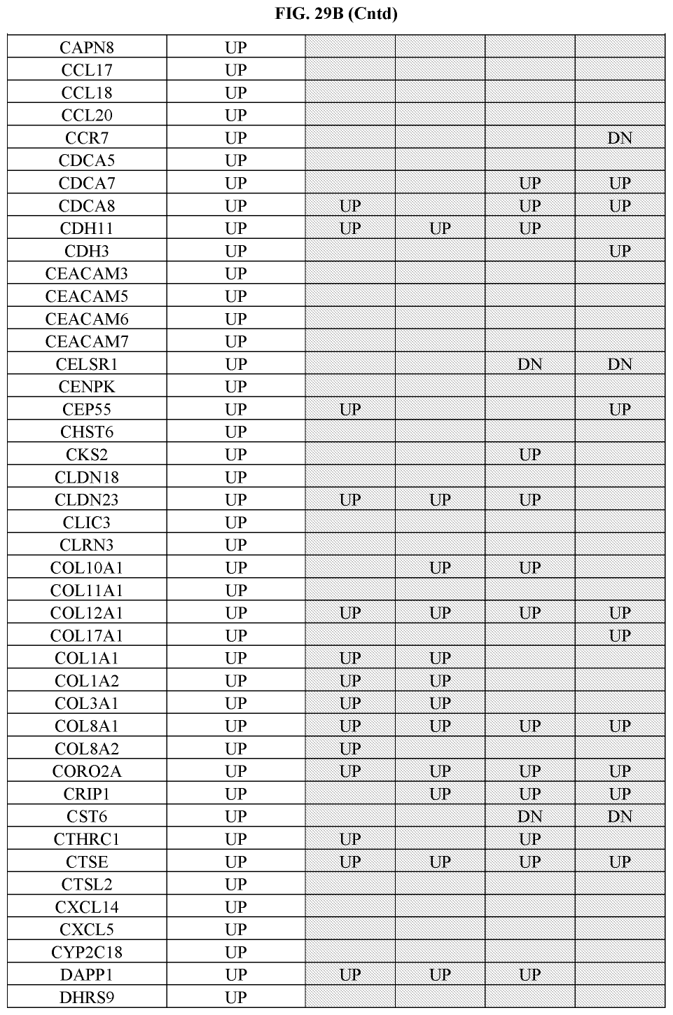

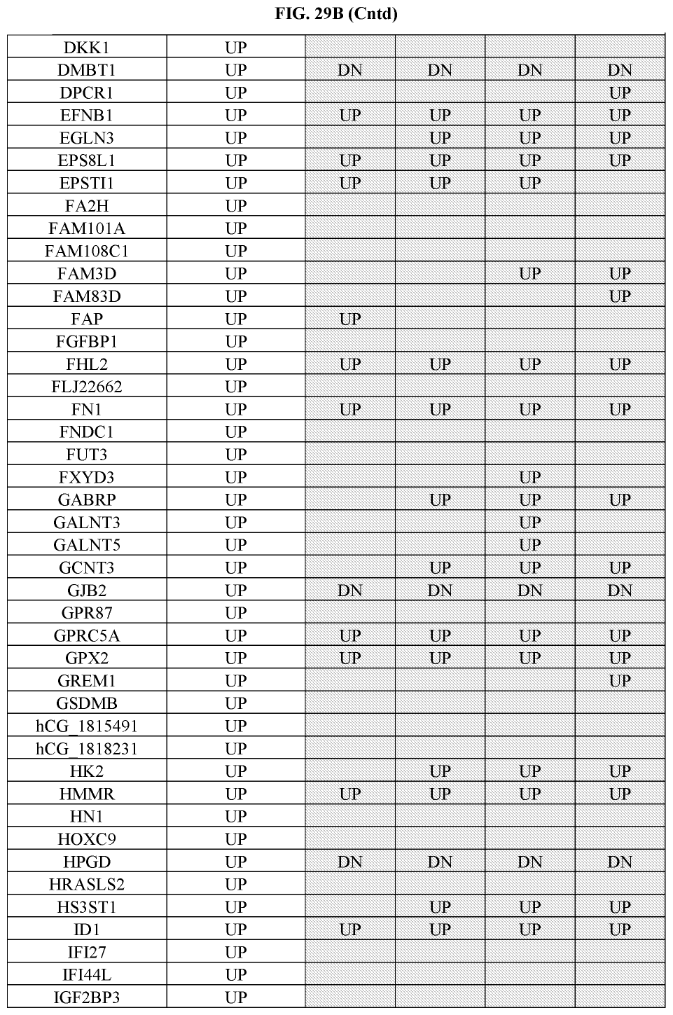

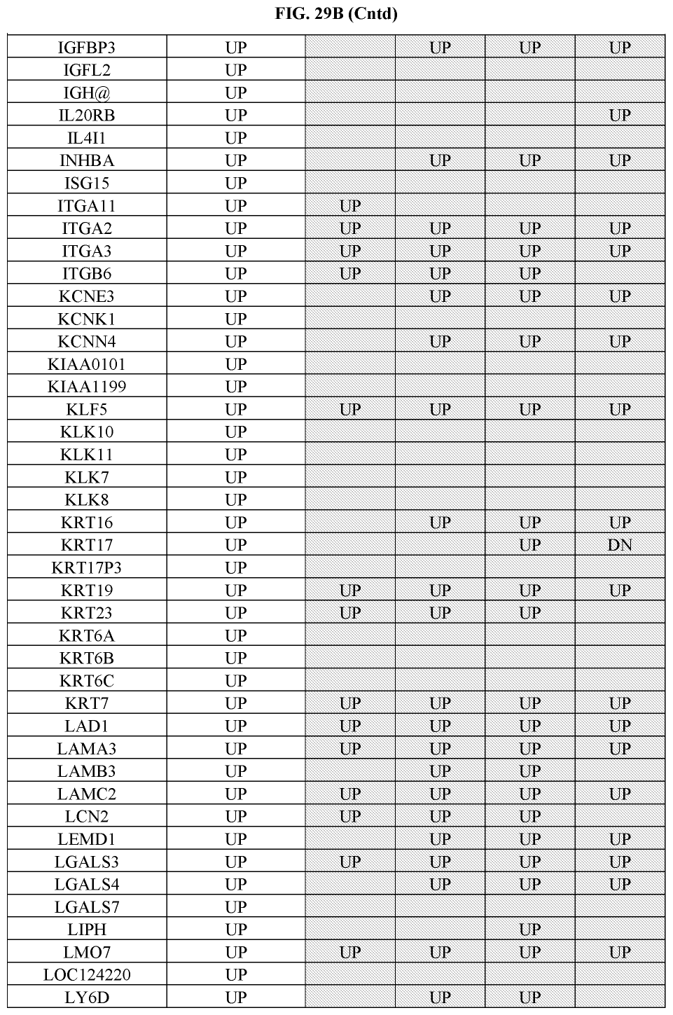

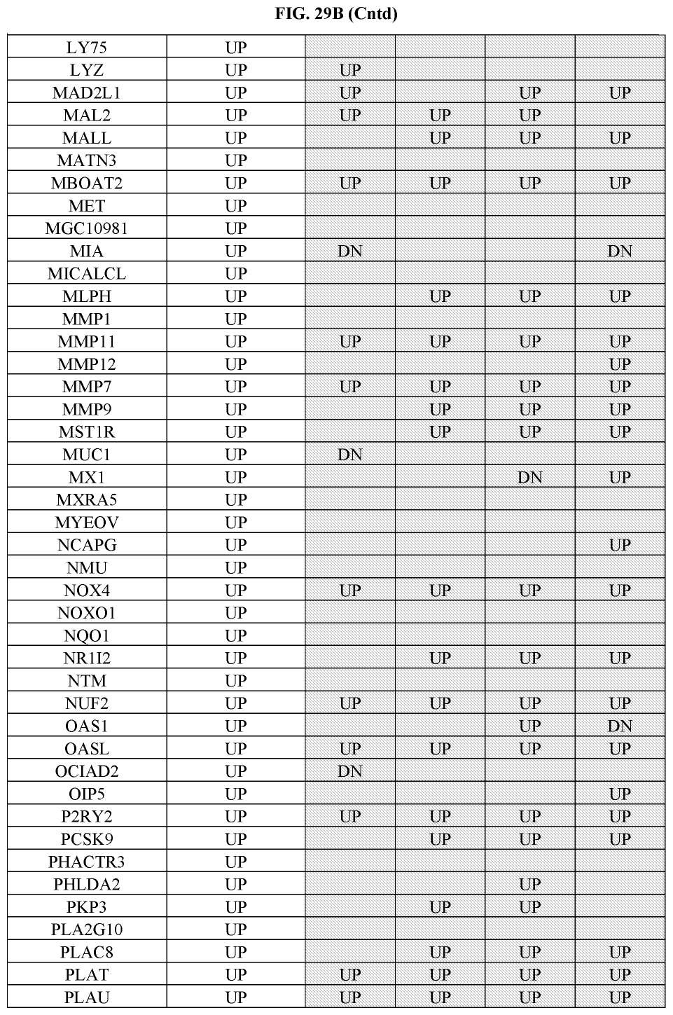

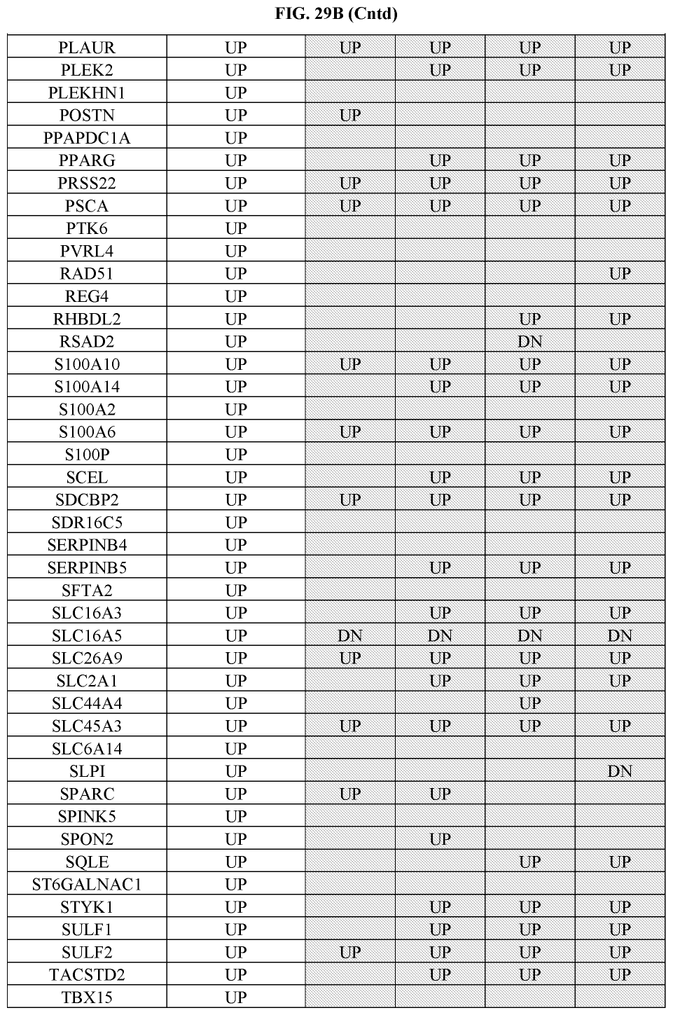

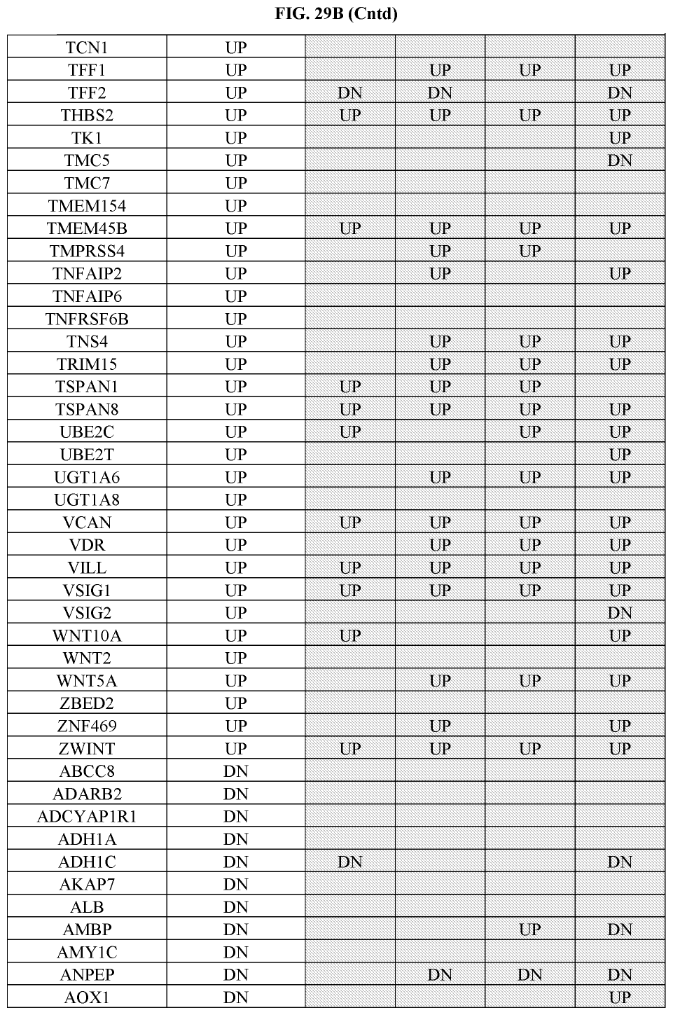

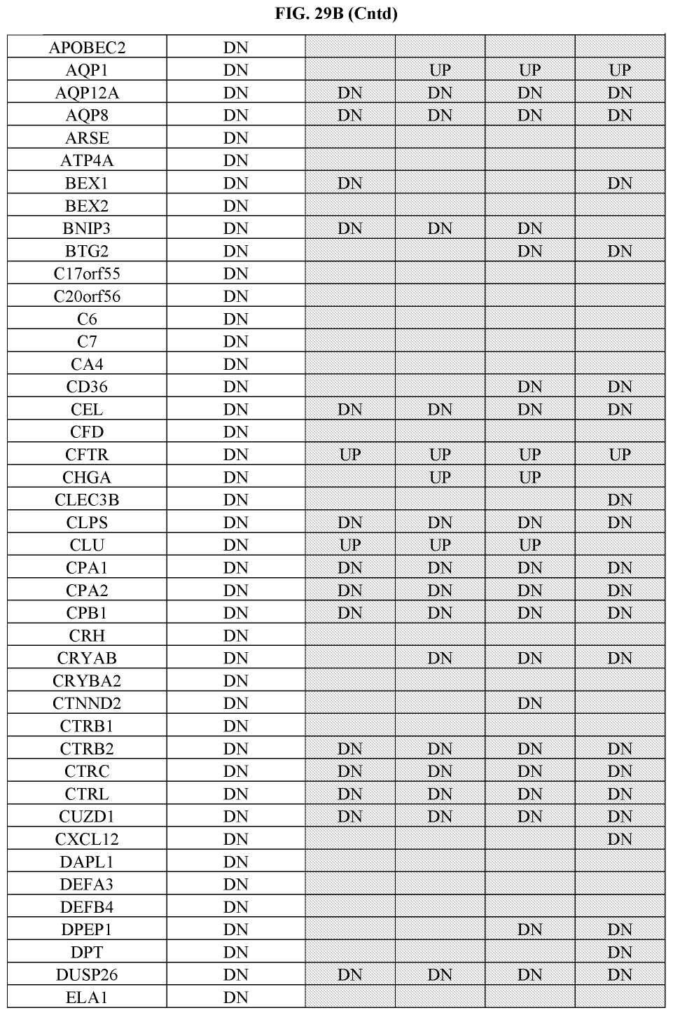

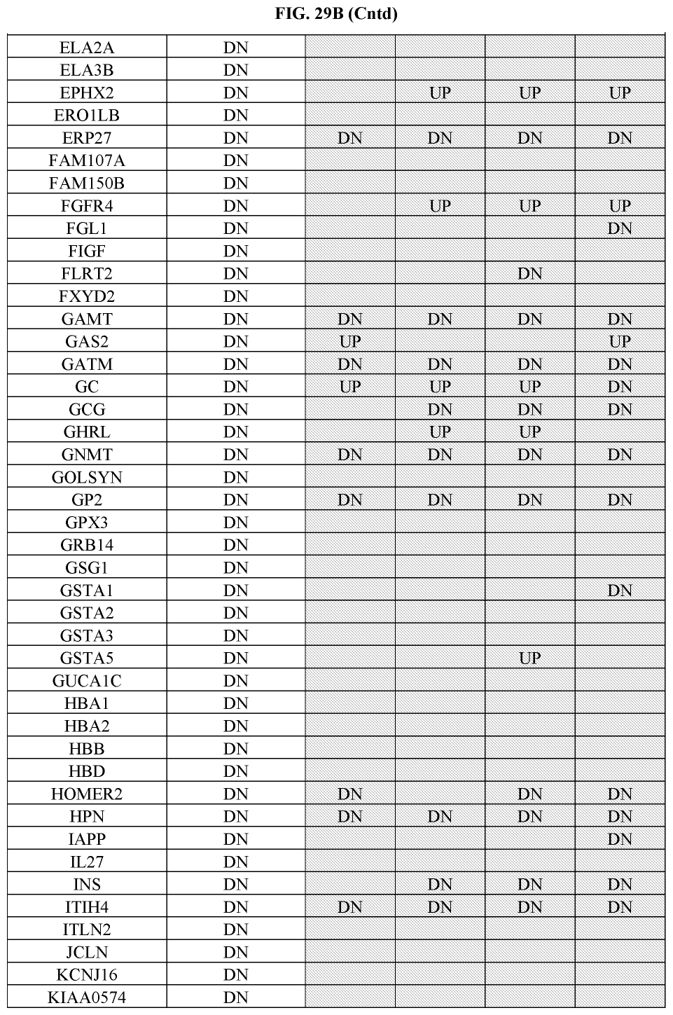

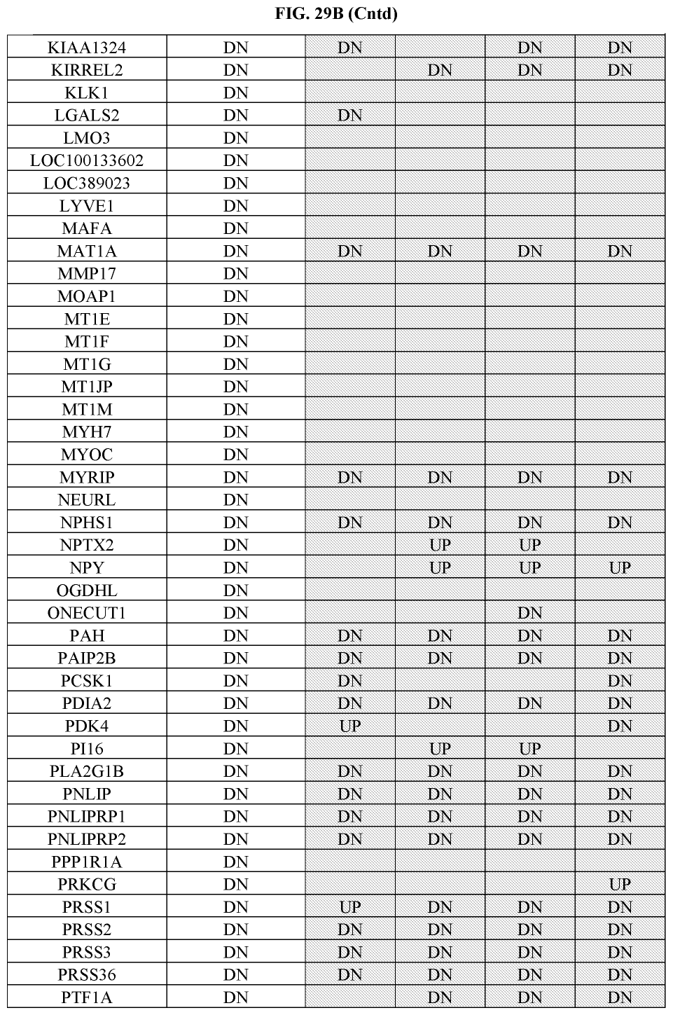

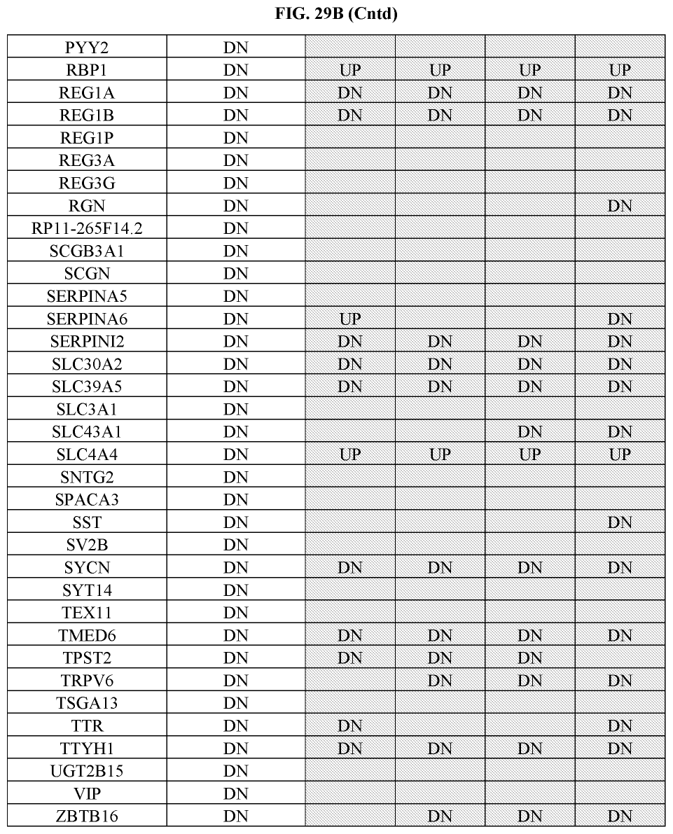

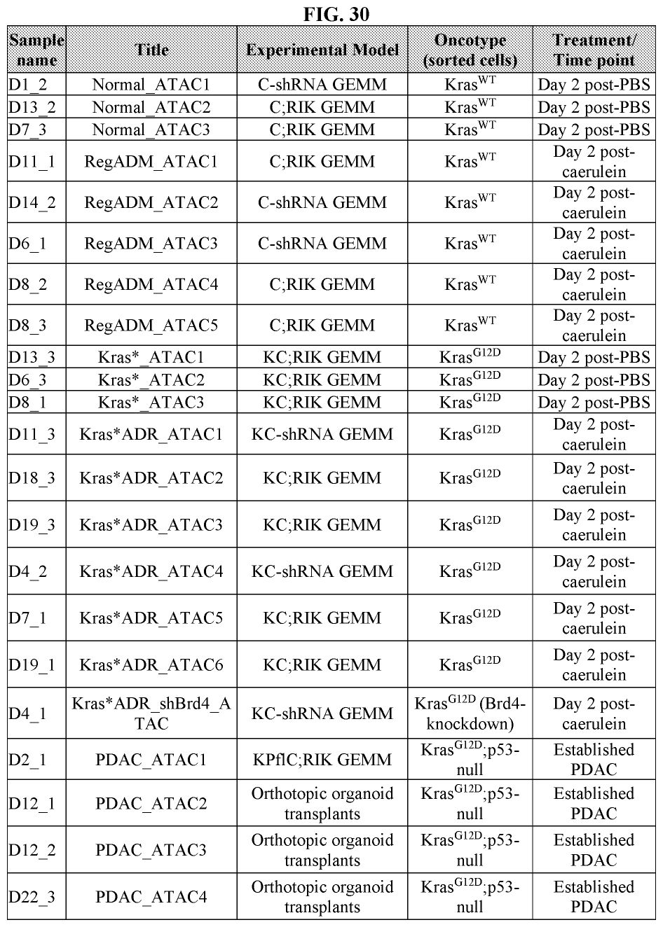

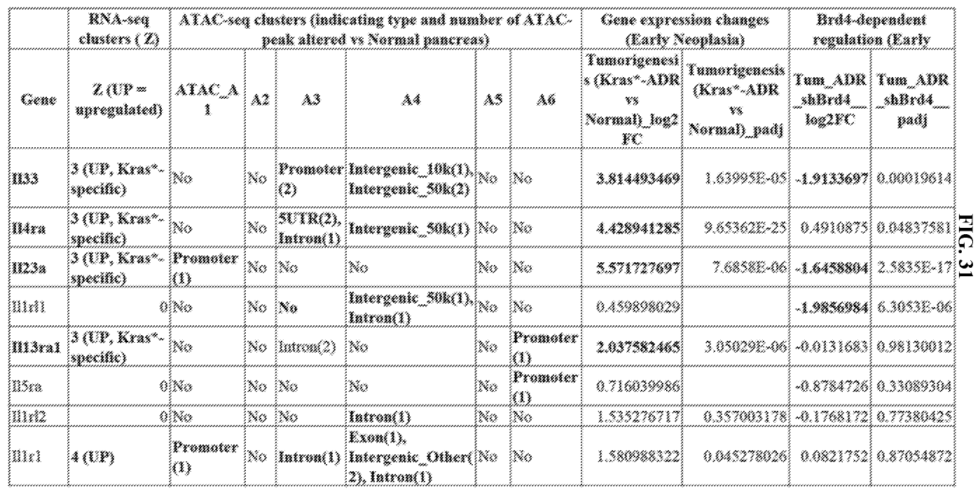

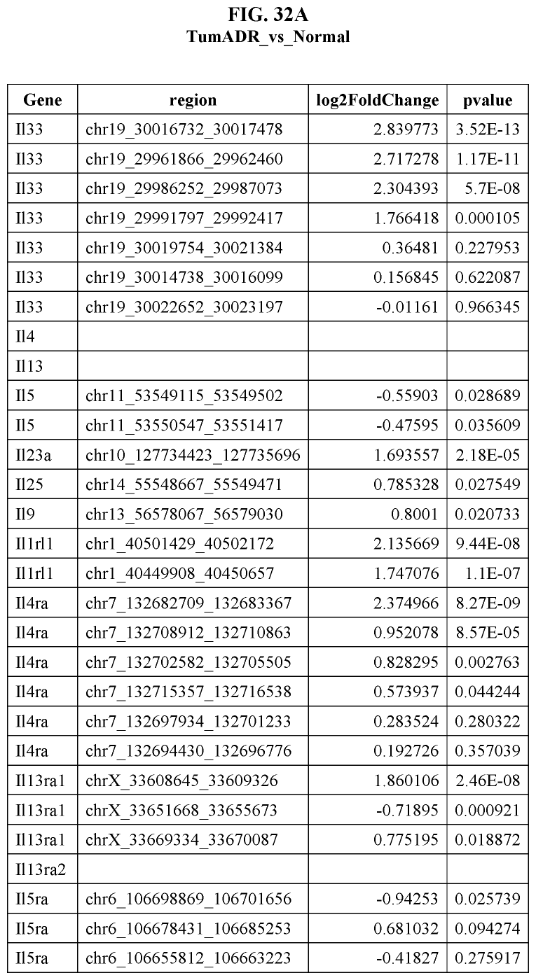

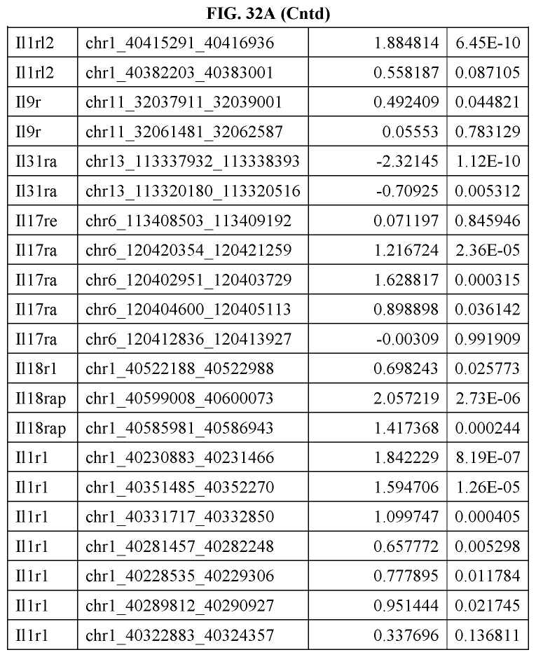

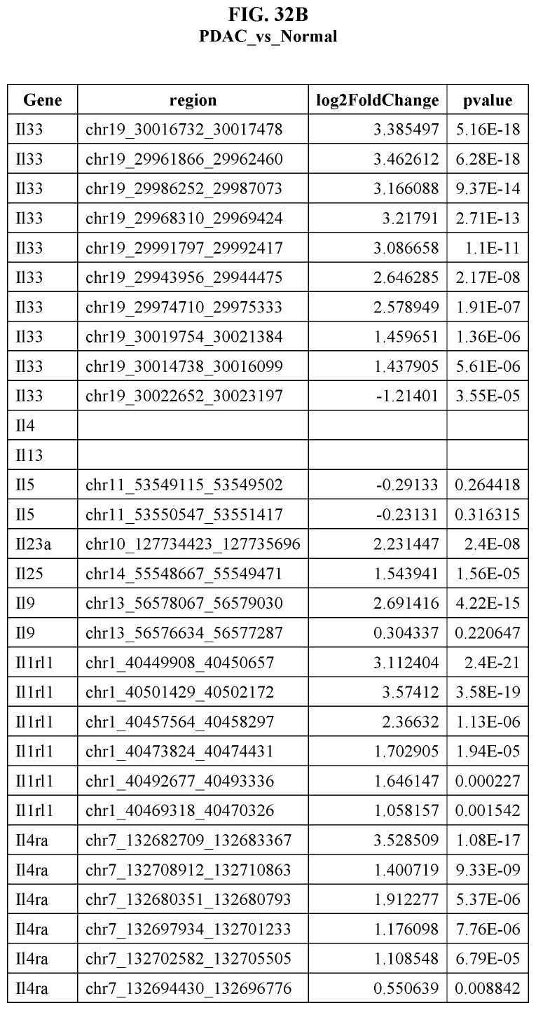

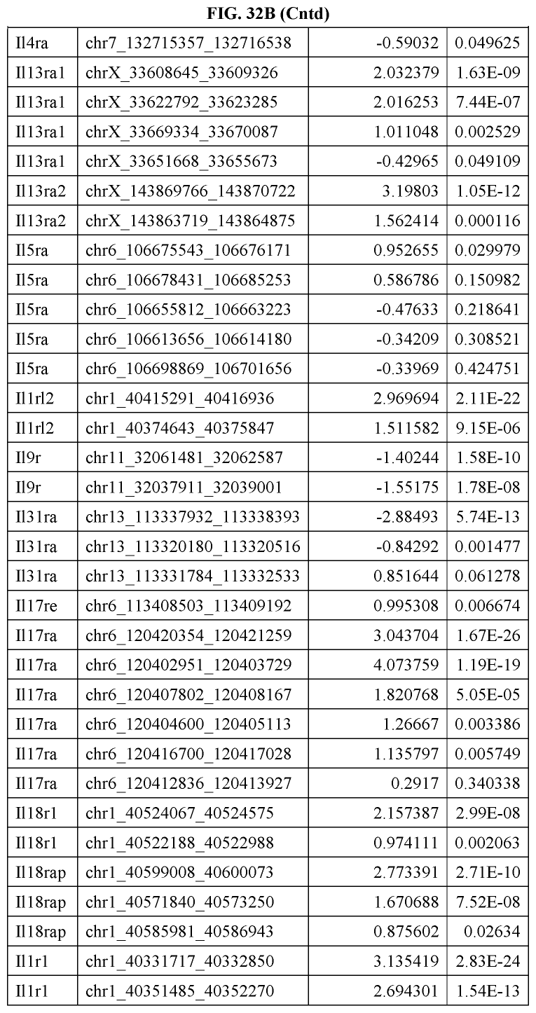

Mapping, peak calling and dynamic peak calling: Fastq files were trimmed with trimGalore and cutadapt, and the filtered, pair-ended reads were aligned to mm9 with bowtie2. Peaks were called over input using MACS2, and only peaks with a p-value of <=0.001 and outside the ENCODE blacklist region were kept. All peaks from all samples were merged by combining peaks within 500 bp of each. The featureCount was used to count the mapped reads for each sample. The resulting peak atlas was normalized using DESeq2 (PeakNorm). For comparison to DepthNorm, samples were normalized to 10 million mapped reads (FIGS. 11B, 22, 23). Normalized bigwig files were created using the normalization factors from DESeq2 as previously described (Pronier et al., JCI Insight 3 (2018)) and bedtools genomeCoverageBed. Dynamic peaks were called if they had an absolute log 2FC>=0.58 and a FDR<=0.1. Peaks were associated with genes based on UCSC.mm9.knownGene using ChIPseeker package. The distance of a peak to the nearest TSS was identified and annotated the peak to that gene. Possible annotations are Promoter-TSS, Exon, 5′ UTR, 3′ UTR, Intron, and Intergenic.

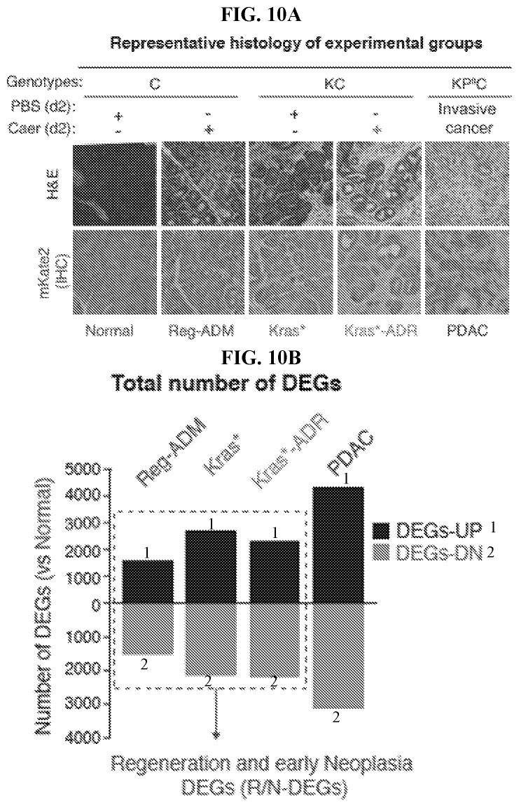

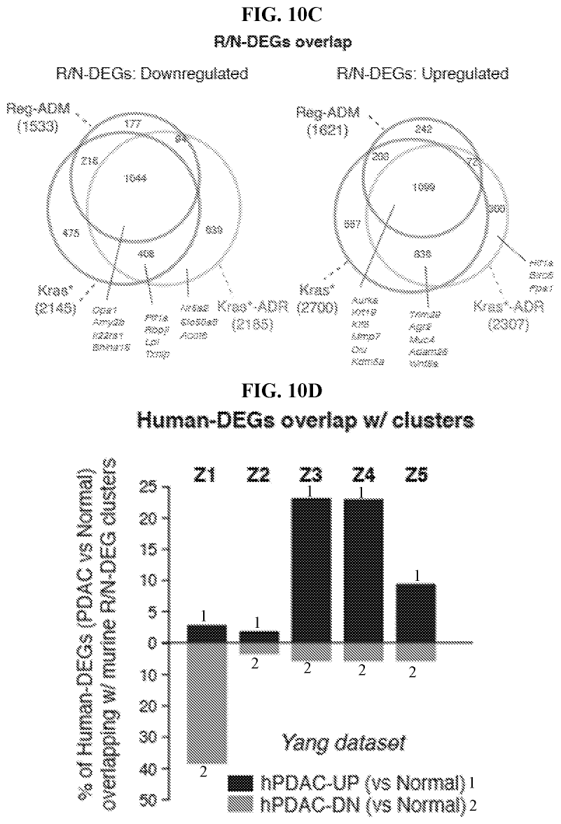

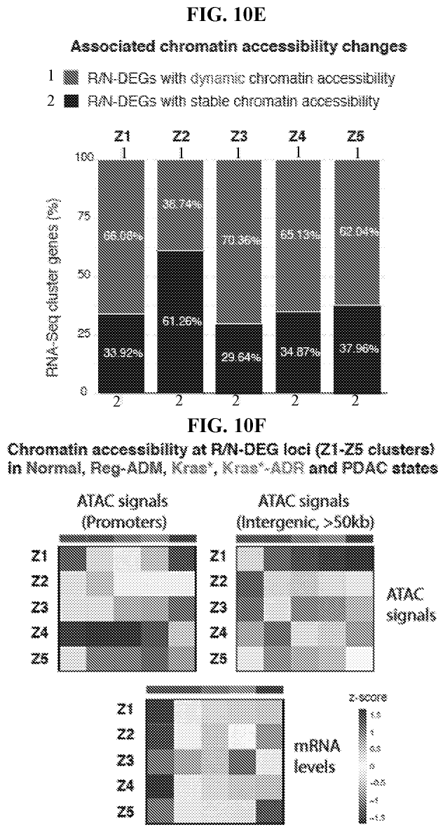

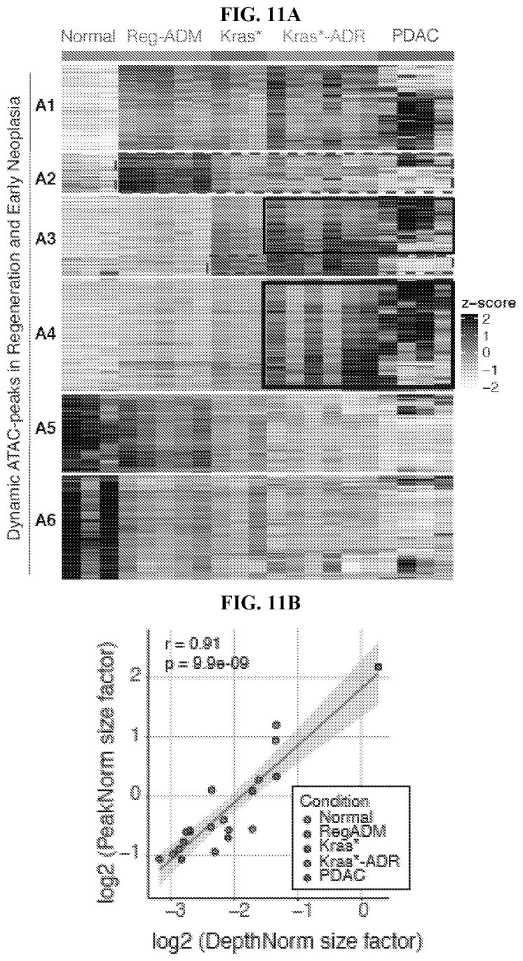

ATAC-seq heatmap clustering and motif enrichment analysis: The dynamic peaks of regeneration and early neoplasia determined by comparing Normal, Reg-ADM, Kras*, and Kras*ADR conditions (as defined in FIGS. 4A and 10A) were clustered using z-score and a kmeans of 6 and plotted using ComplexHeatmap. Each of the resulting clusters was further analysed for genic location (annotatePeaks), and for percentage of peaks with AP1-motifs (annotatePeaks with jun-ap1.motif and mbed) or Nr5a2-bound loci (extracted from GSE34295; (Holmstrom et al., Genes Dev 25, 1674-1679 (2011)).

Reg-ADM and Kras*-ADR Unique peak signatures: To find dynamic peaks unique and shared between regenerative or pro-neoplastic metaplasia compared to normal pancreas, bedtools was used remove, or keep, the unique/shared peaks between Reg-ADM vs normal and Kras*-ADR conditions vs normal pancreas. The unique peaks were sorted by log 2FC, and the top and bottom 500 up- and down-regulated peaks were investigated for motif enrichment using HOMER findMotifGenome.

Integrative analysis of RNA-seq-ATAC-seq data: To investigate chromatin accessibility changes associated with DEGs found in the RNA-Seq analysis, dynamic ATAC-Seq peaks were first averaged across samples within each tissue state. Each peak was separated based on ChIPSeeker annotations (TSS, Promoter, 5′UTR, etc). Peak signals were then first summarised on individual gene level, followed by averaging across individual RNA-Seq clusters (Z1-Z5). Data was z-score normalized for each of the indicated tissue states. RNA-Seq data were averaged over genes within each Z1-Z5 cluster. Data was z-score normalized across each Z1-Z5 cluster. The final average data was represented as heat map using pheatmap package in R.

Isolation, culture and genetic manipulation of pancreatic organoids. To isolate untransformed ductal organoids for the transplantable PDAC tumorigenesis model, normal pancreas from LSL-KrasG12D mice (pure Bl/6N background) minced and digested with 0.012% collagenase XI (C9407, Sigma-Aldrich) and 0.012% Dispase (17105041, Life Technologies) in in HBSS with Mg²⁺ and Ca²⁺ (14025076, Thermo Fisher Scientific) at 37° C. for a maximum of 30 mins. The material was further digested with TrypLE (GIBCO) for 5 min at 37° C., washed twice with DMEM/F12 (Life Technologies) supplemented with 2 mM glutamine and penn-strep, embedded in growth factor reduced matrigel (Corning), and cultured in complete medium, as described in Boj et al 2014. For activation of mutant Kras, organoids haroburing the LSL-KrasG12D allele were transduced with Ad-mCherry-Cre (Vector Biolabs), and Cherry⁺ cells were sorted from single cell organoid suspension by flow cytometry 36h thereafter. Resulting clones were assessed for LSL-KrasG12D recombination by genotyping PCR in genomic DNA using the following primers: 5′ gtc ttt ccc cag cac agt gc 3′ (SEQ ID NO: 19), 5′ ctc ttg cct acg cca cca get c 3′ (SEQ ID NO: 20), and 5′ agc tag cca cca tgg ctt gag taa gtc tgc a 3′ (SEQ ID NO: 21). Validated Cre-recombined clones were then subjected to CRISPR-based inactivation of Trp53 using the PX458 vector (Addgene #48138) and gRNA AGTGAAGCCCTCCGAGTGTC sequence (SEQ ID NO: 22). PX458-sgTrp53 was transduced into organoids by transient transfection using the spinoculation method previously described (O'Rourke et al., 2017), with the modification of using the Effectene transfection reagent (Qiagen). PX458-sgTrp53 introduced cells were sorted by GFP positivity with flow cytometry 36h post-transfection. p53 null status of targeted clones was validated by western blot, using anti-p53 antibody (CMS, Leica Microsystem) and anti-β-actin-peroxidase antibody (Sigma-Aldrich) as normalization control.

Statistical analysis. Statistical analyses were performed using Prism 7 by unpaired two-tailed t-test for all other experimental data. Grouped data are expressed as mean+SEM for the number of biological replicates indicated in the figures or associated legend. Statistically significant differences are indicated with asterisks in figures with the accompanying p-values in the legend. In RNA-seq data, significance for differential gene expression between groups was based on adjusted p-value<0.05. For pathway enrichment analysis of RNA-seq gene clusters, the significance of gene lists was assessed by adjusted p-value and Z-score (Chen et al., 2013) Significance of gene sets from GSEA was based on the normalized enrichment score (NES) and the false discovery rate q-value (FDR q-val). In ATAC-seq data, dynamic peaks were called if they had an absolute log 2FC>=0.58 and a FDR<=0.1. For experiments using chimeric KC-shRen mice, only animals with coat chimerism>95% were included.

All RNA-seq and ATAC have been deposited to GEO under series GSE132330.

### Example 2: shBrd4-GEMMs Enable Spatiotemporal Perturbation of Enhancer Function In Vivo

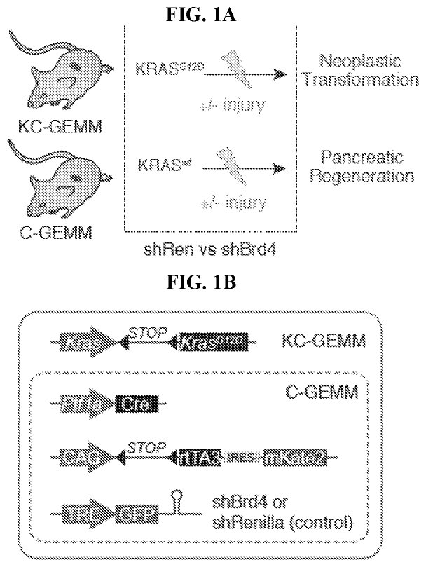

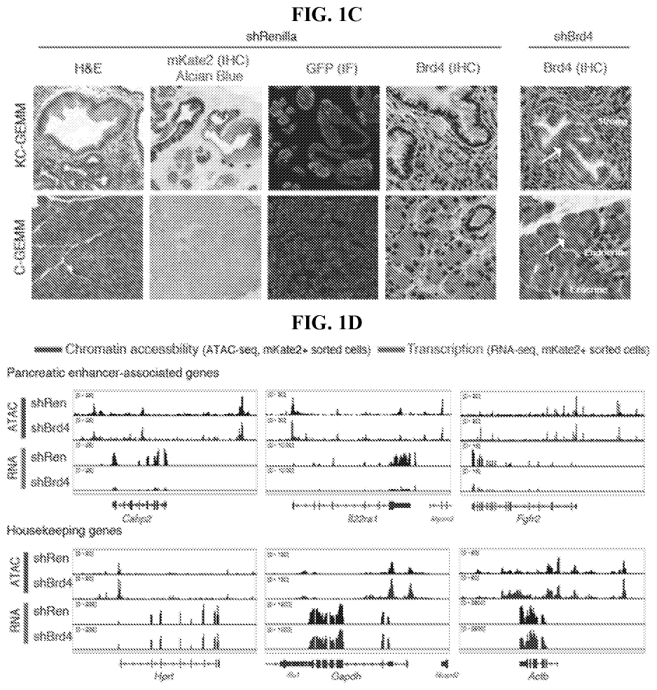

To define the contribution of chromatin mechanisms controlling cell identity to mutant KRAS-driven neoplastic transformation in vivo, autochthonous mouse models that enable lineage-tracing and spatiotemporally-controlled perturbation of the chromatin reader Brd4 in vivo were developed (FIGS. 1A-1B). This approach allows a spatiotemporally-controlled perturbation of enhancer function in vivo. Without wishing to be bound by any particular theory, it was hypothesized that such models would serve as powerful means to reveal biological traits and molecular programs dependent on interpretation of chromatin states during cell fate transitions occurring within their native tissue context.

To this end, a flexible embryonic stem cell (ESC)-based mouse modeling strategy that incorporates lineage tracing and inducible control of gene expression into multi-allelic mice predisposed to PDAC was exploited. To attain tight control of Brd4 activity specifically in the pancreatic epithelium, mice harboring: (i) a pancreas-specific Cre driver (Ptf1a-Cre), (ii) a Cre-activatable LSL-Kras^{G12D} allele and (iii) two additional alleles [LSL-rtTA3-IRES-mKate (RIK) and the collagen homing cassette, (CHC)] that allow for inducible expression of a GFP-linked shRNA targeting Brd4 (shBrd4) in Cre-recombined cells labeled by the fluorescent reporter mKate2 were generated (FIG. 1B). In this model (referred to as KC-shBrd4 below), Cre activation leads to pancreas-specific expression of Kras^{G12D}, rtTA and a linked mKate2 reporter. Upon receiving a doxyxycline (dox)-containing diet, a GFP-linked shRNA targeting Brd4 is induced in mKate2-labeled pancreatic epithelial cells susceptible to mutant Kras-driven transformation. Of note, this mode of Brd4 inactivation is fundamentally distinct from pharmacologic strategies which inhibit BET protein function systemically and fail to deconvolute the effects of disrupting different sets of enhancers in the tumor epithelium and its surrounding microenvironment, known to modulate epithelial states.

In addition, analogous models harboring the Brd4 shRNA without the LSL-Kras^{G12D} allele (referred to as C-shBrd4 below) were generated to compare and contrast the epigenetic requirements of neoplastic transformation vs injury-driven regeneration (FIGS. 1A-1B). To control for on-target effects of RNAi, mice were produced that harbor two different well-validated shRNAs targeting Brd4 (named shBrd4.552 and shBrd4.1448). To control for potential phenotypes linked to dox treatment and off-target perturbations of the RNAi machinery, mice harboring a highly potent but phenotypically neutral shRNA targeting Renilla Luciferase (shRen.713) were produced. Animals derived from all of these models were studied in parallel.

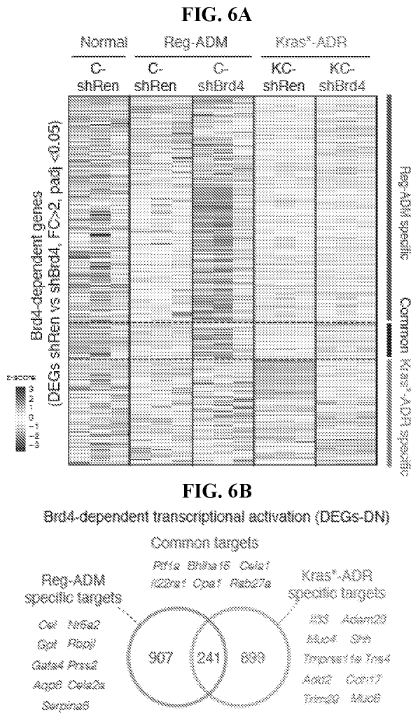

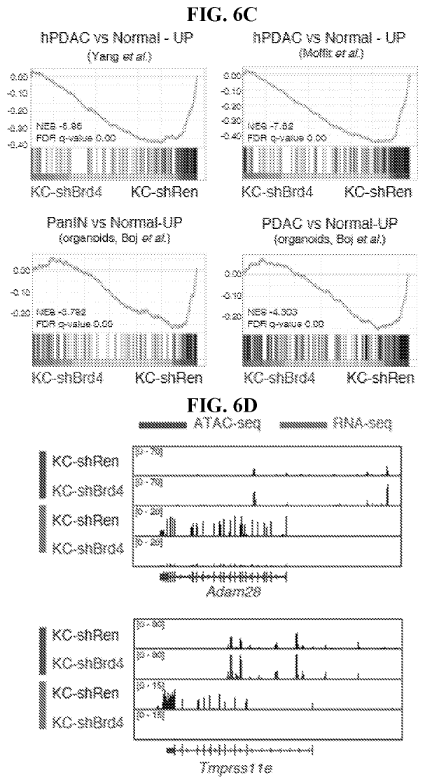

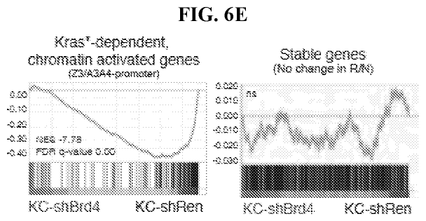

First, histological and molecular analyses were performed to confirm that acute Brd4 knockdown disrupts the expression of genes driven by lineage-specific enhancers in vivo. As expected, histological analysis of pancreata from 4-week-old KC- and C-GEMM mice harboring control (shRen) or Brd4 shRNAs (FIG. 1B) treated with dox showed mKate2/GFP double positivity in either KRAS mutant or wild-type acinar cells, respectively, with shBrd4 tissues showing potent suppression of Brd4 protein in this same cellular compartment while sparing the surrounding microenvironment (FIG. 1C). To assess gene expression and chromatin states in shRen or shBrd4-expressing cells, mKate2/GFP⁺ FACS-sorted cells were isolated from KC-GEMM mice and subjected to RNA-seq and ATAC-seq, respectively. Acute Brd4 suppression reduced the expression of pancreatic enhancer-associated genes described in the developing or adult pancreatic epithelium, effects that occurred without decreasing chromatin accessibility at these loci or involving global effects on transcription (e.g. at housekeeping genes, FIG. 1D bottom panels, and FIGS. 6A-6E). These results validated the GEMM-shBrd4 models as a platform to study the effects of enhancer-associated gene regulation on cellular identity in vivo.

### Example 3: Brd4 is Required for Mutant Kras-Induced Pancreatic Neoplasia but not Metaplasia

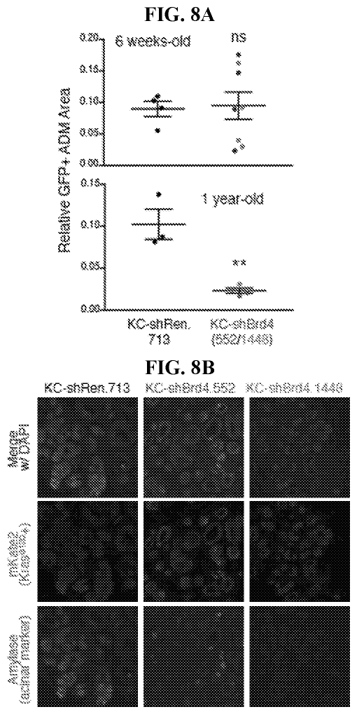

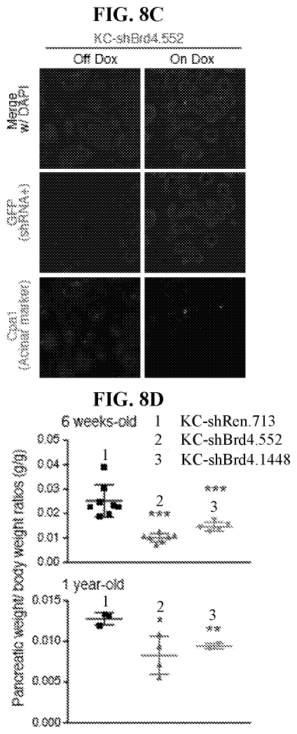

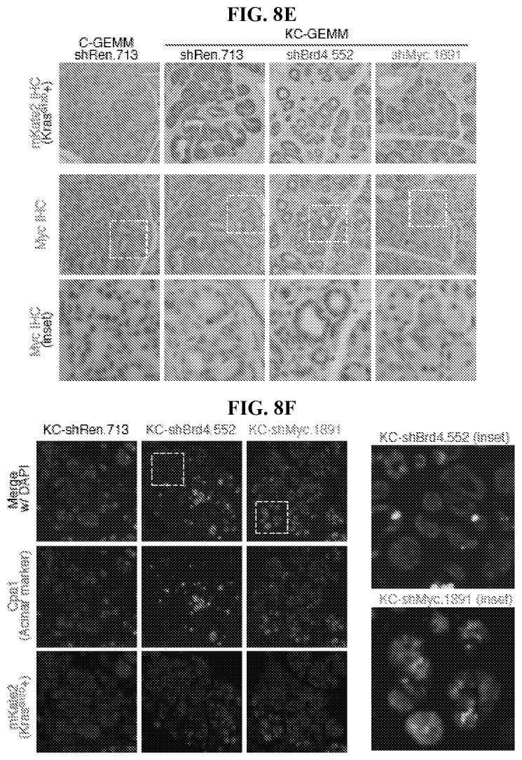

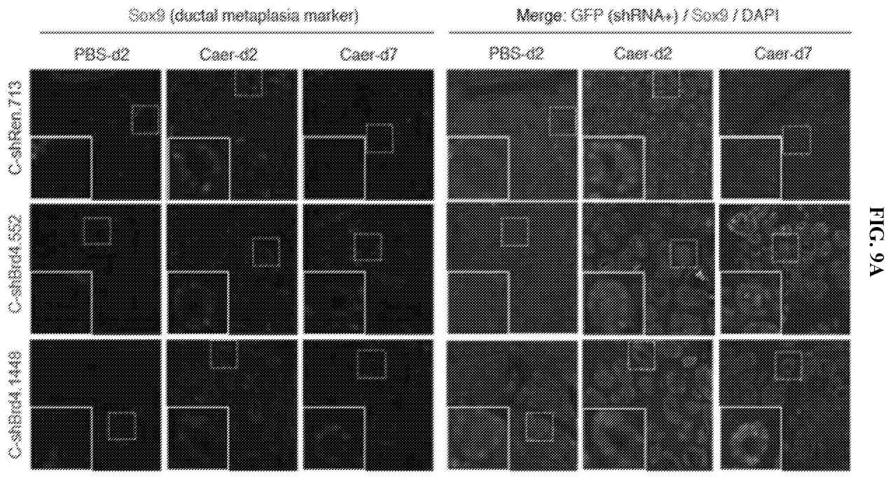

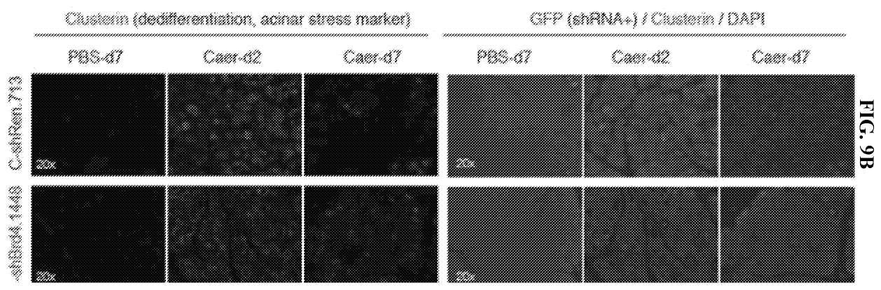

Taking advantage of the above systems, experiments were performed to determine the requirement for Brd4-regulated programs in mutant Kras-driven pancreatic neoplasia, both in settings where oncogenic Kras induces stochastic development of neoplastic cell fate and where transformation is accelerated by tissue injury. To track the effects on stochastic Kras driven neoplasia, KC-shRen or KC-shBrd4 mice were placed on a diet containing doxyxycline (the dox diet) at 10 days of age and analyzed the induction and evolution of metaplasia in 6 week and 1 year-old mice. By 6 weeks, pancreata of KC-shRen mice displayed features of acinar-to-ductal metaplasia (ADM), as assessed by the appearance of duct-like structures with decreased expression of acinar markers (e.g. Cpa1, Amylase) and emergent expression of ductal markers (e.g. Sox9 and Krt19) not normally expressed in acinar cells (FIG. 2A). Interestingly, Brd4 suppression did not impair the mutant Kras-driven conversion of acinar cells into a metaplastic duct-like state and, in fact, appeared to accelerate this transition (FIGS. 2A, 2B; FIGS. 8A, 8B). Histology of pancreata from KC-shBrd4 mice kept off dox resembled that of KC-shRen control pancreata on dox, ruling out potential dox- or strain-dependent effects (FIG. 8C).

In contrast, Brd4 suppression prevented the subsequent neoplastic progression of metaplastic lesions into pancreatic intraepithelial neoplasias (PanINs). At both the 6 week and 1 year old time-points, KC-shBrd4 mice exhibited a marked reduction in PanIN lesions, evidenced by significantly reduced positive co-staining of the acidic mucin marker Alcian Blue and the shRNA-linked GFP (FIGS. 2C, 2D). While the GFP-positive metaplastic lesions present in KC-shRen mice had progressed into PanINs after 1 year (FIGS. 2C-2E; 8A), these lesions were lost in pancreata of KC-shBrd4 mice, which were atrophic and displayed recovery of GFP-negative normal acinar tissue (FIGS. 2C, 8D), common reactions to perturbations compromising exocrine pancreas maintenance. Of note, while Brd4 can regulate Myc expression in some contexts (Delmore et al., Cell 146: 904-917 (2011); Zuber et al., Nature 478: 524-528 (2011)), suppression of Brd4 did not reduce Myc protein levels in this setting (FIG. 8E), and pancreata from mice produced using a similar GEMM-ESC model harboring a potent Myc shRNA exhibited impaired rather than accelerated ADM (FIGS. 8E, 8F). These observations imply that the effects of Brd4 suppression observed during early stage pancreatic neoplasia are Myc-independent.

To explore the requirement for Brd4 in the context of injury-accelerated tumorigenesis, a well-established model of pancreatic injury produced by treatment with the synthetic cholecystokinin analogue caerulein was used. Caerulein triggers acinar autolysis and inflammation, resulting in accelerated neoplasia associated with ADM within 48 h post-treatment and progression into PanIN lesions by 2-3 weeks. 4-week-old KC-shRen or KC-shBrd4 mice were placed on dox diet to acutely induce shRNA expression and, 6 days later, treated with caerulein, such that ADM and PanIN formation were triggered synchronously throughout the pancreas in the presence or absence of epithelial Brd4 (FIG. 2F). Consistent with the above findings, Brd4 suppression accelerated loss of acinar identity and resulting metaplasia but impaired subsequent reprogramming into PanINs, even in this context of injury-accelerated neoplasia (FIG. 2G). Collectively, these data reveal a differential dependency on Brd4 function for mutant Kras-driven metaplasia and subsequent neoplastic transformation, and suggest that the contribution of enhancer-mediated gene regulation to each process is distinct. Specifically, these results using a pancreatic cancer initiation model indicate that the gene expression program controlled by Brd4 is required for mutant Kras-induced pancreatic neoplasia but not metaplasia. These results also point to differential roles of Brd4 and Myc during mutant Kras-driven tumorigenesis.

### Example 4: Brd4 Controls Metaplastic Cell Fate Plasticity During Normal Regeneration

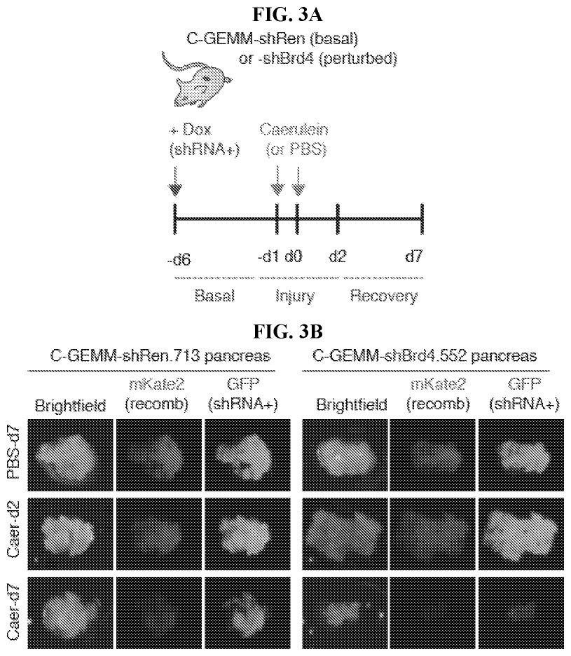

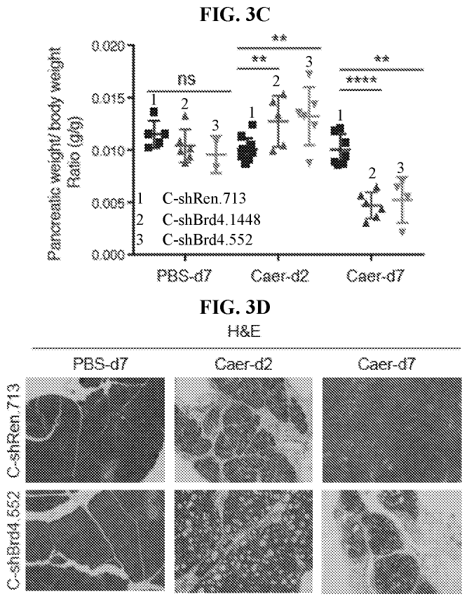

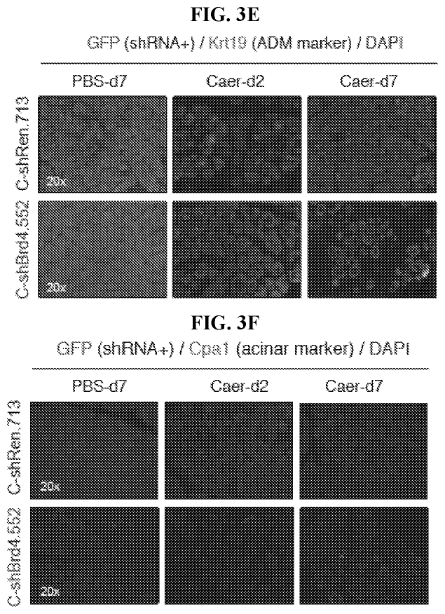

Pancreatic metaplasia is not an exclusive feature of early pancreatic neoplasia but also occurs as part of a physiological regenerative response to tissue injury. To compare the epigenetic requirements of neoplastic vs regenerative epithelial plasticity, GEMMS permitting inducible Brd4 suppression on the background of wild-type Kras (C-shBrd4 and C-shRen) were generated by strain intercrossing, and 4-week-old mice were subjected to dox administration followed by caerulein treatment to induce synchronous ADM in normal pancreas in the presence or absence of epithelial Brd4 (FIG. 3A). In the absence of oncogenic Kras, caerulein typically drives a transient ADM prominent 2 days post-treatment that resolves via re-differentiation and acinar regeneration within the following 5-7 days. Thus, ADM and regeneration were examined in control C-shRen and C-shBrd4 mice at days 2 and 7 post-caeruelin treatment, respectively (FIG. 3A).

In contrast to controls, Brd4-suppressed mice exhibited a rapid reduction of mKate2/GFP⁺ labeled shRNA-expressing cells and pancreatic tissue size between day 2 to day 7 post-caerulein (FIGS. 3B, 3C), indicating a failure to regenerate. Histologically this was coupled with a defect in the reversion of ADM into normal acinar cell morphology in C-shBrd4 mice, which exhibited widespread ADM with significantly enlarged lumens at day 2 that, unlike controls, were still present at day 7 post-caerulein (FIG. 3D). At a molecular level, unresolved ADM in C-shBrd4 mice was confirmed by sustained induction of both ductal markers Sox9 (FIG. 9A) and Krt19 (FIG. 3E) and the immature acinar marker Clusterin (FIG. 9B), as well as by sustained reduction of the acinar marker Cpa1 in GFP⁺ Brd4-suppressed epithelium (FIG. 3F). Accordingly, Brd4 controls metaplastic cell fate plasticity during normal regeneration, and Brd4 is dispensable for injury-induced acinar dedifferentiation and ductal metaplasia. Taken together, these results demonstrate that Brd4 is dispensible for ADM in both regenerative and pro-neoplastic contexts but required for a return to tissue homeostasis or, alternatively, neoplastic progression in the presence of oncogenic Kras. These findings suggests that Brd4-dependent enhancers dictate both regenerative and oncogenic plasticity in vivo.

### Example 5: Regeneration and Neoplasia are Associated with Distinctive Transcriptional and Chromatin States

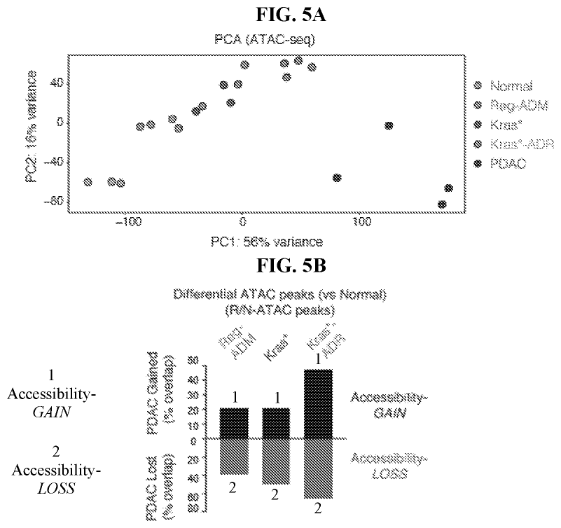

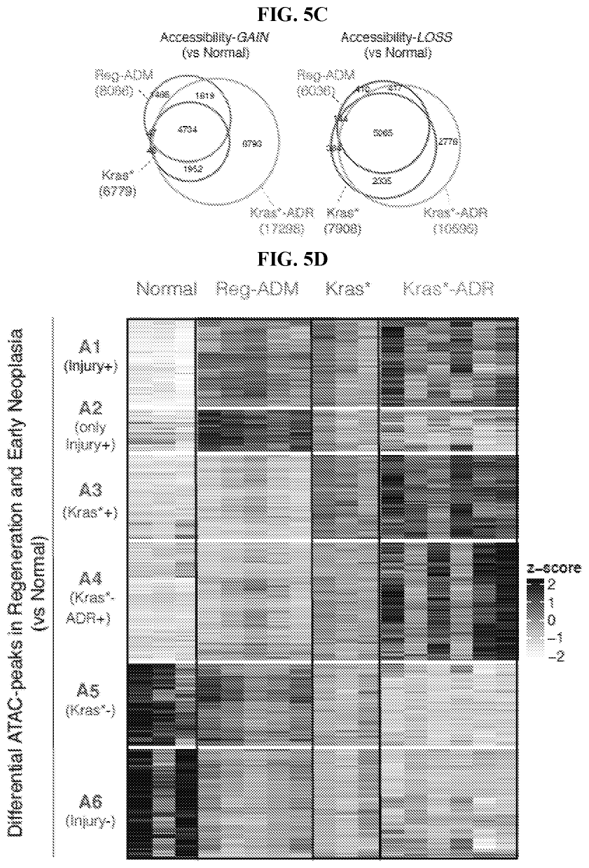

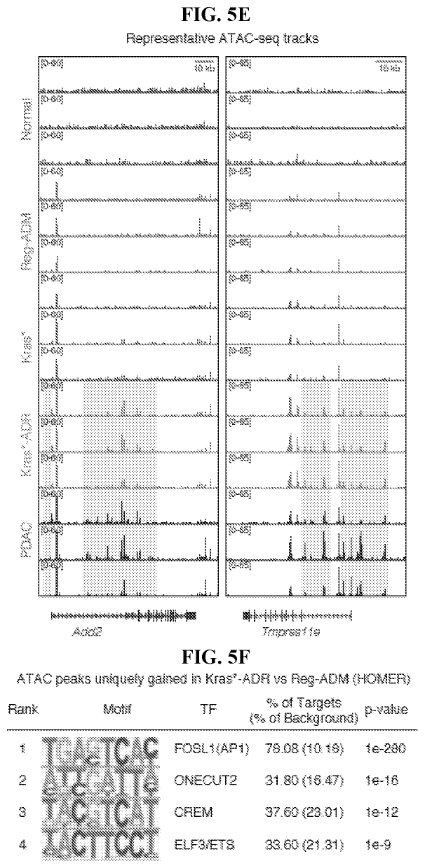

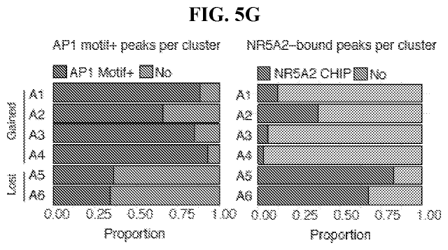

Given the established role of BRD4 in reading chromatin regulatory programs that control cell fate, the transcriptional and chromatin landscapes of lineage-traced pancreatic epithelial cells undergoing regenerative or pro-neoplastic cell fate transitions were next characterized in vivo. Specifically, the mKate2-positive populations isolated by fluorescence-activated cell sorting (FACS) from normal, regenerating, early neoplastic and malignant pancreatic tissues representing the following tissue states (n>=3/each): (i) normal healthy pancreas (Normal), (ii) normal pancreas undergoing regenerative injury-driven ADM (Reg ADM); (iii) Kras-mutated pancreata undergoing stochastic neoplastic transformation (Kras*), and (iv) Kras-mutated pancreata undergoing synchronous neoplastic reprogramming (Kras*-ADR) accelerated by induction of tissue injury were profiled (see FIGS. 4A, 10A for details). With this experimental design, RNA-seq and ATAC-seq analyses were performed at 48 h post-injury, preceding both regeneration and overt tumorigenesis, thereby revealing the regulatory programs that are most likely to direct (and not result from) these cell fate transitions. As a reference for full-blown malignant disease, mKate2-positive cells isolated from established PDAC arising in KP^{fl}C-GEMMS (Kras^{G12D}p53^{Δ/Δ}) were also profiled (FIGS. 4A, 10A). Of note, despite the inherently high RNAase and protease content of pancreatic tissue makes in vivo genomic studies challenging, the conditions for RNA-seq and ATAC-seq analyses from small numbers of cells that are insufficient for ChIP-seq could be established. The resulting data were robust, as independent biological replicate samples (individual mice) clustered tightly together within their respective groups (FIG. 4B and see FIG. 5A below).

Principal component analysis (PCA) of RNA-seq data revealed that populations undergoing regeneration or mutant Kras-dependent neoplastic transformation exhibit transcriptional changes reminiscent of established PDAC, with injury and mutant Kras effects accounting for 74% of total variance vs 9% related to late stage transitions associated with p53 loss (FIG. 4B). Comparison of the differentially expressed genes (DEGs) between normal, regenerating, early neoplastic and malignant murine pancreatic epithelial cells (FIG. 10B) with those present in human PDAC (Moffitt et al., Nat Genet 47, 1168-1178 (2015); Yang et al., Cancer Res 76: 3838-3850 (2016)) showed that up to ˜50% of the gene expression changes distinguishing human PDAC from normal pancreas are recapitulated in murine PDAC, and that virtually all (˜98%) of these alterations are induced already in premalignant KRAS-mutant tumor cells (FIG. 4C, FIGS. 29A and 29B). Thus, despite transcriptional heterogeneity existing across patients, a large fraction of the gene expression programs defining full-blown human PDAC can be triggered at very early stages of neoplasia, revealing molecular pathways with potential therapeutic value for early detection and prevention of pancreatic cancer.

To compare gene expression programs linked to regeneration and early neoplasia (herein referred to as R/N-DEGs, FIGS. 4D and 10C), the associated DEGs were subjected to unsupervised kmeans clustering followed by pathway enrichment analysis. As summarized in FIG. 4D, these analyses identified both shared and context-specific gene clusters that were associated with distinctive biological processes. For example, downregulated genes common to cells undergoing both neoplastic transformation and regeneration included acinar cell specification genes (e.g. Cpa1, Il22ra1, Amy2b) and genes involved in translation (e.g. Rps7, Rpl41) and mitochondrial metabolism (e.g. Mpc1, Idh2) (FIG. 4D, Z1 and Z2 clusters; see also FIG. 10C). In turn, commonly upregulated genes were linked to cellular proliferation (e.g. Aurka, E2f3), ECM remodeling (e.g. Mmp7, Mmp2), chromatin regulation (e.g. Kdm5a, Kdm1a) and metaplasia (e.g. Krt19, Klf5) (FIG. 4D, Z4 and Z5 RNA-seq clusters). Importantly, a large cluster of genes distinguished mutant Kras cells from wild-type counterparts, even during regeneration (Z3 RNA-seq cluster in FIG. 4D). Distinguishing factors included those previously linked to RAS signaling (e.g. Trim29, Lamb3) or to processes dysregulated in human PDAC, including axon guidance (e.g. Sema5a), fibrosis (e.g. Shh), mucins (e.g. Muc6, Muc4), epithelial differentiation (e.g. Klf5), or cholesterol metabolism (e.g. Apob, Ldlr) (FIGS. 4D, 10D).

Integration of the in vivo transcriptional profiling data with ATAC-seq data at R/N-DEG loci revealed that 60% of the genes that undergo changes in expression during regeneration or early neoplasia are associated with consistent changes in chromatin accessibility (FIGS. 10E, 10F). Interestingly, metaplasia-associated genes known to be induced in both regenerating pancreas and early neoplastic lesions (e.g. Krt19, Sox9, Clu or Myc) pre-existed in an open chromatin state in normal acinar cells. In contrast, transcriptional modulation of known acinar (e.g. Cpa1, Mist1/Bhlha15 or Ptf1a) and neoplastic (e.g. Cdh17 or Sema5a) genes was typically associated with distinctive changes in chromatin accessibility (FIG. 4E, and see FIG. 5 below). These data demonstrate that regeneration and neoplastic transformation are associated with distinctive transcriptional and chromatin states, and that neoplastic-specific chromatin alterations are present in early and late-stage pancreatic cancers, but not in normal or regenerative tissues.

The results disclosed herein identify the shared and specific transcriptional programs that underlie regeneration and early neoplasia. Thus, while there are notable similarities between the cell fate transitions accompanying neoplastic transformation and regeneration, the underlying transcriptional programs and chromatin states are distinct.

### Example 6: Injury and Mutant Kras Synergize to Promote Rapid Chromatin Opening at Loci Characteristic of Invasive PDAC

Next the global analysis of chromatin accessibility dynamics across the spectrum of epithelial states captured in the ATAC-Seq data was performed to define the chromatin accessibility landscapes characteristic of normal, regenerating, pre-malignant, and PDAC (FIGS. 4A, 11A). Informatic tools were used to identify open chromatin regions (peaks) gained or lost in each state compared to normal exocrine pancreas, which are referred to herein as accessibility-GAIN and accessibility-LOSS regions, respectively. As shown in FIGS. 5A-5D, large-scale recurrent changes in chromatin accessibility were detected in the settings of regeneration, early neoplasia, and full-blown PDAC. Remarkably, the combined effects of mutant Kras and injury (Kras*-ADR) recapitulated ˜50% of the accessibility-GAIN and -LOSS regions that distinguish fully malignant PDAC cells from normal pancreas (FIG. 5B). These data were robust and reproducible: hence, analysis of ATAC-seq data using different normalization approaches (depth- vs peak-normalized), or from GEMM-ESC models, inbred mice, or orthotopic organoid transplants models, yielded similar results (FIG. 5A; FIGS. 12A-12B and FIG. 30).

While the effects of mutant Kras and injury in inducing accessibility-LOSS were additive, their ability to lead to accessibility-GAIN at a large set of regions appeared synergistic (FIGS. 5B-5D). Accordingly, unsupervised kmeans clustering of all differential ATAC peaks between cells undergoing neoplastic transformation or regeneration vs normal (FIG. 5B) showed peaks (i) commonly gained or lost upon injury or mutant Kras (clusters A1, A6, respectively); (ii) preferentially altered by either injury (cluster A2) or mutant Kras (clusters A3, A5); as well as peaks (iii) uniquely gained upon co-occurrence of both injury and mutant Kras (cluster A4) (FIG. 5D, examples in FIG. 5E). Many accessibility changes appeared to involve distal enhancers, as most de novo peaks arose in non-coding intergenic and intronic regions (FIGS. 11C, 11D), with a larger contribution of proximal-promoter elements found selectively altered in the regeneration setting (Reg-ADM) (FIG. 12E). Strikingly, 67% of the ATAC-seq peaks that uniquely arise in the presence of mutant Kras and injury (Kras*-ADR; cluster A4) were retained late-stage cancer cells (FIG. 11A).

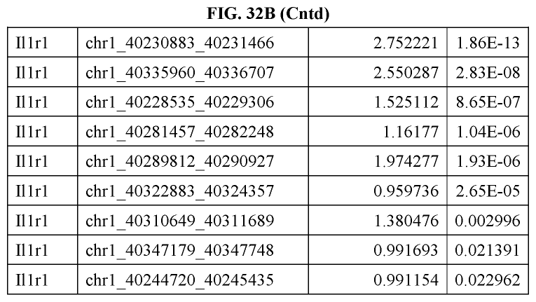

Peaks uniquely gained or lost during early neoplasia versus regeneration (FIG. 11E) were also associated with distinct TF motifs (FIG. 11F). Consistent with the reversible nature of the ADM response associated with regeneration (Reg-ADM), peaks selectively gained in the regeneration context (Reg-ADM) were enriched for motifs linked to the acinar TFs, including Nr5a2. In contrast, peaks enriched for Nr5a2 and other acinar TF binding sites were lost during early neoplasia (Kras*-ADR) (FIGS. 11F, 11G) which, conversely, involved selective gains at loci enriched for AP1 and, to a lesser extent, Onecut motifs (p<10′ and 10¹⁶, respectively) (FIGS. 5F, 11F). Consistently, ATAC LOSS (A5, A6) or GAIN (A1-A4) clusters showed an inverse proportion of experimentally-validated Nr5a2-bound loci and AP1+ motifs (FIG. 5G). In agreement, acinar and AP1 TFs showed consistent differences in expression in Kras*-ADR vs Reg-ADM (FIG. 11G). In this regard, the induction of AP1 factors in early neoplasia stands in contrast to other TFs, such as Foxa1, which are induced in invasive disease (FIG. 1111). therefore, Kras mutation and injury synergize to promote rapid chromatin accessibility gains during early neoplasia. Further, chromatin accessibility dynamics exhibits distinctive features during regeneration and neoplasia. Together, these data suggest that redistribution of open chromatin, from regions defining acinar differentiation to AP1-rich domains, may be a defining feature of early pancreatic neoplasia, possibly endowing incipient tumor cells with functional traits that are only transiently induced (or not induced at all) during tissue regeneration. In sum, mutant Kras and injury promote an altered state that distinguishes neoplastic transformation from regenerative plasticity and that is largely retained in invasive PDAC.

### Example 7: The Brd4-Dependent Transcriptional Programs Required for Regeneration and Neoplasia are Distinct

While the above data indicate that oncogenic Kras and tissue injury cooperate to promote PDAC-relevant chromatin alterations prior to the establishment of PDAC precursor lesions, they do not establish functional causality of the downsteam effector programs. Given that Brd4 was required for both neoplastic and regenerative pancreatic plasticity, it was reasoned that the gene expression programs sensitive to Brd4 perturbation would be highly enriched for factors that mediate these cell fate transitions. Therefore, RNA-seq and ATAC-seq analyses were performed in lineage-traced pancreatic epithelial cells (mKate2/GFP⁺) undergoing regenerative (Reg ADM) or pro-neoplastic (Kras*ADR) metaplasia after acute dox-induced Brd4 suppression. In these experiments, metaplastic transitions were synchronously induced by caerulein treatment initiated 6 days after dox addition, and shRNA-expressing cells were analyzed 2 days later, a timepoint with maximal Brd4 knockdown but prior to the presentation of PanIN or regenerative defects (FIG. 12A).

Consistent with the divergent chromatin states that occur during normal regeneration and early neoplasia, the Brd4-sensitive transcriptional programs observed in each condition were associated with distinct biological processes (FIG. 12B; data not shown). During regeneration, epithelial-specific inactivation of Brd4 reduced levels of key acinar-lineage TF mRNAs (e.g. Nr5a2, Rbpjl, Ptf1a), thus explaining its role in re-establishing acinar identity after injury (FIGS. 6A, 6B; FIG. 12C). In the neoplastic setting, Brd4 inactivation similarly reduced the expression of acinar-associated genes (e.g. Ptf1a, Mist1), but also blunted the activation of an even greater set of genes that were to the mutant Kras and injury context (FIGS. 6A, 6B, 12C). Interestingly, many of the Brd4-sensitive genes in this context were overexpressed in human PDAC samples and tumor organoids compared to normal pancreas tissue and organoids, respectively (FIG. 6C, FIG. 12D), and included known cancer drivers (e.g. Trim29/Atdc, Muc4, Agr2, Shh, Wnt5a, Cdh17, Cdk1 or Fgfr2). Of note, in both the regenerative and neoplastic settings, Brd4 suppression did not prevent the activation of classic metaplasia-associated genes (see Myc and Sox9 in FIG. 12E), a result that was in agreement with its dispensability for ADM.

To rule out that the Brd4-dependent transcriptional programs observed could be produced by depeletion of particular epithelial cell sub-types hypersensitive to Brd4 suppression, parallel ATAC-seq analyses were performed in shRen and shBrd4 cells in KC-GEMM mice at the same time point after dox and injury. This showed retained ATAC peaks associated with the abovementioned downregulation of Brd4-target gene expression (examples in FIG. 6D, FIG. 12E), supporting direct effects of Brd4 inactivation in blunting chromatin interpretation at these loci. Moreover, Brd4 inactivation did not affect the expression of genes that remain unchanged in response to mutant Kras and injury but, importantly, blunted acinar and mutant Kras-specific genes exhibiting chromatin accessibility changes, including known AP1 targets (FIG. 6E, FIGS. 12C-12F). Overall, these analyses identify distinct Brd4-sensitive programs that functionally uncouple the processes of regeneration and tumorigenesis, demonstrating a high level of molecular specificity that may be exploited for early detection and/or treatment of pancreatic cancer.

Accordingly, these results demonstrate that inhibition of factors regulated by Brd4 and tumor-specific chromatin alterations are useful in methods for treating or preventing pancreatic cancer in a subject in need thereof.

### Example 8: Injury-Facilitated Chromatin Dysregulation Activates the Alarmin Cytokine Il-33 and Contributes to Kras-Driven Tumorigenesis

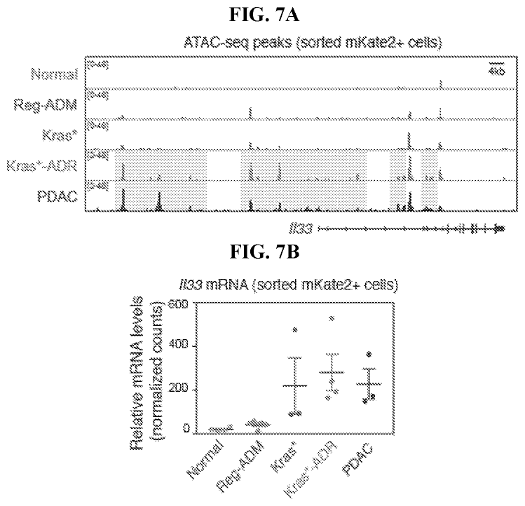

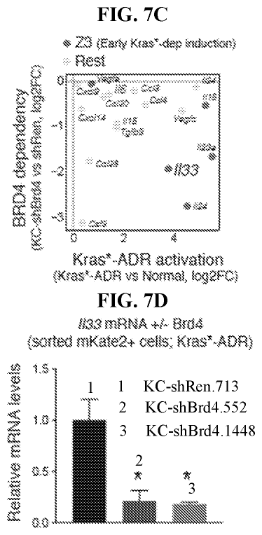

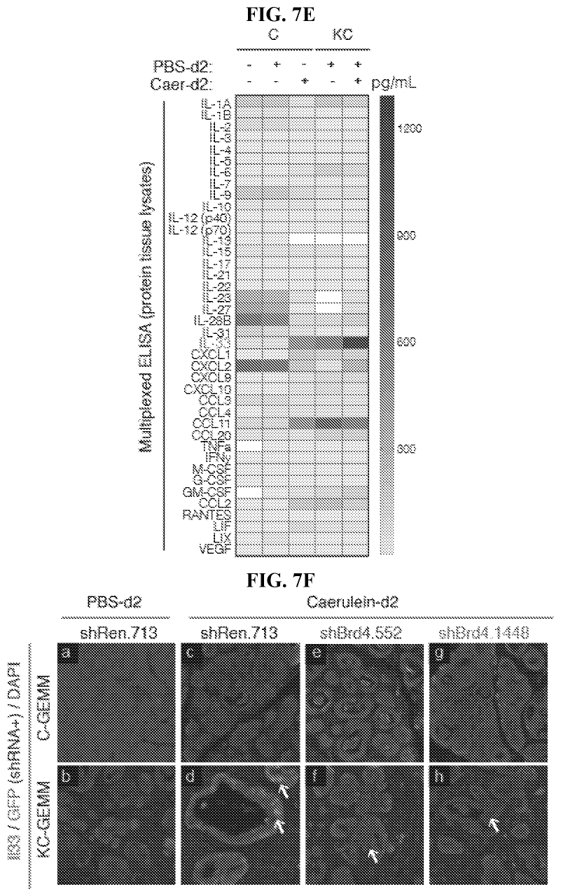

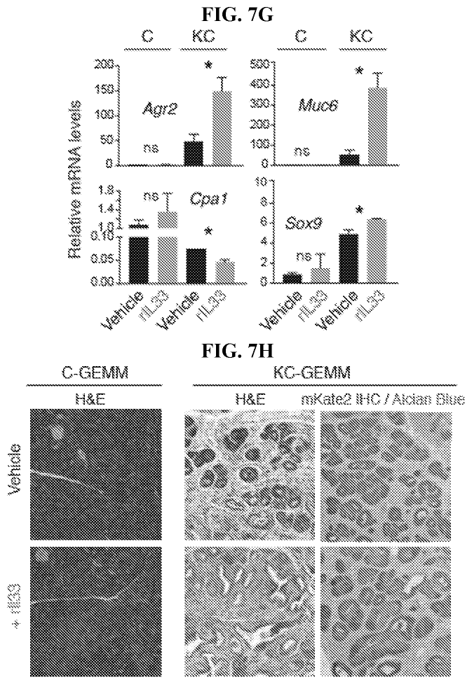

To identify specific mediators of pancreatic tumorigenesis downstream of the described epigenetic alterations, the datasets were then examined for genes showing features of enhancer-dependent activation in early neoplasia but not regeneration (FIG. 13A). One gene that caught attention was Il-33 (FIG. 13B), an ‘alarmin’ cytokine that plays a central role in triggering inflammation and tissue remodeling in response to injury (Millar et al., 2017) and can also influence microenvironmental states in cancer (Larsen et al., 2018). Interestingly, numerous ATAC-seq peaks were selectively gained in the intergenic and intronic regions of the Il-33 locus of Kras-mutant epithelial cells; these novel peaks appeared within 48 hours of tissue injury and were retained in established PDAC (FIG. 7A). This apparent opening of the Il-33 locus was accompanied by a >14 fold increase in Il-33 mRNA in FACS-sorted pancreatic epithelial cells (FIG. 7B) which was Brd4-dependent (FIGS. 7C, 7D). Consistent with these data, a multiplexed immunoassay for 40 different cytokines revealed that Il-33 was the most upregulated cytokine (20-fold increase) in pancreatic tissue undergoing neoplastic transformation (Kras*-ADR) (FIG. 7E, FIG. 13A), and immunohistochemistry confirmed a Brd4-dependent increase in Il-33 protein in the mutant Kras-expressing epithelium under the same conditions (FIG. 7F). Importantly, the activation of epithelial Il-33 was not a general response to inflammation, as the majority of other cytokines examined, including other known injury alarmins (ie. Il25, Il1a, Tslp), were either not found to be similarly induced or not Brd4-dependent (FIG. 7C).

To determine whether the activation of Il-33 by the synergistic effects of mutant Kras and tissue injury functionally contributes to neoplasia, the extent to which exogenous Il-33 could recapitulate the effects of injury in Kras mutant mice was examined. Recombinant mouse Il-33 (rIl-33) was introduced into KC-GEMM (mutant Kras) and C-GEMM (wt Kras) mice by intraperitoneal injection, and mice were analyzed by histology and molecular analyses of cell fate markers in FACS-sorted mKate2⁺ cells 3 weeks later. Remarkably, rIl-33 was sufficient to trigger many of the tumor-promoting outputs of injury in the mutant Kras setting, facilitating the transition of acinar cells into a ductal, mucinous state and the rapid establishment of mucinous pancreatic intraepithelial neoplasia (PanIN) lesions (see “KC” panels in FIGS. 7G, 71I). Importantly, at the same time point, rIl-33 had no detectable effects on the histology or cell fate marker expression in C-GEMM Kras wild-type mice. Thus, these results identify Il-33 as an epigenetically dysregulated gene in the neoplastic pancreatic epithelium and an effector of the neoplastic program. Altogether, these findings indicate that gene-environment interactions can rapidly produce epigenetic states characteristic of invasive disease and, through the induction of key mediators such as Il-33, instruct neoplastic lineage commitment.

These results demonstrate that elevated IL-33 signaling serves as a marker for pancreatic tumorigenesis, and that blockade of IL-33 signaling may be useful in treating pancreatic cancer. Accordingly, the inhibitors of Type 2 cytokine signaling disclosed herein are useful in methods for treating or preventing pancreatic cancer in a subject in need thereof.

### Example 9: Mutant Kras Dependent Activation of Epithelial Th2 Cytokine Receptors During Early Pancreatic Neoplasia

Prompted by the functional experiment revealing mutant Kras-specific effects of IL-33 in the pancreatic epithelium, and in line with the ATAC- and RNA-seq profiling data, specific Th2 receptors (e.g., Il4ra, Il13ra1, Il13ra2, Il17re, Il18r1, Il18rap, Il4ra, Il31ra) become transcriptionally activated in Kras-mutant but not wild-type pancreatic epithelial cells (FIG. 14A). This activation was specific (i.e. not observed for many other cytokine receptors, nor during normal pancreas regeneration) and is associated with the emergence of multiple de novo ATAC peaks (FIG. 14B). Preliminary gain-of-function experiments in mutant Kras pancreatic organoids indicate that these druggable Th2 cytokine receptors are functional and modulate organoid growth in vitro (FIG. 14C).

These data demonstrate that injury drives a coordinated activation of an IL-33-Th2 cytokine signaling axis in the mutant Kras (but not wild-type) pancreatic epithelium that is pharmacologically actionable. Accordingly, the inhibitors of Type 2 cytokine signaling disclosed herein are useful in methods for treating or preventing pancreatic cancer in a subject in need thereof.

### Example 10: Development of New Mouse Models for Spatiotemporally-Controlled Inhibition of Brd4 In Vivo During Pancreatic Tumorigenesis and Normal Pancreatic Regeneration

FIG. 15 shows the approach for doxycyline (dox)-inducible Brd4 suppression in the pancreatic epithelium. As shown in FIG. 1C, Brd4 was effectively downgraded in metaplastic or normal exocrine pancreas (marked by arrows), as validated by Brd4 immunohistochemistry (IHC). KRAS-mutant cells expressing GFP-linked shRNAs effectively undergo acinar-to-ductal metaplasia (ADM), as assessed by GFP (marking shRNA⁺ cells, green) and SOX9 (ADM/dedifferentiation marker, red) co-immunofluorescence (co-IF) (FIGS. 16A-16B), but do not contribute to Alcian Blue-positive mucinous PanIN lesions (FIG. 2C and FIG. 17), not even in the context of injury-accelerated tumorigenesis (see FIG. 2G). As shown in FIG. 18, Myc is expressed in Brd4-suppressed pancreatic epithelial cells undergoing KRAS-driven acinar-to-ductal metaplasia, as assessed by Myc IF (shown at day 2 post-caerulein treatment).

Normal pancreas expressing Brd4-shRNAs effectively undergo injury-driven ADM, as assessed by GFP and CK19 (Krt19) (ADM/dedifferentiation marker) co-IF (FIG. 3E) that, in contrast to shRen-expressing control pancreas, is sustained (see retained Krt19 expression, FIG. 3E) and not coupled with regeneration, as indicated by H&E (FIG. 3D), brightfield and fluorescence images (FIG. 3B), and pancreas-to-body weight ratios (FIG. 19), at the indicated days (“D”) post-caerulein treatment. As shown in FIG. 8E, Brd4 silencing did not impair c-Myc expression in KRAS-mutant metaplasia demonstrating that Brd4 mediates neoplastic reprogramming of acinar cells via Myc-independent mechanisms.

Accordingly, these results demonstrate that inhibition of factors regulated by Brd4 are useful in methods for treating or preventing pancreatic cancer in a subject in need thereof.

### Example 11: Distinct Brd4-Dependent Epigenetic Networks Underlie Pancreatic Tumorigenesis Vs. Injury-Induced Regeneration

FIG. 20 shows the strategy for identifying the Brd4-mediated transcriptional programs in mutant KRAS and wildtype pancreatic epithelial cells. Exploiting the traceable nature of the KC-GEMM-ESC and C-GEMM models, gene expression profiles were generated from FACS-sorted pancreatic epithelial cells (mKate2⁺, GFP⁺, Cd45⁻) isolated from KC-GEMM-ESC or C-GEMM mice (n=3 per group; 2 different Brd4 shRNAs vs. Ren shRNAs). Analyses were performed at early time-points post-dox treatment to enrich for direct effects; and when indicated, dedifferentiation was induced in a synchronous manner throughout the pancreas by acute caerulein treatment.

FIGS. 21A-21B show RNA-Seq data and GSEA plots in FACS-sorted mutant KRAS pancreatic epithelial cells expressing shRNAs against Renilla (shRen) or Brd4 (shBrd4), using published signatures of genes associated with pancreatic tumorigenesis and disease progression in mice and humans. As shown in FIGS. 21C-21D, distinct Brd4 targets are associated with tumor progression and regeneration. Tumor-specific Brd4 targets (including IL-33) were validated by qPCR and IF staining in pancreatic tissues from KC- and C-GEMM animals treated with -shBrd4/-shRen at day 2 post-Caerulein exposure (see FIGS. 24A-24B).

In order to gain mechanistic insight into the epigenetic control of pancreatic tumorigenesis vs regeneration, the chromatin and transcriptional landscapes of sorted pancreatic epithelial cells undergoing mutant KRAS- and/or injury-driven cell identity changes via ATAC-Seq and RNA-Seq were mapped. FIGS. 22A-22C reveal that despite a shared dependency on Brd4, oncogenic and regenerative plasticity display quantitative and qualitative differences in chromatin accessibility dynamics, particularly at distal cis-regulatory regions, that were associated with distinct transcriptional programs.

A synergistic interaction between mutant KRAS and inflammatory insults in shaping the chromatin landscape of pancreatic epithelial cells was identified (FIGS. 23A-23B), which was associated with Brd4-dependent de novo activation of a discrete set of genes involved in oncogenic signaling, immumomodulation and stemness. Importantly, this class of Brd4 targets appeared tumor specific, as they became further activated in advanced murine and human PDAC yet remain silent in normal and regenerating pancreas.

As shown in FIGS. 25A-25D, tumor-specific Brd4 targets (e.g., IL-33) gained chromatin accessibility at distal regulatory elements during pancreatic tumorigenesis. FIG. 25E demonstrates that the elevated IL-33 expression levels observed in pancreatic tumors was dependent on Brd4 expression.

Activation of epithelial IL-33 signaling selectively promoted KRAS-driven tumorigenesis. As shown in FIGS. 26A-26B, epigenetic activation of epithelial cytokine signaling occurred in KRAS-driven ADR and PDAC, but not in injury-induced ADM or normal pancreas.

As shown in FIG. 27A, KC-GEMM mice treated with recombinant IL-33 showed enhanced tumorigenesis and cancer stem cell expansion compared to control KC-GEMM mice that received PBS. Recombinant IL-33 was sufficient to drive expansion of Dclk1⁺ cancer stem cells in vivo. FIGS. 28A-28B show that treatment with recombinant IL-33 accelerates mutant KRAS-driven tumorigenesis, while having no effect in wild-type pancreas. KC-GEMM animals that were subjected to pancreas-specific IL-33 silencing exhibited reduced fibrosis and delayed development of mutant KRAS-driven pancreatic intraepithelial neoplasias compared to non-induced KC-GEMM control animals. See FIG. 27B.

Taken together, these results demonstrate that elevated IL-33 signaling serves as a marker for pancreatic tumorigenesis, and that blockade of IL-33 signaling may be useful in treating or preventing pancreatic cancer. Accordingly, the inhibitors of Type 2 cytokine signaling disclosed herein are useful in methods for treating or preventing pancreatic cancer in a subject in need thereof.

### Example 12: Use of Inhibitors of Type 2 Cytokine Signaling to Treat or Prevent Pancreatic Cancer

KC-GEMM and KPC-GEMM (advanced pancreatic cancer model that includes a p53 mutation) mice will be treated with one or more inhibitors of Type 2 cytokine signaling at varying doses. Exemplary inhibitors of Type 2 cytokine signaling that will be tested include one or more of dupilumab, Etokimab (ANB020), Mepolizumab, reslizumab, benralizumab, lebrikizumab, risankizumab, tocilizumab, tralokinumab, anrukinzumab, AMG317, STATE inhibitors, STAT3 inhibitors, Secukinumab, ustekinumab, guselkumab, and gevokizumab, or mouse-compatible analogs for the murine models of pancreatic cancer. Mouse-compatible Type 2 cytokine inhibitors that will be tested include anti-mouse IL-4 (clone 11B11) (Bio X Cell, West Lebanon N.H.), Mouse IL-13 Neutralizing antibody (clone 8H8) (Invivogen, San Diego Calif.), Mouse IL-33 MAb (Clone 396118) (R&D Systems, Minneapolis, Minn.), anti-mouse/human IL-5 (Clone TRFK5) (Bio X Cell, West Lebanon N.H.) or Mouse ST2/IL-33R antibody (Clone 245707) (R&D Systems, Minneapolis, Minn.). For in vitro treatment experiments, anti-tumor efficacy of the above mentioned inhibitors will be evaluated in human and mouse pancreatic cancer cell lines or organoids systems.

It is anticipated that KC-GEMM mice that are treated with one or more inhibitors of Type 2 cytokine signaling will exhibit reduced fibrosis and/or delayed progression of mutant KRAS-driven pancreatic intraepithelial neoplasias compared to untreated KC-GEMM control animals. In experiments treating advanced pancreatic cancer (KPC-GEMM and transplantable models), it is anticipated that mice treated with one or more inhibitors of Type 2 cytokine signaling will exhibit reduced fibrosis, enhanced anti-tumor immunity, tumor regressions and/or delayed progression. In in vitro drug studies, pancreatic cancer cell lines or organoids treated with one or more inhibitors of Type 2 cytokine signaling are anticipated to exhibit reduced stem-like properties, reduced proliferation and/or undergo cell death.

Taken together, these results will demonstrate that inhibitors of Type 2 cytokine signaling are useful for treating or preventing pancreatic cancer. Accordingly, the inhibitors of Type 2 cytokine signaling disclosed herein are useful in methods for treating or preventing pancreatic cancer in a subject in need thereof.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method for treating or preventing pancreatic cancer in a subject in need thereof comprising administering to the subject an effective amount of an inhibitor of Type 2 cytokine signaling, wherein the subject harbors a KRAS mutation, optionally wherein the KRAS mutation is selected from the group consisting of G12C, G12D, G12V, G12A, G12S, G12R, G13D, G13C, G13S, G13R, G13A, G13V, Q61H, Q61L, Q61R, Q61K, Q61P, and Q61E.

2. (canceled)

3. The method of claim 1, wherein the inhibitor of Type 2 cytokine signaling inhibits a Type 2 cytokine or Type 2 cytokine receptor signaling protein selected from the group consisting of IL-33, IL-4, IL-13, IL-5, IL-23A, IL-17E, IL-9, IL9R, IL1RL1, IL4RA, IL13RA1, IL13RA2, IL5RA, IL1RL2, IL17RA, IL31RA, IL17RE, IL18R1, IL18RAP, and IL1R1.

4. The method of claim 1, wherein the inhibitor of Type 2 cytokine signaling is a small molecule, an inhibitory nucleic acid, a tyrosine kinase inhibitor, a decoy cytokine receptor, or an antibody, or
wherein the inhibitor of Type 2 cytokine signaling is selected from the group consisting of dupilumab, Etokimab (ANB020), Mepolizumab, reslizumab, benralizumab, lebrikizumab, risankizumab, tocilizumab, tralokinumab, anrukinzumab, AMG317, STATE inhibitors, STAT3 inhibitors, Secukinumab, ustekinumab, guselkumab, and gevokizumab.

5. (canceled)

6. The method of claim 1 further comprising administering to the subject an effective amount of a Brd4 inhibitor, optionally wherein the Brd4 inhibitor is selected from the group consisting of (+)-JQ1, I-BET762, OTX015, I-BET151, CPI203, PFI-1, MS436, CPI-0610, RVX2135, FT-1101, BAY1238097, INCB054329, TEN-010, GSK2820151, ZEN003694, BAY-299, BMS-986158, ABBV-075, GS-5829, and PLX51107, or
wherein the Brd4 inhibitor is a small molecule, an inhibitory nucleic acid, or an antibody.

7. (canceled)

8. (canceled)

9. The method of claim 1, wherein the subject harbors a mutation in TP53.

10. The method of claim 1, wherein the pancreatic cancer comprises exocrine tumors or is pancreatic ductal adenocarcinoma.

11. (canceled)

12. The method of claim 1, wherein the subject exhibits elevated expression levels of at least one of IL-33, IL-4, IL-13, IL-5, IL-23A, IL-17E, IL-9, IL9R, IL1RL1, IL4RA, IL13RA1, IL13RA2, IL5RA, IL1RL2, IL17RA, IL31RA, IL17RE, IL18R1, IL18RAP, and IL1R1 compared to that observed in a healthy control subject or a predetermined threshold.

13. The method of claim 1, further comprising sequentially, simultaneously, or separately administering one or more additional therapeutic agents to the subject.

14. A method for selecting pancreatic cancer patients for treatment with an inhibitor of Type 2 cytokine signaling comprising
(a) detecting expression levels or chromatin accessibility levels of a Type 2 cytokine or Type 2 cytokine receptor signaling protein in biological samples obtained from pancreatic cancer patients, wherein the Type 2 cytokine or the Type 2 cytokine receptor signaling protein is selected from the group consisting of IL-33, IL-4, IL-13, IL-5, IL-23A, IL-17E, IL-9, IL9R, IL1RL1, IL4RA, IL13RA1, IL13RA2, IL5RA, IL1RL2, IL17RA, IL31RA, IL17RE, IL18R1, IL18RAP, and IL1R1;
(b) identifying pancreatic cancer patients that exhibit
(i) mRNA/protein expression levels of Type 2 cytokine or Type 2 cytokine receptor signaling protein that are elevated compared to that observed in a healthy control subject or a predetermined threshold, or
(ii) chromatin accessibility levels of Type 2 cytokine or Type 2 cytokine receptor signaling protein that are elevated compared to that observed in a healthy control subject or a predetermined threshold; and
(c) administering an inhibitor of Type 2 cytokine signaling to the pancreatic cancer patients of step (b).

15. The method of claim 14, wherein the inhibitor of Type 2 cytokine signaling is a small molecule, an inhibitory nucleic acid, a tyrosine kinase inhibitor, a decoy cytokine receptor, or an antibody, or wherein the inhibitor of Type 2 cytokine signaling is selected from the group consisting of dupilumab, Etokimab (ANB020), Mepolizumab, reslizumab, benralizumab, lebrikizumab, risankizumab, tocilizumab, tralokinumab, anrukinzumab, AMG317, STAT6 inhibitors, STAT3 inhibitors, Secukinumab, ustekinumab, guselkumab, and gevokizumab.

16. (canceled)

17. The method of claim 14, further comprising administering a Brd4 inhibitor to the pancreatic cancer patients of step (b), optionally wherein the Brd4 inhibitor is a small molecule, an inhibitory nucleic acid, or an antibody or wherein the Brd4 inhibitor is selected from the group consisting of (+)-JQ1, I-BET762, OTX015, I-BET151, CPI203, PFI-1, MS436, CPI-0610, RVX2135, FT-1101, BAY1238097, INCB054329, TEN-010, GSK2820151, ZEN003694, BAY-299, BMS-986158, ABBV-075, GS-5829, and PLX51107.

18. (canceled)

19. (canceled)

20. The method of claim 14, wherein the pancreatic cancer patients harbor a KRAS mutation selected from the group consisting of G12C, G12D, G12V, G12A, G12S, G12R, G13D, G13C, G13S, G13R, G13A, G13V, Q61H, Q61L, Q61R, Q61K, Q61P, and Q61E and/or a mutation in TP53.

21. (canceled)

22. The method of claim 14, wherein the pancreatic cancer patients exhibit exocrine tumors or wherein the pancreatic cancer patients suffer from or are at risk for pancreatic ductal adenocarcinoma.

23. (canceled)

24. The method of claim 14, wherein the expression levels or chromatin accessibility levels of the Type 2 cytokine or the Type 2 cytokine receptor signaling protein are detected via ChIP, MNase, FAIRE, DNAse, ATAC-seq, RT-PCR, Northern Blotting, RNA-Seq, microarray analysis, High-performance liquid chromatography (HPLC), mass spectrometry, immunohistochemistry (IHC), fluorescence in situ hybridization (FISH), Western Blotting, immunoprecipitation, flow cytometry, Immuno-electron microscopy, immunoelectrophoresis, enzyme-linked immunosorbent assays (ELISA), or multiplex ELISA antibody arrays.

25. The method of claim 14, wherein the biological samples are pancreatic cancer specimens, blood, serum, or plasma.

26. A kit comprising at least one inhibitor of Type 2 cytokine signaling and instructions for using the at least one inhibitor of Type 2 cytokine signaling to treat or prevent pancreatic cancer.

27. The kit of claim 26, wherein the inhibitor of Type 2 cytokine signaling is a small molecule, an inhibitory nucleic acid, a tyrosine kinase inhibitor, a decoy cytokine receptor, or an antibody, or
wherein the inhibitor of Type 2 cytokine signaling is selected from the group consisting of dupilumab, Etokimab (ANB020), Mepolizumab, reslizumab, benralizumab, lebrikizumab, risankizumab, tocilizumab, tralokinumab, anrukinzumab, AMG317, STATE inhibitors, STAT3 inhibitors, Secukinumab, ustekinumab, guselkumab, and gevokizumab.

28. The kit of claim 26, wherein the inhibitor of Type 2 cytokine signaling inhibits a Type 2 cytokine or Type 2 cytokine receptor signaling protein selected from the group consisting of IL-33, IL-4, IL-13, IL-5, IL-23A, IL-17E, IL-9, IL9R, IL1RL1, IL4RA, IL13RA1, IL13RA2, IL5RA, IL1RL2, IL17RA, IL31RA, IL17RE, IL18R1, IL18RAP, and IL1R1.

29. (canceled)

30. The kit of claim 26, further comprising at least one Brd4 inhibitor, wherein the at least one Brd4 inhibitor is a small molecule, an inhibitory nucleic acid, or an antibody, or wherein the at least one Brd4 inhibitor is selected from the group consisting of (+)-JQ1, I-BET762, OTX015, I-BET151, CPI203, PFI-1, MS436, CPI-0610, RVX2135, FT-1101, BAY1238097, INCB054329, TEN-010, GSK2820151, ZEN003694, BAY-299, BMS-986158, ABBV-075, GS-5829, and PLX51107.

31. (canceled)

32. The kit of claim 26, further comprising reagents for detecting mRNA or protein expression levels or chromatin accessibility levels of a Type 2 cytokine or Type 2 cytokine receptor signaling in a biological sample.


SEQUENCE LISTING (53 sequences)

SEQ ID NO: 1 - DNA, 20 bp, Artificial Sequence
  Description of Artificial Sequence Synthetic primer
caccctgaaaactttgcccc

SEQ ID NO: 2 - DNA, 25 bp, Artificial Sequence
  Description of Artificial Sequence Synthetic primer
gtatagataagcattataattccta

SEQ ID NO: 3 - DNA, 18 bp, Artificial Sequence
  Description of Artificial Sequence Synthetic primer
tattgttcccatatccat

SEQ ID NO: 4 - DNA, 20 bp, Artificial Sequence
  Description of Artificial Sequence Synthetic primer
ctagtttagacttgattgtg

SEQ ID NO: 5 - DNA, 20 bp, Artificial Sequence
  Description of Artificial Sequence Synthetic primer
gctgcgtctgttgacacatt

SEQ ID NO: 6 - DNA, 20 bp, Artificial Sequence
  Description of Artificial Sequence Synthetic primer
gacttgcaggacagggagac

SEQ ID NO: 7 - DNA, 22 bp, Artificial Sequence
  Description of Artificial Sequence Synthetic primer
acaactgacaagcacctttctc

SEQ ID NO: 8 - DNA, 23 bp, Artificial Sequence
  Description of Artificial Sequence Synthetic primer
gtttgagtatcgtccagtgatgt

SEQ ID NO: 9 - DNA, 20 bp, Artificial Sequence
  Description of Artificial Sequence Synthetic primer
agcccacattccctatcagc

SEQ ID NO: 10 - DNA, 21 bp, Artificial Sequence
  Description of Artificial Sequence Synthetic primer
cacagtggaagattgcgagag

SEQ ID NO: 11 - DNA, 21 bp, Artificial Sequence
  Description of Artificial Sequence Synthetic primer
cagtcttcggcaatgagaact

SEQ ID NO: 12 - DNA, 20 bp, Artificial Sequence
  Description of Artificial Sequence Synthetic primer
gggaagggcactcgaacatc

SEQ ID NO: 13 - DNA, 21 bp, Artificial Sequence
  Description of Artificial Sequence Synthetic primer
cgtgcagcacaagaaagacca

SEQ ID NO: 14 - DNA, 21 bp, Artificial Sequence
  Description of Artificial Sequence Synthetic primer
gcagcgccttgaagatagcat

SEQ ID NO: 15 - DNA, 21 bp, Artificial Sequence
  Description of Artificial Sequence Synthetic primer
tcagtcaacgggggacataaa

SEQ ID NO: 16 - DNA, 21 bp, Artificial Sequence
  Description of Artificial Sequence Synthetic primer
ggggctgtactgcttaaccag

SEQ ID NO: 17 - DNA, 19 bp, Artificial Sequence
  Description of Artificial Sequence Synthetic primer
gctccaagcagatgcagca

SEQ ID NO: 18 - DNA, 18 bp, Artificial Sequence
  Description of Artificial Sequence Synthetic primer
ccggatgtgaggcagcag

SEQ ID NO: 19 - DNA, 20 bp, Artificial Sequence
  Description of Artificial Sequence Synthetic primer
gtctttccccagcacagtgc

SEQ ID NO: 20 - DNA, 22 bp, Artificial Sequence
  Description of Artificial Sequence Synthetic primer
ctcttgcctacgccaccagctc

SEQ ID NO: 21 - DNA, 31 bp, Artificial Sequence
  Description of Artificial Sequence Synthetic primer
agctagccaccatggcttgagtaagtctgca

SEQ ID NO: 22 - DNA, 20 bp, Artificial Sequence
  Description of Artificial Sequence Synthetic oligonucleotide
agtgaagccctccgagtgtc

SEQ ID NO: 23 - DNA, 2685 bp, Homo sapiens
acagagctgcagctcttcagggaagaaatcaaaacaagatcacaagaatactgaaaaatgaagcctaaaatgaagtattcaaccaacaaaatttccacagcaaagtggaagaacacagcaagcaaagccttgtgtttcaagctgggaaaatcccaacagaaggccaaagaagtttgccccatgtactttatgaagctccgctctggccttatgataaaaaaggaggcctgttactttaggagagaaaccaccaaaaggccttcactgaaaacaggtagaaagcacaaaagacatctggtactcgctgcctgtcaacagcagtctactgtggagtgctttgcctttggtatatcaggggtccagaaatatactagagcacttcatgattcaagtatcacaggaatttcacctattacagagtatcttgcttctctaagcacatacaatgatcaatccattacttttgctttggaggatgaaagttatgagatatatgttgaagacttgaaaaaagatgaaaagaaagataaggtgttactgagttactatgagtctcaacacccctcaaatgaatcaggtgacggtgttgatggtaagatgttaatggtaaccctgagtcctacaaaagacttctggttgcatgccaacaacaaggaacactctgtggagctccataagtgtgaaaaaccactgccagaccaggccttctttgtccttcataatatgcactccaactgtgtttcatttgaatgcaagactgatcctggagtgtttataggtgtaaaggataatcatcttgctctgattaaagtagactcttctgagaatttgtgtactgaaaatatcttgtttaagctctctgaaacttagttgatggaaacctgtgagtcttgggttgagtacccaaatgctaccactggagaaggaatgagagataaagaaagagacaggtgacatctaagggaaatgaagagtgcttagcatgtgtggaatgttttccatattatgtataaaaatattttttctaatcctccagttattcttttatttccctctgtataactgcatcttcaatacaagtatcagtatattaaatagggtattggtaaagaaacggtcaacattctaaagagatacagtctgacctttacttttctctagtttcagtccagaaagaacttcatatttagagctaaggccactgaggaaagagccatagcttaagtctctatgtagacagggatccattttaaagagctacttagagaaataattttccacagttccaaacgataggctcaaacactagagctgctagtaaaaagaagaccagatgcttcacagaattatcattttttcaactggaataaaacaccaggtttgtttgtagatgtcttaggcaacactcagagcagatctcccttactgtcaggggatatggaacttcaaaggcccacatggcaagccaggtaacataaatgtgtgaaaaagtaaagataactaaaaaatttagaaaaataaatccagtatttgtaaagtgaataacttcatttctaattgtttaatttttaaaattctgatttttatatattgagtttaagcaaggcattcttacacgaggaagtgaagtaaattttagttcagacataaaatttcacttattaggaatatgtaacatgctaaaacttttttttttttaaagagtactgagtcacaacatgttttagagcatccaagtaccatataatccaactatcatggtaaggccagaaatcttctaacctaccagagcctagatgagacaccgaattaacattaaaatttcagtaactgactgtccctcatgtccatggcctaccatcccttctgaccctggcttccagggacctatgtcttttaatactcactgtcacattgggcaaagttgcttctaatccttatttcccatgtgcacaagtctttttgtattccagcttcctgataacactgcttactgtggaatattcatttgacatctgtctcttttcatttcttttaactaccatgcccttgatatatcttttgcacctgctgaacttcatttctgtatcacctgacctctggatgccaaaacgtttattctgctttgtctgttgtagaattttagataaagctattaatggcaatatttttttgctaaacgtttttgttttttactgtcactagggcaataaaatttatactcaaccatataataacattttttaactactaaaggagtagtttttattttaaagtcttagcaatttctattacaacttttcttagacttaacacttatgataaatgactaacatagtaacagaatctttatgaaatatgaccttttctgaaaatacatacttttacatttctactttattgagacctattagatgtaagtgctagtagaatataagataaaagaggctgagaattaccatacaagggtattacaactgtaaaacaatttatctttgtttcattgttctgtcaataattgttaccaaagagataaaaataaaagcagaatgtatatcatcccatctgaaaaacactaattattgacatgtgcatctgtacaataaacttaaaatgattattaaataatcaaatatatctactacattgtttatattattgaataaagtatattttccaaatgta

SEQ ID NO: 24 - DNA, 615 bp, Homo sapiens
atcgttagcttctcctgataaactaattgcctcacattgtcactgcaaatcgacacctattaatgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctgaatattttaatttatgagtttttgatagctttattttttaagtatttatatatttataactcatcataaaataaagtatatatagaatctaa

SEQ ID NO: 25 - DNA, 1283 bp, Homo sapiens
aagccacccagcctatgcatccgctcctcaatcctctcctgttggcactgggcctcatggcgcttttgttgaccacggtcattgctctcacttgccttggcggctttgcctccccaggccctgtgcctccctctacagccctcagggagctcattgaggagctggtcaacatcacccagaaccagaaggctccgctctgcaatggcagcatggtatggagcatcaacctgacagctggcatgtactgtgcagccctggaatccctgatcaacgtgtcaggctgcagtgccatcgagaagacccagaggatgctgagcggattctgcccgcacaaggtctcagctgggcagttttccagcttgcatgtccgagacaccaaaatcgaggtggcccagtttgtaaaggacctgctcttacatttaaagaaactttttcgcgagggacagttcaactgaaacttcgaaagcatcattatttgcagagacaggacctgactattgaagttgcagattcatttttctttctgatgtcaaaaatgtcttgggtaggcgggaaggagggttagggaggggtaaaattccttagcttagacctcagcctgtgctgcccgtcttcagcctagccgacctcagccttccccttgcccagggctcagcctggtgggcctcctctgtccagggccctgagctcggtggacccagggatgacatgtccctacacccctcccctgccctagagcacactgtagcattacagtgggtgccccccttgccagacatgtggtgggacagggacccacttcacacacaggcaactgaggcagacagcagctcaggcacacttcttcttggtcttatttattattgtgtgttatttaaatgagtgtgtttgtcaccgttggggattggggaagactgtggctgctagcacttggagccaagggttcagagactcagggccccagcactaaagcagtggacaccaggagtccctggtaataagtactgtgtacagaattctgctacctcactggggtcctggggcctcggagcctcatccgaggcagggtcaggagaggggcagaacagccgctcctgtctgccagccagcagccagctctcagccaacgagtaatttattgtttttccttgtatttaaatattaaatatgttagcaaagagttaatatatagaagggtaccttgaacactgggggaggggacattgaacaagttgtttcattgactatcaaactgaagccagaaataaagttggtgacagata

SEQ ID NO: 26 - DNA, 815 bp, Homo sapiens
atgcactttctttgccaaaggcaaacgcagaacgtttcagagccatgaggatgcttctgcatttgagtttgctagctcttggagctgcctacgtgtatgccatccccacagaaattcccacaagtgcattggtgaaagagaccttggcactgctttctactcatcgaactctgctgatagccaatgagactctgaggattcctgttcctgtacataaaaatcaccaactgtgcactgaagaaatctttcagggaataggcacactggagagtcaaactgtgcaagggggtactgtggaaagactattcaaaaacttgtccttaataaagaaatacattgacggccaaaaaaaaaagtgtggagaagaaagacggagagtaaaccaattcctagactacctgcaagagtttcttggtgtaatgaacaccgagtggataatagaaagttgagactaaactggtttgttgcagccaaagattttggaggagaaggacattttactgcagtgagaatgagggccaagaaagagtcaggccttaattttcagtataatttaacttcagagggaaagtaaatatttcaggcatactgacactttgccagaaagcataaaattcttaaaatatatttcagatatcagaatcattgaagtattttcctccaggcaaaattgatatacttttttcttatttaacttaacattctgtaaaatgtctgttaacttaatagtatttatgaaatggttaagaatttggtaaattagtatttatttaatgttatgttgtgttctaataaaacaaaaatagacaactgtt

SEQ ID NO: 27 - DNA, 1127 bp, Homo sapiens
attctgccctcgagcccaccgggaacgaaagagaagctctatctcccctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattca

SEQ ID NO: 28 - DNA, 605 bp, Homo sapiens
aagcgagctccagtccgctgtcaagatgcttctggccatggtccttacctctgccctgctcctgtgctccgtggcaggccaggggtgtccaaccttggcggggatcctggacatcaacttcctcatcaacaagatgcaggaagatccagcttccaagtgccactgcagtgctaatgtgaccagttgtctctgtttgggcattccctctgacaactgcaccagaccatgcttcagtgagagactgtctcagatgaccaataccaccatgcaaacaagatacccactgattttcagtcgggtgaaaaaatcagttgaagtactaaagaacaacaagtgtccatatttttcctgtgaacagccatgcaaccaaaccacggcaggcaacgcgctgacatttctgaagagtcttctggaaattttccagaaagaaaagatgagagggatgagaggcaagatatgaagatgaaatattatttatcctatttattaaatttaaaaagctttctctttaagttgctacaatttaaaaatcaagtaagctactctaaatcagtatcagttgtgattatttgtttaacattgtatgtctttattttgaaataaat

SEQ ID NO: 29 - DNA, 1630 bp, Homo sapiens
acacatcaggggcttgctcttgcaaaaccaaaccacaagacagacttgcaaaagaaggcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgagacatcagggtggcgactctatagactctaggacataaattagaggtctccaaaatcggatctggggctctgggatagctgacccagccccttgagaaaccttattgtacctctcttatagaatatttattacctctgatacctcaacccccatttctatttatttactgagcttctctgtgaacgatttagaaagaagcccaatattataatttttttcaatatttattattttcacctgtttttaagctgtttccatagggtgacacactatggtatttgagtgttttaagataaattataagttacataagggaggaaaaaaaatgttctttggggagccaacagaagcttccattccaagcctgaccacgctttctagctgttgagctgttttccctgacctccctctaatttatcttgtctctgggcttggggcttcctaactgctacaaatactcttaggaagagaaaccagggagcccctttgatgattaattcaccttccagtgtctcggagggattcccctaacctcattccccaaccacttcattcttgaaagctgtggccagcttgttatttataacaacctaaatttggttctaggccgggcgcggtggctcacgcctgtaatcccagcactttgggaggctgaggcgggtggatcacttgaggtcaggagttcctaaccagcctggtcaacatggtgaaaccccgtctctactaaaaatacaaaaattagccgggcatggtggcgcgcacctgtaatcccagctacttgggaggctgaggcaagagaattgcttgaacccaggagatggaagttgcagtgagctgatatcatgcccctgtactccagcctgggtgacagagcaagactctgtctcaaaaaataaaaataaaaataaatttggttctaatagaactcagttttaactagaatttattcaattcctctgggaatgttacattgtttgtctgtcttcatagcagattttaattttgaataaataaatgtatcttattcacatca

SEQ ID NO: 30 - DNA, 815 bp, Homo sapiens; identical to SEQ ID NO: 26

SEQ ID NO: 31 - DNA, 1037 bp, Homo sapiens
aacaggaagcagcttacaaactcggtgaacaactgagggaaccaaaccagagacgcgctgaacagagagaatcaggctcaaagcaagtggaagtgggcagagattccaccaggactggtgcaaggcgcagagccagccagatttgagaagaaggcaaaaagatgctggggagcagagctgtaatgctgctgttgctgctgccctggacagctcagggcagagctgtgcctgggggcagcagccctgcctggactcagtgccagcagctttcacagaagctctgcacactggcctggagtgcacatccactagtgggacacatggatctaagagaagagggagatgaagagactacaaatgatgttccccatatccagtgtggagatggctgtgacccccaaggactcagggacaacagtcagttctgcttgcaaaggatccaccagggtctgattttttatgagaagctgctaggatcggatattttcacaggggagccttctctgctccctgatagccctgtgggccagcttcatgcctccctactgggcctcagccaactcctgcagcctgagggtcaccactgggagactcagcagattccaagcctcagtcccagccagccatggcagcgtctccttctccgcttcaaaatccttcgcagcctccaggcctttgtggctgtagccgcccgggtctttgcccatggagcagcaaccctgagtccctaaaggcagcagctcaaggatggcactcagatctccatggcccagcaaggccaagataaatctaccaccccaggcacctgtgagccaacaggttaattagtccattaattttagtgggacctgcatatgttgaaaattaccaatactgactgacatgtgatgctgacctatgataaggttgagtatttattagatgggaagggaaatttggggattatttatcctcctggggacagtttggggaggattatttattgtatttatattgaattatgtacttttttcaataaagtcttatttttgtggcta

SEQ ID NO: 32 - DNA, 1997 bp, Homo sapiens
gagttgtgaaactgtgggcagaaagttgaggaagaaagaactcaagtacaacccaatgaggttgagatataggctactcttcccaactcagtcttgaagagtatcaccaactgcctcatgtgtggtgaccttcactgtcgtatgccagtgactcatctggagtaatctcaacaacgagttaccaatacttgctcttgattgataaacagaatggggttttggatcttagcaattctcacaattctcatgtattccacagcagcaaagtttagtaaacaatcatggggcctggaaaatgaggctttaattgtaagatgtcctagacaaggaaaacctagttacaccgtggattggtattactcacaaacaaacaaaagtattcccactcaggaaagaaatcgtgtgtttgcctcaggccaacttctgaagtttctaccagctgcagttgctgattctggtatttatacctgtattgtcagaagtcccacattcaataggactggatatgcgaatgtcaccatatataaaaaacaatcagattgcaatgttccagattatttgatgtattcaacagtatctggatcagaaaaaaattccaaaatttattgtcctaccattgacctctacaactggacagcacctcttgagtggtttaagaattgtcaggctcttcaaggatcaaggtacagggcgcacaagtcatttttggtcattgataatgtgatgactgaggacgcaggtgattacacctgtaaatttatacacaatgaaaatggagccaattatagtgtgacggcgaccaggtccttcacggtcaaggatgagcaaggcttttctctgtttccagtaatcggagcccctgcacaaaatgaaataaaggaagtggaaattggaaaaaacgcaaacctaacttgctctgcttgttttggaaaaggcactcagttcttggctgccgtcctgtggcagcttaatggaacaaaaattacagactttggtgaaccaagaattcaacaagaggaagggcaaaatcaaagtttcagcaatgggctggcttgtctagacatggttttaagaatagctgacgtgaaggaagaggatttattgctgcagtacgactgtctggccctgaatttgcatggcttgagaaggcacaccgtaagactaagtaggaaaaatccaattgatcatcatagcatctactgcataattgcagtatgtagtgtatttttaatgctaatcaatgtcctggttatcatcctaaaaatgttctggattgaggccactctgctctggagagacatagctaaaccttacaagactaggaatgatggaaagctctatgatgcttatgttgtctacccacggaactacaaatccagtacagatggggccagtcgtgtagagcactttgttcaccagattctgcctgatgttcttgaaaataaatgtggctataccttatgcatttatgggagagatatgctacctggagaagatgtagtcactgcagtggaaaccaacatacgaaagagcaggcggcacattttcatcctgacccctcagatcactcacaataaggagtttgcctacgagcaggaggttgccctgcactgtgccctcatccagaacgacgccaaggtgatacttattgagatggaggctctgagcgagctggacatgctgcaggctgaggcgcttcaggactccctccagcatcttatgaaagtacaggggaccatcaagtggagggaggaccacattgccaataaaaggtccctgaattctaaattctggaagcacgtgaggtaccaaatgcctgtgccaagcaaaattcccagaaaggcctctagtttgactcccttggctgcccagaagcaatagtgcctgctgtgatgtgcaaaggcatctgagtttgaagctttcctgacttctcctagctggcttatgcccctgcactgaagtgtgaggagcaggaatattaaagggattcaggcctc

SEQ ID NO: 33 - DNA, 3624 bp, Homo sapiens
acttcccgcttgggcgcccggacggcgaatggagcaggggcgcgcagataattaaagatttacacacagctggaagaaatcatagagaagccgggcgtggtggctcatgcctataatcccagcacttttggaggctgaggcgggcagatcacttgagatcaggagttcgagaccagcctggtgccttggcatctcccaatggggtggctttgctctgggctcctgttccctgtgagctgcctggtcctgctgcaggtggcaagctctgggaacatgaaggtcttgcaggagcccacctgcgtctccgactacatgagcatctctacttgcgagtggaagatgaatggtcccaccaattgcagcaccgagctccgcctgttgtaccagctggtttttctgctctccgaagcccacacgtgtatccctgagaacaacggaggcgcggggtgcgtgtgccacctgctcatggatgacgtggtcagtgcggataactatacactggacctgtgggctgggcagcagctgctgtggaagggctccttcaagcccagcgagcatgtgaaacccagggccccaggaaacctgacagttcacaccaatgtctccgacactctgctgctgacctggagcaacccgtatccccctgacaattacctgtataatcatctcacctatgcagtcaacatttggagtgaaaacgacccggcagatttcagaatctataacgtgacctacctagaaccctccctccgcatcgcagccagcaccctgaagtctgggatttcctacagggcacgggtgagggcctgggctcagtgctataacaccacctggagtgagtggagccccagcaccaagtggcacaactcctacagggagcccttcgagcagcacctcctgctgggcgtcagcgtttcctgcattgtcatcctggccgtctgcctgttgtgctatgtcagcatcaccaagattaagaaagaatggtgggatcagattcccaacccagcccgcagccgcctcgtggctataataatccaggatgctcaggggtcacagtgggagaagcggtcccgaggccaggaaccagccaagtgcccacactggaagaattgtcttaccaagctcttgccctgttttctggagcacaacatgaaaagggatgaagatcctcacaaggctgccaaagagatgcctttccagggctctggaaaatcagcatggtgcccagtggagatcagcaagacagtcctctggccagagagcatcagcgtggtgcgatgtgtggagttgtttgaggccccggtggagtgtgaggaggaggaggaggtagaggaagaaaaagggagcttctgtgcatcgcctgagagcagcagggatgacttccaggagggaagggagggcattgtggcccggctaacagagagcctgttcctggacctgctcggagaggagaatgggggcttttgccagcaggacatgggggagtcatgccttcttccaccttcgggaagtacgagtgctcacatgccctgggatgagttcccaagtgcagggcccaaggaggcacctccctggggcaaggagcagcctctccacctggagccaagtcctcctgccagcccgacccagagtccagacaacctgacttgcacagagacgcccctcgtcatcgcaggcaaccctgcttaccgcagcttcagcaactccctgagccagtcaccgtgtcccagagagctgggtccagacccactgctggccagacacctggaggaagtagaacccgagatgccctgtgtcccccagctctctgagccaaccactgtgccccaacctgagccagaaacctgggagcagatcctccgccgaaatgtcctccagcatggggcagctgcagcccccgtctcggcccccaccagtggctatcaggagtttgtacatgcggtggagcagggtggcacccaggccagtgcggtggtgggcttgggtcccccaggagaggctggttacaaggccttctcaagcctgcttgccagcagtgctgtgtccccagagaaatgtgggtttggggctagcagtggggaagaggggtataagcctttccaagacctcattcctggctgccctggggaccctgccccagtccctgtccccttgttcacctttggactggacagggagccacctcgcagtccgcagagctcacatctcccaagcagctccccagagcacctgggtctggagccgggggaaaaggtagaggacatgccaaagcccccacttccccaggagcaggccacagacccccttgtggacagcctgggcagtggcattgtctactcagcccttacctgccacctgtgcggccacctgaaacagtgtcatggccaggaggatggtggccagacccctgtcatggccagtccttgctgtggctgctgctgtggagacaggtcctcgccccctacaacccccctgagggccccagacccctctccaggtggggttccactggaggccagtctgtgtccggcctccctggcaccctcgggcatctcagagaagagtaaatcctcatcatccttccatcctgcccctggcaatgctcagagctcaagccagacccccaaaatcgtgaactttgtctccgtgggacccacatacatgagggtctcttaggtgcatgtcctcttgttgctgagtctgcagatgaggactagggcttatccatgcctgggaaatgccacctcctggaaggcagccaggctggcagatttccaaaagacttgaagaaccatggtatgaaggtgattggccccactgacgttggcctaacactgggctgcagagactggaccccgcccagcattgggctgggctcgccacatcccatgagagtagagggcactgggtcgccgtgccccacggcaggcccctgcaggaaaactgaggcccttgggcacctcgacttgtgaacgagttgttggctgctccctccacagcttctgcagcagactgtccctgttgtaactgcccaaggcatgttttgcccaccagatcatggcccacgtggaggcccacctgcctctgtctcactgaactagaagccgagcctagaaactaacacagccatcaagggaatgacttgggcggccttgggaaatcgatgagaaattgaacttcagggagggtggtcattgcctagaggtgctcattcatttaacagagcttccttaggttgatgctggaggcagaatcccggctgtcaaggggtgttcagttaaggggagcaacagaggacatgaaaaattgctatgactaaagcagggacaatttgctgccaaacacccatgcccagctgtatggctgggggctcctcgtatgcatggaacccccagaataaatatgctcagccaccctgtgggccgggcaatccagacagcaggcataaggcaccagttaccctgcatgttggcccagacctcaggtgctagggaaggcgggaaccttgggttgagtaatgctcgtctgtgtgttttagtttcatcacctgttatctgtgtttgctgaggagagtggaacagaaggggtggagttttgtataaataaagtttctttgtctcttta

SEQ ID NO: 34 - DNA, 3996 bp, Homo sapiens
ccagcccggccgggctccgaggcgagaggctgcatggagtggccggcgcggctctgcgggctgtgggcgctgctgctctgcgccggcggcgggggcgggggcgggggcgccgcgcctacggaaactcagccacctgtgacaaatttgagtgtctctgttgaaaacctctgcacagtaatatggacatggaatccacccgagggagccagctcaaattgtagtctatggtattttagtcattttggcgacaaacaagataagaaaatagctccggaaactcgtcgttcaatagaagtacccctgaatgagaggatttgtctgcaagtggggtcccagtgtagcaccaatgagagtgagaagcctagcattttggttgaaaaatgcatctcacccccagaaggtgatcctgagtctgctgtgactgagcttcaatgcatttggcacaacctgagctacatgaagtgttcttggctccctggaaggaataccagtcccgacactaactatactctctactattggcacagaagcctggaaaaaattcatcaatgtgaaaacatctttagagaaggccaatactttggttgttcctttgatctgaccaaagtgaaggattccagttttgaacaacacagtgtccaaataatggtcaaggataatgcaggaaaaattaaaccatccttcaatatagtgcctttaacttcccgtgtgaaacctgatcctccacatattaaaaacctctccttccacaatgatgacctatatgtgcaatgggagaatccacagaattttattagcagatgcctattttatgaagtagaagtcaataacagccaaactgagacacataatgttttctacgtccaagaggctaaatgtgagaatccagaatttgagagaaatgtggagaatacatcttgtttcatggtccctggtgttcttcctgatactttgaacacagtcagaataagagtcaaaacaaataagttatgctatgaggatgacaaactctggagtaattggagccaagaaatgagtataggtaagaagcgcaattccacactctacataaccatgttactcattgttccagtcatcgtcgcaggtgcaatcatagtactcctgctttacctaaaaaggctcaagattattatattccctccaattcctgatcctggcaagatttttaaagaaatgtttggagaccagaatgatgatactctgcactggaagaagtacgacatctatgagaagcaaaccaaggaggaaaccgactctgtagtgctgatagaaaacctgaagaaagcctctcagtgatggagataatttatttttaccttcactgtgaccttgagaagattcttcccattctccatttgttatctgggaacttattaaatggaaactgaaactactgcaccatttaaaaacaggcagctcataagagccacaggtctttatgttgagtcgcgcaccgaaaaactaaaaataatgggcgctttggagaagagtgtggagtcattctcattgaattataaaagccagcaggcttcaaactaggggacaaagcaaaaagtgatgatagtggtggagttaatcttatcaagagttgtgacaacttcctgagggatctatacttgctttgtgttctttgtgtcaacatgaacaaattttatttgtaggggaactcatttggggtgcaaatgctaatgtcaaacttgagtcacaaagaacatgtagaaaacaaaatggataaaatctgatatgtattgtttgggatcctattgaaccatgtttgtggctattaaaactcttttaacagtctgggctgggtccggtggctcacgcctgtaatcccagcaatttgggagtccgaggcgggcggatcactcgaggtcaggagttccagaccagcctgaccaaaatggtgaaacctcctctctactaaaactacaaaaattaactgggtgtggtggcgcgtgcctgtaatcccagctactcgggaagctgaggcaggtgaattgtttgaacctgggaggtggaggttgcagtgagcagagatcacaccactgcactctagcctgggtgacagagcaagactctgtctaaaaaacaaaacaaaacaaaacaaaacaaaaaaacctcttaatattctggagtcatcattcccttcgacagcattttcctctgctttgaaagccccagaaatcagtgttggccatgatgacaactacagaaaaaccagaggcagcttctttgccaagacctttcaaagccattttaggctgttaggggcagtggaggtagaatgactccttgggtattagagtttcaaccatgaagtctctaacaatgtattttcttcacctctgctactcaagtagcatttactgtgtctttggtttgtgctaggcccccgggtgtgaagcacagaccccttccaggggtttacagtctatttgagactcctcagttcttgccacttttttttttaatctccaccagtcatttttcagaccttttaactcctcaattccaacactgatttccccttttgcattctccctccttcccttccttgtagccttttgactttcattggaaattaggatgtaaatctgctcaggagacctggaggagcagaggataattagcatctcaggttaagtgtgagtaatctgagaaacaatgactaattcttgcatattttgtaacttccatgtgagggttttcagcattgatatttgtgcattttctaaacagagatgaggtggtatcttcacgtagaacattggtattcgcttgagaaaaaaagaatagttgaacctatttctctttctttacaagatgggtccaggattcctcttttctctgccataaatgattaattaaatagcttttgtgtcttacattggtagccagccagccaaggctctgtttatgcttttggggggcatatattgggttccattctcacctatccacacaacatatccgtatatatcccctctactcttacttcccccaaatttaaagaagtatgggaaatgagaggcatttcccccaccccatttctctcctcacacacagactcatattactggtaggaacttgagaactttatttccaagttgttcaaacatttaccaatcatattaatacaatgatgctatttgcaattcctgctcctaggggaggggagataagaaaccctcactctctacaggtttgggtacaagtggcaacctgcttccatggccgtgtagaagcatggtgccctggcttctctgaggaagctggggttcatgacaatggcagatgtaaagttattcttgaagtcagattgaggctgggagacagccgtagtagatgttctactttgttctgctgttctctagaaagaatatttggttttcctgtataggaatgagattaattcctttccaggtattttataattctgggaagcaaaacccatgcctccccctagccatttttactgttatcctatttagatggccatgaagaggatgctgtgaaattcccaacaaacattgatgctgacagtcatgcagtctgggagtggggaagtgatcttttgttcccatcctcttcttttagcagtaaaatagctgagggaaaagggagggaaaaggaagttatgggaatacctgtggtggttgtgatccctaggtcttgggagctcttggaggtgtctgtatcagtggatttcccatcccctgtgggaaattagtaggctcatttactgttttaggtctagcctatgtggattttttcctaacatacctaagcaaacccagtgtcaggatggtaattcttattctttcgttcagttaagtttttcccttcatctgggcactgaagggatatgtgaaacaatgttaacatttttggtagtcttcaaccagggattgtttctgtttaacttcttataggaaagcttgagtaaaataaatattgtctttttgtatgtca

SEQ ID NO: 35 - DNA, 5821 bp, Homo sapiens
aaacaaaacagaaatgcaacgctttagagtacccacggaaaacttgtttaccttgtcaccatgagtaaaagttaattcccactcctgaagagagcaaaccaactctgaaagagagtgaaaatgcagacaagacagttatcagataatggctatctggacgagagattctttcgtttgacagcagtttggttgttgggagttccagttcagctcctgcacagttgctctgtacaaatcctcctccatatttgcttagagaaaacgtgttgccatcccatcatgaaggaagctgcctgagagtttttaaccattacagccgtgatgatgaaagagtgaagaaccgcctctaagttaaaaagtgcacccagagataaggttcgttctcaatgccctgccgctgcttctcatcgcatggccaccgcatttctcaggccaggcacattgagcattggtcctgtgcctgacgctatgctagatgctggggttgcagccacgagcatagacacgacagacacggtcctcgccatcttctgttgagtactggtcggaacaagaggatcgtctgtagacaggctacagattgttttagattgaagtttcctgtcatgttcactcatctttaaatcctcatagtgaaaaaggatatgatcatcgtggcgcatgtattactcatccttttgggggccactgagatactgcaagctgacttacttcctgatgaaaagatttcacttctcccacctgtcaatttcaccattaaagttactggtttggctcaagttcttttacaatggaaaccaaatcctgatcaagagcaaaggaatgttaatctagaatatcaagtgaaaataaacgctccaaaagaagatgactatgaaaccagaatcactgaaagcaaatgtgtaaccatcctccacaaaggcttttcagcaagtgtgcggaccatcctgcagaacgaccactcactactggccagcagctgggcttctgctgaacttcatgccccaccagggtctcctggaacctcaattgtgaatttaacttgcaccacaaacactacagaagacaattattcacgtttaaggtcataccaagtttcccttcactgcacctggcttgttggcacagatgcccctgaggacacgcagtattttctctactataggtatggctcttggactgaagaatgccaagaatacagcaaagacacactggggagaaatatcgcatgctggtttcccaggacttttatcctcagcaaagggcgtgactggcttgcggtgcttgttaacggctccagcaagcactctgctatcaggccctttgatcagctgtttgcccttcacgccattgatcaaataaatcctccactgaatgtcacagcagagattgaaggaactcgtctctctatccaatgggagaaaccagtgtctgcttttccaatccattgctttgattatgaagtaaaaatacacaatacaaggaatggatatttgcagatagaaaaattgatgaccaatgcattcatctcaataattgatgatctttctaagtacgatgttcaagtgagagcagcagtgagctccatgtgcagagaggcagggctctggagtgagtggagccaacctatttatgtgggaaatgatgaacacaagcccttgagagagtggtttgtcattgtgattatggcaaccatctgcttcatcttgttaattctctcgcttatctgtaaaatatgtcatttatggatcaagttgtttccaccaattccagcaccaaaaagtaatatcaaagatctctttgtaaccactaactatgagaaagctgggtccagtgagacggaaattgaagtcatctgttatatagagaagcctggagttgagaccctggaggattctgtgttttgactgtcactttggcatcctctgatgaactcacacatgcctcagtgcctcagtgaaaagaacagggatgctggctcttggctaagaggtgttcagaatttaggcaacactcaatttacctgcgaagcaatacacccagacacaccagtcttgtatctcttaaaagtatggatgcttcatccaaatcgcctcacctacagcagggaagttgactcatccaagcattttgccatgttttttctccccatgccgtacagggtagcacctcctcacctgccaatctttgcaatttgcttgactcacctcagacttttcattcacaacagacagcttttaaggctaacgtccagctgtatttacttctggctgtgcccgtttggctgtttaagctgccaattgtagcactcagctaccatctgaggaagaaagcattttgcatcagcctggagtgaaccatgaacttggattcaagactgtcttttctatagcaagtgagagccacaaattcctcacccccctacattctagaatgatctttttctaggtagattgtgtatgtgtgtgtatgagagagagagagagagagagagagagagaaattatctcaagctccagaggcctgatccaggatacatcatttgaaaccaactaatttaaaagcataatagagctaatatatcaccccatatcaaaagagacgacagattttgttcaaatattatattcttgaaggaagccttaatattctggaaatgtgctgaggaagtcattgagcataattcaatcatgaaagggaccactgaatcaagaatgaactttctgaagagatgtttgctgccaaagacttcagaacaacttgttggtcaagtaatgccagatcaatcctagtctctcaagtttggaaaagtttctacaaggccattccatcaaatatcccaatatacacagaaattcttcttcctaacatcttcatatatgagtctgcatccagattggaggcctttgtggtgggtggtggactgcaggtggaaccaagcacaggtgtgccctgggcagaggtgggcaagaggaaggaacccaaacagtgctgattccacatggcccttggtcaagaagctgaaggcctagaaaggaggtaacagtgcccagaatccccacctggcacactctttaaatgctttctggaggtgttttagtcagttttgatccatcggtggggacacactgactaccatatatttccaggtttgtttgttttcttaactaacctgtaattatccactgtaactagcagagggttgaaccctgggtaataggcacagaaacatcttgaatcttgacaagcctgcactgtcttatagtaatctgtattactgtcccattttatagttgcagttactaaggttcagagaagttgataggaaatggcatagtgaggagttgaataccaggctcaggtaaaagtgaccccagaaatgtgcagaggagtgtcagggaaagctgaggcctgcttaggggtgtggctgagaaggcaacacccatggaggtgctgcaatagttggcaaaatgtttaaaatgtttggtcactcaaatgattcataatctcaaacctgtttttgtttttttttttttttttttttgtggggtgggtggtggagacagtcttgttctgtctcccaggctagagtgtagtgatgtgatcttagctcactacaacctccctgtcctgggctcaagccatcctcctccctcaccctcccaagtagctgggactacaggtgcataacaccattcccagctcttttttgtatttttttttgtaaagctggggttttgccatgctgtctaggttcatctcgaactcctgggctcaagcgatacaccggcctcagccttccaaagtactgggattacaggcatgaaccaccattcctggccaggaaactgtatttgtaatgaacaccttaggtcagaatcagaggcgcctggagggctccttaagacacagaggagtgggctccattcttagagtctctaactgaataggtctgggtgaggtctgaaaagctgcgtttctaacaagttctcaggtgatgctcatgctgctggtctccaggacacactcggagaactgctgctccaggagagtcttatgaataccaaaatttgaaaatcagccgctgaaggaagatgtgacagaaaagtcctgtccccagaaaactgtccttttggggcggcttggagcaccctgtctgatctctttcaaaccacaacccagacagttggcattgtggaaactgcctctgcttaggaggcaggaacagaagcagggagctccccctccacaaatgtggccagctcttcagtgcttggtgtggtgcccacttcagactcctccctccactcagcagccatagatcacctgggctctgtggagctccttgccacacttccctgagctggtaccgttatctggcccttggtatatgtgtttcaggagcaacacaaagtagcccattggcctccttggtggattgggaactccttcctcccttcttgccctgtgcaactcatagtcattctttgggaacccgtaaccatcatcacttcctccatgaagccttcaaaagcagagttggttgatcccgcctctggattctcattctgggcctcatacccgtgttagagctactgcattgtagaatataccattaacagctgggaggcctcccctctagactgcggcttccaggaggatggagcttgtgtgaaatacgtctttttatctgcagtgtctaggacagtatctgacttaagaaaatataaatgtttcttaaaccctctgtcagccctgatacggcaccttgtatataggttagactgaacagtatttggcagagatcctcaaggcctacaataatgacagggggaggaagctgggtatctaatgagaatgcggttagttagaagcaaccaggagacacatgtgcctaacaactgggctactgcaacctggatgtttttgctgccaaagtgtctaaatggttatgtataaattgctgtgtaagaggcatttcagaagtggctcactgtgagtggtctatactgttctcaccagccttgtcatagcaagtcaaagagtttgaaattcacttaacagtaaacaccatcagcagcaggttgatgtaaggtaaagtcatgatattggctcccatctcttgccatctctcactaggacgagtgtagagagtcacttttctcctagagcctccacagccacgggatcagaacaaacatgcttttccaagatatccgttccaatagggatttttagcccaatttctttccttggcatttggaattcactgcgtgcagcatgggggagtgctggggagactggagggtgaaggtcacatttgccaacagcccgaccacctgagcctcattgaaatgcctcagacctcaagctattcatgcaggctggggaagaatccacgtgggcaaagaacatcctgattaaaaatccaaacgttttcatggtgagacttctatgtgtcaggaccatctgctggtggaattaagaagcctgaggtgctaatttgttaaagaaaaagtagatatctggaagtgagaacagagaactgaaaacaatctctgatggcacaagtaaaggtgaaaaacctaattagaataggacttggtgctagcctaaggactgtagctattcctgcttctacatccttcaaattgagtaccagggtgcaaattttttcaaaaccattccttcctttggaattttgtattgtactataagacaatcatgcttatctcctgttttgtaggtaatagatgtgaatataaatatgtttcagaatgagaaatattaaacataaatgacagtgaaaaaaaaaaaaaaa

SEQ ID NO: 36 - DNA, 2565 bp, Homo sapiens
gctcgtgtccctgtcatattttccactctccacgaggtcctgcgcgcttcaatcctgcaggcagcccggtttggggatgtggtccttgctgctctgcgggttgtccatcgcccttccactgtctgtcacagcagatggatgcaaggacatttttatgaaaaatgagatactttcagcaagccagccttttgcttttaattgtacattccctcccataacatctggggaagtcagtgtaacatggtataaaaattctagcaaaatcccagtgtccaaaatcatacagtctagaattcaccaggacgagacttggattttgtttctccccatggaatggggggactcaggagtctaccaatgtgttataaagggtagagacagctgtcatagaatacatgtaaacctaactgtttttgaaaaacattggtgtgacacttccataggtggtttaccaaatttatcagatgagtacaagcaaatattacatcttggaaaagatgatagtctcacatgtcatctgcacttcccgaagagttgtgttttgggtccaataaagtggtataaggactgtaacgagattaaaggggagcggttcactgttttggaaaccaggcttttggtgagcaatgtctcggcagaggacagagggaactacgcgtgtcaagccatactgacacactcagggaagcagtacgaggttttaaatggcatcactgtgagcattacagaaagagctggatatggaggaagtgtccctaaaatcatttatccaaaaaatcattcaattgaagtacagcttggtaccactctgattgtggactgcaatgtaacagacaccaaggataatacaaatctacgatgctggagagtcaataacactttggtggatgattactatgatgaatccaaacgaatcagagaaggggtggaaacccatgtctcttttcgggaacataatttgtacacagtaaacatcaccttcttggaagtgaaaatggaagattatggccttcctttcatgtgccacgctggagtgtccacagcatacattatattacagctcccagctccggattttcgagcttacttgataggagggcttatcgccttggtggctgtggctgtgtctgttgtgtacatatacaacatttttaagatcgacattgttctttggtatcgaagtgccttccattctacagagaccatagtagatgggaagctgtatgacgcctatgtcttataccccaagccccacaaggaaagccagaggcatgccgtggatgccctggtgttgaatatcctgcccgaggtgttggagagacaatgtggatataagttgtttatattcggcagagatgaattccctggacaagccgtggccaatgtcatcgatgaaaacgttaagctgtgcaggaggctgattgtcattgtggtccccgaatcgctgggctttggcctgttgaagaacctgtcagaagaacaaatcgcggtctacagtgccctgatccaggacgggatgaaggttattctcattgagctggagaaaatcgaggactacacagtcatgccagagtcaattcagtacatcaaacagaagcatggtgccatccggtggcatggggacttcacggagcagtcacagtgtatgaagaccaagttttggaagacagtgagataccacatgccgcccagaaggtgtcggccgtttcctccggtccagctgctgcagcacacaccttgctaccgcaccgcaggcccagaactaggctcaagaagaaagaagtgtactctcacgactggctaagacttgctggactgacacctatggctggaagatgacttgttttgctccatgtctcctcattcctacacctattttctgctgcaggatgaggctagggttagcattctagacacccagttgagctcaggcgtagagaagaggaggatgggataagaactggggccatccccatgtcatggtgggtgagagctggggccatccccgtggtcatggagggtgagagctgggggttatccccatggtcatggagggtgagggctggtcgggggaggcatccccaagtcatggtgggtgagagctcggagcatccccatgtcatggtgggtgagatctgggggtatccctgtgtcatggtgggtgagggcgggtggtcatccacatggtcatagtgggtgagagctgggggtatccctacatcatggtgggtgagagctgggagcatccccatgtcatggtgggcgagatctggggggtatccccacgtcatggtggatgagagctgggggaatcaccatgtcatggtgggtgagatcttgggggatcacctgtcatggtgggtgagagctggggggatcacctgtcatggtgggtgagagttgggattcatccccatgtcatggtgggctgagcccacatggaagcctgtgcttggacagcgtatgcccttttctctgtttttccacaatgaacaattaaactgtaaatgttaaaaatatcagtaatttgtgaaataaattttattctcatttgagcaacataaa

SEQ ID NO: 37 - DNA, 8608 bp, Homo sapiens
ctgggcccgggctggaagccggaagcgagcaaagtggagccgactcgaactccaccgcggaaaagaaagcctcagaacgttcgttcgctgcgtccccagccggggccgagccctccgcgacgccagccgggccatgggggccgcacgcagcccgccgtccgctgtcccggggcccctgctggggctgctcctgctgctcctgggcgtgctggccccgggtggcgcctccctgcgactcctggaccaccgggcgctggtctgctcccagccggggctaaactgcacggtcaagaatagtacctgcctggatgacagctggattcaccctcgaaacctgaccccctcctccccaaaggacctgcagatccagctgcactttgcccacacccaacaaggagacctgttccccgtggctcacatcgaatggacactgcagacagacgccagcatcctgtacctcgagggtgcagagttatctgtcctgcagctgaacaccaatgaacgtttgtgcgtcaggtttgagtttctgtccaaactgaggcatcaccacaggcggtggcgttttaccttcagccactttgtggttgaccctgaccaggaatatgaggtgaccgttcaccacctgcccaagcccatccctgatggggacccaaaccaccagtccaagaatttccttgtgcctgactgtgagcacgccaggatgaaggtaaccacgccatgcatgagctcaggcagcctgtgggaccccaacatcaccgtggagaccctggaggcccaccagctgcgtgtgagcttcaccctgtggaacgaatctacccattaccagatcctgctgaccagttttccgcacatggagaaccacagttgctttgagcacatgcaccacatacctgcgcccagaccagaagagttccaccagcgatccaacgtcacactcactctacgcaaccttaaagggtgctgtcgccaccaagtgcagatccagcccttcttcagcagctgcctcaatgactgcctcagacactccgcgactgtttcctgcccagaaatgccagacactccagaaccaattccggactacatgcccctgtgggtgtactggttcatcacgggcatctccatcctgctggtgggctccgtcatcctgctcatcgtctgcatgacctggaggctagctgggcctggaagtgaaaaatacagtgatgacaccaaatacaccgatggcctgcctgcggctgacctgatccccccaccgctgaagcccaggaaggtctggatcatctactcagccgaccaccccctctacgtggacgtggtcctgaaattcgcccagttcctgctcaccgcctgcggcacggaagtggccctggacctgctggaagagcaggccatctcggaggcaggagtcatgacctgggtgggccgtcagaagcaggagatggtggagagcaactctaagatcatcgtcctgtgctcccgcggcacgcgcgccaagtggcaggcgctcctgggccggggggcgcctgtgcggctgcgctgcgaccacggaaagcccgtgggggacctgttcactgcagccatgaacatgatcctcccggacttcaagaggccagcctgcttcggcacctacgtagtctgctacttcagcgaggtcagctgtgacggcgacgtccccgacctgttcggcgcggcgccgcggtacccgctcatggacaggttcgaggaggtgtacttccgcatccaggacctggagatgttccagccgggccgcatgcaccgcgtaggggagctgtcgggggacaactacctgcggagcccgggcggcaggcagctccgcgccgccctggacaggttccgggactggcaggtccgctgtcccgactggttcgaatgtgagaacctctactcagcagatgaccaggatgccccgtccctggacgaagaggtgtttgaggagccactgctgcctccgggaaccggcatcgtgaagcgggcgcccctggtgcgcgagcctggctcccaggcctgcctggccatagacccgctggtcggggaggaaggaggagcagcagtggcaaagctggaacctcacctgcagccccggggtcagccagcgccgcagcccctccacaccctggtgctcgccgcagaggagggggccctggtggccgcggtggagcctgggcccctggctgacggtgccgcagtccggctggcactggcgggggagggcgaggcctgcccgctgctgggcagcccgggcgctgggcgaaatagcgtcctcttcctccccgtggaccccgaggactcgccccttggcagcagcacccccatggcgtctcctgacctccttccagaggacgtgagggagcacctcgaaggcttgatgctctcgctcttcgagcagagtctgagctgccaggcccaggggggctgcagtagacccgccatggtcctcacagacccacacacgccctacgaggaggagcagcggcagtcagtgcagtctgaccagggctacatctccaggagctccccgcagccccccgagggactcacggaaatggaggaagaggaggaagaggagcaggacccagggaagccggccctgccactctctcccgaggacctggagagcctgaggagcctccagcggcagctgcttttccgccagctgcagaagaactcgggctgggacacgatggggtcagagtcagaggggcccagtgcatgagggcggctccccagggaccgcccagatcccagctttgagagaggagtgtgtgtgcacgtattcatctgtgtgtacatgtctgcatgtgtatatgttcgtgtgtgaaatgtaggctttaaaatgtaaatgtctggattttaatcccaggcatccctcctaacttttctttgtgcagcggtctggttatcgtctatccccaggggaatccacacagcccgctcccaggagctaatggtagagcgtccttgaggctccattattcgttcattcagcatttattgtgcacctactatgtggcgggcatttgggataccaagataaattgcatgcggcatggccccagccatgaaggaacttaaccgctagtgccgaggacacgttaaacgaacaggatgggccgggcacggtggctcacgcctgtaatcccagcacactgggaggccgaggcaggtggatcactctgaggtcaggagtttgagccagcctggccaacatggtgaaaccccatctccactaaaaatagaaaaattagccgggcatggtgacacatgcctgtagtcctagctacttgggaggctgaggcaggagaattgcttgaatctgggaggcagaggttgcagtgagccgagattgtgccattgcactgcagcctggatgacagagcgagactctatctcaaaaaaaaaaaaaaaaaaagatggtcacgcgggatgtaaacgctgaatgggccaggtgcagtggctcatgcttgtaatcccagcactttgggaaggcgaggcaggtggattgcttgagctcaggagttcaagaccagcctgggcgacatagtgagacctcatctctacctaaatttttttttagtcagtcatggtggcacatgcctgtagtcccagctactcgggaggctgatgccagatgatcacttgagcccaggaggtagaggctgcagtgagctataatggtaccattgcaatccagcctgggcagcagagtgagaccctgtctcaaaaaaaataaaaaagtagaaagatggagtggaagcctgcccagggttgtgagcatgcacgggaaaggcacccaggtcaggggggatccccgaggagatgcctgagctgaaggattgtggttggggaaagcgtagtcccagcaaggaagcagtttgtgggtaagtgctgggaggtgagtggagtgagcttgtcagggagctgctggtggagcctggaggggaaggagggaggcagtgagagagatcggggtggggggtggggggatgtcgccagagctcaggggtggggacagccttgtgcgcatcagtcctgaggcctggggcacctttcgtctgatgagcctctgcatggagagaggctgagggctaaacacagctggatgtcacctgagttcatttataggaagagagaaatgtcgaggtgaaacgtaaaagcatctggcaggaaggtgagtctgaagccctgcacccgcgttccgactatcagtggggagctgttagcacgtaggattcttcagagcagctgggctggagctcccctgagctcaggaagccccagggtgcaagggcaaggaaatgaggggtggtgggtcagtgaagatctgggcagaccttgtgtggggaaggggtgctgctgtgacttcagggtctgaggtccaaagacagcatttgaaaagaggctctgaagccagtgtttgaagaatttgttcctgaagtacctcctgggggtaggctagaggcttctggcttcagggtcctgaagaacacattgaggtgccgtctgacactggaatagggtgcccttcattcctatgcctgagtccttaactatatttccaacctccagtgaggaggagaagattcggaaatgtgacaggagagcaaacaggacagtttgcatgtgtgtgtgcgcacacatacatgtgcgtgaaagattatcaataaaagtgcataaatttgttgatctggtaagagtttctagcaggaaggtcgagccacttactgtaggtcaagaagttgctagttgcggagttttttcttgcagttagactttacctagtggtagcagggccaccaaagctctgtgtcccagatggtgtatggcccataatccacccaacagcagcaaaggaccaggcaaaggagaacaggagcagaagcctcccagccactagccttttgggctcagtctctccaataatcctggagaggggcttcgttgggtctggacacctaccatgcattctgtgacctttccctagcttccaataaataactgtttgacgcccagagtacaggataccacaatgcactcttcctgcgtagagcacatgttcccatctgctcccattcctcaggaaccttgaattctagctctgctggcctttgagcccatgccagtaaatgtcctgatgggcattgcctactatctccagggcagctgcctttgtcctcctaacagctttattggagtacagttcacttaccatacaatccacaattgaccctgcacaatttgatgccggtttagtatagtcacagttcagcagccatcagcacagtcagtcttagagtttactacccccaaaagaaatccagccccccttagtcaccaccccaacctccccatccctaggcacccctaggctactttgatctctgtagacttgcctcttctggacatgacatagagaaaggagtcataaattctccaaggtgtctgtttcttctttaatgtcattccctgtttctcctcacattccctccccatttcctgggcccagtctcacactggtccttgcttaccctaaatgctattaattccatcactctgagtatggtgtttgctgtccgctgaatgccaagagcttcaagagtgtgtgtaaataaagccacacctttatttttgtattattctgaaccatggctaataaattgtttcaccaagaaatgtctctctaagaacaggtgccctccacgctgtgcccctcccacctcttcagctcgtctcctgagtgtgcagaggtggttccggttgggaaagaagcagcggagcatctaaccatgcctgtgtccaggccgattatgcacgcagccaccaacaagctcccaactcccgcgtagagtttcatgactttttcctgcctactatcttgatcctagttttttttttgttttttttttttttaaggaataattactttgattcaaaaccagtttctcttttctgcataggaaggtccttgaaggtgtttagggtctaaaaagggtggtgttcggtctctgaaacatccattcagcagtttgagctgggatctctgaatgcaagggtatgatggatatacttctttcttgcttttgttgtgttttggttttttgtttgtttttaagtcagggtctctctgtcaccaggctgtattacagtggtgcaatcatggctcactgcagcctcgacctcccaggctcaagccatctttccacctcagcctgccagtggctagaactacaggcgtgcaccactgtgcccggctaatttgtgtgtatatattttgtagaaatggggtttcaccatgttgtccaggctggtcacgaactcctgggctcaagccatctgcccgcctcatcctcccaaagtgctgggattataggcctgagcccaccgtgcctggcctttcctgtttatctttgaaaattaaatagggcataagagagaagaagatgtacttacaatgcagtgggtggttttaactctatagcctttgggctctgtggttggtgctccccttcctaaataaatgaggtgtatgcagggccctcttctgccttagcgccctgccagctgggactccagcaaggcccggggcacctgaggacagagtgagatggagggccgctgctccagcagccgggcctgcatcccacaagtcaactgtgtcggacagaggatccttacaaagaagaggcagcagggttgggggctggccagctgctcgtccgccctaggtagcttgctcatctgtaaagtgggtggggcaggagttcccacctcatggggtcctggcaagcctgcagtatccccgagtggcaccagcctgcttctggggcagagcagtttgtgccccctgaggtaccactgatcctctttccctgctattaggtattgctctcttcctccggtgtttgccttttcagattatagaagtaatatgtgttcccatatttggcgtctctcaggagctcaggaagtacttggctgagtgaacatgtccattgtggaaaaatggcaacaatatggattccatgggtatattttatagaagaatatgaagaaaagcagctacccctaaacccattgcacaagctgttcatgttaattctgtacccgacgctttccccacggggcctcccctcactctgaaatggcatccaggtccatcttgccctccacctctgcatggctctccatgccccatcgcctctcccagatcctagcactgggtccacactctcgccctgtccatttaggttgatgaaagcaggcagtcacccgggtgggccagtcttgcctgtgggaggaacatgcagtctcctgtctcatggtttgaagtgtgccaggaagcctggcccagcccacctccccctggagtccttcccaggaggaataaccccttaggtcattgactataagatgagttcgctcactggatccttcctctctgatgagacaggaagaaggtacacagtgaccaggtaggaggaggagagggagtagaaaggagggatgcgggtggctggtccctgcatttgcctgcttccctgcacgggtgtcccactggccgcctctgctcaccagtgtcatgggattctctcagaagatgaaaacagcccctgcttttttgctagaatggctgagctttcatggaaaggaagctggacccaagcaacagccgactaccgaaggttgcctggagcagtgcagatgtgggaggaagaagggccttggtgcacactggcttttcttcctgactgcaatgtggcattgtgccagctacctcctctttctcggcctcaggaaaatggagagaaagcagccctgaaggtggctgtgacgagggaaggggcagagggcctgacagtcaaccacgcgctatattttcctgttcttccttagggcaagaactgcatggccagactcaggcaaggcctaggtgtgggctgggcattgcctacacgtgaagagatcactccgcgtccctactgcacctgtcacaaagtgccttctgatatgcctggcaaaccaaaatcggtgagcgccagcttgcttccctagaagacatttctaaatattcataacatgcttgctcaaatcaatcaccttattttacatccgctccagggagaaatgaagacatggtcctacgttgttctgtaattattttctatgtaaattttgttccttgttacaattatatatgtcttaggggaaaggaccatttcacatgtgtcacctcatgtgattctcaccacagccctgtgattgctcctgttttataaataatgacatagttccagttgatggccaaagccacagctaacgagaggcagagagagctcaggctcccaggagcttccactctcagaccttgcctcccgggctgccctgagtgaaacgcctgcttagcatttggcacagccagaagcagcaagctagggtcacaacacagagaggggctgtgtaatactggctgcctctgtgctaagaaaaaaaaaaaatcactgtgtgtttgtttattttggtgcaggcccagtgttcttgcttagacttaatactacccttcatgttaaaataaaaccaaacaaaaacccat

SEQ ID NO: 38 - DNA, 5183 bp, Homo sapiens
agttcccggccgcgagggcgggcgcagcttgtggccggcggccggagccgactcggagcgcgcggcgccggccgggaggagccggagagcggccgggccgggcggtgggggcgccggcctgccccgcgcgccccagggagcggcaggaatgtgacaatcgcgcgcccgcgcaccgaagcactcctcgctcggctcctagggctctcgcccctctgagctgagccgggttccgcccggggctgggatcccatcaccctccacggccgtccgtccaggtagacgcaccctctgaagatggtgactccctcctgagaagctggaccccttggtaaaagacaaggccttctccaagaagaatatgaaagtgttactcagacttatttgtttcatagctctactgatttcttctctggaggctgataaatgcaaggaacgtgaagaaaaaataattttagtgtcatctgcaaatgaaattgatgttcgtccctgtcctcttaacccaaatgaacacaaaggcactataacttggtataaagatgacagcaagacacctgtatctacagaacaagcctccaggattcatcaacacaaagagaaactttggtttgttcctgctaaggtggaggattcaggacattactattgcgtggtaagaaattcatcttactgcctcagaattaaaataagtgcaaaatttgtggagaatgagcctaacttatgttataatgcacaagccatatttaagcagaaactacccgttgcaggagacggaggacttgtgtgcccttatatggagttttttaaaaatgaaaataatgagttacctaaattacagtggtataaggattgcaaacctctacttcttgacaatatacactttagtggagtcaaagataggctcatcgtgatgaatgtggctgaaaagcatagagggaactatacttgtcatgcatcctacacatacttgggcaagcaatatcctattacccgggtaatagaatttattactctagaggaaaacaaacccacaaggcctgtgattgtgagcccagctaatgagacaatggaagtagacttgggatcccagatacaattgatctgtaatgtcaccggccagttgagtgacattgcttactggaagtggaatgggtcagtaattgatgaagatgacccagtgctaggggaagactattacagtgtggaaaatcctgcaaacaaaagaaggagtaccctcatcacagtgcttaatatatcggaaattgaaagtagattttataaacatccatttacctgttttgccaagaatacacatggtatagatgcagcatatatccagttaatatatccagtcactaatttccagaagcacatgattggtatatgtgtcacgttgacagtcataattgtgtgttctgttttcatctataaaatcttcaagattgacattgtgctttggtacagggattcctgctatgattttctcccaataaaagcttcagatggaaagacctatgacgcatatatactgtatccaaagactgttggggaagggtctacctctgactgtgatatttttgtgtttaaagtcttgcctgaggtcttggaaaaacagtgtggatataagctgttcatttatggaagggatgactacgttggggaagacattgttgaggtcattaatgaaaacgtaaagaaaagcagaagactgattatcattttagtcagagaaacatcaggcttcagctggctgggtggttcatctgaagagcaaatagccatgtataatgctcttgttcaggatggaattaaagttgtcctgcttgagctggagaaaatccaagactatgagaaaatgccagaatcgattaaattcattaagcagaaacatggggctatccgctggtcaggggactttacacagggaccacagtctgcaaagacaaggttctggaagaatgtcaggtaccacatgccagtccagcgacggtcaccttcatctaaacaccagttactgtcaccagccactaaggagaaactgcaaagagaggctcacgtgcctctcgggtagcatggagaagttgccaagagttctttaggtgcctcctgtcttatggcgttgcaggccaggttatgcctcatgctgacttgcagagttcatggaatgtaactatatcatcctttatccctgaggtcacctggaatcagattattaagggaataagccatgacgtcaatagcagcccagggcacttcagagtagagggcttgggaagatcttttaaaaaggcagtaggcccggtgtggtggctcacgcctataatcccagcactttgggaggctgaagtgggtggatcaccagaggtcaggagttcgagaccagcccagccaacatggcaaaaccccatctctactaaaaatacaaaaatgagctaggcatggtggcacacgcctgtaatcccagctacacctgaggctgaggcaggagaattgcttgaaccggggagacggaggttgcagtgagccgagtttgggccactgcactctagcctggcaacagagcaagactccgtctcaaaaaaagggcaataaatgccctctctgaatgtttgaactgccaagaaaaggcatggagacagcgaactagaagaaagggcaagaaggaaatagccaccgtctacagatggcttagttaagtcatccacagcccaagggcggggctatgccttgtctggggaccctgtagagtcactgaccctggagcggctctcctgagaggtgctgcaggcaaagtgagactgacacctcactgaggaagggagacatattcttggagaactttccatctgcttgtattttccatacacatccccagccagaagttagtgtccgaagaccgaattttattttacagagcttgaaaactcacttcaatgaacaaagggattctccaggattccaaagttttgaagtcatcttagctttccacaggagggagagaacttaaaaaagcaacagtagcagggaattgatccacttcttaatgctttcctccctggcatgaccatcctgtcctttgttattatcctgcattttacgtctttggaggaacagctccctagtggcttcctccgtctgcaatgtcccttgcacagcccacacatgaaccatccttcccatgatgccgctcttctgtcatcccgctcctgctgaaacacctcccaggggctccacctgttcaggagctgaagcccatgctttcccaccagcatgtcactcccagaccacctccctgccctgtcctccagcttcccctcgctgtcctgctgtgtgaattcccaggttggcctggtggccatgtcgcctgcccccagcactcctctgtctctgctcttgcctgcacccttcctcctcctttgcctaggaggccttctcgcattttctctagctgatcagaattttaccaaaattcagaacatcctccaattccacagtctctgggagactttccctaagaggcgacttcctctccagccttctctctctggtcaggcccactgcagagatggtggtgagcacatctgggaggctggtctccctccagctggaattgctgctctctgagggagaggctgtggtggctgtctctgtccctcactgccttccaggagcaatttgcacatgtaacatagatttatgtaatgctttatgtttaaaaacattccccaattatcttatttaatttttgcaattattctaattttatatatagagaaagtgacctattttttaaaaaaatcacactctaagttctattgaacctaggacttgagcctccatttctggcttctagtctggtgttctgagtacttgatttcaggtcaataacggtcccccctcactccacactggcacgtttgtgagaagaaatgacattttgctaggaagtgaccgagtctaggaatgcttttattcaagacaccaaattccaaacttctaaatgttggaattttcaaaaattgtgtttagattttatgaaaaactcttctactttcatctattctttccctagaggcaaacatttcttaaaatgtttcattttcattaaaaatgaaagccaaatttatatgccaccgattgcaggacacaagcacagttttaagagttgtatgaacatggagaggacttttggtttttatatttctcgtatttaatatgggtgaacaccaacttttatttggaataataattttcctcctaaacaaaaacacattgagtttaagtctctgactcttgcctttccacctgctttctcctgggcccgctttgcctgcttgaaggaacagtgctgttctggagctgctgttccaacagacagggcctagctttcatttgacacacagactacagccagaagcccatggagcagggatgtcacgtcttgaaaagcctattagatgttttacaaatttaattttgcagattattttagtctgtcatccagaaaatgtgtcagcatgcatagtgctaagaaagcaagccaatttggaaacttaggttagtgacaaaattggccagagagtgggggtgatgatgaccaagaattacaagtagaatggcagctggaatttaaggagggacaagaatcaatggataagcgtgggtggaggaagatccaaacagaaaagtgcaaagttattccccatcttccaagggttgaattctggaggaagaagacacattcctagttccccgtgaacttcctttgacttattgtccccactaaaacaaaacaaaaaacttttaatgccttccacattaattagattttcttgcagtttttttatggcatttttttaaagatgccctaagtgttgaagaagagtttgcaaatgcaacaaaatatttaattaccggttgttaaaactggtttagcacaatttatattttccctctcttgcctttcttatttgcaataaaaggtattgagccattttttaaatgacatttttgataaattatgtttgtactagttgatgaaggagttttttttaacctgtttatataattttgcagcagaagccaaattttttgtatattaaagcaccaaattcatgtacagcatgcatcacggatcaatagactgtacttattttccaataaaattttcaaactttgtactgtta

SEQ ID NO: 39 - DNA, 5198 bp, Homo sapiens
attctttggaatactactgctagaagtctgacttaagacccagcttatgggccacatggcacccagctgcttctgcagagaaggcaggccactgatgggtacagcaaagtgtggtgctgctggccaagccaaagacccgtgtaggatgactgggcctctgccccttgtgggtgttgccactgtgcttgagtgcctggtgaagaatgtgatgggatcactagcatgtctgcggagagcggccctgggacgagattgagaaatctgccagtaatgggggatggactagaaacttcccaaatgtctacaacacaggcccaggcccaaccccagccagccaacgcagccagcaccaaccccccgcccccagagacctccaaccctaacaagcccaagaggcagaccaaccaactgcaatacctgctcagagtggtgctcaagacactatggaaacaccagtttgcatggcctttccagcagcctgtggatgccgtcaagctgaacctccctgattactataagatcattaaaacgcctatggatatgggaacaataaagaagcgcttggaaaacaactattactggaatgctcaggaatgtatccaggacttcaacactatgtttacaaattgttacatctacaacaagcctggagatgacatagtcttaatggcagaagctctggaaaagctcttcttgcaaaaaataaatgagctacccacagaagaaaccgagatcatgatagtccaggcaaaaggaagaggacgtgggaggaaagaaacagggacagcaaaacctggcgtttccacggtaccaaacacaactcaagcatcgactcctccgcagacccagacccctcagccgaatcctcctcctgtgcaggccacgcctcaccccttccctgccgtcaccccggacctcatcgtccagacccctgtcatgacagtggtgcctccccagccactgcagacgcccccgccagtgcccccccagccacaacccccacccgctccagctccccagcccgtacagagccacccacccatcatcgcggccaccccacagcctgtgaagacaaagaagggagtgaagaggaaagcagacaccaccacccccaccaccattgaccccattcacgagccaccctcgctgcccccggagcccaagaccaccaagctgggccagcggcgggagagcagccggcctgtgaaacctccaaagaaggacgtgcccgactctcagcagcacccagcaccagagaagagcagcaaggtctcggagcagctcaagtgctgcagcggcatcctcaaggagatgtttgccaagaagcacgccgcctacgcctggcccttctacaagcctgtggacgtggaggcactgggcctacacgactactgtgacatcatcaagcaccccatggacatgagcacaatcaagtctaaactggaggcccgtgagtaccgtgatgctcaggagtttggtgctgacgtccgattgatgttctccaactgctataagtacaaccctcctgaccatgaggtggtggccatggcccgcaagctccaggatgtgttcgaaatgcgctttgccaagatgccggacgagcctgaggagccagtggtggccgtgtcctccccggcagtgccccctcccaccaaggttgtggccccgccctcatccagcgacagcagcagcgatagctcctcggacagtgacagttcgactgatgactctgaggaggagcgagcccagcggctggctgagctccaggagcagctcaaagccgtgcacgagcagcttgcagccctctctcagccccagcagaacaaaccaaagaaaaaggagaaagacaagaaggaaaagaaaaaagaaaagcacaaaaggaaagaggaagtggaagagaataaaaaaagcaaagccaaggaacctcctcctaaaaagacgaagaaaaataatagcagcaacagcaatgtgagcaagaaggagccagcgcccatgaagagcaagccccctcccacgtatgagtcggaggaagaggacaagtgcaagcctatgtcctatgaggagaagcggcagctcagcttggacatcaacaagctccccggcgagaagctgggccgcgtggtgcacatcatccagtcacgggagccctccctgaagaattccaaccccgacgagattgaaatcgactttgagaccctgaagccgtccacactgcgtgagctggagcgctatgtcacctcctgtttgcggaagaaaaggaaacctcaagctgagaaagttgatgtgattgccggctcctccaagatgaagggcttctcgtcctcagagtcggagagctccagtgagtccagctcctctgacagcgaagactccgaaacagagatggctccgaagtcaaaaaagaaggggcaccccgggagggagcagaagaagcaccatcatcaccaccatcagcagatgcagcaggccccggctcctgtgccccagcagccgcccccgcctccccagcagcccccaccgcctccacctccgcagcagcaacagcagccgccacccccgcctcccccaccctccatgccgcagcaggcagccccggcgatgaagtcctcgcccccacccttcattgccacccaggtgcccgtcctggagccccagctcccaggcagcgtctttgaccccatcggccacttcacccagcccatcctgcacctgccgcagcctgagctgccccctcacctgccccagccgcctgagcacagcactccaccccatctcaaccagcacgcagtggtctctcctccagctttgcacaacgcactaccccagcagccatcacggcccagcaaccgagccgctgccctgcctcccaagcccgcccggcccccagccgtgtcaccagccttgacccaaacacccctgctcccacagccccccatggcccaacccccccaagtgctgctggaggatgaagagccacctgccccacccctcacctccatgcagatgcagctgtacctgcagcagctgcagaaggtgcagccccctacgccgctactcccttccgtgaaggtgcagtcccagcccccaccccccctgccgcccccaccccacccctctgtgcagcagcagctgcagcagcagccgccaccacccccaccaccccagccccagcctccaccccagcagcagcatcagccccctccacggcccgtgcacttgcagcccatgcagttttccacccacatccaacagcccccgccaccccagggccagcagcccccccatccgcccccaggccagcagccacccccgccgcagcctgccaagcctcagcaagtcatccagcaccaccattcaccccggcaccacaagtcggacccctactcaaccggtcacctccgcgaagccccctccccgcttatgatacattccccccagatgtcacagttccagagcctgacccaccagtctccaccccagcaaaacgtccagcctaagaaacaggagctgcgtgctgcctccgtggtccagccccagcccctcgtggtggtgaaggaggagaagatccactcacccatcatccgcagcgagcccttcagcccctcgctgcggccggagccccccaagcacccggagagcatcaaggcccccgtccacctgccccagcggccggaaatgaagcctgtggatgtcgggaggcctgtgatccggcccccagagcagaacgcaccgccaccaggggcccctgacaaggacaaacagaaacaggagccgaagactccagttgcgcccaaaaaggacctgaaaatcaagaacatgggctcctgggccagcctagtgcagaagcatccgaccaccccctcctccacagccaagtcatccagcgacagcttcgagcagttccgccgcgccgctcgggagaaagaggagcgtgagaaggccctgaaggctcaggccgagcacgctgagaaggagaaggagcggctgcggcaggagcgcatgaggagccgagaggacgaggatgcgctggagcaggcccggcgggcccatgaggaggcacgtcggcgccaggagcagcagcagcagcagcgccaggagcaacagcagcagcagcaacagcaagcagctgcggtggctgccgccgccaccccacaggcccagagctcccagccccagtccatgctggaccagcagagggagttggcccggaagcgggagcaggagcgaagacgccgggaagccatggcagctaccattgacatgaatttccagagtgatctattgtcaatatttgaagaaaatcttttctgagcgcacctaggtggcttctgactttgattttctggcaaaacattgactttccatagtgttaggggcggtggtggaggtgggatcagcggccaggggatgcctcagggcctggccctcctgcatgctatgcccggggcaggcctgacgggcagctgaggattgcagagcctgtctgccttacggccagtcggacagacgtcccgccacccaccacccctcacaggacgtccgctcagcacacgccttgttacgagcaagtgccggctggacccaagccctgcatccccacatgcggggcagaggcccttctctccgccaaatgtctacacagtatacacaggacatcgttgctgccgccgtgactggttttctgtccccaagaacgtgacgttcgtgatgtcctgcccgccgggagtctttccccacaccccagccatcgccgcccgctcccaggaggccagggcaggcctgcgtgggctggaggcgggcgaggccggcccaccccctcgctggcactgactttgccttgaacagaccccccgaccctcccccacaagcctttaattgagagccgctctctgtaagtgtttgcttgtgcaaaagggaatagtgccgtggaggtgtgtgtgtccatggcatccggagcgaggcgactgtcctgcgtgggtagccctcggccggggagtgaggccaccaaccaaagtcagttccttcccacctgtgtttctgtttcgtttttttttttcttttttttctatatatattttttgttgaattctattttatttttaattctctcttctcctccagacacaatggcactgcttatctccgaaatggtgtgatcgtctcctcattgagcagcggctgccaccgcgctgtgggta

SEQ ID NO: 40 - DNA, 137 bp, Artificial Sequence
  Description of Artificial Sequence Synthetic polynucleotide
  modified_base (1)..(20): a, c, t, g, unknown or other
nnnnnnnnnnnnnnnnnnnngtttttgtactctcaagatttagaaataaatcttgcagaagctacaaagataaggcttcatgccgaaatcaacaccctgtcattttatggcagggtgttttcgttatttaatttttt

SEQ ID NO: 41 - DNA, 123 bp, Artificial Sequence
  Description of Artificial Sequence Synthetic polynucleotide
  modified_base (1)..(20): a, c, t, g, unknown or other
nnnnnnnnnnnnnnnnnnnngtttttgtactctcagaaatgcagaagctacaaagataaggcttcatgccgaaatcaacaccctgtcattttatggcagggtgttttcgttatttaatttttt

SEQ ID NO: 42 - DNA, 110 bp, Artificial Sequence
  Description of Artificial Sequence Synthetic polynucleotide
  modified_base (1)..(20): a, c, t, g, unknown or other
nnnnnnnnnnnnnnnnnnnngtttttgtactctcagaaatgcagaagctacaaagataaggcttcatgccgaaatcaacaccctgtcattttatggcagggtgttttttt

SEQ ID NO: 43 - DNA, 102 bp, Artificial Sequence
  Description of Artificial Sequence Synthetic polynucleotide
  modified_base (1)..(20): a, c, t, g, unknown or other
nnnnnnnnnnnnnnnnnnnngttttagagctagaaatagcaagttaaaataaggctagtccgttatcaacttgaaaaagtggcaccgagtcggtgctttttt

SEQ ID NO: 44 - DNA, 88 bp, Artificial Sequence
  Description of Artificial Sequence Synthetic oligonucleotide
  modified_base (1)..(20): a, c, t, g, unknown or other
nnnnnnnnnnnnnnnnnnnngttttagagctagaaatagcaagttaaaataaggctagtccgttatcaacttgaaaaagtgttttttt

SEQ ID NO: 45 - DNA, 76 bp, Artificial Sequence
  Description of Artificial Sequence Synthetic oligonucleotide
  modified_base (1)..(20): a, c, t, g, unknown or other
nnnnnnnnnnnnnnnnnnnngttttagagctagaaatagcaagttaaaataaggctagtccgttatcatttttttt

SEQ ID NO: 46 - DNA, 1335 bp, Homo sapiens
ggcttgctgaaaataaaatcaggactcctaacctgctccagtcagcctgcttccacgaggcctgtcagtcagtgccccacttgtgactgagtgtgcagtgcccagcatgtaccaggtcagtgcagagggctgcctgagggctgtgctgagagggagaggagcagagatgctgctgagggtggagggaggccaagctgccaggtttggggctgggggccaagtggagtgagaaactgggatcccagggggagggtgcagatgagggagcgacccagattaggtgaggacagttctctcattagccttttcctacaggtggttgcattcttggcaatggtcatgggaacccacacctacagccactggcccagctgctgccccagcaaagggcaggacacctctgaggagctgctgaggtggagcactgtgcctgtgcctcccctagagcctgctaggcccaaccgccacccagagtcctgtagggccagtgaagatggacccctcaacagcagggccatctccccctggagatatgagttggacagagacttgaaccggctcccccaggacctgtaccacgcccgttgcctgtgcccgcactgcgtcagcctacagacaggctcccacatggacccccggggcaactcggagctgctctaccacaaccagactgtcttctaccggcggccatgccatggcgagaagggcacccacaagggctactgcctggagcgcaggctgtaccgtgtttccttagcttgtgtgtgtgtgcggccccgtgtgatgggctagccggacctgctggaggctggtccctttttgggaaacctggagccaggtgtacaaccacttgccatgaagggccaggatgcccagatgcttggcccctgtgaagtgctgtctggagcagcaggatcccgggacaggatggggggctttggggaaagcctgcacttctgcacattttgaaaagagcagctgctgcttagggccgccggaagctggtgtcctgtcattttctctcaggaaaggttttcaaagttctgcccatttctggaggccaccactcctgtctcttcctcttttcccatcccctgctaccctggcccagcacaggcactttctagatatttcccccttgctggagaagaaagagcccctggttttatttgtttgtttactcatcactcagtgagcatctactttgggtgcattctagtgtagttactagtcttttgacatggatgattctgaggaggaagctgttattgaatgtatagagatttatccaaataaatatctttatttaaaaatgaaaaaaaaaaaaaaaaaaaa

SEQ ID NO: 47 - DNA, 2726 bp, Homo sapiens
acagcctccttctgcttaggaacaccagacagcactccagcactctgcttggggggcattcgaaacagcaaaatcactcataaaaggcaaaaaattgcaaaaaaaaatagtaataaccagcatggcactaaatagaccatgaaaagacatgtgtgtgcagtatgaaaattgagacaggaaggcagagtgtcagcttgttccacctcagctgggaatgtgcatcaggcaactcaagtttttcaccacggcatgtgtctgtgaatgtccgcaaaacattctctctccccagccttcatgtgttaacctggggatgatgtggacctgggcactgtggatgctcccctcactctgcaaattcagcctggcagctctgccagctaagcctgagaacatttcctgtgtctactactataggaaaaatttaacctgcacttggagtccaggaaaggaaaccagttatacccagtacacagttaagagaacttacgcttttggagaaaaacatgataattgtacaaccaatagttctacaagtgaaaatcgtgcttcgtgctcttttttccttccaagaataacgatcccagataattataccattgaggtggaagctgaaaatggagatggtgtaattaaatctcatatgacatactggagattagagaacatagcgaaaactgaaccacctaagattttccgtgtgaaaccagttttgggcatcaaacgaatgattcaaattgaatggataaagcctgagttggcgcctgtttcatctgatttaaaatacacacttcgattcaggacagtcaacagtaccagctggatggaagtcaacttcgctaagaaccgtaaggataaaaaccaaacgtacaacctcacggggctgcagccttttacagaatatgtcatagctctgcgatgtgcggtcaaggagtcaaagttctggagtgactggagccaagaaaaaatgggaatgactgaggaagaagctccatgtggcctggaactgtggagagtcctgaaaccagctgaggcggatggaagaaggccagtgcggttgttatggaagaaggcaagaggagccccagtcctagagaaaacacttggctacaacatatggtactatccagaaagcaacactaacctcacagaaacaatgaacactactaaccagcagcttgaactgcatctgggaggcgagagcttttgggtgtctatgatttcttataattctcttgggaagtctccagtggccaccctgaggattccagctattcaagaaaaatcatttcagtgcattgaggtcatgcaggcctgcgttgctgaggaccagctagtggtgaagtggcaaagctctgctctagacgtgaacacttggatgattgaatggtttccggatgtggactcagagcccaccaccctttcctgggaatctgtgtctcaggccacgaactggacgatccagcaagataaattaaaacctttctggtgctataacatctctgtgtatccaatgttgcatgacaaagttggcgagccatattccatccaggcttatgccaaagaaggcgttccatcagaaggtcctgagaccaaggtggagaacattggcgtgaagacggtcacgatcacatggaaagagattcccaagagtgagagaaagggtatcatctgcaactacaccatcttttaccaagctgaaggtggaaaaggattctccaagacagtcaattccagcatcttgcagtacggcctggagtccctgaaacgaaagacctcttacattgttcaggtcatggccagcaccagtgctgggggaaccaacgggaccagcataaatttcaagacattgtcattcagtgtctttgagattatcctcataacttctctgattggtggaggccttcttattctcattatcctgacagtggcatatggtctcaaaaaacccaacaaattgactcatctgtgttggcccaccgttcccaaccctgctgaaagtagtatagccacatggcatggagatgatttcaaggataagctaaacctgaaggagtctgatgactctgtgaacacagaagacaggatcttaaaaccatgttccacccccagtgacaagttggtgattgacaagttggtggtgaactttgggaatgttctgcaagaaattttcacagatgaagccagaacgggtcaggaaaacaatttaggaggggaaaagaatgggtatgtgacctgccccttcaggcctgattgtcccctggggaaaagttttgaggagctcccagtttcacctgagattccgcccagaaaatcccaatacctacgttcgaggatgccagaggggacccgcccagaagccaaagagcagcttctcttttctggtcaaagtttagtaccagatcatctgtgtgaggaaggagccccaaatccatatttgaaaaattcagtgacagccagggaatttcttgtgtctgaaaaacttccagagcacaccaagggagaagtctaaatgcgaccatagcatgagaccctcggggcctcagtgtggatggcccttgccagagaagatgtcaagactcggcacgcagcgcttgcttggccctgccacatcctgcctaggttaaagtttcccctgccccttgagctgccagttgaacttggtcggcaaagatgcgaccttgtactgggaagaagggatggtgataagcccgagttttgtaaaggaa

SEQ ID NO: 48 - DNA, 2679 bp, Homo sapiens
gtgttcgctgctgcacagcaaggccctgccacccaccttcaggccatgcagccatgttccgggagccctaattgcacagaagcccatggggagctccagactggcagccctgctcctgcctctcctcctcatagtcatcgacctctctgactctgctgggattggctttcgccacctgccccactggaacacccgctgtcctctggcctcccacacggatgacagtttcactggaagttctgcctatatcccttgccgcacctggtgggccctcttctccacaaagccttggtgtgtgcgagtctggcactgttcccgctgtttgtgccagcatctgctgtcaggtggctcaggtcttcaacggggcctcttccacctcctggtgcagaaatccaaaaagtcttccacattcaagttctataggagacacaagatgccagcacctgctcagaggaagctgctgcctcgtcgtcacctgtctgagaagagccatcacatttccatcccctccccagacatctcccacaagggacttcgctctaaaaggacccaaccttcggatccagagacatgggaaagtcttcccagattggactcacaaaggcatggaggacccgagttctcctttgatttgctgcctgaggcccgggctattcgggtgaccatatcttcaggccctgaggtcagcgtgcgtctttgtcaccagtgggcactggagtgtgaagagctgagcagtccctatgatgtccagaaaattgtgtctgggggccacactgtagagctgccttatgaattccttctgccctgtctgtgcatagaggcatcctacctgcaagaggacactgtgaggcgcaaaaaatgtcccttccagagctggccagaagcctatggctcggacttctggaagtcagtgcacttcactgactacagccagcacactcagatggtcatggccctgacactccgctgcccactgaagctggaagctgccctctgccagaggcacgactggcataccctttgcaaagacctcccgaatgccacagctcgagagtcagatgggtggtatgttttggagaaggtggacctgcacccccagctctgcttcaagttctcttttggaaacagcagccatgttgaatgcccccaccagactgggtctctcacatcctggaatgtaagcatggatacccaagcccagcagctgattcttcacttctcctcaagaatgcatgccaccttcagtgctgcctggagcctcccaggcttggggcaggacactttggtgccccccgtgtacactgtcagccaggcccggggctcaagcccagtgtcactagacctcatcattcccttcctgaggccagggtgctgtgtcctggtgtggcggtcagatgtccagtttgcctggaagcacctcttgtgtccggatgtctcttacagacacctggggctcttgatcctggcactgctggccctcctcaccctactgggtgttgttctggccctcacctgccggcgcccacagtcaggcccgggcccagcgcggccagtgctcctcctgcacgcggcggactcggaggcgcagcggcgcctggtgggagcgctggctgaactgctacgggcagcgctgggcggcgggcgcgacgtgatcgtggacctgtgggaggggaggcacgtggcgcgcgtgggcccgctgccgtggctctgggcggcgcggacgcgcgtagcgcgggagcagggcactgtgctgctgctgtggagcggcgccgaccttcgcccggtcagcggccccgacccccgcgccgcgcccctgctcgccctgctccacgctgccccgcgcccgctgctgctgctcgcttacttcagtcgcctctgcgccaagggcgacatccccccgccgctgcgcgccctgccgcgctaccgcctgctgcgcgacctgccgcgtctgctgcgggcgctggacgcgcggcctttcgcagaggccaccagctggggccgccttggggcgcggcagcgcaggcagagccgcctagagctgtgcagccggctcgaacgagaggccgcccgacttgcagacctaggttgagcagagctccaccgcagtcccgggtgtctgcggccgcaacgcaacggacactggctggaaccccggaatgagccttcgaccctgaaatccttggggtgcctcgaggacgactggccgaaaagccgcattccctgcctcacaggccggaagtcccagcccagtccccgcgcgcgtccctcttcctcctcatactttcccttgactgagagctcctctaacccctgttctgatgggggagggcggtcttcccacttcctctccagaactccagaaagagcagtgtgcttatgcttcagtccaggctggagaggttggggccggggtagggaggcaggagccatgtcagttctgaaggagggtgaggcggtgggggattgcagggggcggctgagagaaaacctccttgggggccagggattccctttcccactctgaggctctggccagagggagagaggactctggacctaggaaaagaggcttttggctccaggtggtcaggacagtgggggttgggggtggggtgggtgggtgctggcggtggggaccaagatccggaaagatgaataaagacaaacatgacaaactaa

SEQ ID NO: 49 - DNA, 3661 bp, Homo sapiens
tcaggaggcggagatcgctgcttctcacctactttctgaacttggcctccgcagtcgcgacctggcgtgaaggaggagctgccgcccccgccccagcctcggggacgcctctctgaagagaagccatttgaagcagaatccaaaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttcctcgagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttaatcttcagaaacagtgaacgccaaaaagaactcaagatattctggggactgagcatatgaacctgttcataacaaaggctgtgactcgaaataattaactttgtcaaaatcctgctcacaatttgaagatgaaacttgtcattaggttggcgggaatgagactaaagattgcgctgtgggctgtggtcacgtgctcccagaagacctggaattcaaaagaaatggagctattctttttctccctctttcataactggatgcagctgctcatactcaatcccatattcagcaagtgtgaagctggacgtgatgcaaaataaccgatgccctacaaaaagggcgcatctttaagagttttaatgccagtgcttaattcgaatgaggggattttaagtgtctgaagaggcattttctagggaccagtgggtgactgagtaactgaaatgctgctttcactccctaacaccatggatctggttgtgcataggatgtgggaggaggggctggcagggccgccttcagaggctgcagggcctcagcctcaggatgcatttaatgtatcctggccacagttgcagccaacggttcttgaaagctcggtaaggccctgcaacgcagagcctgcttatgtggatctatttatgggaacttcttaaaaggaccccagaatagctctttatctttcacaagagacacaaattctaattgagttaattatctgggcctttcactttggatgctctgaaacatttgttgattttgtgtgaatgtttatatcaaaatgtttgccaggttgtattagccattgaatagcaaaaaactgatagttacttgcttgttttttaaaaattacatattaaaaatgcccttggcataaggcagcatggtgtggcagttaagagatgggctgtgcagcccatcctgagctccagtcctgagtttgctacttacttctgtggcctctggaaccttatccaacctcttggtgcttcagtttcctcatctgtgaaattagaatttataataattgcacctacctcccaggggtaactaaatgaataaatataataaagtacttacagtggttcctgacacagactcagcactccgtcagtgttgccatgactatttttattatcattattaatgattacttagatcaattatttagcagtggactaatggaagctacagagcagggaagggaagcagatctagggaggaaggcagttttgatttgaggaggtttgcacatgtagagaagcatactggagaagcatatccagagggcgaaagatatctctccattgtgcatctgcctcttttgacgttggaagacacatgtcttactccccaaagggagcccagcactgggagccttcttgatgatctcaaaaataatagctattcaagaaaatcaccaagtgactgtgaaaccgtcagttcggaaggctggttagaacatgtgggagcaacatgaatgttctacaaaagtttaaagcagagattgtttcaaatgggtgtagtagatattactgaaaaccaaaaaagagtgagattgtcagtgtaagaatgtgatttaatgtttgtagtgcttacaattttgtgtaccaactggatgactaaaaagagtaaaataatttaattaatagctcatattttatgtgtgaaaacatgttagtgaacatatataatcaaaatagatttcattgctattgcatagtctctaatacatagaatgattttgcttttctcttttattatacttgctttaaaatacttgaaatatattttgcattaaatgcatttcaagttaaatgtcttaaatgtatacattagatgtgtgttttaaaatgcataaaacacgttgaaatacattaatgaaccatt

SEQ ID NO: 50 - DNA, 2672 bp, Homo sapiens
gtatctctctggataggaagaaatatagtagaaccctttgaaaatggatattttcacatattttcgttcagatacaaaagctggcagttactgaaataaggacttgaagttccttcctcttttttttatgtcttaagagcaggaaataaagagacagctgaaggtgtagccttgaccaactgaaagggaaatcttcatcctctgaaaaaacatatgtgattctcaaaaaacgcatctggaaaattgataaagaagcgattctgtagattctcccagcgctgttgggctctcaattccttctgtgaaggacaacatatggtgatggggaaatcagaagctttgagaccctctacacctggatatgaatcccccttctaatacttaccagaaatgaaggggatactcagggcagagttctgaatctcaaaacactctactctggcaaaggaatgaagttattggagtgatgacaggaacacgggagaacaatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtacaagaatggaaaactcctctctgtggaaaggagcaaccgaatcgtagtggatgaagtttatgactatcaccagggcacatatgtatgtgattacactcagtcggatactgtgagttcgtggacagtcagagctgttgttcaagtgagaaccattgtgggagacactaaactcaaaccagatattctggatcctgtcgaggacacactggaagtagaacttggaaagcctttaactattagctgcaaagcacgatttggctttgaaagggtctttaaccctgtcataaaatggtacatcaaagattctgacctagagtgggaagtctcagtacctgaggcgaaaagtattaaatccactttaaaggatgaaatcattgagcgtaatatcatcttggaaaaagtcactcagcgtgatcttcgcaggaagtttgtttgctttgtccagaactccattggaaacacaacccagtccgtccaactgaaagaaaagagaggagtggtgctcctgtacatcctgcttggcaccatcgggaccctggtggccgtgctggcggcgagtgccctcctctacaggcactggattgaaatagtgctgctgtaccggacctaccagagcaaggatcagacgcttggggataaaaaggattttgatgctttcgtatcctatgcaaaatggagctcttttccaagtgaggccacttcatctctgagtgaagaacacttggccctgagcctatttcctgatgttttagaaaacaaatatggatatagcctgtgtttgcttgaaagagatgtggctccaggaggagtgtatgcagaagacattgtgagcattattaagagaagcagaagaggaatatttatcttgagccccaactatgtcaatggacccagtatctttgaactacaagcagcagtgaatcttgccttggatgatcaaacactgaaactcattttaattaagttctgttacttccaagagccagagtctctacctcatctcgtgaaaaaagctctcagggttttgcccacagttacttggagaggcttaaaatcagttcctcccaattctaggttctgggccaaaatgcgctaccacatgcctgtgaaaaactctcagggattcacgtggaaccagctcagaattacctctaggatttttcagtggaaaggactcagtagaacagaaaccactgggaggagctcccagcctaaggaatggtgaaatgagccctggagccccctccagtccagtccctgggatagagatgttgctggacagaactcacagctctgtgtgtgtgtgttcaggctgataggaaattcaaagagtctcctgccagcaccaagcaagcttgatggacaatggagtgggattgagactgtggtttagagcctttgatttcctggactggactgacggcgagtgaattctctagaccttgggtactttcagtacacaacacccctaagatttcccagtggtccgagcagaatcagaaaatacagctacttctgccttatggctagggaactgtcatgtctaccatgtattgtacatatgactttatgtatacttgcaatcaaataaatattattttattagaaa

SEQ ID NO: 51 - DNA, 2038 bp, Homo sapiens
gctgtgcacccagagatagttgggtgacaaatcacctccaggttggggatgcctcagacttgtgatgggactgggcagatgcatctgggaaggctggaccttggagagtgaggccctgaggcgagacatgggcacctggctcctggcctgcatctgcatctgcacctgtgtctgcttgggagtctctgtcacaggggaaggacaagggccaaggtctagaaccttcacctgcctcaccaacaacattctcaggatcgattgccactggtctgccccagagctgggacagggctccagcccctggctcctcttcaccagcaaccaggctcctggcggcacacataagtgcatcttgcggggcagtgagtgcaccgtcgtgctgccacctgaggcagtgctcgtgccatctgacaatttcaccatcactttccaccactgcatgtctgggagggagcaggtcagcctggtggacccggagtacctgccccggagacacgttaagctggacccgccctctgacttgcagagcaacatcagttctggccactgcatcctgacctggagcatcagtcctgccttggagccaatgaccacacttctcagctatgagctggccttcaagaagcaggaagaggcctgggagcaggcccagcacagggatcacattgtcggggtgacctggcttatacttgaagcctttgagctggaccctggctttatccatgaggccaggctgcgtgtccagatggccacactggaggatgatgtggtagaggaggagcgttatacaggccagtggagtgagtggagccagcctgtgtgcttccaggctccccagagacaaggccctctgatcccaccctgggggtggccaggcaacacccttgttgctgtgtccatctttctcctgctgactggcccgacctacctcctgttcaagctgtcgcccagggtgaagagaatcttctaccagaacgtgccctctccagcgatgttcttccagcccctctacagtgtacacaatgggaacttccagacttggatgggggcccacggggccggtgtgctgttgagccaggactgtgctggcaccccacagggagccttggagccctgcgtccaggaggccactgcactgctcacttgtggcccagcgcgtccttggaaatctgtggccctggaggaggaacaggagggccctgggaccaggctcccggggaacctgagctcagaggatgtgctgccagcagggtgtacggagtggagggtacagacgcttgcctatctgccacaggaggactgggcccccacgtccctgactaggccggctcccccagactcagagggcagcaggagcagcagcagcagcagcagcagcaacaacaacaactactgtgccttgggctgctatgggggatggcacctctcagccctcccaggaaacacacagagctctgggcccatcccagccctggcctgtggcctttcttgtgaccatcagggcctggagacccagcaaggagttgcctgggtgctggctggtcactgccagaggcctgggctgcatgaggacctccagggcatgttgctcccttctgtcctcagcaaggctcggtcctggacattctaggtccctgactcgccagatgcatcatgtccattttgggaaaatggactgaagtttctggagcccttgtctgagactgaacctcctgagaaggggcccctagcagcggtcagaggtcctgtctggatggaggctggaggctcccccctcaacccctctgctcagtgcctgtggggagcagcctctaccctcagcatcctggccacaagttcttccttccattgtcccttttctttatccctgacctctctgagaagtggggtgtggtctctcagctgttctgccctcatacccttaaagggccagcctgggcccagtggacacaggtaaggcaccatgaccacctggtgtgacctctctgtgccttactgaggcacctttctagagattaaaaggggcttgatggctgtt

SEQ ID NO: 52 - protein, 6 aa, Artificial Sequence
  Description of Artificial Sequence Synthetic peptide
  MOD_RES (2)..(2): Phosphotyrosine
PYLKTK

SEQ ID NO: 53 - protein, 6 aa, Artificial Sequence
  Description of Artificial Sequence Synthetic peptide
  MOD_RES (1)..(1): Phosphotyrosine
YLPQTV